SEQUENCE LISTING (73 sequences)

SEQ ID NO: 1 - DNA, 1731 bp, Bordetella pertussis
atggattccaacctaatgtcttccgtttccacctccgccatccttggcggcgaaaacttcgccgacctgttcgcagaaagcctcaagagccaggacatgaagtccggcgaggtcatcagcgcagaagtcgtgcgcgtcgaccacaacttcgtggtcgtcaacgccggcctgaagtccgaagcgctgattcccctggaagagttcctcaacgaccagggcgaactcgaagttcaacccggcgacttcgtctcggtggcgatcgattcgctggagaacggctacggcgacaccatcctgtcgcgcgaccgcgccaagcgtctgtcggcctggctgcaactggagcaggccctcgagaacggcgagctggtcaccggcacgatcaccggcaaggtcaagggcggcctgaccgtcatgaccaacggcatccgcgcgttcctgcccggttcgctggtcgacctgcgtccggtcaaggacaccacgccgtacgaaggcaagaccctcgaattcaaggtcatcaagctggaccgcaagcgcaacaacgtcgtgctgtcgcgccgccaggtgctggaagccagcatgggcgaagagcgccagaagctgctcgagacgctgcacgaaggcgcggtggtcaagggcgtggtcaagaacatcaccgactacggcgcgttcgtcgacctgggcggcatcgatggcctgctgcacatcaccgacatggcctggcgccgtgtgcgtcacccgtccgaagtcctgcaagtgggtcaggaagtcgaagccaaggtgctcaagttcgaccaggaaaagagccgcgtctccctgggcgtcaagcagctgggcgaagatccgtgggtgggcctggctcgccgctacccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaagcagtgccgccagaacccgtgggaagagttcgccaccaacttcaagcgtggtgacaaggtccgcggcgccatcaagtcgatcaccgacttcggcgtgttcgtcggcctgcccggcggcatcgacggcctggtccatctgtccgacctgtcgtggacggaatcgggcgaggaagccgtgcgcaacttcaagaagggcgacgagctggaagccgtggtgctgggcatcgataccgagaaagagcgcatctcgctgggtatcaagcagctcgaaggcgacccgttcaacaacttcgttgccacgcacgacaagggcgccgttgttccgggcaccatcaagtcggtcgagcccaagggcgccgtgatcaccctgtcggtggatgtggaaggctacctgcgcgcctccgagatctcctcgggccgcgtcgaagacgctaccaccgtgctgaaggctggcgagaacatcgaagccatgatcgtcaacatcgaccgcaaggcgcgttcgatccagctgtcgatcaaggcccgcgataacgccgagacggccgagaccatccagcgcatgtccgaggcgagcgcttcgtcgggtacgacgaacttgggcgcgctgctcaaggccaagctggaccaacagcgcaacgacggttga

SEQ ID NO: 2 - DNA, 1674 bp, Escherichia coli
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggattggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcctggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtaa

SEQ ID NO: 3 - DNA, 1650 bp, Haemophilus influenzae
  misc_feature (338)..(338): n is a, c, g, or t
  misc_feature (576)..(576): n is a, c, g, or t
atgtcagaatcttttgctcaactttttgaagaatcattaaaagtccttgaaactcgtcaaggttcaatcgttagcggtactgtagtagctatccaaaaaggctttgtgcttgttgatgcaggtttaaaatctgagtctgcaattccagttgctgaattcttaaatgcacaaggcgaacttgaaatccaagttggcgatactgtaaatgttgcattagatgcagttgaagatggtttcggtgaaactaaactttctcgtgagaaagcggttcgtcacgaatcttggattgaattagaaaaagcttacgaagaaaaagcgaccgttatcggtttaatcancggcaaagtgaaaggtggcttcacagttgagttaaacggtgttcgtgcattcttaccaggttctttagttgatactcgtccagcgcgtgaagcagatcacttacttggtaaagaattagaattcaaagtaatcaaattagatcaaaaacgtaacaacgttgttgtttctcgtcgtgcagtaattgaatctgaaaacagccaagaacgtgaacaagtattagaaaatcttgtggaaggntcagaagttaaaggtgtcgttaaaaacttaactgagtacggtgcattcgttgatcttggtggtgttgatggtttattacacatcacagatatggcttggaaacgtgttaaacacccaagtgaaatcgtaaatgttggcgatgaagttactgttaaagtattaaaattcgataaagatcgtactcgtgtatctttaggcttaaaacaattaggtcaagatccatgggcagcaattgctgaaaatcatccagtaaacagcaaattaacaggtaaagtaactaacttaactgactatggctgtttcgttgaaatcttagatggcgttgaaggtttagttcacgtttctgaaatggattggactaataaaaatatccacccatctaaagtggttagcttaggcgatactgtagaagtaatggtattagaaattgatgaagaacgtcgtcgtatttctttaggcttaaaacaatgtaaagctaacccttggactcagtttgctgatactcacaacaaaggcgataaagtaacgggtaaaatcaagtctatcactgatttcggtatcttcatcggtcttgaaggtggtatcgatggtttagttcacttatctgatatttcttggagtatttcaggcgaagaagctgttcgtcaatacaaaaaaggtgacgaagtttcagctgttgtattagctgttgatgcagtaaaagaacgtatctctttaggtattaaacaacttgaagaagatccattcaataactttgtcgcaatcaacaaaaaaggtgcggtagtatctgcaactgttgttgaagcagatgctaaaggtgctaaagttgaattagcaggtggtgttgaaggttatatccgttctgctgatttaacaagtgaagtagctgttggtgatgtcgttgaagcgaaatacactggagtagatcgtaaatctcgcattgttcacttatcagtgaaagcaaaagatcaagctgaagaagctgcggcagttgcaagtgtaaataacaaacaagaagatattgttattccaaatgcaatggctgaagcatttaaagcagctaaaggtgaataa

SEQ ID NO: 4 - DNA, 1686 bp, Neisseria gonorrhoeae
atgtctatggaaaattttgctcagctgttggaagaaagctttaccctgcaagaaatgaacccgggtgaggtgattaccgctgaagtagtggcaatcgaccaaaacttcgttaccgtaaacgcaggtctgaaatcagaatccctgatcgatgtagctgaattcaaaaacgctcaaggcgaaattgaagttaaagtcggcgacttcgttaccgttaccatcgaatccgtcgaaaacggcttcggcgaaaccaaactgtcccgcgaaaaagccaaacgcgcagccgattggatcgctttggaagaagccatggaaaacggcaacatcctgtccggcatcatcaacggtaaagtcaaaggcggcctgaccgttatgatcagcagcatccgcgcattcctgccgggttctttggtcgacgtacgtcccgttaaagacacttcccattttgaaggcaaagagatcgaattcaaagtgatcaaactggacaaaaaacgcaacaacgtcgttgtttcccgccgcgccgttttggaagccactttgggtgaagaacgcaaagccctgctggaaaacctgcaagaaggctccgtcatcaaaggcatcgtcaaaaatatcaccgactacggcgcattcgttgacctgggcggcatcgacggcctgctgcacatcaccgatttggcatggcgtcgcgtgaaacacccgagcgaagtcttggaagtcggtcaggaagttgaagccaaagtattgaaattcgaccaagaaaaacaacgtgtttccttgggtatgaaacaactgggcgaagatccttggagcggtctgacccgccgttatccgcaagccacccgcctgttcggcaaagtatccaacctgaccgactacggcgcattcgtcgaaatcgaacaaggcatcgaaggtttggtacacgtctccgaaatggactggaccaacaaaaacgtacacccgagcaaagtcgtacaactgggtgacgaagtcgaagtcatgattttggaaatcgacgaaggccgccgccgtatctctttgggtatgaaacaatgccaagccaatccttgggaagaatttgccgccaaccacaacaaaggagacaaaatctccggtgcggttaaatccattaccgatttcggcgtattcgtcggcctgcccggcggcatcgacggtctggttcacctgtccgacctgtcttggaccgaatccggcgaagaagccgtacgcaaatacaaaaaaggagaagaagtcgaagccgtcgtattggcaatcgatgtggaaaaagaacgcatctccttgggtatcaaacaactggaaggcgatcctttcggcaacttcatcagcgtgaacgacaaaggttctttggttaaaggttccgtgaaatctgttgacgccaaaggcgctgttatcgccctgtctgacgaagtagaaggctacctgcctgcttccgaatttgcagccgaccgcgttgaagacttgaccaccaaactgaaagaaggcgacgaagttgaagccgtcatcgttaccgttgaccgcaaaaaccgcagcatcaaactttccgttaaagccaaagatgccaaagaaagccgcgaagcactgaactccgtcaatgccgccgccaatgcgaatgccggtaccaccagcttgggcgacctgctgaaagccaaactctccggcgaacaagaataa

SEQ ID NO: 5 - DNA, 1686 bp, Neisseria meningitidis
atgtctatggaaaattttgctcagctgttggaagaaagctttaccctgcaagaaatgaacccgggtgaggtgattaccgctgaagtagtggcaatcgaccaaaacttcgttaccgtaaacgcaggtctgaaatcagaatccctgattgatgtagctgaattcaaaaacgctcaaggcgaaattgaagttaaagtcggcgacttcgttaccgttaccatcgaatccgtcgaaaacggcttcggcgaaaccaaactgtcccgcgaaaaagccaaacgtgcagccgattggattgccctggaagaagccatggaaaacggcgacatcctgtccggcatcatcaacggaaaagtcaaaggcggcctgaccgttatgattagcagcatccgcgcattcctgccgggttctttggtcgacgtacgtcctgtaaaagacacttctcacttcgaaggcaaagagatcgaattcaaagtgatcaaactggacaaaaaacgcaacaacgtcgttgtttcccgccgcgccgttctggaagccactttgggtgaagaacgcaaagccctgctggaaaacctgcaagaaggctccgtcatcaaaggcatcgttaaaaacattaccgattacggtgcattcgttgacttgggcggcatcgacggtctgttgcacatcaccgatttggcatggcggcgcgtgaaacacccgagtgaagtcttggaagtcggtcaggaagttgaagccaaagtattgaaattcgaccaagaaaaacaacgcgtttccttgggtatgaaacaactgggcgaagatccttggagcggtctgacccgccgttatcctcaaggcacccgcctgttcggcaaagtatccaacctgaccgactacggcgcattcgtcgaaatcgaacaaggcatcgaaggtttggtacacgtctccgaaatggactggaccaacaaaaacgtacacccgagcaaagtcgtacaactgggcgacgaagtcgaagtcatgattttggaaatcgacgaaggccgccgccgtatctctttgggtatgaaacaatgccaagccaatccttgggaagaatttgccgccaaccacaacaaaggcgacaaaatctccggcgcggttaaatccattaccgatttcggcgtattcgtcggcctgcccggcggcatcgacggtttggttcacctgtccgacctgtcctggaccgaatccggcgaagaagccgtacgcaaatacaaaaaaggcgaagaagtcgaagccgtcgtattggcaatcgacgtggaaaaagaacgcatctccttgggtatcaaacaactggaaggcgatccgttcggcaacttcatcagcgtgaacgacaaaggttctttggttaaaggttccgtgaaatctgttgacgccaaaggtgctgttatcgccctgtctgacgaagtagaaggctacctgcctgcttccgaatttgcagccgaccgcgttgaagatttgaccaccaaactgaaagaaggcgacgaagttgaagccgtcatcgttaccgttgaccgcaaaaaccgcagcatcaaactttccgttaaagccaaagatgccaaagaaagccgcgaagcactgaactccgtcaatgccgccgccaatgcgaatgccggcaccaccagcttgggcgacctgctgaaagccaaactctccggcgaacaagaataa

SEQ ID NO: 6 - DNA, 1680 bp, Pseudomonas aeruginosa
atgagcgaaagcttcgcagaactctttgaagaaagtctgaaatccctcgacatgcagccgggtgccatcatcaccggcatcgtggtcgacatcgatggtgactgggtcaccgtccatgccggtctgaaatccgagggcgtcatcccggtcgagcagttctacaacgaacagggcgagctgaccatcaaggtgggtgacgaagtccacgtcgcactggacgcggtagaagacggctttggcgagaccaagctgtcccgcgagaaagccaagcgcgccgagagctggattgttctggaagcggctttcgctgccgacgaagtggtcaagggcgtcatcaacggcaaggtcaagggcggtttcaccgtcgacgtcaacggcatccgcgcgttcctgccgggttctctggtcgacgttcgtccggttcgcgacaccacccacctggaaggcaaagagctcgagttcaaggtcatcaagctcgaccagaagcgcaacaacgttgtcgtttcccgccgcagcgtcctggaagccgagaacagcgccgagcgtgaagctctgctggaatcgctgcaggaaggccagcaggtcaaaggtatcgtcaagaacctcaccgactacggcgcattcgtggacctgggcggcgtagacggcctgctacacatcaccgacatggcctggaagcgcatcaagcatccgtccgagatcgtcaacgttggcgacgagatcgacgtcaaggtcctgaagttcgaccgcgagcgcaaccgtgtatccctgggcctgaagcaactgggcgaagacccgtgggttgccatcaaggcgcgttacccggaaggtacccgcgtcatggcccgcgtcaccaacctcaccgactacggctgcttcgccgagctggaagagggcgtggaaggcctggtacacgtctccgaaatggactggaccaacaagaacatccatccgtcgaaagtcgtccaggttggcgatgaagtggaagttcaggttctggacatcgacgaagagcgtcgtcgtatctccctgggtatcaagcagtgcaaatccaacccgtgggaagacttctccagccagttcaacaagggtgaccgtatctccggtaccatcaagtcgatcaccgacttcggtatcttcatcggtctggacggcggcatcgacggcctggtccacctgtccgacatctcctggaacgaagtcggcgaagaagccgtacgtcgcttcaagaagggcgacgagctggaaaccgtcatcctgtcggtcgatccggagcgcgagcgcatctccctgggcatcaagcagctggaagacgatccgttctccaactacgcgtccctgcacgagaaaggcagcatcgtccgcggtaccgtgaaggaagtcgacgccaagggcgctgtcatcagcctgggcgacgacatcgaaggtatcctgaaggcttccgaaatcagccgtgaccgcgtcgaagacgcgcgcaacgtcctgaaggaaggcgaggaagtcgaagccaagatcatcagcatcgaccgcaagagccgcgtcatcagcctctccgtcaagtccaaggacgtcgacgacgagaaggacgcaatgaaagaactgcgtaagcaggaagtagaaagcgctggtccgaccaccatcggtgatctgatccgtgctcagatggagaatcagggctaa

SEQ ID NO: 7 - DNA, 2092 bp, Bordetella pertussis
atggattccaacctaatgtcttccgtttccacctccgccatccttggcggcgaaaacttcgccgacctgttcgcagaaagcctcaagagccaggacatgaagtccggcgaggtcatcagcgcagaagtcgtgcgcgtcgaccacaacttcgtggtcgtcaacgccggcctgaagtccgaagcgctgattcccctggaagagttcctcaacgaccagggcgaactcgaagttcaacccggcgacttcgtctcggtggcgatcgattcgctggagaacggctacggcgacaccatcctgtcgcgcgaccgcgccaagcgtctgtcggcctggctgcaactggagcaggccctcgagaacggcgagctggtcaccggcacgatcaccggcaaggtcaagggcggcctgaccgtcatgaccaacggcatccgcgcgttcctgcccggttcgctggtcgacctgcgtccggtcaaggacaccacgccgtacgaaggcaagaccctcgaattcaaggtcatcaagctggaccgcaagcgcaacaacgtcgtgctgtcgcgccgccaggtgctggaagccagcatgggcgaagagcgccagaagctgctcgagacgctgcacgaaggcgcggtggtcaagggcgtggtcaagaacatcaccgactacggcgcgttcgtcgacctgggcggcatcgatggcctgctgcacatcaccgacatggcctggcgccgtgtgcgtcacccgtccgaagtcctgcaagtgggtcaggaagtcgaagccaaggtgctcaagttcgaccaggaaaagagccgcgtctccctgggcgtcaagcagctgggcgaagatccgtgggtgggcctggctcgccgctacccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaagcagtgccgccagaacccgtgggaagagttcgccaccaacttcaagcgtggtgacaaggtccgcggcgccatcaagtcgatcaccgacttcggcgtgttcgtcggcctgcccggcggcatcgacggcctggtccatctgtccgacctgtcgtggacggaatcgggcgaggaagccgtgcgcaacttcaagaagggcgacgagctggaagccgtggtgctgggcatcgataccgagaaagagcgcatctcgctgggtatcaagcagctcgaaggcgacccgttcaacaacttcgttgccacgcacgacaagggcgccgttgttccgggcaccatcaagtcggtcgagcccaagggcgccgtgatcaccctgtcggtggatgtggaaggctacctgcgcgcctccgagatctcctcgggccgcgtcgaagacgctaccaccgtgctgaaggctggcgagaacatcgaagccatgatcgtcaacatcgaccgcaaggcgcgttcgatccagctgtcgatcaaggcccgcgataacgccgagacggccgagaccatccagcgcatgtccgaggcgagcgcttcgtcgggtacgacgaacttgggcgcgctgctcaaggccaagctggaccaacagcgcaacgacggttgacgtgaccaagtcggagctgatcgccgcgctggcggcccgctatcctcagctggccgctcgcgacaccgattacgctgtcaagaccatgctcgatgcaatgacccaggccctggcctcgggtcagcgcatcgaaatccgcgggtttggcagcttttcgctgtcgcagcgctctccccgtatcgggcgcaatccgaagtcgggcgaacaagtgctggtgcctggcaagcaggtgccgcacttcaaggccggcaaggagctgcgcgagtgggtcgatctggttggcaacgatcagggcgacgactcgtccaacgggtcgtccgacccgctgcaatcggtcatggatatgcatgccatgcactga

SEQ ID NO: 8 - DNA, 2118 bp, Escherichia coli
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggattggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcctggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtaattctctgactcttcgggatttttattccgaagtttgttgagtttacttgacagattgcaggtttcgtcctgtaatcaagcactaagggcggctacggccgcccttaatcaatgcagcaacagcagccgcttaatttgcctttaaggaaccggaggaatcatgaccaagtcagaattgatagaaagacttgccacccagcaatcgcacattcccgccaagacggttgaagatgcagtaaaagagatgctggagcatatggcctcgactcttgcgcagggcgagcgtattgaaatccgcggtttcggcagtttctctttgcactaccgcgcaccacgtaccggacgtaatccgaagactggcgataaagtagaactggaaggaaaatacgttcctcactttaaacctggtaaagaactgcgcgatcgcgccaatatttacggttaa

SEQ ID NO: 9 - DNA, 2011 bp, Neisseria meningitidis
atgtctatggaaaattttgctcagctgttggaagaaagctttaccctgcaagaaatgaacccgggtgaggtgattaccgctgaagtagtggcaatcgaccaaaacttcgttaccgtaaacgcaggtctgaaatcagaatccctgattgatgtagctgaattcaaaaacgctcaaggcgaaattgaagttaaagtcggcgacttcgttaccgttaccatcgaatccgtcgaaaacggcttcggcgaaaccaaactgtcccgcgaaaaagccaaacgtgcagccgattggattgccctggaagaagccatggaaaacggcgacatcctgtccggcatcatcaacggaaaagtcaaaggcggcctgaccgttatgattagcagcatccgcgcattcctgccgggttctttggtcgacgtacgtcctgtaaaagacacttctcacttcgaaggcaaagagatcgaattcaaagtgatcaaactggacaaaaaacgcaacaacgtcgttgtttcccgccgcgccgttctggaagccactttgggtgaagaacgcaaagccctgctggaaaacctgcaagaaggctccgtcatcaaaggcatcgttaaaaacattaccgattacggtgcattcgttgacttgggcggcatcgacggtctgttgcacatcaccgatttggcatggcggcgcgtgaaacacccgagtgaagtcttggaagtcggtcaggaagttgaagccaaagtattgaaattcgaccaagaaaaacaacgcgtttccttgggtatgaaacaactgggcgaagatccttggagcggtctgacccgccgttatcctcaaggcacccgcctgttcggcaaagtatccaacctgaccgactacggcgcattcgtcgaaatcgaacaaggcatcgaaggtttggtacacgtctccgaaatggactggaccaacaaaaacgtacacccgagcaaagtcgtacaactgggcgacgaagtcgaagtcatgattttggaaatcgacgaaggccgccgccgtatctctttgggtatgaaacaatgccaagccaatccttgggaagaatttgccgccaaccacaacaaaggcgacaaaatctccggcgcggttaaatccattaccgatttcggcgtattcgtcggcctgcccggcggcatcgacggtttggttcacctgtccgacctgtcctggaccgaatccggcgaagaagccgtacgcaaatacaaaaaaggcgaagaagtcgaagccgtcgtattggcaatcgacgtggaaaaagaacgcatctccttgggtatcaaacaactggaaggcgatccgttcggcaacttcatcagcgtgaacgacaaaggttctttggttaaaggttccgtgaaatctgttgacgccaaaggtgctgttatcgccctgtctgacgaagtagaaggctacctgcctgcttccgaatttgcagccgaccgcgttgaagatttgaccaccaaactgaaagaaggcgacgaagttgaagccgtcatcgttaccgttgaccgcaaaaaccgcagcatcaaactttccgttaaagccaaagatgccaaagaaagccgcgaagcactgaactccgtcaatgccgccgccaatgcgaatgccggcaccaccagcttgggcgacctgctgaaagccaaactctccggcgaacaagaataaggttgcagacatgacaaagtctgagttaatggttcgtttggcagaagtgtttgccgccaaaaacggcacgcatcttctggcaaaagacgtagagtacagcgtaaaagtcttggttgacaccatgactagatcgcttgcccgaggtcaacgcatcgaaatccgcggtttcggcagcttcgatttgaaccatcgtcctgcccgcatcggtcgcaatcccaaaaccggcgagcgtgtggaagtacctgaaaaacatgtaccccacttcaagcccggtaaagaattgcgcgagcgggtcgacttggctttaaaagaaaatgccaattaa

SEQ ID NO: 10 - DNA, 2011 bp, Neisseria gonorrhoeae
atgtctatggaaaattttgctcagctgttggaagaaagctttaccctgcaagaaatgaacccgggtgaggtgattaccgctgaagtagtggcaatcgaccaaaacttcgttaccgtaaacgcaggtctgaaatcagaatccctgatcgatgtagctgaattcaaaaacgctcaaggcgaaattgaagttaaagtcggcgacttcgttaccgttaccatcgaatccgtcgaaaacggcttcggcgaaaccaaactgtcccgcgaaaaagccaaacgcgcagccgattggatcgctttggaagaagccatggaaaacggcaacatcctgtccggcatcatcaacggtaaagtcaaaggcggcctgaccgttatgatcagcagcatccgcgcattcctgccgggttctttggtcgacgtacgtcccgttaaagacacttcccattttgaaggcaaagagatcgaattcaaagtgatcaaactggacaaaaaacgcaacaacgtcgttgtttcccgccgcgccgttttggaagccactttgggtgaagaacgcaaagccctgctggaaaacctgcaagaaggctccgtcatcaaaggcatcgtcaaaaatatcaccgactacggcgcattcgttgacctgggcggcatcgacggcctgctgcacatcaccgatttggcatggcgtcgcgtgaaacacccgagcgaagtcttggaagtcggtcaggaagttgaagccaaagtattgaaattcgaccaagaaaaacaacgtgtttccttgggtatgaaacaactgggcgaagatccttggagcggtctgacccgccgttatccgcaagccacccgcctgttcggcaaagtatccaacctgaccgactacggcgcattcgtcgaaatcgaacaaggcatcgaaggtttggtacacgtctccgaaatggactggaccaacaaaaacgtacacccgagcaaagtcgtacaactgggtgacgaagtcgaagtcatgattttggaaatcgacgaaggccgccgccgtatctctttgggtatgaaacaatgccaagccaatccttgggaagaatttgccgccaaccacaacaaaggagacaaaatctccggtgcggttaaatccattaccgatttcggcgtattcgtcggcctgcccggcggcatcgacggtctggttcacctgtccgacctgtcttggaccgaatccggcgaagaagccgtacgcaaatacaaaaaaggagaagaagtcgaagccgtcgtattggcaatcgatgtggaaaaagaacgcatctccttgggtatcaaacaactggaaggcgatcctttcggcaacttcatcagcgtgaacgacaaaggttctttggttaaaggttccgtgaaatctgttgacgccaaaggcgctgttatcgccctgtctgacgaagtagaaggctacctgcctgcttccgaatttgcagccgaccgcgttgaagacttgaccaccaaactgaaagaaggcgacgaagttgaagccgtcatcgttaccgttgaccgcaaaaaccgcagcatcaaactttccgttaaagccaaagatgccaaagaaagccgcgaagcactgaactccgtcaatgccgccgccaatgcgaatgccggtaccaccagcttgggcgacctgctgaaagccaaactctccggcgaacaagaataaggttgcagacatgacaaagtctgagttaatggttcgcttggcagaagtatttgccgccaaaaacggcacgcatcttctggcaaaagacgtagagtacagcgtaaaagtcttggttgacaccatgacccgatcgcttgcccgaggtcaacgcatcgaaattcgcggtttcggcagcttcgatttgaaccatcgtcctgcccgcatcggtcgcaatcccaaaaccggcgagcgcgtggaagtacctgaaaaacatgtaccccacttcaagcctggtaaagaattgcgcgagcgggtcgacttggctttaaaagaaaatgccaattaa

SEQ ID NO: 11 - DNA, 2057 bp, Haemophilus influenzae
  misc_feature (338)..(338): n is a, c, g, or t
  misc_feature (576)..(576): n is a, c, g, or t
atgtcagaatcttttgctcaactttttgaagaatcattaaaagtccttgaaactcgtcaaggttcaatcgttagcggtactgtagtagctatccaaaaaggctttgtgcttgttgatgcaggtttaaaatctgagtctgcaattccagttgctgaattcttaaatgcacaaggcgaacttgaaatccaagttggcgatactgtaaatgttgcattagatgcagttgaagatggtttcggtgaaactaaactttctcgtgagaaagcggttcgtcacgaatcttggattgaattagaaaaagcttacgaagaaaaagcgaccgttatcggtttaatcancggcaaagtgaaaggtggcttcacagttgagttaaacggtgttcgtgcattcttaccaggttctttagttgatactcgtccagcgcgtgaagcagatcacttacttggtaaagaattagaattcaaagtaatcaaattagatcaaaaacgtaacaacgttgttgtttctcgtcgtgcagtaattgaatctgaaaacagccaagaacgtgaacaagtattagaaaatcttgtggaaggntcagaagttaaaggtgtcgttaaaaacttaactgagtacggtgcattcgttgatcttggtggtgttgatggtttattacacatcacagatatggcttggaaacgtgttaaacacccaagtgaaatcgtaaatgttggcgatgaagttactgttaaagtattaaaattcgataaagatcgtactcgtgtatctttaggcttaaaacaattaggtcaagatccatgggcagcaattgctgaaaatcatccagtaaacagcaaattaacaggtaaagtaactaacttaactgactatggctgtttcgttgaaatcttagatggcgttgaaggtttagttcacgtttctgaaatggattggactaataaaaatatccacccatctaaagtggttagcttaggcgatactgtagaagtaatggtattagaaattgatgaagaacgtcgtcgtatttctttaggcttaaaacaatgtaaagctaacccttggactcagtttgctgatactcacaacaaaggcgataaagtaacgggtaaaatcaagtctatcactgatttcggtatcttcatcggtcttgaaggtggtatcgatggtttagttcacttatctgatatttcttggagtatttcaggcgaagaagctgttcgtcaatacaaaaaaggtgacgaagtttcagctgttgtattagctgttgatgcagtaaaagaacgtatctctttaggtattaaacaacttgaagaagatccattcaataactttgtcgcaatcaacaaaaaaggtgcggtagtatctgcaactgttgttgaagcagatgctaaaggtgctaaagttgaattagcaggtggtgttgaaggttatatccgttctgctgatttaacaagtgaagtagctgttggtgatgtcgttgaagcgaaatacactggagtagatcgtaaatctcgcattgttcacttatcagtgaaagcaaaagatcaagctgaagaagctgcggcagttgcaagtgtaaataacaaacaagaagatattgttattccaaatgcaatggctgaagcatttaaagcagctaaaggtgaataattaattcacgtaataaggctgggctgatgtccagccttatttgttaggagttattgctaatatatttgtgtaatttattatcaatagatcttaatttaagtaatattaaggaagtatagacgatgactaagtcagaacttatggaaaaattgtcagcaaaacagccaactttacctgcaaaagaaattgaaaatatggtaaaaggtattttagagtttatttctcaatctcttgaaaatggtgatcgtgttgaagttcgaggttttggtagcttttctttacatcatcggcaaccacgtttgggaagaaatccgaaaacaggcgattcagtaaacttatcagctaagtctgttccatattttaaagcgggtaaagaattaaaagctcgagtggatgttcaggcttaa

SEQ ID NO: 12 - DNA, 2101 bp, Pseudomonas aeruginosa
atgagcgaaagcttcgcagaactctttgaagaaagtctgaaatccctcgacatgcagccgggtgccatcatcaccggcatcgtggtcgacatcgatggtgactgggtcaccgtccatgccggtctgaaatccgagggcgtcatcccggtcgagcagttctacaacgaacagggcgagctgaccatcaaggtgggtgacgaagtccacgtcgcactggacgcggtagaagacggctttggcgagaccaagctgtcccgcgagaaagccaagcgcgccgagagctggattgttctggaagcggctttcgctgccgacgaagtggtcaagggcgtcatcaacggcaaggtcaagggcggtttcaccgtcgacgtcaacggcatccgcgcgttcctgccgggttctctggtcgacgttcgtccggttcgcgacaccacccacctggaaggcaaagagctcgagttcaaggtcatcaagctcgaccagaagcgcaacaacgttgtcgtttcccgccgcagcgtcctggaagccgagaacagcgccgagcgtgaagctctgctggaatcgctgcaggaaggccagcaggtcaaaggtatcgtcaagaacctcaccgactacggcgcattcgtggacctgggcggcgtagacggcctgctacacatcaccgacatggcctggaagcgcatcaagcatccgtccgagatcgtcaacgttggcgacgagatcgacgtcaaggtcctgaagttcgaccgcgagcgcaaccgtgtatccctgggcctgaagcaactgggcgaagacccgtgggttgccatcaaggcgcgttacccggaaggtacccgcgtcatggcccgcgtcaccaacctcaccgactacggctgcttcgccgagctggaagagggcgtggaaggcctggtacacgtctccgaaatggactggaccaacaagaacatccatccgtcgaaagtcgtccaggttggcgatgaagtggaagttcaggttctggacatcgacgaagagcgtcgtcgtatctccctgggtatcaagcagtgcaaatccaacccgtgggaagacttctccagccagttcaacaagggtgaccgtatctccggtaccatcaagtcgatcaccgacttcggtatcttcatcggtctggacggcggcatcgacggcctggtccacctgtccgacatctcctggaacgaagtcggcgaagaagccgtacgtcgcttcaagaagggcgacgagctggaaaccgtcatcctgtcggtcgatccggagcgcgagcgcatctccctgggcatcaagcagctggaagacgatccgttctccaactacgcgtccctgcacgagaaaggcagcatcgtccgcggtaccgtgaaggaagtcgacgccaagggcgctgtcatcagcctgggcgacgacatcgaaggtatcctgaaggcttccgaaatcagccgtgaccgcgtcgaagacgcgcgcaacgtcctgaaggaaggcgaggaagtcgaagccaagatcatcagcatcgaccgcaagagccgcgtcatcagcctctccgtcaagtccaaggacgtcgacgacgagaaggacgcaatgaaagaactgcgtaagcaggaagtagaaagcgctggtccgaccaccatcggtgatctgatccgtgctcagatggagaatcagggctaagtctctgatccatcatgaaaaagggcggcctaggccgccctttttcgttttccccttcttggacctgttcaaagactgatcagcatgctaaaagagacctgagctgatctagccgcttgaaaaagaagggaaaaccatgaccaagtcggagttgatcgaacggatcgttacccatcaggggcaactgtccgcgaaggatgtcgagttggcaatcaagaccatgctggagcaaatgtcccaggccctggcgaccggggaccggatcgagatccgtggcttcggcagcttttccttgcactaccgcgccccgcgcgtcggtcgcaaccccaagaccggggagtcggtacgcctcgacggcaagttcgtgccgcacttcaagccgggcaaggagttgcgggatcgggtcaacgagccggagtga

SEQ ID NO: 13 - protein, 576 aa, Bordetella pertussis
MDSNLMSSVSTSAILGGENFADLFAESLKSQDMKSGEVISAEVVRVDHNFVVVNAGLKSEALIPLEEFLNDQGELEVQPGDFVSVAIDSLENGYGDTILSRDRAKRLSAWLQLEQALENGELVTGTITGKVKGGLTVMTNGIRAFLPGSLVDLRPVKDTTPYEGKTLEFKVIKLDRKRNNVVLSRRQVLEASMGEERQKLLETLHEGAVVKGVVKNITDYGAFVDLGGIDGLLHITDMAWRRVRHPSEVLQVGQEVEAKVLKFDQEKSRVSLGVKQLGEDPWVGLARRYPQGTRLFGKVTNLTDYGAFVEVEAGIEGLVHVSEMDWTNKNVDPRKVVTLGEEVEVMVLEIDEDRRRISLGMKQCRQNPWEEFATNFKRGDKVRGAIKSITDFGVFVGLPGGIDGLVHLSDLSWTESGEEAVRNFKKGDELEAVVLGIDTEKERISLGIKQLEGDPFNNFVATHDKGAVVPGTIKSVEPKGAVITLSVDVEGYLRASEISSGRVEDATTVLKAGENIEAMIVNIDRKARSIQLSIKARDNAETAETIQRMSEASASSGTTNLGALLKAKLDQQRNDG

SEQ ID NO: 14 - protein, 557 aa, Escherichia coli
MTESFAQLFEESLKEIETRPGSIVRGVVVAIDKDVVLVDAGLKSESAIPAEQFKNAQGELEIQVGDEVDVALDAVEDGFGETLLSREKAKRHEAWITLEKAYEDAETVTGVINGKVKGGFTVELNGIRAFLPGSLVDVRPVRDTLHLEGKELEFKVIKLDQKRNNVVVSRRAVIESENSAERDQLLENLQEGMEVKGIVKNLTDYGAFVDLGGVDGLLHITDMAWKRVKHPSEIVNVGDEITVKVLKFDRERTRVSLGLKQLGEDPWVAIAKRYPEGTKLTGRVTNLTDYGCFVEIEEGVEGLVHVSEMDWTNKNIHPSKVVNVGDVVEVMVLDIDEERRRISLGLKQCKANPWQQFAETHNKGDRVEGKIKSITDFGIFIGLDGGIDGLVHLSDISWNVAGEEAVREYKKGDEIAAVVLQVDAERERISLGVKQLAEDPFNNWVALNKKGAIVTGKVTAVDAKGATVELADGVEGYLRASEASRDRVEDATLVLSVGDEVEAKFTGVDRKNRAISLSVRAKDEADEKDAIATVNKQEDANFSNNAMAEAFKAAKGE

SEQ ID NO: 15 - protein, 549 aa, Haemophilus influenzae
  misc_feature (113)..(113): Xaa can be any naturally occurring amino acid
MSESFAQLFEESLKVLETRQGSIVSGTVVAIQKGFVLVDAGLKSESAIPVAEFLNAQGELEIQVGDTVNVALDAVEDGFGETKLSREKAVRHESWIELEKAYEEKATVIGLIXGKVKGGFTVELNGVRAFLPGSLVDTRPAREADHLLGKELEFKVIKLDQKRNNVVVSRRAVIESENSQEREQVLENLVEGSEVKGVVKNLTEYGAFVDLGGVDGLLHITDMAWKRVKHPSEIVNVGDEVTVKVLKFDKDRTRVSLGLKQLGQDPWAAIAENHPVNSKLTGKVTNLTDYGCFVEILDGVEGLVHVSEMDWTNKNIHPSKVVSLGDTVEVMVLEIDEERRRISLGLKQCKANPWTQFADTHNKGDKVTGKIKSITDFGIFIGLEGGIDGLVHLSDISWSISGEEAVRQYKKGDEVSAVVLAVDAVKERISLGIKQLEEDPFNNFVAINKKGAVVSATVVEADAKGAKVELAGGVEGYIRSADLTSEVAVGDVVEAKYTGVDRKSRIVHLSVKAKDQAEEAAAVASVNNKQEDIVIPNAMAEAFKAAKGE

SEQ ID NO: 16 - protein, 561 aa, Neisseria gonorrhoeae
MSMENFAQLLEESFTLQEMNPGEVITAEVVAIDQNFVTVNAGLKSESLIDVAEFKNAQGEIEVKVGDFVTVTIESVENGFGETKLSREKAKRAADWIALEEAMENGNILSGIINGKVKGGLTVMISSIRAFLPGSLVDVRPVKDTSHFEGKEIEFKVIKLDKKRNNVVVSRRAVLEATLGEERKALLENLQEGSVIKGIVKNITDYGAFVDLGGIDGLLHITDLAWRRVKHPSEVLEVGQEVEAKVLKFDQEKQRVSLGMKQLGEDPWSGLTRRYPQATRLFGKVSNLTDYGAFVEIEQGIEGLVHVSEMDWTNKNVHPSKVVQLGDEVEVMILEIDEGRRRISLGMKQCQANPWEEFAANHNKGDKISGAVKSITDFGVFVGLPGGIDGLVHLSDLSWTESGEEAVRKYKKGEEVEAVVLAIDVEKERISLGIKQLEGDPFGNFISVNDKGSLVKGSVKSVDAKGAVIALSDEVEGYLPASEFAADRVEDLTTKLKEGDEVEAVIVTVDRKNRSIKLSVKAKDAKESREALNSVNAAANANAGTTSLGDLLKAKLSGEQE

SEQ ID NO: 17 - protein, 561 aa, Neisseria meningitidis
MSMENFAQLLEESFTLQEMNPGEVITAEVVAIDQNFVTVNAGLKSESLIDVAEFKNAQGEIEVKVGDFVTVTIESVENGFGETKLSREKAKRAADWIALEEAMENGDILSGIINGKVKGGLTVMISSIRAFLPGSLVDVRPVKDTSHFEGKEIEFKVIKLDKKRNNVVVSRRAVLEATLGEERKALLENLQEGSVIKGIVKNITDYGAFVDLGGIDGLLHITDLAWRRVKHPSEVLEVGQEVEAKVLKFDQEKQRVSLGMKQLGEDPWSGLTRRYPQGTRLFGKVSNLTDYGAFVEIEQGIEGLVHVSEMDWTNKNVHPSKVVQLGDEVEVMILEIDEGRRRISLGMKQCQANPWEEFAANHNKGDKISGAVKSITDFGVFVGLPGGIDGLVHLSDLSWTESGEEAVRKYKKGEEVEAVVLAIDVEKERISLGIKQLEGDPFGNFISVNDKGSLVKGSVKSVDAKGAVIALSDEVEGYLPASEFAADRVEDLTTKLKEGDEVEAVIVTVDRKNRSIKLSVKAKDAKESREALNSVNAAANANAGTTSLGDLLKAKLSGEQE

SEQ ID NO: 18 - protein, 559 aa, Pseudomonas aeruginosa
MSESFAELFEESLKSLDMQPGAIITGIVVDIDGDWVTVHAGLKSEGVIPVEQFYNEQGELTIKVGDEVHVALDAVEDGFGETKLSREKAKRAESWIVLEAAFAADEVVKGVINGKVKGGFTVDVNGIRAFLPGSLVDVRPVRDTTHLEGKELEFKVIKLDQKRNNVVVSRRSVLEAENSAEREALLESLQEGQQVKGIVKNLTDYGAFVDLGGVDGLLHITDMAWKRIKHPSEIVNVGDEIDVKVLKFDRERNRVSLGLKQLGEDPWVAIKARYPEGTRVMARVTNLTDYGCFAELEEGVEGLVHVSEMDWTNKNIHPSKVVQVGDEVEVQVLDIDEERRRISLGIKQCKSNPWEDFSSQFNKGDRISGTIKSITDFGIFIGLDGGIDGLVHLSDISWNEVGEEAVRRFKKGDELETVILSVDPERERISLGIKQLEDDPFSNYASLHEKGSIVRGTVKEVDAKGAVISLGDDIEGILKASEISRDRVEDARNVLKEGEEVEAKIISIDRKSRVISLSVKSKDVDDEKDAMKELRKQEVESAGPTTIGDLIRAQMENQG

SEQ ID NO: 19 - DNA, 213 bp, Bordetella pertussis
gacatgaagtccggcgaggtcatcagcgcagaagtcgtgcgcgtcgaccacaacttcgtggtcgtcaacgccggcctgaagtccgaagcgctgattcccctggaagagttcctcaacgaccagggcgaactcgaagttcaacccggcgacttcgtctcggtggcgatcgattcgctggagaacggctacggcgacaccatcctgtcgcgcgac

SEQ ID NO: 20 - DNA, 213 bp, Bordetella pertussis
gccctcgagaacggcgagctggtcaccggcacgatcaccggcaaggtcaagggcggcctgaccgtcatgaccaacggcatccgcgcgttcctgcccggttcgctggtcgacctgcgtccggtcaaggacaccacgccgtacgaaggcaagaccctcgaattcaaggtcatcaagctggaccgcaagcgcaacaacgtcgtgctgtcgcgccgc

SEQ ID NO: 21 - DNA, 219 bp, Bordetella pertussis
acgctgcacgaaggcgcggtggtcaagggcgtggtcaagaacatcaccgactacggcgcgttcgtcgacctgggcggcatcgatggcctgctgcacatcaccgacatggcctggcgccgtgtgcgtcacccgtccgaagtcctgcaagtgggtcaggaagtcgaagccaaggtgctcaagttcgaccaggaaaagagccgcgtctccctgggcgtcaag

SEQ ID NO: 22 - DNA, 225 bp, Bordetella pertussis
cgctacccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaag

SEQ ID NO: 23 - DNA, 225 bp, Bordetella pertussis
aacttcaagcgtggtgacaaggtccgcggcgccatcaagtcgatcaccgacttcggcgtgttcgtcggcctgcccggcggcatcgacggcctggtccatctgtccgacctgtcgtggacggaatcgggcgaggaagccgtgcgcaacttcaagaagggcgacgagctggaagccgtggtgctgggcatcgataccgagaaagagcgcatctcgctgggtatcaag

SEQ ID NO: 24 - DNA, 222 bp, Bordetella pertussis
acgcacgacaagggcgccgttgttccgggcaccatcaagtcggtcgagcccaagggcgccgtgatcaccctgtcggtggatgtggaaggctacctgcgcgcctccgagatctcctcgggccgcgtcgaagacgctaccaccgtgctgaaggctggcgagaacatcgaagccatgatcgtcaacatcgaccgcaaggcgcgttcgatccagctgtcgatcaag

SEQ ID NO: 25 - DNA, 360 bp, Bordetella pertussis
gtgaccaagtcggagctgatcgccgcgctggcggcccgctatcctcagctggccgctcgcgacaccgattacgctgtcaagaccatgctcgatgcaatgacccaggccctggcctcgggtcagcgcatcgaaatccgcgggtttggcagcttttcgctgtcgcagcgctctccccgtatcgggcgcaatccgaagtcgggcgaacaagtgctggtgcctggcaagcaggtgccgcacttcaaggccggcaaggagctgcgcgagtgggtcgatctggttggcaacgatcagggcgacgactcgtccaacgggtcgtccgacccgctgcaatcggtcatggatatgcatgccatgcactga

SEQ ID NO: 26 - DNA, 285 bp, Escherichia coli
atgaccaagtcagaattgatagaaagacttgccacccagcaatcgcacattcccgccaagacggttgaagatgcagtaaaagagatgctggagcatatggcctcgactcttgcgcagggcgagcgtattgaaatccgcggtttcggcagtttctctttgcactaccgcgcaccacgtaccggacgtaatccgaagactggcgataaagtagaactggaaggaaaatacgttcctcactttaaacctggtaaagaactgcgcgatcgcgccaatatttacggttaa

SEQ ID NO: 27 - DNA, 285 bp, Haemophilus influenzae
atgactaagtcagaacttatggaaaaattgtcagcaaaacagccaactttacctgcaaaagaaattgaaaatatggtaaaaggtattttagagtttatttctcaatctcttgaaaatggtgatcgtgttgaagttcgaggttttggtagcttttctttacatcatcggcaaccacgtttgggaagaaatccgaaaacaggcgattcagtaaacttatcagctaagtctgttccatattttaaagcgggtaaagaattaaaagctcgagtggatgttcaggcttaa

SEQ ID NO: 28 - DNA, 297 bp, Neisseria gonorrhoeae
atggttcgcttggcagaagtatttgccgccaaaaacggcacgcatcttctggcaaaagacgtagagtacagcgtaaaagtcttggttgacaccatgacccgatcgcttgcccgaggtcaacgcatcgaaattcgcggtttcggcagcttcgatttgaaccatcgtcctgcccgcatcggtcgcaatcccaaaaccggcgagcgcgtggaagtacctgaaaaacatgtaccccacttcaagcctggtaaagaattgcgcgagcgggtcgacttggctttaaaagaaaatgccaattaa

SEQ ID NO: 29 - DNA, 315 bp, Neisseria meningitidis
atgacaaagtctgagttaatggttcgtttggcagaagtgtttgccgccaaaaacggcacgcatcttctggcaaaagacgtagagtacagcgtaaaagtcttggttgacaccatgactagatcgcttgcccgaggtcaacgcatcgaaatccgcggtttcggcagcttcgatttgaaccatcgtcctgcccgcatcggtcgcaatcccaaaaccggcgagcgtgtggaagtacctgaaaaacatgtaccccacttcaagcccggtaaagaattgcgcgagcgggtcgacttggctttaaaagaaaatgccaattaa

SEQ ID NO: 30 - DNA, 285 bp, Pseudomonas aeruginosa
atgaccaagtcggagttgatcgaacggatcgttacccatcaggggcaactgtccgcgaaggatgtcgagttggcaatcaagaccatgctggagcaaatgtcccaggccctggcgaccggggaccggatcgagatccgtggcttcggcagcttttccttgcactaccgcgccccgcgcgtcggtcgcaaccccaagaccggggagtcggtacgcctcgacggcaagttcgtgccgcacttcaagccgggcaaggagttgcgggatcgggtcaacgagccggagtga

SEQ ID NO: 31 - protein, 119 aa, Bordetella pertussis
MTKSELIAALAARYPQLAARDTDYAVKTMLDAMTQALASGQRIEIRGFGSFSLSQRSPRIGRNPKSGEQVLVPGKQVPHFKAGKELREWVDLVGNDQGDDSSNGSSDPLQSVMDMHAMH

SEQ ID NO: 32 - protein, 94 aa, Escherichia coli
MTKSELIERLATQQSHIPAKTVEDAVKEMLEHMASTLAQGERIEIRGFGSFSLHYRAPRTGRNPKTGDKVELEGKYVPHFKPGKELRDRANIYG

SEQ ID NO: 33 - protein, 94 aa, Haemophilus influenzae
MTKSELMEKLSAKQPTLPAKEIENMVKGILEFISQSLENGDRVEVRGFGSFSLHHRQPRLGRNPKTGDSVNLSAKSVPYFKAGKELKARVDVQA

SEQ ID NO: 34 - protein, 98 aa, Neisseria gonorrhoeae
MVRLAEVFAAKNGTHLLAKDVEYSVKVLVDTMTRSLARGQRIEIRGFGSFDLNHRPARIGRNPKTGERVEVPEKHVPHFKPGKELRERVDLALKENAN

SEQ ID NO: 35 - protein, 104 aa, Neisseria meningitidis
MTKSELMVRLAEVFAAKNGTHLLAKDVEYSVKVLVDTMTRSLARGQRIEIRGFGSFDLNHRPARIGRNPKTGERVEVPEKHVPHFKPGKELRERVDLALKENAN

SEQ ID NO: 36 - protein, 94 aa, Pseudomonas aeruginosa
MTKSELIERIVTHQGQLSAKDVELAIKTMLEQMSQALATGDRIEIRGFGSFSLHYRAPRVGRNPKTGESVRLDGKFVPHFKPGKELRDRVNEPE

SEQ ID NO: 37 - DNA, 2876 bp, Artificial
  B. pertussis 50D6 clone nt sequence rpsA gene
atggattccaacctaatgtcttccgtttccacctccgccatccttggcggcgaaaacttcgccgacctgttcgcagaaagcctcaagagccaggacatgaagtccggcgaggtcatcagcgcagaagtcgtgcgcgtcgaccacaacttcgtggtcgtcaacgccggcctgaagtccgaagcgctgattcccctggaagagttcctcaacgaccagggcgaactcgaagttcaacccggcgacttcgtctcggtggcgatcgattcgctggagaacggctacggcgacaccatcctgtcgcgcgaccgcgccaagcgtctgtcggcctggctgcaactggagcaggccctcgagaacggcgagctggtcaccggcacgatcaccggcaaggtcaagggcggcctgaccgtcatgaccaacggcatccgcgcgttcctgcccggttcgctggtcgacctgcgtccggtcaaggacaccacgccgtacgaaggcaagaccctcgaattcaaggtcatcaagctggaccgcaagcgcaacaacgtcgtgctgtcgcgccgccaggtgctggaagccagcatgggcgaagagcgccagaagctgctcgagacgctgcacgaaggcgcggtggtcaagggcgtggtcaagaacatcaccgactacggcgcgttcgtcgacctgggcggcatcgatggcctgctgcacatcaccgacatggcctggcgccgtgtgcgtcacccgtccgaagtcctgcaagtgggtcaggaagtcgaagccaaggtgctcaagttcgaccaggaaaagagccgcgtctccctgggcgtcaagcagctgggcgaagatccgtgggtgggcctggctcgccgctacccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaagcagtgccgccagaacccgtgggaagagttcgccaccaacttcaagcgtggtgacaaggtccgcggcgccatcaagtcgatcaccgacttcggcgtgttcgtcggcctgcccggcggcatcgacggcctggtccatctgtccgacctgtcgtggacggaatcgggcgaggaagccgtgcgcaacttcaagaagggcgacgagctggaagccgtggtgctgggcatcgataccgagaaagagcgcatctcgctgggtatcaagcagctcgaaggctgtctcttatacacatctgacgtcttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaataattaattggggaccctagaggtccccttttttattttaaaaattttttcacaaaacggtttacaagcataactagtgcggccgcaagcttgcatgcctgcaggtcgactctagaggatccccgggtaccgagctcgaattcgcgcggccgcggcctaggcggccttaattaaagatgtgtataagagacaggctcgaaggcgacccgttcaacaacttcgttgccacgcacgacaagggcgccgttgttccgggcaccatcaagtcggtcgagcccaagggcgccgtgatcaccctgtcggtggatgtggaaggctacctgcgcgcctccgagatctcctcgggccgcgtcgaagacgctaccaccgtgctgaaggctggcgagaacatcgaagccatgatcgtcaacatcgaccgcaaggcgcgttcgatccagctgtcgatcaaggcccgcgataacgccgagacggccgagaccatccagcgcatgtccgaggcgagcgcttcgtcgggtacgacgaacttgggcgcgctgctcaaggccaagctggaccaacagcgcaacgacggttgac

SEQ ID NO: 38 - DNA, 3236 bp, Artificial
  B. pertussis 50D6 clone nt sequence rpsA operon
atggattccaacctaatgtcttccgtttccacctccgccatccttggcggcgaaaacttcgccgacctgttcgcagaaagcctcaagagccaggacatgaagtccggcgaggtcatcagcgcagaagtcgtgcgcgtcgaccacaacttcgtggtcgtcaacgccggcctgaagtccgaagcgctgattcccctggaagagttcctcaacgaccagggcgaactcgaagttcaacccggcgacttcgtctcggtggcgatcgattcgctggagaacggctacggcgacaccatcctgtcgcgcgaccgcgccaagcgtctgtcggcctggctgcaactggagcaggccctcgagaacggcgagctggtcaccggcacgatcaccggcaaggtcaagggcggcctgaccgtcatgaccaacggcatccgcgcgttcctgcccggttcgctggtcgacctgcgtccggtcaaggacaccacgccgtacgaaggcaagaccctcgaattcaaggtcatcaagctggaccgcaagcgcaacaacgtcgtgctgtcgcgccgccaggtgctggaagccagcatgggcgaagagcgccagaagctgctcgagacgctgcacgaaggcgcggtggtcaagggcgtggtcaagaacatcaccgactacggcgcgttcgtcgacctgggcggcatcgatggcctgctgcacatcaccgacatggcctggcgccgtgtgcgtcacccgtccgaagtcctgcaagtgggtcaggaagtcgaagccaaggtgctcaagttcgaccaggaaaagagccgcgtctccctgggcgtcaagcagctgggcgaagatccgtgggtgggcctggctcgccgctacccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaagcagtgccgccagaacccgtgggaagagttcgccaccaacttcaagcgtggtgacaaggtccgcggcgccatcaagtcgatcaccgacttcggcgtgttcgtcggcctgcccggcggcatcgacggcctggtccatctgtccgacctgtcgtggacggaatcgggcgaggaagccgtgcgcaacttcaagaagggcgacgagctggaagccgtggtgctgggcatcgataccgagaaagagcgcatctcgctgggtatcaagcagctcgaaggctgtctcttatacacatctgacgtcttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaataattaattggggaccctagaggtccccttttttattttaaaaattttttcacaaaacggtttacaagcataactagtgcggccgcaagcttgcatgcctgcaggtcgactctagaggatccccgggtaccgagctcgaattcgcgcggccgcggcctaggcggccttaattaaagatgtgtataagagacaggctcgaaggcgacccgttcaacaacttcgttgccacgcacgacaagggcgccgttgttccgggcaccatcaagtcggtcgagcccaagggcgccgtgatcaccctgtcggtggatgtggaaggctacctgcgcgcctccgagatctcctcgggccgcgtcgaagacgctaccaccgtgctgaaggctggcgagaacatcgaagccatgatcgtcaacatcgaccgcaaggcgcgttcgatccagctgtcgatcaaggcccgcgataacgccgagacggccgagaccatccagcgcatgtccgaggcgagcgcttcgtcgggtacgacgaacttgggcgcgctgctcaaggccaagctggaccaacagcgcaacgacggttgacgtgaccaagtcggagctgatcgccgcgctggcggcccgctatcctcagctggccgctcgcgacaccgattacgctgtcaagaccatgctcgatgcaatgacccaggccctggcctcgggtcagcgcatcgaaatccgcgggtttggcagcttttcgctgtcgcagcgctctccccgtatcgggcgcaatccgaagtcgggcgaacaagtgctggtgcctggcaagcaggtgccgcacttcaaggccggcaaggagctgcgcgagtgggtcgatctggttggcaacgatcagggcgacgactcgtccaacgggtcgtccgacccgctgcaatcggtcatggatatgcatgccatgcactga

SEQ ID NO: 39 - protein, 481 aa, Artificial
  B. pertussis 50D6 clone aa sequence S1 protein
MDSNLMSSVSTSAILGGENFADLFAESLKSQDMKSGEVISAEVVRVDHNFVVVNAGLKSEALIPLEEFLNDQGELEVQPGDFVSVAIDSLENGYGDTILSRDRAKRLSAWLQLEQALENGELVTGTITGKVKGGLTVMTNGIRAFLPGSLVDLRPVKDTTPYEGKTLEFKVIKLDRKRNNVVLSRRQVLEASMGEERQKLLETLHEGAVVKGVVKNITDYGAFVDLGGIDGLLHITDMAWRRVRHPSEVLQVGQEVEAKVLKFDQEKSRVSLGVKQLGEDPWVGLARRYPQGTRLFGKVTNLTDYGAFVEVEAGIEGLVHVSEMDWTNKNVDPRKVVTLGEEVEVMVLEIDEDRRRISLGMKQCRQNPWEEFATNFKRGDKVRGAIKSITDFGVFVGLPGGIDGLVHLSDLSWTESGEEAVRNFKKGDELEAVVLGIDTEKERISLGIKQLEGCLLYTSDVLCLKISDVTLHKIKIYHHEQ

SEQ ID NO: 40 - protein, 481 aa, Artificial
  B. pertussis 77D8 clone aa sequence S1 protein
MDSNLMSSVSTSAILGGENFADLFAESLKSQDMKSGEVISAEVVRVDHNFVVVNAGLKSEALIPLEEFLNDQGELEVQPGDFVSVAIDSLENGYGDTILSRDRAKRLSAWLQLEQALENGELVTGTITGKVKGGLTVMTNGIRAFLPGSLVDLRPVKDTTPYEGKTLEFKVIKLDRKRNNVVLSRRQVLEASMGEERQKLLETLHEGAVVKGVVKNITDYGAFVDLGGIDGLLHITDMAWRRVRHPSEVLQVGQEVEAKVLKFDQEKSRVSLGVKQLGEDPWVGLARRYPQGTRLFGKVTNLTDYGAFVEVEAGIEGLVHVSEMDWTNKNVDPRKVVTLGEEVEVMVLEIDEDRRRISLGMKQCRQNPWEEFATNFKRGDKVRGAIKSITDFGVFVGLPGGIDGLVHLSDLSWTESGEEAVRNFKKGDELEAVVLGIDTEKERISLGIKQLEGDPFNNFVATHDKGAVVPGTIKSVSYTHL

SEQ ID NO: 41 - protein, 558 aa, Artificial
  B. pertussis 83G6 clone aa sequence S1 protein
MDSNLMSSVSTSAILGGENFADLFAESLKSQDMKSGEVISAEVVRVDHNFVVVNAGLKSEALIPLEEFLNDQGELEVQPGDFVSVAIDSLENGYGDTILSRDRAKRLSAWLQLEQALENGELVTGTITGKVKGGLTVMTNGIRAFLPGSLVDLRPVKDTTPYEGKTLEFKVIKLDRKRNNVVLSRRQVLEASMGEERQKLLETLHEGAVVKGVVKNITDYGAFVDLGGIDGLLHITDMAWRRVRHPSEVLQVGQEVEAKVLKFDQEKSRVSLGVKQLGEDPWVGLARRYPQGTRLFGKVTNLTDYGAFVEVEAGIEGLVHVSEMDWTNKNVDPRKVVTLGEEVEVMVLEIDEDRRRISLGMKQCRQNPWEEFATNFKRGDKVRGAIKSITDFGVFVGLPGGIDGLVHLSDLSWTESGEEAVRNFKKGDELEAVVLGIDTEKERISLGIKQLEGDPFNNFVATHDKGAVVPGTIKSVEPKGAVITLSVDVEGYLRASEISSGRVEDATTVLKAGENIEAMIVNIDRKARSIQLSIKARDNAETAETIQRMSEAVSYTHL

SEQ ID NO: 42 - DNA, 3751 bp, Artificial
  Escherichia coli mutant Ins RpsA #1
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggattggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcctggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatgagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtaattctctgactcttcgggatttttattccgaagtttgttgagtttacttgacagattgcaggtttcgtcctgtaatcaagcactaagggcggctacggccgcccttaatcaatgcagcaacagcagccgcttaatttgcctttaaggaaccggaggaatcatgaccaagtcagaattgatagaaagacttgccacccagcaatcgcacattcccgccaagacggttgaagatgcagtaaaagagatgctggagcatatggcctcgactcttgcgcagggcgagcgtattgaaatccgcggtttcggcagtttctctttgcactaccgcgcaccacgtaccggacgtaatccgaagactggcgataaagtagaactggaaggaaaatacgttcctcactttaaacctggtaaagaactgcgcgatcgcgccaatatttacggttaagttttttactcaaacttgaacgagagaaaagcacctgtcgggtgcttttttcatttctctaatctggaactggaagctgcctcgcagagttttgaacagttttcaccctttcgttaaattcttctgaatatgcctcggggaacgcaaaattcccac

SEQ ID NO: 43 - DNA, 3381 bp, Artificial
  Escherichia coli mutant RpsA 3′ #2
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggattggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcctggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgccatatgaatatcctccttagttcctattccgaagttcctattctctagaaagtataggaacttcagagcgcttttgaagctggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaggggatcttgaagttcctattccgaagttcctattctctagaaagtataggaacttcgaagcagctccagcctacacgactcttcgggatttttattccgaagtttgttgagtttacttgacagattgcaggtttcgtcctgtaatcaagcactaagggcggctacggccgcccttaatcaatgcagcaacagcagccgcttaatttgcctttaaggaaccggaggaatcatgaccaagtcagaattgatagaaagacttgccacccagcaatcgcacattcccgccaagacggttgaagatgcagtaaaagagatgctggagcatatggcctcgactcttgcgcagggcgagcgtattgaaatccgcggtttcggcagtttctctttgcactaccgcgcaccacgtaccggacgtaatccgaagactggcgataaagtagaactggaaggaaaatacgttcctcactttaaacctggtaaagaactgcgcgatcgcgccaatatttacggttaagttttttactcaaacttgaacgagagaaaagcacctgtcgggtgcttttttcatttctctaatctggaactggaagctgcctcgcagagttttgaacagttttcaccctttcgttaaattcttctgaatatgcctcggggaacgcaaaattcccac

SEQ ID NO: 44 - protein, 449 aa, Artificial
  Escherichia coli mutant Ins RpsA #1 S1 protein
MTESFAQLFEESLKEIETRPGSIVRGVVVAIDKDVVLVDAGLKSESAIPAEQFKNAQGELEIQVGDEVDVALDAVEDGFGETLLSREKAKRHEAWITLEKAYEDAETVTGVINGKVKGGFTVELNGIRAFLPGSLVDVRPVRDTLHLEGKELEFKVIKLDQKRNNVVVSRRAVIESENSAERDQLLENLQEGMEVKGIVKNLTDYGAFVDLGGVDGLLHITDMAWKRVKHPSEIVNVGDEITVKVLKFDRERTRVSLGLKQLGEDPWVAIAKRYPEGTKLTGRVTNLTDYGCFVEIEEGVEGLVHVSEMDWTNKNIHPSKVVNVGDVVEVMVLDIDEERRRISLGLKQCKANPWQQFAETHNKGDRVEGKIKSITDFGIFIGLDGGIDGLVHLSDISWNVAGEEAVREYKKGDEIAAVVLQVDAERERISLGVKQLACRLELLRSSYTF

SEQ ID NO: 45 - protein, 438 aa, Artificial
  Escherichia coli mutant deltaRpsA 3′ #2 S1 protein
MTESFAQLFEESLKEIETRPGSIVRGVVVAIDKDVVLVDAGLKSESAIPAEQFKNAQGELEIQVGDEVDVALDAVEDGFGETLLSREKAKRHEAWITLEKAYEDAETVTGVINGKVKGGFTVELNGIRAFLPGSLVDVRPVRDTLHLEGKELEFKVIKLDQKRNNVVVSRRAVIESENSAERDQLLENLQEGMEVKGIVKNLTDYGAFVDLGGVDGLLHITDMAWKRVKHPSEIVNVGDEITVKVLKFDRERTRVSLGLKQLGEDPWVAIAKRYPEGTKLTGRVTNLTDYGCFVEIEEGVEGLVHVSEMDWTNKNIHPSKVVNVGDVVEVMVLDIDEERRRISLGLKQCKANPWQQFAETHNKGDRVEGKIKSITDFGIFIGLDGGIDGLVHLSDISWNVAGEEAVREYKKGDEIAAVVLQVDAERERISLGVKQLAI

SEQ ID NO: 46 - DNA, 2908 bp, Artificial
  Neisseria meningitidis NG7 construct
atgtctatggaaaattttgctcagctgttggaagaaagctttaccctgcaagaaatgaacccgggtgaggtgattaccgctgaagtagtggcaatcgaccaaaacttcgttaccgtaaacgcaggtctgaaatcagaatccctgattgatgtagctgaattcaaaaacgctcaaggcgaaattgaagttaaagtcggcgacttcgttaccgttaccatcgaatccgtcgaaaacggcttcggcgaaaccaaactgtcccgcgaaaaagccaaacgtgcagccgattggattgccctggaagaagccatggaaaacggcgacatcctgtccggcatcatcaacggaaaagtcaaaggcggcctgaccgttatgattagcagcatccgcgcattcctgccgggttctttggtcgacgtacgtcctgtaaaagacacttctcacttcgaaggcaaagagatcgaattcaaagtgatcaaactggacaaaaaacgcaacaacgtcgttgtttcccgccgcgccgttctggaagccactttgggtgaagaacgcaaagccctgctggaaaacctgcaagaaggctccgtcatcaaaggcatcgttaaaaacattaccgattacggtgcattcgttgacttgggcggcatcgacggtctgttgcacatcaccgatttggcatggcggcgcgtgaaacacccgagtgaagtcttggaagtcggtcaggaagttgaagccaaagtattgaaattcgaccaagaaaaacaacgcgtttccttgggtatgaaacaactgggcgaagatccttggagcggtctgacccgccgttatcctcaaggcacccgcctgttcggcaaagtatccaacctgaccgactacggcgcattcgtcgaaatcgaacaaggcatcgaaggtttggtacacgtctccgaaatggactggaccaacaaaaacgtacacccgagcaaagtcgtacaactgggcgacgaagtcgaagtcatgattttggaaatcgacgaaggccgccgccgtatctctttgggtatgaaacaatgccaagccaatccttgggaagaatttgccgccaaccacaacaaaggcgacaaaatctccggcgcggttaaatccattaccgatttcggcgtattcgtcggcctgcccggcggcatcgacggtttggttcacctgtccgacctgtcctggaccgaatccggcgaagaagccgtacgcaaatacaaaaaaggcgaagaagtcgaagccgtcgtattggcaatcgacgtggaaaaagaacgcatctccttgggtatcaaacaactggaagtcaaccgtgatatagattgaaaagtggatagatttatgatatagtggatagatttatgatataatgagttatcaacaaatcggaatttacggaggataaatgatgcaattcacaaagattgatataaataattggacacgaaaagagtatttcgaccactattttggcaatacgccctgcacatatagtatgacggtaaaactcgatatttctaagttgaaaaaggatggaaaaaagttatacccaactcttttatatggagttacaacgatcatcaatcgacatgaagagttcaggaccgcattagatgaaaacggacaggtaggcgttttttcagaaatgctgccttgctacacagtttttcataaggaaactgaaaccttttcgagtatttggactgagtttacagcagactatactgagtttcttcagaactatcaaaaggatatagacgcttttggtgaacgaatgggaatgtccgcaaagcctaatcctccggaaaacactttccctgtttctatgataccgtggacaagctttgaaggctttaacttaaatctaaaaaaaggatatgactatctactgccgatatttacgtttgggaagtattatgaggagggcggaaaatactatattcccttatcgattcaagtgcatcatgccgtttgtgacggctttcatgtttgccgttttttggatgaattacaagacttgctgaataaataaaatcccagtttgtcgcactgataaaaaccctttaggaactaaagggcgcacttctatactctctgtcgagagtagtgcgtcctgcagatctgttgacgggcccggtaccgcggccgccttaagacgcgtggatcatgccgtctgaacatatttcagacggctaactggaaggcgatccgttcggcaacttcatcagcgtgaacgacaaaggttctttggttaaaggttccgtgaaatctgttgacgccaaaggtgctgttatcgccctgtctgacgaagtagaaggctacctgcctgcttccgaatttgcagccgaccgcgttgaagatttgaccaccaaactgaaagaaggcgacgaagttgaagccgtcatcgttaccgttgaccgcaaaaaccgcagcatcaaactttccgttaaagccaaagatgccaaagaaagccgcgaagcactgaactccgtcaatgccgccgccaatgcgaatgccggcaccaccagcttgggcgacctgctgaaagccaaactctccggcgaacaagaataaggttgcagacatgacaaagtctgagttaatggttcgtttggcagaagtgtttgccgccaaaaacggcacgcatcttctggcaaaagacgtagagtacagcgtaaaagtcttggttgacaccatgactagatcgcttgcccgaggtcaacgcatcgaaatccgcggtttcggcagcttcgatttgaaccatcgtcctgcccgcatcggtcgcaatcccaaaaccggcgagcgtgtggaagtacctgaaaaacatgtaccccacttcaagcccggtaaagaattgcgcgagcgggtcgacttggctttaaaagaaaatgccaattaa

SEQ ID NO: 47 - DNA, 2529 bp, Artificial
  Neisseria meningitidis NG8 construct
atgtctatggaaaattttgctcagctgttggaagaaagctttaccctgcaagaaatgaacccgggtgaggtgattaccgctgaagtagtggcaatcgaccaaaacttcgttaccgtaaacgcaggtctgaaatcagaatccctgattgatgtagctgaattcaaaaacgctcaaggcgaaattgaagttaaagtcggcgacttcgttaccgttaccatcgaatccgtcgaaaacggcttcggcgaaaccaaactgtcccgcgaaaaagccaaacgtgcagccgattggattgccctggaagaagccatggaaaacggcgacatcctgtccggcatcatcaacggaaaagtcaaaggcggcctgaccgttatgattagcagcatccgcgcattcctgccgggttctttggtcgacgtacgtcctgtaaaagacacttctcacttcgaaggcaaagagatcgaattcaaagtgatcaaactggacaaaaaacgcaacaacgtcgttgtttcccgccgcgccgttctggaagccactttgggtgaagaacgcaaagccctgctggaaaacctgcaagaaggctccgtcatcaaaggcatcgttaaaaacattaccgattacggtgcattcgttgacttgggcggcatcgacggtctgttgcacatcaccgatttggcatggcggcgcgtgaaacacccgagtgaagtcttggaagtcggtcaggaagttgaagccaaagtattgaaattcgaccaagaaaaacaacgcgtttccttgggtatgaaacaactgggcgaagatccttggagcggtctgacccgccgttatcctcaaggcacccgcctgttcggcaaagtatccaacctgaccgactacggcgcattcgtcgaaatcgaacaaggcatcgaaggtttggtacacgtctccgaaatggactggaccaacaaaaacgtacacccgagcaaagtcgtacaactgggcgacgaagtcgaagtcatgattttggaaatcgacgaaggccgccgccgtatctctttgggtatgaaacaatgccaagccaatccttgggaagaatttgccgccaaccacaacaaaggcgacaaaatctccggcgcggttaaatccattaccgatttcggcgtattcgtcggcctgcccggcggcatcgacggtttggttcacctgtccgacctgtcctggaccgaatccggcgaagaagccgtacgcaaatacaaaaaaggcgaagaagtcgaagccgtcgtattggcaatcgacgtggaaaaagaacgcatctccttgggtatcaaacaactggaagtcaaccgtgatatagattgaaaagtggatagatttatgatatagtggatagatttatgatataatgagttatcaacaaatcggaatttacggaggataaatgatgcaattcacaaagattgatataaataattggacacgaaaagagtatttcgaccactattttggcaatacgccctgcacatatagtatgacggtaaaactcgatatttctaagttgaaaaaggatggaaaaaagttatacccaactcttttatatggagttacaacgatcatcaatcgacatgaagagttcaggaccgcattagatgaaaacggacaggtaggcgttttttcagaaatgctgccttgctacacagtttttcataaggaaactgaaaccttttcgagtatttggactgagtttacagcagactatactgagtttcttcagaactatcaaaaggatatagacgcttttggtgaacgaatgggaatgtccgcaaagcctaatcctccggaaaacactttccctgtttctatgataccgtggacaagctttgaaggctttaacttaaatctaaaaaaaggatatgactatctactgccgatatttacgtttgggaagtattatgaggagggcggaaaatactatattcccttatcgattcaagtgcatcatgccgtttgtgacggctttcatgtttgccgttttttggatgaattacaagacttgctgaataaataaaatcccagtttgtcgcactgataaaaaccctttaggaactaaagggcgcacttctatactctctgtcgagagtagtgcgtcctgcagatctgttgacgggcccggtaccgcggccgccttaagacgcgtggatcatgccgtctgaacatatttcagacggctaggttgcagacatgacaaagtctgagttaatggttcgtttggcagaagtgtttgccgccaaaaacggcacgcatcttctggcaaaagacgtagagtacagcgtaaaagtcttggttgacaccatgactagatcgcttgcccgaggtcaacgcatcgaaatccgcggtttcggcagcttcgatttgaaccatcgtcctgcccgcatcggtcgcaatcccaaaaccggcgagcgtgtggaagtacctgaaaaacatgtaccccacttcaagcccggtaaagaattgcgcgagcgggtcgacttggctttaaaagaaaatgccaattaa

SEQ ID NO: 48 - DNA, 2728 bp, Artificial
  Neisseria meningitidis NG9 construct
atgtctatggaaaattttgctcagctgttggaagaaagctttaccctgcaagaaatgaacccgggtgaggtgattaccgctgaagtagtggcaatcgaccaaaacttcgttaccgtaaacgcaggtctgaaatcagaatccctgattgatgtagctgaattcaaaaacgctcaaggcgaaattgaagttaaagtcggcgacttcgttaccgttaccatcgaatccgtcgaaaacggcttcggcgaaaccaaactgtcccgcgaaaaagccaaacgtgcagccgattggattgccctggaagaagccatggaaaacggcgacatcctgtccggcatcatcaacggaaaagtcaaaggcggcctgaccgttatgattagcagcatccgcgcattcctgccgggttctttggtcgacgtacgtcctgtaaaagacacttctcacttcgaaggcaaagagatcgaattcaaagtgatcaaactggacaaaaaacgcaacaacgtcgttgtttcccgccgcgccgttctggaagccactttgggtgaagaacgcaaagccctgctggaaaacctgcaagaaggctccgtcatcaaaggcatcgttaaaaacattaccgattacggtgcattcgttgacttgggcggcatcgacggtctgttgcacatcaccgatttggcatggcggcgcgtgaaacacccgagtgaagtcttggaagtcggtcaggaagttgaagccaaagtattgaaattcgaccaagaaaaacaacgcgtttccttgggtatgaaacaactgggcgaagatccttggagcggtctgacccgccgttatcctcaaggcacccgcctgttcggcaaagtatccaacctgaccgactacggcgcattcgtcgaaatcgaacaaggcatcgaaggtttggtacacgtctccgaaatggactggaccaacaaaaacgtacacccgagcaaagtcgtacaactgggcgacgaagtcgaagtcatgattttggaaatcgacgaaggccgccgccgtatctctttgggtatgaaacaatgccaagccaatccttgggaagaatttgccgccaaccacaacaaaggcgacaaaatctccggcgcggttaaatccattaccgatttcggcgtattcgtcggcctgcccggcggcatcgacggtttggttcacctgtccgacctgtcctggaccgaatccggcgaagaagccgtacgcaaatacaaaaaaggcgaagaagtcgaagccgtcgtattggcaatcgacgtggaaaaagaacgcatctccttgggtatcaaacaactggaaggcgatccgttcggcaacttcatcagcgtgaacgacaaaggttctttggttaaaggttccgtgaaatctgttgacgccaaaggtgctgttatcgccctgtctgacgaagtagaaggctacctgcctgcttccgaatttgcagccgaccgcgttgaagatttgaccaccaaactgaaagaaggcgacgaagttgaagccgtcatcgttaccgttgaccgcaaaaaccgcagcatcaaactttccgttaaagccaaagatgccaaagaaagccgcgaagcactgaactccgtcaatgccgccgccaatgcgaatgccggcaccaccagcttgggcgacctgctgaaagccaaactctccggcgaacaagaataaggttgcagacagtcaaccgtgatatagattgaaaagtggatagatttatgatatagtggatagatttatgatataatgagttatcaacaaatcggaatttacggaggataaatgatgcaattcacaaagattgatataaataattggacacgaaaagagtatttcgaccactattttggcaatacgccctgcacatatagtatgacggtaaaactcgatatttctaagttgaaaaaggatggaaaaaagttatacccaactcttttatatggagttacaacgatcatcaatcgacatgaagagttcaggaccgcattagatgaaaacggacaggtaggcgttttttcagaaatgctgccttgctacacagtttttcataaggaaactgaaaccttttcgagtatttggactgagtttacagcagactatactgagtttcttcagaactatcaaaaggatatagacgcttttggtgaacgaatgggaatgtccgcaaagcctaatcctccggaaaacactttccctgtttctatgataccgtggacaagctttgaaggctttaacttaaatctaaaaaaaggatatgactatctactgccgatatttacgtttgggaagtattatgaggagggcggaaaatactatattcccttatcgattcaagtgcatcatgccgtttgtgacggctttcatgtttgccgttttttggatgaattacaagacttgctgaataaataaaatcccagtttgtcgcactgataaaaaccctttaggaactaaagggcgcacttctatactctctgtcgagagtagtgcgtcctgcagatctgttgacgggcccggtaccgcggccgccttaagacgcgtggatcatgccgtctgaacatatttcagacggctaaaccatcgtcctgcccgcatcggtcgcaatcccaaaaccggcgagcgtgtggaagtacctgaaaaacatgtaccccacttcaagcccggtaaagaattgcgcgagcgggtcgacttggctttaaaagaaaatgccaattaa

SEQ ID NO: 49 - protein, 444 aa, Artificial
  S1 protein sequence in Neisseria meningitidis NG7 construct
MSMENFAQLLEESFTLQEMNPGEVITAEVVAIDQNFVTVNAGLKSESLIDVAEFKNAQGEIEVKVGDFVTVTIESVENGFGETKLSREKAKRAADWIALEEAMENGDILSGIINGKVKGGLTVMISSIRAFLPGSLVDVRPVKDTSHFEGKEIEFKVIKLDKKRNNVVVSRRAVLEATLGEERKALLENLQEGSVIKGIVKNITDYGAFVDLGGIDGLLHITDLAWRRVKHPSEVLEVGQEVEAKVLKFDQEKQRVSLGMKQLGEDPWSGLTRRYPQGTRLFGKVSNLTDYGAFVEIEQGIEGLVHVSEMDWTNKNVHPSKVVQLGDEVEVMILEIDEGRRRISLGMKQCQANPWEEFAANHNKGDKISGAVKSITDFGVFVGLPGGIDGLVHLSDLSWTESGEEAVRKYKKGEEVEAVVLAIDVEKERISLGIKQLEVNRDID

SEQ ID NO: 50 - protein, 444 aa, Artificial; identical to SEQ ID NO: 49
  S1 protein sequence in Neisseria meningitidis NG8 construct

SEQ ID NO: 51 - DNA, 3362 bp, Artificial
  Bordetella pertussis mutant C7
atggattccaacctaatgtcttccgtttccacctccgccatccttggcggcgaaaacttcgccgacctgttcgcagaaagcctcaagagccaggacatgaagtccggcgaggtcatcagcgcagaagtcgtgcgcgtcgaccacaacttcgtggtcgtcaacgccggcctgaagtccgaagcgctgattcccctggaagagttcctcaacgaccagggcgaactcgaagttcaacccggcgacttcgtctcggtggcgatcgattcgctggagaacggctacggcgacaccatcctgtcgcgcgaccgcgccaagcgtctgtcggcctggctgcaactggagcaggccctcgagaacggcgagctggtcaccggcacgatcaccggcaaggtcaagggcggcctgaccgtcatgaccaacggcatccgcgcgttcctgcccggttcgctggtcgacctgcgtccggtcaaggacaccacgccgtacgaaggcaagaccctcgaattcaaggtcatcaagctggaccgcaagcgcaacaacgtcgtgctgtcgcgccgccaggtgctggaagccagcatgggcgaagagcgccagaagctgctcgagacgctgcacgaaggcgcggtggtcaagggcgtggtcaagaacatcaccgactacggcgcgttcgtcgacctgggcggcatcgatggcctgctgcacatcaccgacatggcctggcgccgtgtgcgtcacccgtccgaagtcctgcaagtgggtcaggaagtcgaagccaaggtgctcaagttcgaccaggaaaagagccgcgtctccctgggcgtcaagcagctgggcgaagatccgtgggtgggcctggctcgccgctacccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaagcagtgccgccagaacccgtgggaagagttcgccaccaacttcaagcgtggtgacaaggtccgcggcgccatcaagtcgatcaccgacttcggcgtgttcgtcggcctgcccggcggcatcgacggcctggtccatctgtccgacctgtcgtggacggaatcgggcgaggaagccgtgcgcaacttcaagaagggcgacgagctggaagccgtggtgctgggcatcgataccgagaaagagcgcatctcgctgggtatcaagcagctcgaactagtgtcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctgttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgacactagtaggcgacccgttcaacaacttcgttgccacgcacgacaagggcgccgttgttccgggcaccatcaagtcggtcgagcccaagggcgccgtgatcaccctgtcggtggatgtggaaggctacctgcgcgcctccgagatctcctcgggccgcgtcgaagacgctaccaccgtgctgaaggctggcgagaacatcgaagccatgatcgtcaacatcgaccgcaaggcgcgttcgatccagctgtcgatcaaggcccgcgataacgccgagacggccgagaccatccagcgcatgtccgaggcgagcgcttcgtcgggtacgacgaacttgggcgcgctgctcaaggccaagctggaccaacagcgcaacgacggttgacgtgaccaagtcggagctgatcgccgcgctggcggcccgctatcctcagctggccgctcgcgacaccgattacgctgtcaagaccatgctcgatgcaatgacccaggccctggcctcgggtcagcgcatcgaaatccgcgggtttggcagcttttcgctgtcgcagcgctctccccgtatcgggcgcaatccgaagtcgggcgaacaagtgctggtgcctggcaagcaggtgccgcacttcaaggccggcaaggagctgcgcgagtgggtcgatctggttggcaacgatcagggcgacgactcgtccaacgggtcgtccgacccgctgcaatcggtcatggatatgcatgccatgcactga

SEQ ID NO: 52 - DNA, 3002 bp, Artificial
  Bordetella pertussis mutant C9
atggattccaacctaatgtcttccgtttccacctccgccatccttggcggcgaaaacttcgccgacctgttcgcagaaagcctcaagagccaggacatgaagtccggcgaggtcatcagcgcagaagtcgtgcgcgtcgaccacaacttcgtggtcgtcaacgccggcctgaagtccgaagcgctgattcccctggaagagttcctcaacgaccagggcgaactcgaagttcaacccggcgacttcgtctcggtggcgatcgattcgctggagaacggctacggcgacaccatcctgtcgcgcgaccgcgccaagcgtctgtcggcctggctgcaactggagcaggccctcgagaacggcgagctggtcaccggcacgatcaccggcaaggtcaagggcggcctgaccgtcatgaccaacggcatccgcgcgttcctgcccggttcgctggtcgacctgcgtccggtcaaggacaccacgccgtacgaaggcaagaccctcgaattcaaggtcatcaagctggaccgcaagcgcaacaacgtcgtgctgtcgcgccgccaggtgctggaagccagcatgggcgaagagcgccagaagctgctcgagacgctgcacgaaggcgcggtggtcaagggcgtggtcaagaacatcaccgactacggcgcgttcgtcgacctgggcggcatcgatggcctgctgcacatcaccgacatggcctggcgccgtgtgcgtcacccgtccgaagtcctgcaagtgggtcaggaagtcgaagccaaggtgctcaagttcgaccaggaaaagagccgcgtctccctgggcgtcaagcagctgggcgaagatccgtgggtgggcctggctcgccgctacccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaagcagtgccgccagaacccgtgggaagagttcgccaccaacttcaagcgtggtgacaaggtccgcggcgccatcaagtcgatcaccgacttcggcgtgttcgtcggcctgcccggcggcatcgacggcctggtccatctgtccgacctgtcgtggacggaatcgggcgaggaagccgtgcgcaacttcaagaagggcgacgagctggaagccgtggtgctgggcatcgataccgagaaagagcgcatctcgctgggtatcaagcagctcgaaggcgacccgttcaacaacttcgttgccacgcacgacaagggcgccgttgttccgggcaccatcaagtcggtcgagcccaagggcgccgtgatcaccctgtcggtggatgtggaaggctacctgcgcgcctccgagatctcctcgggccgcgtcgaagacgctaccaccgtgctgaaggctggcgagaacatcgaagccatgatcgtcaacatcgaccgcaaggcgcgttcgatccagctgtcgatcaaggcccgcgataacgccgagacggccgagaccatccagcgcatgtccgaggcgagcgcttcgtcgggtacgacgaacttgggcgcgctgctcaaggccaagctggaccaacagcgcaacgacggttgacactagtgtcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctgttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgacactagt

SEQ ID NO: 53 - protein, 470 aa, Artificial
  S1 protein sequence in Bordetella pertussis C7 construct
MDSNLMSSVSTSAILGGENFADLFAESLKSQDMKSGEVISAEVVRVDHNFVVVNAGLKSEALIPLEEFLNDQGELEVQPGDFVSVAIDSLENGYGDTILSRDRAKRLSAWLQLEQALENGELVTGTITGKVKGGLTVMTNGIRAFLPGSLVDLRPVKDTTPYEGKTLEFKVIKLDRKRNNVVLSRRQVLEASMGEERQKLLETLHEGAVVKGVVKNITDYGAFVDLGGIDGLLHITDMAWRRVRHPSEVLQVGQEVEAKVLKFDQEKSRVSLGVKQLGEDPWVGLARRYPQGTRLFGKVTNLTDYGAFVEVEAGIEGLVHVSEMDWTNKNVDPRKVVTLGEEVEVMVLEIDEDRRRISLGMKQCRQNPWEEFATNFKRGDKVRGAIKSITDFGVFVGLPGGIDGLVHLSDLSWTESGEEAVRNFKKGDELEAVVLGIDTEKERISLGIKQLELVSTCRGGGESHVVSQNL

SEQ ID NO: 54 - protein, 571 aa, Bordetella pertussis
MSSVSTSAILGGENFADLFAESLKSQDMKSGEVISAEVVRVDHNFVVVNAGLKSEALIPLEEFLNDQGELEVQPGDFVSVAIDSLENGYGDTILSRDRAKRLSAWLQLEQALENGELVTGTITGKVKGGLTVMTNGIRAFLPGSLVDLRPVKDTTPYEGKTLEFKVIKLDRKRNNVVLSRRQVLEASMGEERQKLLETLHEGAVVKGVVKNITDYGAFVDLGGIDGLLHITDMAWRRVRHPSEVLQVGQEVEAKVLKFDQEKSRVSLGVKQLGEDPWVGLARRYPQGTRLFGKVTNLTDYGAFVEVEAGIEGLVHVSEMDWTNKNVDPRKVVTLGEEVEVMVLEIDEDRRRISLGMKQCRQNPWEEFATNFKRGDKVRGAIKSITDFGVFVGLPGGIDGLVHLSDLSWTESGEEAVRNFKKGDELEAVVLGIDTEKERISLGIKQLEGDPFNNFVATHDKGAVVPGTIKSVEPKGAVITLSVDVEGYLRASEISSGRVEDATTVLKAGENIEAMIVNIDRKARSIQLSIKARDNAETAETIQRMSEASASSGTTNLGALLKAKLDQQRNDG

SEQ ID NO: 55 - DNA, 1674 bp, Moraxella catarrhalis
atggtatcatttgctgaactgtttgaagcaagccaatcagaacaaggtcttaacattgagcgtggctcagtgatttcaggaacagtcgttgctattgacagcgattggattacagttgatacaggtcttaaatctgaaggtatcgttgctcgtgaagagttcttaaacgaagctggcgagcttgaagtggcagtaggcgacagcattgatgttgtagttgaagctgtagataacggcatgggtcaaactgtgctttcacgcgaaaaagcgaagcgtgaagaaagctggcgtgcgcttgaacaactacacgaaaatgatgaaatcgttaaaggcgttatttcatctaaagttaaaggcggtttcactgttgagatcggttcagtgcgtgcattccttccagggtcattggtagatgttcgcccagttcgtgaaacgacacacctagaaggtaaagagcttgaatttaaagtcattaaaattgataaacaacgcaacaatatcgttgtatcacgccgtgctgttatggaagctgaaaattcagcagagcgtgatgagctactagccaagctagaagagggcatggaagtagaaggtatcgttaagaatctaactgaatatggtgcatttgttgatttaggcggtattgatggtctattgcacatcaccgatatggcgtggaagcgtatcaagcacccatcagaagcggtagaagttggtcaagaccttaaagtaaaagttcttaaatttgaccgtgagcgtaaccgtgttagcttgggtctaaaacaattgggtgctgacccatggactgaagttgagcaaacttatccagtgggtaccatcaccaaagcacgtgtaaccaacctaactgattatggctgctttgctgaaatcgctgaaggtattgaaggtttggtgcatgtctctgaaatggatcatacgaacaaaaatattcacccatcaaaagttgttcaagtgggcgatgaggtgaatgtgatggttcttgagattgatggtgagcgtcgccgtattagccttggtatcaaacaaaccatgccaaatccatgggctgagtttgataaaaaccatgaaaaaggtcaaaaaatcagcggtaccatcaaatcaatcactgactttggtctgtttattggcctagaaggtggtattgatggtttggtacacctgtctgatatttcatggactgagtctggtgaagaagccatccgcaattactcaaaaggtgatactgttgaggcggtcgtattgtctgttgatgcagaagcaaaccgtatcagcctaggcattaagcagctaaactctgatccatttaatgaatacttgctatcaaatgatcgtggtgctatcgttaaaggtacagtaaaagaagtggatgctaaaggtgcggtgattactttggccgatgaagttgaaggttatttgcgtgttgcagatattgctcgtgagcgtaccgaagatgcctctaaagtcttgagcgttggtgatgaagtagaagctaaaattgtcggcgttgaccgcaaatctcgcaatatcagcctatcaatcaaggcaaaagatgaagctgatgagcgtcaagccattaaagagctttctaatacagcagctgaaacacagccaaaaacacttggcgatattttcggtgagcagcttaaaggttga

SEQ ID NO: 56 - DNA, 1311 bp, Moraxella catarrhalis
atggtatcatttgctgaactgtttgaagcaagccaatcagaacaaggtcttaacattgagcgtggctcagtgatttcaggaacagtcgttgctattgacagcgattggattacagttgatacaggtcttaaatctgaaggtatcgttgctcgtgaagagttcttaaacgaagctggcgagcttgaagtggcagtaggcgacagcattgatgttgtagttgaagctgtagataacggcatgggtcaaactgtgctttcacgcgaaaaagcgaagcgtgaagaaagctggcgtgcgcttgaacaactacacgaaaatgatgaaatcgttaaaggcgttatttcatctaaagttaaaggcggtttcactgttgagatcggttcagtgcgtgcattccttccagggtcattggtagatgttcgcccagttcgtgaaacgacacacctagaaggtaaagagcttgaatttaaagtcattaaaattgataaacaacgcaacaatatcgttgtatcacgccgtgctgttatggaagctgaaaattcagcagagcgtgatgagctactagccaagctagaagagggcatggaagtagaaggtatcgttaagaatctaactgaatatggtgcatttgttgatttaggcggtattgatggtctattgcacatcaccgatatggcgtggaagcgtatcaagcacccatcagaagcggtagaagttggtcaagaccttaaagtaaaagttcttaaatttgaccgtgagcgtaaccgtgttagcttgggtctaaaacaattgggtgctgacccatggactgaagttgagcaaacttatccagtgggtaccatcaccaaagcacgtgtaaccaacctaactgattatggctgctttgctgaaatcgctgaaggtattgaaggtttggtgcatgtctctgaaatggatcatacgaacaaaaatattcacccatcaaaagttgttcaagtgggcgatgaggtgaatgtgatggttcttgagattgatggtgagcgtcgccgtattagccttggtatcaaacaaaccatgccaaatccatgggctgagtttgataaaaaccatgaaaaaggtcaaaaaatcagcggtaccatcaaatcaatcactgactttggtctgtttattggcctagaaggtggtattgatggtttggtacacctgtctgatatttcatggactgagtctggtgaagaagccatccgcaattactcaaaaggtgatactgttgaggcggtcgtattgtctgttgatgcagaagcaaaccgtatcagcctaggcattaagcagcta

SEQ ID NO: 57 - protein, 557 aa, Moraxella catarrhalis
MVSFAELFEASQSEQGLNIERGSVISGTVVAIDSDWITVDTGLKSEGIVAREEFLNEAGELEVAVGDSIDVVVEAVDNGMGQTVLSREKAKREESWRALEQLHENDEIVKGVISSKVKGGFTVEIGSVRAFLPGSLVDVRPVRETTHLEGKELEFKVIKIDKQRNNIVVSRRAVMEAENSAERDELLAKLEEGMEVEGIVKNLTEYGAFVDLGGIDGLLHITDMAWKRIKHPSEAVEVGQDLKVKVLKFDRERNRVSLGLKQLGADPWTEVEQTYPVGTITKARVTNLTDYGCFAEIAEGIEGLVHVSEMDHTNKNIHPSKVVQVGDEVNVMVLEIDGERRRISLGIKQTMPNPWAEFDKNHEKGQKISGTIKSITDFGLFIGLEGGIDGLVHLSDISWTESGEEAIRNYSKGDTVEAVVLSVDAEANRISLGIKQLNSDPFNEYLLSNDRGAIVKGTVKEVDAKGAVITLADEVEGYLRVADIARERTEDASKVLSVGDEVEAKIVGVDRKSRNISLSIKAKDEADERQAIKELSNTAAETQPKTLGDIFGEQLKG

SEQ ID NO: 58 - protein, 437 aa, Moraxella catarrhalis
MVSFAELFEASQSEQGLNIERGSVISGTVVAIDSDWITVDTGLKSEGIVAREEFLNEAGELEVAVGDSIDVVVEAVDNGMGQTVLSREKAKREESWRALEQLHENDEIVKGVISSKVKGGFTVEIGSVRAFLPGSLVDVRPVRETTHLEGKELEFKVIKIDKQRNNIVVSRRAVMEAENSAERDELLAKLEEGMEVEGIVKNLTEYGAFVDLGGIDGLLHITDMAWKRIKHPSEAVEVGQDLKVKVLKFDRERNRVSLGLKQLGADPWTEVEQTYPVGTITKARVTNLTDYGCFAEIAEGIEGLVHVSEMDHTNKNIHPSKVVQVGDEVNVMVLEIDGERRRISLGIKQTMPNPWAEFDKNHEKGQKISGTIKSITDFGLFIGLEGGIDGLVHLSDISWTESGEEAIRNYSKGDTVEAVVLSVDAEANRISLGIKQL

SEQ ID NO: 59 - DNA, 2186 bp, Moraxella catarrhalis
atggtatcatttgctgaactgtttgaagcaagccaatcagaacaaggtcttaacattgagcgtggctcagtgatttcaggaacagtcgttgctattgacagcgattggattacagttgatacaggtcttaaatctgaaggtatcgttgctcgtgaagagttcttaaacgaagctggcgagcttgaagtggcagtaggcgacagcattgatgttgtagttgaagctgtagataacggcatgggtcaaactgtgctttcacgcgaaaaagcgaagcgtgaagaaagctggcgtgcgcttgaacaactacacgaaaatgatgaaatcgttaaaggcgttatttcatctaaagttaaaggcggtttcactgttgagatcggttcagtgcgtgcattccttccagggtcattggtagatgttcgcccagttcgtgaaacgacacacctagaaggtaaagagcttgaatttaaagtcattaaaattgataaacaacgcaacaatatcgttgtatcacgccgtgctgttatggaagctgaaaattcagcagagcgtgatgagctactagccaagctagaagagggcatggaagtagaaggtatcgttaagaatctaactgaatatggtgcatttgttgatttaggcggtattgatggtctattgcacatcaccgatatggcgtggaagcgtatcaagcacccatcagaagcggtagaagttggtcaagaccttaaagtaaaagttcttaaatttgaccgtgagcgtaaccgtgttagcttgggtctaaaacaattgggtgctgacccatggactgaagttgagcaaacttatccagtgggtaccatcaccaaagcacgtgtaaccaacctaactgattatggctgctttgctgaaatcgctgaaggtattgaaggtttggtgcatgtctctgaaatggatcatacgaacaaaaatattcacccatcaaaagttgttcaagtgggcgatgaggtgaatgtgatggttcttgagattgatggtgagcgtcgccgtattagccttggtatcaaacaaaccatgccaaatccatgggctgagtttgataaaaaccatgaaaaaggtcaaaaaatcagcggtaccatcaaatcaatcactgactttggtctgtttattggcctagaaggtggtattgatggtttggtacacctgtctgatatttcatggactgagtctggtgaagaagccatccgcaattactcaaaaggtgatactgttgaggcggtcgtattgtctgttgatgcagaagcaaaccgtatcagcctaggcattaagcagctaaactctgatccatttaatgaatacttgctatcaaatgatcgtggtgctatcgttaaaggtacagtaaaagaagtggatgctaaaggtgcggtgattactttggccgatgaagttgaaggttatttgcgtgttgcagatattgctcgtgagcgtaccgaagatgcctctaaagtcttgagcgttggtgatgaagtagaagctaaaattgtcggcgttgaccgcaaatctcgcaatatcagcctatcaatcaaggcaaaagatgaagctgatgagcgtcaagccattaaagagctttctaatacagcagctgaaacacagccaaaaacacttggcgatattttcggtgagcagcttaaaggttgattgtcctgaatatgattaagcctattaaaaagagcggtgttggtcatcgctctttttattgcagtaaattgattcaatatcatttttaacggtttgaatgtattttgaatgtgtgacattttaacaaaaaaattattttctttttttgtgttttaagttattatttccagcttttttgataaaaagcttgatttgtgattgaggtaggtcgtatgcaagcggtaataaataaatctaatttgattgcaaatttggcatcagtctgtgaagagttagaagaagatgttgttgatgaagcagtacgcctaatgattgcgatgatggtcaatgagttggtgtatgatgggcgtattgaagttagaggttttggtagcttttgtttacatcaccgttctgcacgcattgctcgtaacccacgcacaggcgagagtgtctctgttaaggccaaagccactccttactttaagcctggtaaggcactgcgtgaatcggtgaatttggttaatgactga

SEQ ID NO: 60 - DNA, 1674 bp, Moraxella catarrhalis; identical to SEQ ID NO: 55

SEQ ID NO: 61 - DNA, 300 bp, Moraxella catarrhalis
atgcaagcggtaataaataaatctaatttgattgcaaatttggcatcagtctgtgaagagttagaagaagatgttgttgatgaagcagtacgcctaatgattgcgatgatggtcaatgagttggtgtatgatgggcgtattgaagttagaggttttggtagcttttgtttacatcaccgttctgcacgcattgctcgtaacccacgcacaggcgagagtgtctctgttaaggccaaagccactccttactttaagcctggtaaggcactgcgtgaatcggtgaatttggttaatgactga

SEQ ID NO: 62 - protein, 99 aa, Moraxella catarrhalis
MQAVINKSNLIANLASVCEELEEDVVDEAVRLMIAMMVNELVYDGRIEVRGFGSFCLHHRSARIARNPRTGESVSVKAKATPYFKPGKALRESVNLVND

SEQ ID NO: 63 - DNA, 1674 bp, Escherichia coli; identical to SEQ ID NO: 2

SEQ ID NO: 64 - protein, 557 aa, Escherichia coli; identical to SEQ ID NO: 14

SEQ ID NO: 65 - DNA, 1314 bp, Escherichia coli
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggattggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcctggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcataa

SEQ ID NO: 66 - protein, 437 aa, Escherichia coli
MTESFAQLFEESLKEIETRPGSIVRGVVVAIDKDVVLVDAGLKSESAIPAEQFKNAQGELEIQVGDEVDVALDAVEDGFGETLLSREKAKRHEAWITLEKAYEDAETVTGVINGKVKGGFTVELNGIRAFLPGSLVDVRPVRDTLHLEGKELEFKVIKLDQKRNNVVVSRRAVIESENSAERDQLLENLQEGMEVKGIVKNLTDYGAFVDLGGVDGLLHITDMAWKRVKHPSEIVNVGDEITVKVLKFDRERTRVSLGLKQLGEDPWVAIAKRYPEGTKLTGRVTNLTDYGCFVEIEEGVEGLVHVSEMDWTNKNIHPSKVVNVGDVVEVMVLDIDEERRRISLGLKQCKANPWQQFAETHNKGDRVEGKIKSITDFGIFIGLDGGIDGLVHLSDISWNVAGEEAVREYKKGDEIAAVVLQVDAERERISLGVKQLA

SEQ ID NO: 67 - DNA, 35 bp, Artificial Sequence
  V-PIPE Univ-F primer (pET15 vector amplification)
taacgcgacttaattggccagtgtgccggtctccg

SEQ ID NO: 68 - DNA, 29 bp, Artificial Sequence
  pET15notagRv (pET15 vector amplification)
catggtatatctccttcttaaagttaaac

SEQ ID NO: 69 - DNA, 37 bp, Artificial Sequence
  rpsAfw (Forward primer for rpsA FL and TR amplification)
aaggagatataccatgactgaatcttttgctcaactc

SEQ ID NO: 70 - DNA, 34 bp, Artificial Sequence
  rpsAFLrv (Reverse primer for rpsA FL amplification)
aattaagtcgcgttactcgcctttagctgctttg

SEQ ID NO: 71 - DNA, 36 bp, Artificial Sequence
  rpsATRrv (Reverse primer for rpsA TR and TR amplification)
aattaagtcgcgttatgcgagctgtttaacgcccag

SEQ ID NO: 72 - DNA, 20 bp, Artificial Sequence
  T7prom (Colony screening)
taatacgactcactataggg

SEQ ID NO: 73 - DNA, 21 bp, Artificial Sequence
  Seq Pet rv (Colony screening)
gatatccggatatagttcctc

(19) United States
(12) Patent Application Publication
(10) Pub. No.: US 2024/0082379 A1
(43) Pub. Date: Mar. 14, 2024

(54) Hyper-Blebbing Bacteria

(71) Applicant: GLAXOSMITHKLINE BIOLOGICALS SA, Rixensart (BE)

(72) Inventors: Scilla Buccato, Siena (IT); Maria Giuliani, Siena (IT); Alfredo Pezzicoli, Siena (IT); Marco Spinsanti, Siena (IT)

(21) Appl. No.: 18/256,749

(22) PCT Filed: Dec. 10, 2021
(86) PCT No.: PCT/EP2021/085316

§ 371 Date: Jun. 9, 2023

(30) Foreign Application Priority Data
Dec. 11, 2020 (EP) 20213527.3

(51) Int. Cl. A61K 39/095; A61K 39/02; A61K 39/102; A61K 39/104; A61K 39/108; A61K 39/112; C12N 15/70; C12N 15/74; C12P 1/04

(52) U.S. Cl. CPC A61K 39/095; A61K 39/0258; A61K 39/0275; A61K 39/099; A61K 39/102; A61K 39/104; A61K 39/1045; A61K 39/105; A61K 39/107; C12N 15/70; C12N 15/74; C12P 1/04

(57) ABSTRACT

The present invention relates to the field of hyper-blebbing Gram-negative bacterial cells which are genetically modified by modifying the rpsA gene, the rpsA operon and/or 30S ribosomal protein S1 and to native outer membrane vesicles (nOMVs) obtained or obtainable from said genetically modified bacterial cells.

### FIELD OF THE INVENTION

This invention relates to novel, genetically-modified Gram-negative bacteria and their use in the preparation and manufacture of native outer membrane vesicles (nOMV).

### BACKGROUND TO THE INVENTION

Gram-negative bacteria spontaneously release bleb-like particles of outer cell wall membrane referred to as native outer membrane vesicles (nOMV) (Kulp A. et al, Annu Rev Microbiol, 2010, 64:163-84), which can be used for diverse biotechnological applications (Ellis T N and Kuehn M J, Microbiol Mol Biol Rev. 2010 March; 74(1):81-94), such as in the field of OMV-based vaccines (Acevedo R et al, Front. Immunol, 2014, 5:121). As a matter of fact, recent evidences showed that an OMV-based vaccine can protect against Bordetella pertussis infection by inducing humoral immunogenicity (Gasperini G et al., Mol Cell Proteomics, 2018 February; 17(2):205-215) but, although B. pertussis naturally releases nOMV during liquid growth, the amount produced is insufficient for a large-scale vaccine process. Outer membrane vesicles may also be produced artificially, for example by detergent extraction (referred to as dOMV) or by genetically engineering bacteria to exhibit a hyper-blebbing phenotype, wherein, as a consequence of the genetic modification, larger quantities of outer membrane bud off thereby providing a practical source of membrane material.

For commercial vaccine production, a hyper-blebbing phenotype is usually achieved by deletion of genes encoding key membrane structural components, such as gna33 or tolR (Berlanda Scorza F et al, PLos One, 2012; 7(6):e35616). However, Gram-negative bacteria are highly genetically diverse and a mutation that induces hyper-blebbing in a first species may not be possible in a second species (e.g., because the gene is already absent from the wild type strain), or may not induce hyper-blebbing in a second species (e.g., because of different biochemical mechanisms governing membrane structure). When working with new species (i.e., species in which a hyper-blebbing mutation has not been applied) it is necessary to functionally screen for potential hyper-blebbing mutations by, for example, directed mutation of genes linked to hyper-blebbing in other species or random mutation and testing. It would therefore be advantageous to identify genetic modifications that induce a hyper-blebbing phenotype in as great a number of species types possible and, ideally, in all Gram-negative bacteria.

Since nOMV are particularly suited for vaccine development, it is an object of the invention to provide methods for producing increased amounts of nOMV in a diversity of Gram-negative species and genera.

### SUMMARY OF THE INVENTION

The inventors have surprisingly discovered new hyper-blebbing Gram-negative bacterial cells by modifications of the rpsA gene(s) (encoding 30S ribosomal protein S1), rpsA operon(s) and/or 30S ribosomal protein(s) S1 rather than by disruption of genes and/or operons encoding a membrane structural component and/or by disruption of a membrane structural component.

The genetic modifications described herein offer several advantages compared to known hyper-blebbing mutations. For example, rpsA (encoding 30S ribosomal protein S1) is broadly expressed and conserved across bacteria genera and species (Machulin A V et al., PloS one, 2019 Aug. 22; 14(8):e0221370), in contrast to known genes whose manipulation is involved in hyper-blebbing, which may vary substantially across bacterial genera and species. Thus, the present invention may be applied to any Gram-negative bacterial cells encoding a 30S ribosomal protein S1.

Of note, mutations resulting in hyper-blebbing of Gram-negative bacterial cells were found to be advantageous for bacterial survival in lethal environmental stresses (Manning A J and Kuehn M J, BMC Microbiol, 2011 Dec. 1; 11:258). In particular, nOMV release was described to increase with accumulation of misfolded proteins involved in regulation of cell wall stability (McBroom A J and Kuehn M J, Mol Microbiol, 2007 January; 63(2):545-58). Surprisingly, 30S ribosomal protein S1 is not associated with membrane stability. Thus, the genetic modifications described herein are linked to a mechanism of regulation of nOMV release, putatively transcriptional regulation by ribosome activity specialization (Xue S and Barna M, Nat Rev Mol Cell Biol, 2012 May 23; 13(6):355-69).

The rpsA gene is highly conserved in bacteria species and encodes the 30S ribosomal protein S1, which is involved in protein translation and autogenous regulation of ribosomal protein synthesis in bacteria (Aseev L V et al., RNA, 2008 September; 14(9):1882-94). Accordingly, it is implicated in modulating the concentration and the folding of those proteins that belong to its pathway. The deletion of the C-terminal domain of the 30S ribosomal protein S1, resulting from mutations within the 3′-region of the rpsA gene, is described as not lethal in E. coli (Schnier J, J Biol Chem, 1986 Sep. 5; 261(25):11866-71; Boni I V et al., J Bacteriol 2000 October; 182(20):5872-9). However, there are no previous reports that modifications of the rpsA gene, rpsA operon or 30S ribosomal protein S1 have an impact on the release of nOMV into the culture medium.

The ihfB gene, which encodes the beta subunit of the integration host factor (IHF) protein, is located immediately downstream rpsA. In some bacteria (e.g., B. pertussis) there is a very short intergenic region between rpsA and ihfB, which could suggest an operon organization; while for others (e.g., E. coli) there is a longer intergenic region which might contain another promoter dedicated for ihfB expression. Therefore, the two genes are likely to be co-transcribed is some Gram-negative bacteria, such as B. pertussis.

### DESCRIPTION OF DRAWINGS/FIGURES

FIG. 1: Random mutants screening results by fluorescence analysis. WT and random mutant strains were grown in deep-well plates until late stationary growth phase at 37° C. and 350 rpm. At least two experiments were performed for each clone. The optical density of cell cultures was monitored at 600 nm, while vesicles fluorescence in supernatants was measured by FM4-64 dye staining. Random mutants fluorescence intensity is normalized to the optical density (Specific yield) of each culture and expressed with respect to the WT strain (Fluorescence Intensity Over Wild type—FIOW).

FIG. 2: Confirmation of increased GMMA release in microscale growth. Increased nOMV release was confirmed by multiple micro-scale tests for 8 clones. Vesicles concentration in the culture supernatant was revealed by fluorescence analysis using FM4-64 dye.

FIG. 3: Schematic organization of rpsA gene. Domains are reported by boxes. Tn5 insertions in 50D6, 83G6, 77D8 clones are indicated by arrows.

FIG. 4: GMMA release from random mutants in small scale growth. (A) WT and mutant strains were grown in supplemented SS medium (50 ml), using 250 ml-baffled flasks with vented cup, at 37° C. and 180 rpm. Purified vesicles were prepared from late stationary phase of bacterial growth. (B) FM4-64 dye staining of culture supernatants, expressing the amount of GMMA release as FIOW normalized on optical density (Specific Yield). (C and D) Analysis of release of random mutants GMMA in small scale growth. WT and mutant strains were grown in 250 ml-baffled flasks with vented cup at 37° C. and 350 rpm. Purified vesicles were prepared from late stationary phase and quantified by NTA analysis. Results are expressed both as absolute values (C), and with respect to WT (FIOW) normalized to optical density (D).

FIG. 5: GMMA release by random mutants in large scale growth. WT and two random clones (50D6 and 66D1) were grown in supplemented SS medium (300 ml), using 2 L-buffled flasks, at 37° C. and 180 rpm. Growth profiles are graphically represented (A). Purified vesicles were prepared after approximately 72 hours of bacterial growth, and quantified by Lowry assay (B): total protein yield is normalized to the optical density of each culture and expressed with respect to the WT strain (FIOW), by NTA: particles concentration is expressed as absolute value (D) or normalized to the optical density of each culture and expressed with respect to the WT strain (FIOW) (E); and FM4-64 (C): fluorescence intensity is normalized to the optical density of each culture and expressed with respect to the WT strain (FIOW). The results are expressed with respect to both optical density and WT values.

FIG. 6: Protein profile of supernatants and purified vesicles from 50D6 (rpsA mutant) and 66D1 clones. The vesicle content of WT and mutant supernatants derived from small scale cultures (left panel) and isolated nOMVs (right panel) were quantified by FM4-64 and Lowry assay, respectively. Samples were loaded on a 4-12% precast acrylamide gel (5 μg vesicles loaded, fluorescence-normalized supernatants) for electrophoresis at room temperature and constant voltage. The proteins were visualized through Coomassie staining for nOMV and silver staining for supernatants. Molecular mass ladder is labelled on the left.

FIG. 7: Western blot analysis of culture supernatants. To confirm that supernatant preparation contained GMMA, fluorescence-intensity (FI)-normalised supernatants from WT and mutant large-scale cultures were separated on SDS-PAGE and blotted onto nitrocellulose membrane for immunoblot analysis with anti-OMV mouse serum. Molecular mass ladder is labelled on the left.

FIG. 8: TEM images. Purified nOMV were isolated from cultures of WT and mutants at late stationary phase and analysed by TEM with negative staining. Scale bars, 200 nm.

FIG. 9: B. pertussis clean mutant validation. WT and mutant strains were grown in deep-well plates until late stationary growth phase at 37° C. and 350 rpm. The optical density of cell cultures was monitored at 600 nm, while vesicles fluorescence in supernatants was measured by FM4-64 dye staining. Fluorescence intensity is normalized to the optical density of each culture and expressed with respect to the WT strain (FIOW). Data reported are average of 16 independent cultures (8 single colonies for each mutant in two experiments)

FIG. 10: S1 alignment. B. pertussis (Bp) and E. coli (Ec) 30S ribosomal protein S1 sequences are aligned using Clustal Omega online software (HyperTextTransferProtocolSecure://WorldWideWeb.ebi.ac.uk/Tools/msa/clustalo/). Asterisks indicate identities, dashes indicate gaps and spaces indicate mismatches. The first amino acid interrupted in the mutants is reported in the two sequences in bold and underlined.

FIG. 11: GMMA yields from mutant strains (FIOW) at 37° C. Fluorescence intensity was measured directly on cell-free spent supernatants and compared to wild type either normalized on culture volume (Volumetric Yield) or on optical density (Specific Yield). Data reported are average values from two independent experiments.

FIG. 12: GMMA purification from mutant strains (FIOW). GMMA yield was evaluated after purification and compared to wild type either normalized on culture volume (Volumetric Yield) or on optical density (Specific Yield). Data reported are average values from two independent experiments.

FIG. 13: GMMA protein profiles. GMMA purified from Ec BL21(DE3) wild type and mutants from two independent experiments (I: lanes 1-5; II: lanes: 6-10) were analysed in Coomassie stained SDS-PAGE. Five micrograms of total proteins were loaded for all samples but the ΔihfB #3 due to the very low concentration.

FIG. 14: Growth profiles of Ec mutants at 30° C. and 25° C. Growth curves were recorded monitoring OD at 600 nm.

FIG. 15: GMMA yields from mutant strains (FIOW) at 30° C. and 25° C. Fluorescence intensity was measured directly on cell-free supernatants of different mutants and compared to wild type either normalized on culture volume (Volumetric Yield) or on optical density (Specific Yield). Data reported are average values from two independent experiments.

FIG. 16: GMMA yields from rpsA and ihfB mutant strains in Moraxella catarrhalis.

FIG. 17: Negative staining electron microscopy of GMMA samples from rpsA and ihfB mutant strains in Moraxella catarrhalis.

FIG. 18: RpsA overexpression analysis. Total and soluble protein profiles were analyzed on Coomassie stained SDS-PAGE to evaluate RpsA FL and TR overexpression. Expected molecular weight was about 61 and 48 kDa respectively.

FIG. 19: GMMA yields from RpsA overexpressing strains (FIOW). Fluorescence intensity was measured directly on cell-free spent supernatants of different mutants and compared to wild type transformed with empty vector either normalized on culture volume (Volumetric Yield) or on final biomass (Specific Yield). Data reported are average values from three technical replicates.

FIG. 20: GMMA protein profiles. Protein profiles were analyzed on Coomassie stained SDS-PAGE to compare protein patterns.

FIG. 21: Purified GMMA yields from RpsA overexpressing strains (FIOW). Purified GMMA from different strains were quantified by FM4-64 staining and compared to wild type transformed with empty vector and normalized on final biomass (Specific Yield). Data reported are average values from three technical replicates.

### DETAILED DESCRIPTION OF THE INVENTION

The inventors have discovered that modifications of rpsA gene, rpsA operon and/or 30S ribosomal protein S1 can increase the nOMV released by a Gram-negative bacterial cell relative to an unmodified bacterial cell. By “unmodified bacterial cell” we mean or include an otherwise equivalent bacterial cell that does not comprise a modified rpsA gene, a modified rpsA operon and/or a modified 30S ribosomal protein S1.

Accordingly, a first aspect of the invention provides a genetically modified Gram-negative bacterial cell comprising a modified rpsA gene, a modified rpsA operon and/or a modified 30S ribosomal protein S1. By “modified” (also “modification”) we mean or include any biological material, including but not limited to genes, operons, proteins and cells, which is altered in any way whatsoever. A biological material may be modified chemically or genetically. Modifications may include any mutation, any alteration of the expression of a gene product (such as upregulation and downregulation) or a combination thereof (such as the alteration of the expression of a mutated gene product).

### The Bacterial Cell

The genetically modified Gram-negative bacterial cell may be of any family, such as species selected from the group consisting of Enterobacteriaceae, Neisseriaceae, Helicobacteraceae, Campylobacteraceae, Yersiniaceae, Vibrionaceae, Pasteurellaceae, Alcaligenaceae, Pseudomonadaceae and Moraxellaceae.

Alternatively or additionally, the genetically modified Gram-negative bacterial cell is a species of the genera selected from the group consisting of Escherichia, Shigella, Neisseria, Moraxella, Bordetella, Borrelia, Brucella, Chlamydia, Haemophilus, Legionella, Pseudomonas, Yersinia, Helicobacter, Salmonella, Vibrio, and the like. For example, the Gram-negative bacterial cell may be selected from the group consisting of Bordetella pertussis, Borrelia burgdorferi, Brucella melitensis, Brucella ovws, Chlamydia psittaci, Chlamydia trachomatis, Moraxella catarrhalis, Escherichia coli, Haemophilus influenzae (including non-typeable stains), Legionella pneumophila, Neisseria gonorrhoeae, Neisseria meningitidis, Neisseria lactamica, Pseudomonas aeruginosa, Yersinia enterocolitica, Helicobacter pylori, Salmonella enterica (including serovars typhi and typhimurium, as well as serovars paratyphi and enteritidis), Shigella (including S. dysenteriae, S. flexneri, S. boydii, S. sonnei), Vibrio cholerae, etc.

Alternatively or additionally, the Gram-negative bacterial cell is Bordetella pertussis, Neisseria (such as Neisseria meningitidis or Neisseria gonorrhoeae), Salmonella (such as Salmonella typhi or Salmonella typhimurium), Shigella (such as S. dysenteriae, S. flexneri, S. boydii or S. sonnei), Escherichia coli (including extraintestinal pathogenic strains), and Haemophilus influenzae (for example non-typeable Heamophilus influenzae or NtHI).

Alternatively or additionally, the genetically modified Gram-negative bacterial cell is a B. pertussis strain, such as a Tohama I strain. Alternatively or additionally, the genetically modified Gram-negative bacteria is a B. pertussis strain expressing a genetically detoxified pertussis toxoid, for example expressing the genetically detoxified pertussis toxoid PT 9K/129G. In particular, a B. pertussis strain that comprises a subunit 1 gene which has been modified to include the mutations R9K and E129G and that expresses the genetically detoxified pertussis toxoid PT-9K/129G may be used as described in WO/2020/094580, whose disclosure is incorporated by reference herein. Alternatively or additionally, the use of B. pertussis strains in which the ArnT gene has been knocked-out or deleted is also advantageous. OMVs derived from such strains comprise Lipid A that has a modified structure without glucosamine (GlcN) substitutions on the distal phosphate groups of the core structure. As a result, these OMVs exhibit a decreased level of TLR4 activation when compared to OMVs derived from strains with a functional ArnT gene as described in WO/2020/094580, whose disclosure is incorporated by reference herein.

Alternatively or additionally, the genetically modified Gram-negative bacterial cell is an N. meningitidis strain, such as a serogroup B strain. Alternatively or additionally, the N. meningitidis strain is of a serogroup other than B, such as one of: A, C, W135, Y or X. The strain may be of any serotype (e.g., 1, 2a, 2b, 4, 14, 15, 16, etc.), any serosubtype (e.g., P.1.1, P.1.2, P.1.3, P1.4, P.1.5, P.1.6, P.1.7, P.1.9, P.1.10, P.1.12, P.1.13, P.1.14, P.1.15, P.1.16, P.1.9, P.1.22a), and any immunotype (e.g., L1; L2; L3; L3,7; L3,7,9; L10; etc.) as described in Tondella M L, et al., J Clin Microbiol. 2000 September; 38(9):3323-8. Alternatively or additionally meningococci may be from any suitable lineage, including hyperinvasive and hypervirulent lineages, including the hypervirulent lineages selected from the group consisting of: subgroup I; subgroup III; subgroup IV-1; ET-5 complex; ET-37 complex; A4 cluster; lineage 3. Alternatively or additionally, the N. meningitidis strain is NZ98/254 (B:4:P1.7-2.4), or another strain with the P1.4 PorA serosubtype.

Alternatively or additionally, the genetically modified Gram-negative bacterial cell is an N. gonorrhoeae strain, such as any porBIa or porBIb strain. Alternatively or additionally, the N. gonorrhoeae strain is an FA1090 strain, or an F62 strain. Alternatively or additionally, the genetically modified Gram-negative bacterial cell is an E. coli strain, such as a BL21, a BL21(DE3), a C43 (DE3), and a DH5alpha strain. Alternatively or additionally, the genetically modified Gram-negative bacterial cell is not an E. coli strain, such as a MB3001, CSR603, JM105CAG18478, IQ646, ENSO, or ENSO-xTIR strain. Alternatively or additionally, the genetically modified Gram-negative bacterial cell is a P. aeruginosa strain, such as a PA01 strain. Alternatively or additionally, the genetically modified Gram-negative bacterial cell is an H. influenzae strain, such as an Hib ATCC 10211, Hib Rd_KW20 HI1220, an NTHi R2866, or an NTHi 86028NP strain.

Alternatively or additionally, the genetically modified Gram-negative bacterial cells are mutant strains which have been manipulated to enhance vesicles production, to express one or more desired antigen(s), such as one or more exogenous antigen(s), and/or to knockout or modify an undesired gene (e.g., one which encodes a toxin or which encodes an enzyme involved in generating a toxic product, such as endotoxin). For example, the genetically modified Gram-negative bacterial cell may be further genetically engineered by one or more processes selected from the following group: (a) a process of down-regulating expression of immunodominant variable or non-protective antigens, (b) a process of up-regulating expression of protective outer membrane protein (OMP) antigens, (c) a process of down-regulating a gene involved in rendering the lipid A portion of LPS toxic, (d) a process of up-regulating a gene involved in rendering the lipid A portion of LPS less toxic, and (e) a process of genetically modifying the Gram-negative bacterial cell to express a heterologous antigen.

The genetically modified Gram-negative bacterial cell of the invention may be capable of proliferation. By “capable of proliferation” we mean or include the capability to undergo cell division to generate progeny cells. The genetically modified Gram-negative bacterial cell may be capable of proliferation under standard conditions for the genus and/or species of the bacterial cell (see, for example, Peleg M and Corradini M G, Crit Rev Food Sci Nutr, 2011 December; 51(10):917-45 which is incorporated by reference herein) or other conditions that so permit, such as when cultured at a suitable temperature (e.g., 25° C., 30° C., 37° C., 42° C.), in aerobic or anaerobic environment, in media which provide nutrients. Culture media may be solid or liquid and preferably comprise salts and nutrient components (e.g., carbon sources, vitamins, co-factors). The culture media may contain serum. The culture medium preferably provides a suitable osmolarity. The culture media may be supplemented with antibiotics where appropriate for each strain, for example, it can be supplemented with streptomycin, kanamycin, nalidixic acid, chloramphenicol, and/or ampicillin. For examples, suitable growth conditions for B. pertussis Tohama I strain are Regan-Lowe Charcoal agar plates for three-five days at 37° C. or modified Stainer-Scholte medium (SS) supplemented with 0.4% (w/v) L-cysteine monohydrochloride, 0.1% (w/v) FeSO₄, 0.4% (w/v) ascorbic acid, 0.04% (w/v) nicotinic acid, 1% (w/v) reduced glutathione using 2 mL 96-deepwell plates for microscale cultures (600 μl), and 0.25-2 L baffled flasks with vented cup for small and large scale cultures (50-300 ml) at 37° C. in orbital shakers, agitating microplates at 350 rpm and flasks at 180 rpm. For example, suitable growth conditions for E. coli BL21(DE3) are Luria Bertani (LB) medium or HTMC medium in rotary shakers at 25° C., 30° C. and 37° C. and 180 rpm for 8 hours, 16 hours or until late stationary phase.

The growth of the genetically modified Gram-negative bacterial cell is determined according to biomass, turbidity and/or nutrient uptake. Methods of assessing bacterial growth and proliferation are known in the art (Peleg M and Corradini M G, Crit Rev Food Sci Nutr, 2011 December; 51(10):917-45), for example, the measurement of optical density (OD) at 600 nm, for assessing bacterial growth in liquid cultures, such as by measuring OD at 600 nm by Infinity 200 PRO spectrophotometer (TECAN), and colony forming units (CFU), for assessing bacterial growth in solid media.

Alternatively or additionally, the genetically modified Gram-negative bacterial cell is capable of generating a biomass which is at least 10% of the biomass of the unmodified bacterial cell grown in the same culture conditions, for example, 20%, 30%, 40%, 50%, 60%, 70%, 80%, 85%, 90%, 91%, 92%, 93%, 94%, 95%, 96%, 97%, 98%, 99% or 100% of the biomass of a culture of the unmodified cell grown in the same culture conditions. For example, biomass is measured when a culture reaches stationary phase. Alternatively or additionally, the genetically modified Gram-negative bacterial cell is capable of achieving a turbidity which is at least 10% of the turbidity of the unmodified bacterial cell grown in the same culture conditions, for example, 20%, 30%, 40%, 50%, 60%, 70%, 80%, 85%, 90%, 91%, 92%, 93%, 94%, 95%, 96%, 97%, 98%, 99% or 100% of the turbidity of a culture of the unmodified bacterial cell grown in the same culture conditions. For example, turbidity is measured when a culture reaches stationary phase. Turbidity may be measured by methods common in the art, such as by measuring optical density (OD) at 600 nm. For example, turbidity is measured by incubating a pre-inoculum of 5 ml medium (e.g., a suitable medium for each strain, such as LB) for 16-18 hours at 25° C. or 30° C. or for 8 hours at 37° C. at 180 rpm (pre-culture), diluting the pre-cultures 1:100 in 50 mL medium (e.g., a suitable medium for each strain, such as HTMC) in disposable baffled 250 mL flasks with vented cup, incubating at 25° C., 30° C. and 37° C., respectively, at 180 rpm shaking until stationary phase, and measuring the OD at 600 nm with an Infinity 200 PRO spectrophotometer (TECAN). Alternatively or additionally, a culture of the Gram-negative bacterial cell is capable of achieving a nutrient uptake which is at least 10% of the nutrient uptake of a culture of the unmodified bacterial cell grown in the same culture conditions, for example, 20%, 30%, 40%, 50%, 60%, 70%, 80%, 85%, 90%, 91%, 92%, 93%, 94%, 95%, 96%, 97%, 98%, 99% or 100% of the nutrient uptake of a culture of the unmodified bacterial cell grown in the same culture conditions. Alternatively or additionally, the genetically modified Gram-negative bacterial cell is capable of reaching stationary phase earlier than the unmodified bacterial cell grown in the same culture conditions. Alternatively or additionally, the genetically modified Gram-negative bacterial cell is capable of reaching stationary phase at the same time of the unmodified bacterial cell grown in the same culture conditions. Alternatively or additionally, the genetically modified Gram-negative bacterial cell is capable of reaching stationary phase no later than 120 hours after the unmodified bacterial cell grown in the same culture conditions, for example, no later than 1, 2, 3, 4, 5, 6, 7, 8, 9, 10, 12, 15, 20, 25, 30, 40, 50, 60, 70, 80, 90, 100, or 110 hours after the unmodified bacterial cell grown in the same culture conditions. For example, the capability of reaching stationary phase is assessed by turbidity measurement. For example, turbidity is measured by incubating a pre-inoculum of 5 ml medium (e.g., a suitable medium for each strain, such as LB) for 16-18 hours at 25° C. or 30° C. or for 8 hours at 37° C. at 180 rpm (pre-culture), diluting the pre-cultures 1:100 in 50 mL medium (e.g., a suitable medium for each strain, such as HTMC) in disposable baffled 250 mL flasks with vented cup, incubating at 25° C., 30° C. and 37° C., respectively, at 180 rpm shaking until stationary phase, and measuring the OD at 600 nm with an Infinity 200 PRO spectrophotometer (TECAN).

Alternatively or additionally, the genetically modified Gram-negative bacterial cell comprises a modified rpsA gene, wherein the modified rpsA gene(s) comprises one or more mutation(s) relative to a wild-type rpsA gene, wherein the one or more mutation(s) may be located within the coding region of the rpsA gene and/or within the non-coding region of the rpsA gene. Alternatively or additionally, the one or more mutation(s) may alter rpsA gene product by means of altering its expression and/or coding for a mutated protein. By “mutation” we mean or include an alteration in the nucleotide sequence of a polynucleotide and/or an alteration in the amino acid sequence of a polypeptide. Mutations of a polynucleotide may be selected from the group consisting of a deletion, such as the deletion of one or more nucleotides; an insertion, such as the insertion of one or more nucleotides; the one or more nucleotides which are inserted may be exogenous, such as nucleotides originating outside a wild type bacterial cell, or endogenous, such as a copy of a portion of a gene of a wild type bacterial cell (for example a duplication); and/or a substitution, such as the substitution of one or more nucleotides. Deletion, insertion and substitution mutations of a polynucleotide may suitably result in a missense or nonsense mutation in the polypeptide encoded by the polynucleotide harbouring said mutations. Deletion and insertion mutations of a polynucleotide may suitably result in a frameshift mutation in the polypeptide encoded by the polynucleotide harbouring said mutations. Mutations of a polypeptide may be selected from the group consisting of a deletion, such as the deletion of one or more amino acids; an insertion, such as the insertion of one or more amino acids and/or the insertion of an exogenous sequence; and/or a substitution, such as the substitution of one or more amino acids. Amino acid substitutions may or may not be conservative, i.e., the replacement of one amino acid with another amino acid which has a related side chain. Mutations of a polypeptide may suitably modify the biological activity of the polypeptide. By “post-translational modification” we mean or include covalent modification of a polypeptide following its biosynthesis, including, but not limited to, phosphorylation, glycosylation, acylation, acetylation, lipidation, disulphide bond formation, and proteolytic cleavage.

Alternatively or additionally, the one or more mutation(s) may comprise or consist of the insertion of one or more nucleotide(s), wherein the one or more nucleotide(s) inserted is selected from the group consisting of a transposon mobile element, a selection marker, for example, an antibiotic resistance cassette, a gene encoding a fluorescent protein, or a gene encoding an antitoxin, and/or a fragment of the rpsA gene. By “fragment of the rpsA gene” we mean or include a nucleotide sequence of the rpsA gene consisting of at least 10 nucleotides, for example at least 12, 15, 20, 25, 30, 40, 50, 60, 70, 80, 90, 100, 150, 200 nucleotides, wherein the rpsA gene is identical or is not identical to the rpsA gene encoded by the unmodified bacterial cell and/or by the wild type bacterial cell, for example wherein the rpsA gene is an rpsA gene encoded by a Gram-negative bacterial cell of a different species relative to the unmodified bacterial cell. By “wild type bacterial cell” we mean or include a bacterial cell that has not been modified either chemically or genetically in any way whatsoever.

Alternatively or additionally, the one or more mutation(s) was made using, or is obtained or obtainable by a method selected from the group consisting of site directed mutagenesis, a recombinase-mediated method, a λ-red recombinase-mediated method, a prophage-based approach, a mobile group II introns knock out, transposon-mediated genome editing and CRISPR-Cas genome editing. Alternatively or additionally, the insertion of a transposon mobile element is made using, or is obtained or obtainable by transposon-mediated genome editing. Alternatively or additionally, the transposon mobile element is selected from the group consisting of a Tn5 transposon, a Tn10 transposon, a Tn3 transposon, a Tn7 transposon, an IS5376 transposon, a bacteriophage MuA transposon, an IS200/IS605 transposon, and an IS91 transposon. Alternatively or additionally, the transposon mobile element is a Tn5 transposon.

Alternatively or additionally, the modified rpsA gene(s) comprises or consists of a nucleotide sequence having at least 60% sequence identity to a wild-type rpsA, for example, at least 70%, 80%, 85%, 90%, 91%, 92%, 93%, 94%, 95%, 96%, 97%, 98%, 99%, or 100% sequence identity to a wild-type rpsA. The wild-type rpsA may be selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 55 and SEQ ID NO: 60.

Alternatively or additionally, the one or more mutation(s) relative to a wild-type rpsA gene occur in a designated nucleotide sequence. Alternatively or additionally, the one or more mutation(s) relative to a wild type rpsA gene does not occur in a designated nucleotide sequence, for example wherein the Gram-negative bacterial cell is E. coli. The designated nucleotide sequence may be defined based on the nucleotides encoding 30S ribosomal protein S1 “R” domains. By “R” domain we mean or include an oligonucleotide/oligosaccharide-binding (OB) motif of approximatively 70 amino acids, which is folded into a five-stranded antiparallel P barrel. Multiple “R” domains can be arranged in a consecutive fashion within 30S ribosomal protein S1, which can comprise between one and six domains (Machulin A V et al., PloS one, 2019 Aug. 22; 14(8):e0221370). “R” domains are connected by strands of 10-15 amino acids. “R” domains may be designated using suitable sequence alignment tools. The designated sequence may comprise or consist of from 1 to 10 nucleotides upstream of the 3′-end of the R3 domain relative to the wild-type rpsA gene, for example, from 11 to 20, from 21 to 50, or from 51 and 100 nucleotides upstream of the 3′-end of the R3 domain relative to the wild-type rpsA gene. Alternatively or additionally, the designated sequence may comprise or consist of from 1 to 100 nucleotides upstream of the 3′-end of the R3 domain relative to the wild-type rpsA gene, for example, from 10 to 50, or from 20 to 30 nucleotides upstream of the 3′-end of the R3 domain relative to the wild-type rpsA gene. Alternatively or additionally, the designated sequence may comprise or consist of from 1% to 10% of the nucleotides relative to the wild-type rpsA gene, for example, from 11% to 20%, from 21% to 30% relative the nucleotides of the wild-type rpsA gene, wherein the designated region is located between the 5′-end relative to the wild-type rpsA gene and the 3′-end of the R3 domain relative to the wild-type rpsA gene. Alternatively or additionally, the designated sequence may comprise or consist of from 1% to 30% of the nucleotides relative to the wild-type rpsA gene, for example, from 5% to 25%, from 10% to 20% of the nucleotides relative to the wild-type rpsA gene, wherein the designated region is located between the 5′-end relative the wild-type RpsA gene and the 3′-end of the R3 domain relative to the wild-type rpsA gene. Alternatively or additionally, the designated sequence may comprise or consist of from 1 and 10 nucleotides downstream of the 3′-end of the R3 domain relative to the wild-type rpsA gene, for example, from 11 and 20, from 21 to 50, or from 51 to 100 nucleotides downstream of the 3′-end of the R3 domain relative to wild-type rpsA gene. Alternatively or additionally, the designated sequence may comprise or consist of from 1 to 100 nucleotides downstream of the 3′-end of the R3 domain relative to the wild-type rpsA gene, for example, from 10 to 50, or from 20 to 30, nucleotides downstream of the 3′-end of the R3 domain relative to the wild-type rpsA gene. Alternatively or additionally, the designated sequence may comprise or consist of from 1 and 10 nucleotides downstream of the 5′-end of the R4 domain relative to the wild-type rpsA gene, for example, from 11 and 20, from 21 to 50, or from 51 to 100 nucleotides downstream of the 5′-end of the R4 domain relative to wild-type rpsA gene. Alternatively or additionally, the designated sequence may comprise or consist of from 1 to 100 nucleotides downstream of the 5′-end of the R4 domain relative to the wild-type rpsA gene, for example, from 10 to 50, or from 20 to 30, nucleotides downstream of the 5′-end of the R4 domain relative to the wild-type rpsA gene. Alternatively or additionally, the designated sequence may comprise or consist of from 1 and 10 nucleotides downstream of the 3′-end of the R4 domain relative to the wild-type rpsA gene, for example, from 11 and 20, from 21 to 50, or from 51 to 100 nucleotides downstream of the 3′-end of the R4 domain relative to wild-type rpsA gene. Alternatively or additionally, the designated sequence may comprise or consist of from 1 to 100 nucleotides downstream of the 3′-end of the R4 domain relative to the wild-type rpsA gene, for example, from 10 to 50, or from 20 to 30, nucleotides downstream of the 3′-end of the R4 domain relative to the wild-type rpsA gene. Alternatively or additionally, the designated sequence may comprise or consist of from 1% to 10% of the nucleotides relative to the wild-type rpsA gene, for example, from 11% to 20%, from 21% to 30% of the nucleotides relative to the wild-type rpsA gene, wherein the designated region is located between the 3′-end relative to the R3 domain of the wild-type rpsA gene and the 3′-end relative to the wild-type rpsA gene. Alternatively or additionally, the designated sequence may comprise or consist of from 1% to 30%, of the nucleotides relative to the wild-type rpsA gene, for example, from 5% to 25%, from 10% to 20% of the nucleotides relative to the wild-type rpsA gene, wherein the designated region is located between the 3′-end relative to the R3 domain of the wild-type rpsA gene and the 3′-end relative to the wild-type rpsA gene. Alternatively or additionally, the designated sequence may comprise or consist of from 1 to 5 nucleotides upstream of the 3′-end relative to the wild-type rpsA gene, for example, from 6 to 10, from 11 to 20, from 21 to 30, from 31 to 40, from 41 to 50, from 51 to 100, from 101 to 150, from 151 to 200, from 201 to 250, from 251 to 300, from 301 to 350, from 351 to 400, from 401 to 450, from 451 to 500 nucleotides upstream of the 3′-end relative to the wild-type rpsA gene. Alternatively or additionally, the designated sequence may comprise or consist of from 1 to 500 nucleotides upstream the 3′-end relative to the wild-type rpsA gene, for example, from 50 to 450, from 100 to 400, from 150 to 380, from 200 to 378, or from 250 to 376 nucleotides upstream the 3′-end relative to the wild-type rpsA gene. By “upstream of the 3-end” relative to a designated nucleotide sequence (e.g. the wild-type rpsA gene or a domain, such as domain 1, domain 2, R1, R2, R3 or R4), we mean or include the nucleotide up to and including the last nucleotide of the designated sequence. By “downstream of the 3-end” relative to a designated nucleotide sequence (e.g. the wild-type rpsA gene or a domain, such as domain 1, domain 2, R1, R2, R3 or R4), we mean or include the nucleotide following the last nucleotide of the designated sequence. By “upstream of the 5-end” relative to a designated nucleotide sequence (e.g. the wild-type rpsA gene or a domain, such as domain 1, domain 2, R1, R2, R3 or R4), we mean or include the nucleotide up to and including the first nucleotide of the designated sequence. By “downstream of the 5-end” relative to a designated nucleotide sequence (e.g. the wild-type rpsA gene or a domain, such as domain 1, domain 2, R1, R2, R3 or R4), we mean or include the nucleotide following the first nucleotide of the designated sequence.

Alternatively or additionally, the one or more mutation(s) relative to a wild-type rpsA gene comprises one or more mutation(s) in a region corresponding to a region encoding the R4 domain of the B. pertussis 30S ribosomal protein S1 protein and/or a region encoding the portion of the B. pertussis 30S ribosomal S1 protein between the R3 and R4 domains.

The one or more mutation(s) are in a region “corresponding” to a specific domain, portion or position if the one or more mutation(s) is in an amino acid or nucleotide sequence that aligns to the specific domain, portion or position. Whether or not an amino acid or nucleotide sequence aligns to a specific domain, portion or position may be determined using standard sequence alignment tools. Optionally, the R4 domain of B. pertussis is encoded by a nucleotide sequence of SEQ ID NO: 24. Optionally, the portion of the B. pertussis 30S ribosomal protein S1 between the R3 and R4 domains is encoded by a nucleotide sequence of nucleotides 1348 to 1383 of SEQ ID NO: 1.

Alternatively or additionally, the one or more mutation(s) relative to a wild-type rpsA gene comprises one or more mutation(s) that causes an increased release of nOMVs compared to an unmodified Gram-negative bacterial cell. A mutation causes an increased release of nOMVs if, when it is introduced into a gene in a Gram-negative bacterial cell, the Gram-negative bacterial cell releases more nOMVs compared to an equivalent Gram-negative bacterial cell that is identical but for the mutation. The level of nOMV release may be evaluated by a parameter selected from the group consisting of the absolute amount of nOMVs, the concentration of nOMVs in the supernatant (after spinning), the absolute amount of nOMVs relative to the number of bacterial cells, and the concentration of nOMVs relative to the number of bacterial cell, as described in more detail below.

Alternatively or additionally, the one or more mutation(s) causes an at least 1.2-fold, 1.4-fold, 1.6-fold, 1.8-fold, 2.0-fold or 2.5-fold increased release of nOMVs compared to an unmodified Gram-negative bacterial cell. Alternatively or additionally, the one or more mutation(s) causes an at least 2.0-fold increase in release of nOMVs compared to an unmodified Gram-negative bacterial cell. Alternatively or additionally, the one or more mutation(s) comprises mutation(s) that change the amino acid sequence of the encoded 30S ribosomal protein S1 protein. Alternatively or additionally, the one or more mutation(s) comprises mutations that change the encoded amino acid sequence of a region of the 30S ribosomal protein S1 protein that corresponds to the R4 domain of B. pertussis. Alternatively or additionally, the one or more mutation(s) comprises mutations that change the encoded 30S ribosomal protein S1 protein to modify and/or delete at least 5, at least 10, at least 25, at least 50, at least 75, at least 100, at least 110, at least 125, between 5 and 150, between 25 and 130, between 100 and 130, or between 125 and 130 amino acids. Alternatively or additionally, the one or more mutation(s) comprises mutations that change the encoded 30S ribosomal protein S1 protein to delete at least 5, at least 10, at least 25, at least 50, at least 75, at least 100, at least 110, at least 125, between 5 and 150, between 25 and 130, between 100 and 130, or between 125 and 130 amino acids. Alternatively or additionally, the modified rpsA gene encodes a modified 30S ribosomal protein S1 protein as described herein. Alternatively or additionally, the one or more mutation(s) comprises mutation or deletion of at least 15, at least 30, at least 45, at least 60, or at least 75 nucleotides of the region corresponding to nucleotides 1655 to 1731 of the B. pertussis rpsA gene. Optionally, the B. pertussis rpsA gene is a gene having a nucleotide sequence of SEQ ID NO: 1. Alternatively or additionally, the one or more mutation(s) comprises mutation or deletion of at least 60, at least 90, at least 150, at least 225, or at least 300 nucleotides of the region corresponding to nucleotides 1424 to 1731 of the B. pertussis rpsA gene. Alternatively or additionally, the one or more mutation(s) comprises mutation or deletion of at least 60, at least 90, at least 150, at least 225, at least 300, or at least 360 nucleotides of the region corresponding to nucleotides 1355 to 1731 of the B. pertussis rpsA gene.

Alternatively or additionally, the one or more mutation(s) alter the rpsA gene product by (a) altering its expression and/or (b) coding for a mutated 30S ribosomal protein S1.

Alternatively or additionally, the genetically modified Gram-negative bacterial cell comprises a modified rpsA operon. Alternatively or additionally, the modified rpsA operon(s) comprises or consists of an nucleotide sequence having at least 60% sequence identity to a wild-type rpsA operon, for example, at least 70%, 80%, 85%, 90%, 91%, 92%, 93%, 94%, 95%, 96%, 97%, 98%, 99%, or 100% sequence identity to a wild-type rpsA operon. The wild-type rpsA operon may be selected from the group consisting of SEQ ID NO: 7 SEQ ID NO: 8, SEQ ID NO: 9, SEQ ID NO: 10, SEQ ID NO: 11, and SEQ ID NO: 12.

Alternatively or additionally, the genetically modified Gram-negative bacterial cell comprises a modified 30S ribosomal protein S1. Alternatively or additionally, the modified 30S ribosomal protein(s) S1 comprises one or more mutation(s) relative to a wild-type 30S ribosomal protein S1 and/or comprises one or more post-translational modification(s) relative to a wild-type 30S ribosomal protein S1.

Alternatively or additionally, the one more mutation(s) relative to a wild-type 30S ribosomal protein S1 comprises one or more mutation(s) in a region corresponding to the R4 domain of B. pertussis 30S ribosomal protein S1 and/or a region corresponding to the portion of the B. pertussis 30S ribosomal protein S1 between the R3 and R4 domains. Optionally, the R4 domain of B. pertussis is encoded by a nucleotide sequence of SEQ ID NO: 24. Optionally, the portion of the B. pertussis 30S ribosomal protein S1 between the R3 and R4 domains is encoded by a nucleotide sequence of nucleotides 1348 to 1383 of SEQ ID NO: 1.

Alternatively or additionally, the one or more mutation(s) relative to a wild-type 30S ribosomal protein S1 comprises mutation or deletion of at least 5, at least 10, at least 25, at least 50, at least 75, at least 100, at least 110, at least 125, between 5 and 150, between 25 and 130, between 100 and 130, or between 125 and 130 amino acids. Alternatively or additionally, the one or more mutation(s) relative to a wild-type 30S ribosomal protein S1 comprises mutation or deletion of at least 5, at least 10, at least 15, at least 20, or at least 25, amino acids of the region corresponding to amino acids 550 to 576 of the B. pertussis 30S ribosomal protein S1. Alternatively or additionally, the one or more mutation(s) relative to a wild-type 30S ribosomal protein S1 comprises mutation or deletion of at least 20, at least 30, at least 50, at least 75, or at least 100 amino acids of the region corresponding to amino acids 473 to 576 of the B. pertussis 30S ribosomal protein S1. Alternatively or additionally, the one or more mutation(s) relative to a wild-type 30S ribosomal protein S1 comprises mutation or deletion of at least 20, at least 30, at least 50, at least 100, or at least 120 amino acids of the region corresponding to amino acids 450 to 576 of the B. pertussis 30S ribosomal protein S1. Alternatively or additionally, the one or more mutation(s) relative to a wild-type 30S ribosomal protein S1 comprises truncation of at least 20, at least 30, at least 50, at least 100, or at least 120 consecutive amino acids from the C-terminal end of the 30S ribosomal protein S1. Alternatively or additionally, the one or more mutation(s) relative to a wild-type 30S ribosomal protein S1 comprises mutation or deletion of amino acids corresponding to amino acids 560 to 576, 552 to 576, 500 to 576 485 to 576, 460 to 576, or 453 to 576 of the B. pertussis 30S ribosomal protein S1. Optionally, the B. pertussis 30S ribosomal protein S1 has an amino acid sequence of SEQ ID NO: 13.

Alternatively or additionally, the modified 30S ribosomal protein(s) S1 is downregulated relative to the 30S ribosomal protein S1 of an unmodified bacterial cell. By “downregulation” or “downregulated” we mean or include expression of a gene product at a level lower than that expressed by an unmodified bacterial. By “lower level” we mean or include any lower level with statistical significance with respect to a control, for example, a level that is at least 2-fold lower with respect to a control. For example, downregulation can be achieved by genetic modification(s) which include altering or modifying regulatory sequences or sites associated with expression of a gene of interest (e.g., modifying promoters or regulatory sequences), modifying the chromosomal location of a gene of interest, altering nucleic acid sequences adjacent to a gene of interest such as a ribosome binding site, modifying proteins (e.g., regulatory proteins, suppressors, enhancers, transcriptional activators) involved in transcription of a gene of interest and/or translation of a gene product, or any other conventional means of deregulating expression of a gene of interest which are known in the art (including but not limited to use of antisense nucleic acid molecules). Downregulation is suitably evaluated with the method disclosed in Jocelyn E. Krebs, Elliott S. Goldstein, Stephen T. Kilpatrick, Lewin's Genes XII, 2017, whose disclosure is incorporated herein by reference.

Alternatively or additionally, the genetically modified Gram-negative bacterial cell comprises a modified 30S ribosomal protein S1, wherein the modified 30S ribosomal protein(s) S1 is truncated relative to a wild-type 30S ribosomal protein S1. Alternatively or additionally, the modified 30S ribosomal protein(s) S1 is not truncated relative to a wild-type 30S ribosomal protein S1, for example, wherein the Gram-negative bacterial cell is E. coli. By “truncated” we mean or include a variant of a wild-type polypeptide which lacks at least a fragment of the wild-type polypeptide. Such fragment may be located at the N-terminus or at the C-terminus of the wild-type polypeptide or may be located between the N-terminus and the C-terminus of the polypeptide. Such fragment may be at least 7 amino acid long, for example, at least 8, 9, 10, 15, 20, 25, 30, 35, 40, 45, 50, 60, 70, 80, 100, 150, 200 amino acid long.

Alternatively or additionally, the modified 30S ribosomal protein(s) S1 is encoded by a modified rpsA gene(s). Alternatively or additionally, the modified rpsA gene(s) is chromosomic, or extra-chromosomic, for example, the modified rpsA gene(s) is encoded by a plasmid or by a cosmid.

Alternatively or additionally, the modified 30S ribosomal protein(s) S1 comprises or consists of an amino acid sequence having at least 60% sequence identity to a wild-type S1, for example, at least 70%, 80%, 85%, 90%, 91%, 92%, 93%, 94%, 95%, 96%, 97%, 98%, 99%, or 100% sequence identity to a wild-type 30S ribosomal protein S1. The wild-type 30S ribosomal protein S1 may be selected from the group consisting of SEQ ID NO: 13, SEQ ID NO: 14, SEQ ID NO: 15, SEQ ID NO: 16, SEQ ID NO: 17, SEQ ID NO: 18 and SEQ ID NO: 57. Alternatively or additionally, the genetically modified Gram-negative bacterial cell comprises a modified 30S ribosomal protein S1, wherein the modified 30S ribosomal protein(s) S1 comprises or consists of an amino acid sequence having at least 90%, at least 95%, at least 98%, at least 99%, or 100% identity to a fragment of a wild-type 30S ribosomal protein S1 corresponding to amino acids 1 to 452 of the B. pertussis 30S ribosomal protein S1. Alternatively or additionally, the genetically modified Gram-negative bacterial cell comprises a modified 30S ribosomal protein S1, wherein the modified 30S ribosomal protein(s) S1 comprises or consists of an amino acid sequence having at least at least 98%, at least 99%, or 100% identity to a fragment of a wild-type 30S ribosomal protein S1 corresponding to amino acids 1 to 453 of the B. pertussis 30S ribosomal protein S1.

Alternatively or additionally, the modified 30S ribosomal protein(s) S1 comprises or consists of “R” domains selected from the group consisting of an R1 domain or a fragment thereof, an R2 domain or a fragment thereof, an R3 domain or a fragment thereof and an R4 domain or a fragment thereof; an R1 domain or a fragment thereof, an R2 domain or a fragment thereof, and an R3 domain or a fragment thereof; an R2 domain or a fragment thereof, an R3 domain or a fragment thereof, and an R4 domain or a fragment thereof; an R2 domain or a fragment thereof, and an R3 domain or a fragment thereof; an R3 domain or a fragment thereof, and an R4 domain or a fragment thereof; and an R3 domain or a fragment thereof.

Alternatively or additionally, the 30S ribosomal protein S1 may consist of 6 “R” domains. Such domains are designated as “domain 1”, “domain 2”, “R1”, “R2”, “R3”, and “R4” from the N-terminus to the C-terminus. Domain 1 and 2 are involved in protein-ribosome and protein-protein interactions. “R” domains may have RNA binding property, which can be specific for single-stranded regions (Bycroft M et al, Cell, 1997 Jan. 24; 88(2):235-42; Duval M et al., PloS Biol, 2013 December; 11(12):e1001731). Also, C-terminal domains of 30S ribosomal protein S1 can bind RNA fragments with conserved residues in the RNA binding region (Fan Y et al, Biochem and Biophys Res Commun, 2017 May 27; 487(2):268-273) and the deletion of the R4 domain is described as not lethal in E. coli (Schnier J et al., J Biol Chem, 1986 Sep. 5; 261(25):11866-71). The skilled person can assess which “R” domains of a 30S ribosomal protein S1 consisting of 1, 2, 3, 4, 5 or more “R” domains correspond to “domain 1”, “domain 2”, “R1”, “R2”, “R3”, and “R4” of a six-domain 30S ribosomal protein S1 using alignment tools known in the art (Machulin A V et al., PloS one, 2019 Aug. 22; 14(8):e0221370).

Alternatively or additionally, domain 1 is encoded by the nucleotide sequence of SEQ ID NO: 19 or by a variant thereof. Alternatively or additionally, domain 2 is encoded by the nucleotide sequence of SEQ ID NO: 20 or by a variant thereof. Alternatively or additionally, R1 is encoded by the nucleotide sequence of SEQ ID NO: 21 or by a variant thereof. Alternatively or additionally, R2 is encoded by the nucleotide sequence of SEQ ID NO: 22 or by a variant thereof. Alternatively or additionally, R3 is encoded by the nucleotide sequence of SEQ ID NO: 23 or by a variant thereof. Alternatively or additionally, R4 is encoded by the nucleotide sequence of SEQ ID NO: 24 or by a variant thereof. By “variant” we mean or include a sequence having at least 60% sequence identity to a given sequence, for example, at least 70%, 80%, 85%, 90%, 91%, 92%, 93%, 94%, 95%, 96%, 97%, 98%, 99%, or 100% sequence identity to a given sequence. By “fragment of an R domain” we mean of include a nucleotide sequence which is at least 9 nucleotides long, for example at least 12, 15, 18, 21, 24, 30, 36, 42, 48, 54, 60, 72, 81, 90, 99, 120, 150, 180, 210 nucleotides long and/or a polypeptide sequence which is at least 3 amino acids long, for example at least 4, 5, 6, 7, 8, 10, 12, 14, 16, 18, 20, 22, 27, 30, 33, 40, 50, 60, 70 nucleotides long.

Alternatively or additionally, the modified 30S ribosomal protein(s) S1 may comprise or consist of an amino acid sequence having an R1 domain, when present, with least 60% sequence identity to a wild-type S1, for example, at least 70%, 80%, 85%, 90%, 91%, 92%, 93%, 94%, 95%, 96%, 97%, 98%, 99%, or 100% sequence identity to a wild-type R1 domain. Alternatively or additionally, the modified S1 may comprise or consist of an amino acid sequence having an R2 domain, when present, with least 60% sequence identity to a wild-type S1, for example, at least 70%, 80%, 85%, 90%, 92%, 95%, 96%, 97%, 98%, 99%, or 100% sequence identity to a wild-type R2 domain. Alternatively or additionally, the modified S1 may comprise or consist of an amino acid sequence having an R3 domain with least 60% sequence identity to a wild-type S1, for example, at least 70%, 80%, 85%, 90%, 92%, 95%, 96%, 97%, 98%, 99%, or 100% sequence identity to a wild-type R3 domain. Alternatively or additionally, the modified S1 may comprise or consist of an amino acid sequence having an R4 domain, when present, with least 40% sequence identity to a wild-type S1, for example, at least 50%, 60%, 70%, 80%, 85%, 90%, 91%, 92%, 93%, 94%, 95%, 96%, 97%, 98%, 99%, or 100% sequence identity to a wild-type R4 domain.

Alternatively or additionally, the modified 30S ribosomal protein(s) S1 may comprise an amino acid sequence upstream of the first and/or the last amino acid of the R1, R2, R3 and/or R4 domain, when present, having at least 60%, 70%, 80%, 85%, 90%, 91%, 92%, 93%, 94%, 95%, 96%, 97%, 98%, 99%, or 100% sequence identity to the amino acid sequence upstream of the first and/or the last residue of the R1, R2, R3 and/or R4 domain of a wild-type 30S ribosomal protein S1.

Alternatively or additionally, the modified 30S ribosomal protein S1 was modified using, or is obtained or obtainable by genome editing, gene silencing, fragmenting the RNA post-transcriptionally and/or post-translational modification.

Alternatively or additionally, the genetically modified Gram-negative bacterial cell comprises a modified ihfB gene and/or a modified Integration host factor (IHF) protein.

Alternatively or additionally, the modified ihfB gene(s) comprises one or more mutation(s) relative to a wild-type ihfB gene, wherein the one or more mutation(s) are located within the coding region of the ihfB gene and/or within the non-coding region of the ihfB gene. Alternatively or additionally, the one or more mutation(s) may alter ihfB gene product by means of altering its expression and/or coding for a mutated protein. Alternatively or additionally, the modified ihfB gene(s) is knocked-out relative to a wild-type ihfB gene. Alternatively or additionally, the modified ihfB gene(s) comprises or consists of an amino acid sequence having at least 60% sequence identity to a wild-type ihfB gene, for example, at least 70%, 80%, 85%, 90%, 91%, 92%, 93%, 94%, 95%, 96%, 97%, 98%, 99%, or 100% sequence identity to a wild-type ihfB gene. The wild-type ihfB gene may be selected from the group consisting of SEQ ID NO: 25, SEQ ID NO: 26, SEQ ID NO: 27, SEQ ID NO: 28, SEQ ID NO: 29, and SEQ ID NO: 30.

Alternatively or additionally, the one or more mutation(s) relative to a wild-type IHF protein comprises a deletion of at least 20, at least 30, at least 40, at least 50, at least 60, at least 70, at least 80, at least 90, at least 100, at least 110, between 50 and 119, or between 80 and 119 amino acids. Alternatively or additionally, the one or more mutation(s) relative to a wild-type IHF protein comprises a deletion of at least 20, at least 30, at least 40, at least 50, at least 60, at least 70, at least 80, at least 100, at least 110, between 50 and 119, or between 80 and 119 contiguous amino acids. Alternatively or additionally, the one or more mutation(s) relative to a wild-type IHF protein causes an increased release of nOMVs compared to an unmodified Gram-negative bacterial cell. A mutation causes an increased release of nOMVs if, when it is introduced into a gene in a Gram-negative bacterial cell, the Gram-negative bacterial cell releases more nOMVs compared to an equivalent Gram-negative bacterial cell that is identical but for the mutation. The level of nOMV release may be evaluated by a parameter selected from the group consisting of the absolute amount of nOMVs, the concentration of nOMVs in the supernatant (after spinning), the absolute amount of nOMVs relative to the number of bacterial cells, and the concentration of nOMVs relative to the number of bacterial cell, as described in more detail below.

Alternatively or additionally, the one or more mutation(s) relative to a wild-type IHF protein causes an at least 1.2-fold, at least 1.4-fold, at least 1.6-fold, at least 1.8-fold, at least 2.0-fold, or at least 2.5-fold increased release of nOMVs compared to an unmodified Gram-negative bacterial cell. Alternatively or additionally, the Gram-negative bacterial cell is a species of bacteria which naturally co-transcribes an ihfB gene and an rpsA gene.

Alternatively or additionally, the modified IHF protein(s) comprises one or more mutation(s) relative to a wild-type IHF protein. Alternatively or additionally, the modified IHF protein(s) comprises one or more post-translational modification(s) relative to a wild-type IHF protein. Alternatively or additionally, the modified IHF protein(s) comprises or consists of an amino acid sequence having at least 60% sequence identity to a wild-type IHF protein, for example, at least 70%, 80%, 85%, 90%, 91%, 92%, 93%, 94%, 95%, 96%, 97%, 98%, 99%, or 100% sequence identity to a wild-type IHF protein. The wild-type IHF protein may be selected from the group consisting of SEQ ID NO: 31, SEQ ID NO: 32, SEQ ID NO: 33, SEQ ID NO: 34, SEQ ID NO: 35, SEQ ID NO: 36 and SEQ ID NO: 62.

Alternatively or additionally, the modified IHF protein(s) is downregulated relative to the IHF protein of an unmodified bacterial cell. Alternatively or additionally, the modified IHF protein(s) is encoded by the modified ihfB gene(s).

Alternatively or additionally, the genetically modified Gram-negative bacterial cell comprises a wild-type rpsA gene. Alternatively or additionally, the genetically modified Gram-negative bacterial cell comprises a wild-type rpsA gene and does not comprise a modified rpsA gene but expresses a modified 30S ribosomal protein S1 (for example due to post-transcriptional modifications or due to modifications of the rpsA operon). Alternatively or additionally, the genetically modified Gram-negative bacterial cell comprises a wild-type rpsA gene and a modified rpsA gene. Alternatively or additionally, the genetically modified Gram-negative bacterial cell comprises a wild-type 30S ribosomal protein S1 and a modified 30S ribosomal protein S1. Alternatively or additionally, the genetically modified Gram-negative bacterial cell does not comprise a wild-type rpsA gene. Alternatively or additionally, the genetically modified Gram-negative bacterial cell does not comprise a wild-type 30S ribosomal protein S1.

Alternatively or additionally, the modified rpsA gene comprises or consists of a nucleotide sequences having at least 60% sequence identity to SEQ ID NO: 37 or SEQ ID NO: 56, for example, at least 70%, 80%, 85%, 90%, 92%, 95%, 96%, 97%, 98%, 99%, or 100% sequence identity to SEQ ID NO: 37 or SEQ ID NO: 56.

Alternatively or additionally, the modified rpsA operon comprises or consists of a nucleotide sequence having at least 60% sequence identity to SEQ ID NO: 38, SEQ ID NO: 51 or SEQ ID NO: 59, for example, at least 70%, 80%, 85%, 90%, 91%, 92%, 93%, 94%, 95%, 96%, 97%, 98%, 99%, or 100% sequence identity to SEQ ID NO: 38, SEQ ID NO: 51 or SEQ ID NO: 59.

Alternatively or additionally, the modified 30S ribosomal protein S1 comprises or consists of an amino acid sequence having at least 60% sequence identity to a polypeptide selected from the group consisting of SEQ ID NO: 39, SEQ ID NO: 40, SEQ ID NO: 41, and SEQ ID NO: 53 for example, at least 70%, 80%, 85%, 90%, 91%, 92%, 93%, 94%, 95%, 96%, 97%, 98%, 99%, or 100% sequence identity to a polypeptide selected from the group consisting of SEQ ID NO: 39, SEQ ID NO: 40, SEQ ID NO: 41, and SEQ ID NO: 53.

Alternatively or additionally, the modified rpsA operon comprises or consists of a nucleotide sequence having at least 60% sequence identity to SEQ ID NO: 42 or SEQ ID NO: 43, for example, at least 70%, 80%, 85%, 90%, 91%, 92%, 93%, 94%, 95%, 96%, 97%, 98%, 99%, or 100% sequence identity to SEQ ID NO: 42 or SEQ ID NO: 43.

Alternatively or additionally, the modified 30S ribosomal protein S1 comprises or consists of an amino acid sequence having at least 60% sequence identity to SEQ ID NO: 44 or SEQ ID NO: 45, for example, at least 70%, 80%, 85%, 90%, 91%, 92%, 93%, 94%, 95%, 96%, 97%, 98%, 99%, or 100% sequence identity to SEQ ID NO: 44 or SEQ ID NO: 45.

Alternatively or additionally, the modified rpsA operon comprises or consists of a nucleotide sequence having at least 60% sequence identity to a nucleotide sequence selected from the group consisting of SEQ ID NO: 46, SEQ ID NO: 47, and SEQ ID NO: 48, for example, at least 70%, 80%, 85%, 90%, 91%, 92%, 93%, 94%, 95%, 96%, 97%, 98%, 99%, or 100% sequence identity to a nucleotide sequence selected from the group consisting of SEQ ID NO: 46, SEQ ID NO: 47, and SEQ ID NO: 48.

Alternatively or additionally, the modified 30S ribosomal protein S1 comprises or consists of an amino acid sequence having at least 60% sequence identity to SEQ ID NO: 49 or SEQ ID NO: 50, for example, at least 70%, 80%, 85%, 90%, 91%, 92%, 93%, 94%, 95%, 96%, 97%, 98%, 99%, or 100% sequence identity to SEQ ID NO: 49 or SEQ ID NO: 50.

The genetically modified Gram-negative bacterial cell of the invention is capable of releasing nOMV. Alternatively or additionally, the genetically modified Gram-negative bacterial cell is capable of releasing greater quantities of nOMVs compared to an unmodified bacterial cell. Alternatively or additionally, the genetically modified Gram-negative bacterial cell is capable of secreting greater quantities of nOMVs compared to a wild-type bacterial cell.

For example, the genetically modified Gram-negative bacterial cell, compared to an unmodified bacterial cell, is capable of releasing at least 2.0-fold more nOMVs when grown in liquid culture, for example, 2.5-fold more, 3.0-fold more, 3.5-fold more, 4.0-fold more, 4.5-fold more, 5.0-fold more, 5.5-fold more, 6.0-fold more, 10-fold more, 20-fold more, 30-fold more, 40-fold more, 50-fold more, 60-fold more, 70-fold more, 80-fold more, 90-fold more, or 100-fold more nOMVs when growing in liquid culture. A 2-fold increase on nOMV release, relative to unmodified bacterial cells is considered as significant in the art (Van der Westhuizen W A et al., Heliyon, 2019 Jul. 1; 5(7):e02014). For example, the release of nOMVs is suitably evaluated by a parameter selected from the group consisting of the absolute amount of nOMVs, the concentration of nOMVs in the supernatant (after spinning), the absolute amount of nOMVs relative to the number of bacterial cells, and the concentration of nOMVs relative to the number of bacterial cell. For example, the protein and lipid content of OMVs can be evaluated by Lowry assay (Markwell M A et al., Anal Biochem, 1978 Jun. 15; 87(1):206-10) and by using a fluorescent dye such as FM4-64 (McBroom A J et al., J Bacteriol, 2006 August; 188(15):5385-92), respectively. For example, a Lowry method is carried out on 96-Flat Bottom Transparent Polystyrene plates using triplicate aliquots of each sample and employing bovine serum albumin (BSA) to perform a standard curve. 5 μl of each undiluted and PBS-diluted sample and BSA are added to each well and combined with freshly made DC™ Protein Assay Reagent (BioRad). Lowry reagent S is mixed at 2% (v/v) with Lowry reagent A and the mixture is aliquoted 25 μl/well. Then 200 μl Lowry reagent B are added to each sample, and the plate are incubated for 15 min at room temperature. The absorbance at 750 nm is measured against a PBS/reagent blank using Infinity 200 PRO spectrophotometer (TECAN). The nOMV protein content may be expressed as “total OMV protein yield by Lowry” (mg/L or mg/ml), “specific protein yield of OMV by Lowry” (mg/L/OD or mg/mL/OD) and “OMV protein yield over wildtype” (specific protein yield of mutant OMV/specific protein yield of WT OMV). In one preferred embodiment, the release of nOMVs is evaluated as “total OMV protein yield by Lowry” (mg/L or mg/ml).

For example, the release of nOMVs in culture supernatant is quantified with the fluorescent dye FM4-64 by mixing 5 μl of FM4-64 dye (100 μg/ml) with 45 μl of 0.22 μm-filtered supernatant in a 96-flat bottom black polystyrene plate and immediately vortexing. Air bubbles are removed by spinning the plate for few seconds and FM4-64 fluorescence is measured by an Infinity 200 PRO plate reader (TECAN) with excitation at 515 nm and emission at 640 nm. FM4-64 probe mixed with medium is used as baseline and subtracted to sample reads. nOMVs release may be expressed as fluorescence intensity over wild type (FIOW) change [such as (mg/L/OD)mutant/(mg/L/OD)wt or (mg/ml/OD)mutant/(mg/ml/OD)wt] volumetric yield (mg/L).

Alternatively or additionally, vesicle concentration can be assessed by nanoparticle tracking analysis (NTA) as previously described (Olaya-Abril A et al., Proteomics, 2014). A suitable method is of performing NTA employs a NanoSight NS300 (Malvern Ltd). Purified nOMVs, or double filtered supernatant of bacterial cultures, are loaded into the measurement chamber at 1:200-1:5000 dilution range in 0.02 μm filtered D-PBS. Measurements are performed in flow mode by capturing 5 measurements of 60 sec, with a flow rate of 20 (˜2.1 μL/min), yielding 60-90 particles per frame. All measurements are performed at room temperature, and the captured results are analysed using NanoSight NTA 3.2 software. Before sample measurement, it is confirmed that the MilliQ diluent contains less than 1.0 particle per frame by measuring the MilliQ diluent for 60 sec. vesicle concentration may be expressed as “total particles by NTA” (particle number/ml), “specific particles by NTA” (particle number/ml/OD) or as “particle number over wildtype” (particle number/ml/OD)mutant/(particle number/ml/OD)wt.

Alternatively or additionally, the Gram-negative bacterial cell does not comprise modifications causing deletion of the C-terminal domain of a 30S ribosomal protein S1, for example resulting from mutations within the 3′-region of the rpsA gene. Alternatively or additionally, the Gram-negative bacterial cell does not comprise modified rpsA gene(s), rpsA operon(s) and/or 30S ribosomal protein(s) S1 as described in Schnier J, J Biol Chem, 1986 Sep. 5; 261(25):11866-71, the disclosure of which is incorporated by reference herein. Alternatively or additionally, the Gram-negative bacterial cell does not comprise modified rpsA gene(s), rpsA operon(s) and/or 30S ribosomal protein(s) S1 as described in Boni I V et al., J Bacteriol 2000 October; 182(20):5872-9, the disclosure of which is incorporated by reference herein.

In a second aspect, the invention provides a method of generating a genetically modified Gram-negative bacterial cell, comprising a step of modifying a wild-type rpsA gene, operon, RNA and/or 30S ribosomal protein S1, such that the modification causes the genetically modified Gram-negative bacterial cell, when grown in culture medium, to release greater quantities of nOMVs into the medium than the unmodified bacterial cell. In a third aspect, the invention provides a method of generating a genetically modified Gram-negative bacterial cell, comprising a step of providing a mutant 30S ribosomal protein S1, such that the modification causes the genetically modified Gram-negative bacterial cell, when grown in culture medium, to release greater quantities of nOMVs into the medium than the unmodified bacterial cell.

Furthermore, it is provided a cell culture comprising one or more genetically modified Gram-negative bacterial cells of the invention.

### OMVs

The term “native outer membrane vesicles” or “nOMVs” herein indicates outer membrane vesicles spontaneously released by Gram-negative bacteria in the culture medium. nOMVs are not extracted by detergents or denaturing agents. The nOMVs of the invention present the outer membrane proteins (OMP) and/or lipopolysaccharide (LPS) in their native conformation and correct orientation in the natural membrane environment, and usually lack the cytoplasmatic components. On the contrary, the term “detergent extracted OMC” or “dOMV” encompasses a variety of proteoliposomic vesicles obtained by disruption of the outer membrane of a Gram-negative bacterium typically by a detergent extraction process to form vesicles therefrom.

The term “generalized module for membrane antigens” or “GMMA” may also be used to refer to nOMVs obtained from mutant bacteria.

nOMVs may be obtained from a culture of the genetically modified Gram-negative bacterial cell of the invention. They may be obtained from bacteria grown in broth or in solid medium culture, for example by separating the bacterial cells from the culture medium (e.g. by filtration or by a low-speed centrifugation to pellet the cells), and separating an outer membrane fraction from cytoplasmic molecules (e.g. by filtration, by differential precipitation or aggregation of outer membranes and/or nOMVs, by affinity separation methods using ligands that specifically recognize outer membrane molecules, or by a high-speed centrifugation that pellets outer membranes and/or nOMVs).

A useful process for nOMV preparation involves ultrafiltration of crude nOMVs, rather than instead of high speed centrifugation. The process may involve a step of ultracentrifugation after the ultrafiltration takes place.

Preparations of nOMVs used in the present invention will generally be substantially free from whole bacteria, whether living or dead. The size of the vesicles means that they can readily be separated from whole bacteria by filtration e.g. as typically used for filter sterilisation.

In one fourth aspect, the invention provides a process for preparing nOMVs, comprising the steps of inoculating a culture vessel containing a nutrient medium suitable for growth of the genetically modified Gram-negative bacterial cell, culturing the genetically modified Gram-negative bacterial cell under conditions which permit the release of nOMVs into the medium by the Gram-negative bacterial cell, recovering nOMVs from the medium; and mixing the nOMVs with a pharmaceutically acceptable diluent or carrier. Alternatively or additionally, after recovering the nOMVs, the process may comprise a further step of sterile-filtering the preparation of nOMVs.

A fifth aspect of the invention provides an nOMV obtained or obtainable from the genetically modified bacterial cell of the invention, or from a genetically modified Gram-negative bacterial cell obtained or obtainable by the method of the invention, or by the process of the invention.

A sixth aspect of the invention provides a for preparing nOMVs, comprising the steps of inoculating a culture vessel containing a nutrient medium suitable for growth of a genetically modified Gram-negative bacterial cell, culturing the genetically modified Gram-negative bacterial cell under conditions which permit the release of nOMVs into the medium by the bacteria, wherein the conditions comprise addition of the modified 30S ribosomal protein S1 described herein, recovering nOMVs from the medium, and mixing the nOMVs with a pharmaceutically acceptable diluent or carrier.

### Pharmaceutical Methods

In a sixth aspect, the invention provides an immunogenic composition comprising the nOMVs obtained or obtainable from the Gram-negative bacterial cell of the invention. Alternatively or additionally, the composition may further comprise one or more additional antigens from the same or different pathogen.

The immunogenic compositions of the invention may further comprise a pharmaceutically acceptable carrier. Typical pharmaceutically acceptable carriers include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc.

Additionally, auxiliary substances, such as wetting or emulsifying agents, pH buffering substances, and the like, may be present. Sterile pyrogen-free, phosphate-buffered physiologic saline is a typical carrier.

Compositions of the invention may be in aqueous form (i.e., solutions or suspensions) or in a dried form (e.g., lyophilised). If a dried vaccine is used then it will be reconstituted into a liquid medium prior to injection.

The compositions of the invention may be prepared in various forms. For example, the compositions may be prepared as injectables, either as liquid solutions or suspensions. The composition may be prepared for pulmonary administration e.g. as an inhaler, using a fine powder or a spray. The composition may be prepared as a suppository or pessary. The composition may be prepared for nasal, aural or ocular administration e.g., as spray, drops, gel or powder. Compositions of the invention will generally be administered directly to a patient. Direct delivery may be accomplished by parenteral injection (e.g., subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used.

Compositions of the invention will generally include a buffer. A phosphate buffer is typical.

Compositions of the invention may be administered in conjunction with other immunoregulatory agents. In particular, compositions may include one or more adjuvants. Such adjuvants include, but are not limited to mineral-containing compositions. Mineral containing compositions suitable for use as adjuvants in the invention include mineral salts, such as aluminium salts and calcium salts (or mixtures thereof). Calcium salts include calcium phosphate. Aluminium salts include hydroxides, phosphates, sulfates, etc., with the salts taking any suitable form (e.g. gel, crystalline, amorphous, etc.). Adsorption to these salts is preferred. The adjuvants known as aluminium hydroxide and aluminium phosphate may be used.

### Medical Uses

The nOMV of the invention is capable of eliciting an immune response to the bacterial cell they have been obtained from when administered to a mammal. The immune response elicited by the nOMV may be directed toward or against one or more bacterial protein antigens present in the nOMV. The immune response may be a cellular or a humoral immune response. Particularly, the immune response is an antibody response. By “antibody response” to an nOMV, such as an antibody response toward or against one or more bacterial protein antigens present in the nOMV, we mean or include the capability to induce an immune response in a subject that generates (e.g., stimulates the release of) antibodies capable of binding to an nOMV or to one or more bacterial protein antigens present in the nOMV. It is preferred that the binding moiety is capable of binding in vivo, i.e., under the physiological conditions in which the nOMV or the one or more bacterial protein antigens present in the nOMV exists on or inside of a subject's body. Such binding specificity may be determined by methods well known in the art, such as e.g. ELISA, immunohistochemistry, immunoprecipitation, Western blots and flow cytometry using nOMVs of bacterial cells.

Alternatively or additionally, the immune response is a T-cell immune response that can neutralise the infection and/or virulence of the bacterial cell. Alternatively or additionally, the immune response is an immune-activating response, for example, a protective immune response. The nOMV may be capable of eliciting an in vitro protective immune response and/or an in vivo protective immune response when administered to a subject. By “immune-activating response” we mean and/or include that the nOMV induces or is capable of inducing an immune response in a subject that does not result in suppressing or terminating inflammation or inflammatory signals and, preferably, results in the activation or enhancement of inflammation or inflammatory signals (e.g., cytokines). Compositions of the invention are immunogenic and are more preferably vaccine compositions. The nOMVs, compositions and vaccines of the invention may either be prophylactic (i.e., to prevent infection) or therapeutic (i.e., to treat infection), but will typically be prophylactic.

In a seventh aspect, the invention provides nOMVs, immunogenic compositions or vaccines for use in raising an immune response in a vertebrate, preferably in a mammal, comprising administering an nOMV, immunogenic composition or vaccine of the invention to the vertebrate. The nOMV, immunogenic composition and vaccine is preferably able to raise an immune response in a vertebrate, preferably in a mammal. The immune response is preferably protective and preferably induces an antibody response in the host, i.e., a protective antibody response.

In an eighth aspect, the invention also provides a method for raising an immune response in a vertebrate, preferably in a mammal, comprising administering the nOMV, immunogenic composition or vaccine of the invention to the mammal. The immune response is preferably protective and preferably induces an antibody response in the host, i.e., a protective antibody response.

The mammal is preferably a human. Where the vaccine is for prophylactic use, the human is preferably a child (e.g., a toddler or infant, particularly a neonate) or a teenager. A vaccine intended for children may also be administered to adults e.g. to assess safety, dosage, immunogenicity, etc. A preferred class of humans for treatment are females of child-bearing age (e.g., teenagers and above). Another preferred class is pregnant females.

A ninth aspect of the invention provides a composition of the invention for use in medicine. A tenth aspect of the invention provides the use of an nOMV, a composition, or the vaccine of the invention in the manufacture of a medicament for raising an immune response in a vertebrate, preferably in a mammal. These uses and methods are preferably for the prevention and/or treatment of a disease caused by the bacterial cell from which the nOMV was obtained, or from a bacterial cell of the same genus or species of the bacterial cell from which the nOMV was obtained, such as B. pertussis, M. catarrhalis, N. meningitidis, N. gonorrhoeae, E. coli, P. aeruginosa, H. influenzae.

The invention may be used to elicit systemic and/or mucosal immunity. The immunogenic compositions of the invention may be administered in single or multiple doses.

### GENERAL

To facilitate an understanding of the present invention, a number of terms and phrases are defined below. Art-recognized synonyms or alternatives of the following terms and phrases (including past, present, etc. tenses), even if not specifically described, are contemplated.

As used in the present disclosure and claims, the singular forms “a,” “an,” and “the” include plural forms unless the context clearly dictates otherwise; i.e., “a” means “one or more” unless indicated otherwise.

The terms “about” or “approximately” mean roughly, around, or in the regions of. The terms “about” or “approximately” further mean within an acceptable contextual error range for the particular value as determined by one of ordinary skill in the art, which will depend in part on how the value is measured or determined, i.e., the limitations of the measurement system or the degree of precision required for a particular purpose, e.g. the amount of a nutrient within a feeding formulation. When the terms “about” or “approximately” are used in conjunction with a numerical range, it modifies that range by extending the boundaries above and below the numerical values set forth. For example “between about 0.2 and 5.0 mg/ml” means the boundaries of the numerical range extend below 0.2 and above 5.0 so that the particular value in question achieves the same functional result as within the range. For example, “about” and “approximately” can mean within 1 or more than 1 standard deviation as per the practice in the art. Alternatively, “about” and “approximately” can mean a range of up to 20%, preferably up to 10%, more preferably up to 5%, and more preferably up to 1% of a given value.

The term “and/or” as used in a phrase such as “A and/or B” is intended to include “A and B”, “A or B”, “A” and “B”. Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specified otherwise, all of the designations “A %-B %”, “A-B %”, “A % to B %”, “A to B %”, “A %-B”, “A % to BB are given their ordinary and customary meaning. In some embodiments, these designations are synonyms.

The terms “substantially” or “substantial” mean that the condition described or claimed functions in all important aspects as the standard described. Thus, “substantially free” is meant to encompass conditions that function in all important aspects as free conditions, even if the numerical values indicate the presence of some impurities or substances. “Substantial” generally means a value greater than 90%, preferably greater than 95%, most preferably greater than 99%. Where particular values are used in the specification and in the claims, unless otherwise stated, the term “substantially” means with an acceptable error range for the particular value.

An “effective amount” means an amount sufficient to cause the referenced effect or outcome. An “effective amount” can be determined empirically and in a routine manner using known techniques in relation to the stated purpose.

As used herein, references to “percentage sequence identity” between a query nucleotide or amino acid sequence and a nucleotide or subject amino acid sequence are understood to refer to the value of identity that is calculated using a suitable algorithm or software program known in the art to perform pairwise sequence alignment. A query nucleotide or amino acid sequence may be described by a nucleotide or amino acid sequence identified in one or more claims herein. The query sequence may be 100% identical to the subject sequence, or it may include up to a certain integer number of nucleotide or amino acid alterations (e.g. point mutations, substitutions, deletions, insertions etc.) as compared to the subject sequence, such that the % identity is less than 100%. For example, the query sequence is at least 60, 70, 80, 85, 90, 91, 92, 93, 94, 95, 96, 97, 98, or 99% identical to the subject sequence. In order to calculate percent identity, the query and subject sequences may be compared and aligned for maximum correspondence over a designated region (e.g. a region of at least about 40, 45, 50, 55, 60, 65 or more nucleotide or amino acids in length, and can be up to the full length of the subject nucleotide or amino acid sequence). The alignment may be determined by the Needleman-Wunsch global alignment algorithm which is disclosed in Needleman & Wunsch (1970) 1. Mol. Biol. 48, 443-453 and is implemented in the publicly available global alignment tool EMBOSS Needle (HyperTextTransferProtocolSecure://WorldWideWeb.ebi.ac.uk/Tools/psa/). The alignment may be determined with a gap open penalty of 10 and a gap extension penalty of 0.5, BLOSUM matrix of 62. An alternative or additional global alignment tool implementing the Needleman-Wunsch global alignment algorithm is EMBOSS Stretcher.

Unless otherwise provided, the term “polypeptide” refers to polypeptides of any length capable to act as a selected antigen. The amino acid polymer forming the polypeptide of the invention, may be linear or branched, it may comprise modified amino acids, and it may be interrupted by non-amino acids. The term also encompasses an amino acid polymer that has been modified naturally or by intervention; for example, disulphide bond formation, glycosylation, lipidation, acetylation, phosphorylation, or any other manipulation or modification, such as conjugation with a labelling component. Also included within the definition are, for example, polypeptides containing one or more analogues of an amino acid (including, for example, unnatural amino acids, etc.), as well as other modifications known in the art. Polypeptides can occur as single chains or associated chains.

The term “comprising” encompasses “including” as well as “consisting”, e.g., a composition “comprising” X may consist exclusively of X or may include something additional, e.g., X+Y.

### EXAMPLES

### Example 1. Genetic Engineering of B. pertussis: Materials and Methods

### Bacterial Strains and Growth Conditions

B. pertussis Tohama I PT-9K/129G (PTg) which carries a genetically detoxified pertussis toxin was used in this study. Bacteria were grown on Regan-Lowe Charcoal (Becton Dickinson) agar plates for three-five days at 37° C. Liquid cultures of Bp were performed in modified Stainer-Scholte medium (SS) supplemented with 0.4% (w/v) L-cysteine monohydrochloride (SIGMA), 0.1% (w/v) FeSO₄ (AROS ORGANICS), 0.4% (w/v) ascorbic acid (SIGMA), 0.04% (w/v) nicotinic acid (SIGMA), 1% (w/v) reduced glutathione (SIGMA) using 2 mL 96-deepwell plates (Eppendorf) for microscale cultures (600 μl), and 0.25-2 L baffled flasks with vented cup (Corning) for small and large scale cultures (50-300 ml).

Bacterial cultures were grown at 37° C. in orbital shakers, agitating microplates at 350 rpm and flasks at 180 rpm. Cultures absorbance at 600 nm was monitored during the entire growth. Bacterial cultures were withdrawn when stationary phase was reached by centrifugation at 4000×g for 30′ at 4° C. (microscale cultures) or at 10000×g for 20′ at 4° C. (flask cultures), pellets were frozen at −20° C. for genomic DNA purification and supernatants were double filtered with 0.22 μM Stericup® vacuum filter bottles (Millipore) and stored at 4° C. for further processing.

Microscale bacterial growths were monitored by absorbance measurement of culture aliquots (OD at 600 nm) by Infinity 200 PRO spectrophotometer (TECAN).

pSORTP1, a derivative of pRTP1 plasmid, E. coli S17-1λpir (Biomedal) donor strains and pUT-MINI-Tn5-Km^{R} vector (Biomedal) were used for B. pertussis conjugation.

E. coli cells were routinely grown in Luria Bertani (LB) medium (Becton Dickinson) in rotary shakers at 37° C. and 180 rpm for 16 h. Media were supplemented with SIGMA antibiotics where appropriate for each strain: streptomycin 400 μg/ml, kanamycin 25-50 μg/ml, nalidixic acid 20 μg/ml, ampicillin 50-100 μg/ml.

Identification of Novel Hyper Blebbing Mutations in B. pertussis

Transposon insertion sites in bacterial genomes were identified by Random Amplification of Transposon Ends (RATE) (Karlyshev A V et al., Biotechniques, 2000 June; 28(6):1078, 1080, 1082). RATE is a two-steps process represented by RATE PCR and sequencing of RATE products. In brief, a single bacterial CFU was inoculated into 50 μl of water and incubated for 10 min at 99° C. The PCR was performed using Q5® High-Fidelity DNA Polymerase (NEB), and the following heat program: 1 cycle (94° C. for 1 min); 30 cycles (94° C. for 30 sec; 55° C. for 30 sec; 72° C. for 30 sec); 30 cycles (94° C. for 30 sec; 30° C. for 30 sec; 72° C. for 30 sec) including Taq spyking; 30 cycles (94° C. for 30 sec; 55° C. for 30 sec; 72° C. for 1 min); 1 cycle (72° C. for 5 min). RATE PCR products were visualized by electrophoresis run on 1% agarose gel. Positive PCR products were purified by QIAquick PCR purification kit (QIAgen) following procedure, then sequenced by p3730xl DNA Analyzer (Thermofisher).

### Genomic DNA Purification

Genomic DNA was purified from the bacteria pellets from microscale growth of Bp clones harbouring transposon insertions (TN5). Genomic DNA was isolated by GenElute Bacterial Genomic DNA Kit (SIGMA) according to the manufacturer's instructions for Gram negative bacteria. DNA elution was performed in 100 μl of pre-warmed nuclease free water. The purified DNA concentration was determined by measurement at NanoDrop ND-1000 spectrophotometer (EuroClone). All samples were diluted to 10 ng/μl and anaysed by NGS sequencing at Miseq (Illumina).

### GMMA Purification by Ultracentrifugation

Cell-free supernatants from B. pertussis or E. coli cultures were subjected to ultracentrifugation in polycarbonate centrifuge tubes (Beckman) at 175,000×g for 3 hours and 4° C., the resulting GMMA pellet was washed with Dulbecco's Phosphate-Buffered Saline (D-PBS), further ultracentrifuged at 175,000×g for 1 hour at 4° C. The pellet was resuspended into 200 μl D-PBS by overnight shaking at 4° C. Any DNA contamination was removed from GMMA samples by a final 2 h-treatment at room temperature with 100 U benzonase enzyme (Roche). Purified GMMA were finally filtered using 0.22 μm pore size filters (Millipore).

### GMMA Purification by Tangential Flow Filtration (TFF)

Isolation and concentration of extracellular vesicles from large volumes of cell culture supernatants (300 ml) was obtained by TFF. Samples were filtered using 0.22 μm pore size filters (Millipore), incubated with 100 U Benzonase at 4° C. for 48 hours, then concentrated to 200 ml in a TFF system (Sartorius) using hollow fiber filter module with 300 kDa membrane pore size (Sartorius). In all TFF steps, the transmembrane pressure was set at 0.45 psi. The column was primed with 500 ml Milli-Q water, and samples were diafiltrated with 4 L filtered PBS 1× to remove protein contaminants and simultaneously make a buffer exchange. Vesicles were further concentrated to 50 ml in TFF, diafiltrated with 2 L filtered PBS 1×, concentrated to roughly 15 ml in TFF, and finally filtered using 0.22 μm pore size filters (Millipore).

### GMMA Proteins Quantification by Lowry Assay

The protein content from GMMA and supernatant samples was determined with modified Lowry method (Markwell M A et al., Anal Biochem, 1978 Jun. 15; 87(1):206-10). The assay was carried out on 96-Flat Bottom Transparent Polystyrene plates (Costar) using triplicate aliquots of each sample and employing BSA (Biorad) to perform a standard curve. 5 μl of each undiluted and PBS-diluted sample and BSA was add to each well and combined with freshly made DC™ Protein Assay Reagent (BioRad). Lowry reagent S was mixed at 2% (v/v) with Lowry reagent A and the mixture was aliquoted 25 μl/well. Then 200 μl Lowry reagent B was added to each sample, and the plate was incubated for 15 min at room temperature. The absorbance at 750 nm was measured against a PBS/reagent blank using Infinity 200 PRO spectrophotometer (TECAN).

### GMMA Lipids Quantification by FM4-64 Dye Staining

The fluorescent dye FM4-64 (McBroom A J et al., J Bacteriol, 2006 August; 188(15):5385-92) was used as a quantitative marker of GMMA in the culture supernatant. 5 μl of FM4-64 dye (Molecular Probes, 100 μg/ml) were mixed to 45 μl of 0.22 μm-filtered supernatant in a 96-flat bottom black polystyrene plate (Costar), and immediately vortexed. Air bubbles were removed by spinning the plate for few seconds and FM4-64 fluorescence was measured by an Infinity 200 PRO plate reader (TECAN) with excitation at 515 nm and emission at 640 nm. FM4-64 probe mixed with medium was used as baseline and subtracted to sample reads. Fluorescence intensity (FI) values were determined in cultures dilution and clone-specific production of vesicles was calculated by dividing the fluorescence values to cultures OD₆₀₀ₙₘ registered at the harvesting. Relative vesiculation was determined by the ratio between the specific fluorescence of clones respect to that of the wild type strain in each experimental group.

### Nanoparticle Tracking Analysis (NTA)

A NanoSight NS300 (Malvern Ltd) was used to determine membrane particles size and concentration as previously described (Olaya-Abril A et al., J Proteomics, 2014 Jun. 25; 106:46-60). Purified GMMA, or double filtered supernatant of bacterial cultures, were loaded into the measurement chamber at 1:200-1:5000 dilution range in 0.02 μm filtered D-PBS. Measurements were performed in flow mode by capturing 5 measurements of 60 sec, with a flow rate of 20 (˜2.1 μL/min), yielding 60-90 particles per frame. All measurements were performed at room temperature, and the captured results were analysed using NanoSight NTA 3.2 software. Before sample measurement, we confirmed that the MilliQ diluent contained less than 1.0 particle per frame by measuring the MilliQ diluent for 60 sec. All measures were performed at room temperature.

### SDS-PAGE

Cell free culture supernatants and purified GMMA fractions were analysed by SDS-PAGE (4-12% resolving gel, Thermofisher), as described previously (Laemmli U K, Nature 1970 Aug. 15; 227(5259):680-5). Cleavage of structural proteins during the assembly of the head of bacteriophage T4. Molecular weight estimation of samples was performed by Novex Sharp Pre-stained Protein Standard (Thermofisher). The gel was subsequently used for coomassie staining by SimplyBlue SafeStain (Thermofisher) or for silver staining analysis by silver Stain kit (Thermofisher). The staining was performed according to the manufacturer instructions.

### Immunoblot Analysis

Proteins contained in the supernatant were separated on SDS-polyacrilamide gel, transferred to a nitrocellulose membrane by I-blot mini system (Thermofisher), then blocked with PBS+0.1% v/v Tween 20 buffer supplemented with 5% non-fat dry milk (Sigma). The membrane was probed with anti-whole Bp OMV mouse serum as primary antibody and treated with anti-mouse IgG conjugated with HRP (Sigma) as the secondary antibody. The chemiluminescent signals were developed with SuperSignal West Pico PLUS chemiluminescent substrate (Thermofisher).

### Transmission Electron Microscopy (TEM)

nOMVs were visualized by TEM analysis with negative staining. The sample (5 μl) was loaded for 30 seconds onto a glow discharged copper 300-square mesh grid (Agar Scientific), blotted the excess, and the grid was negatively stained using NanoW (Nanoprobes) for 30 seconds. Micrographs were acquired using a Tecnai G2 spirit (FEI Thermofisher), and vesicles images were acquired using a Veleta CCD (Emsis).

B. pertussis Mutants Validation Design

Mutant constructs were designed to generate recombinant pSORTP1 plasmids harbouring the construct sequences mapping the Tn5 mutations at pSORTP1 restriction sites. Each construct contains restriction sites, upstream and downstream Tohama I genome regions of each gene to be mutated, and the kanamicin resistance cassette (KanR) for transconjugants selection.

Recombinant plasmids were purified by EZNA Plasmid Mini Kit II (Omega) as described by the producer, and then used to transform E. coli S17-1λpir conjugative donor strain according to manufacturer instructions.

### Example 2. Increased OMV Release of B. pertussis rpsA Mutants

A random mutagenesis approach implementing the use of a transposon Tn5 library screened in microscale plate format was developed to identify mutations possibly leading to increased nOMV release. Trans-conjugants were generated by a novel conjugation protocol that was optimized to produce a Tn5 mutant library of 4×10⁶ single mutants/conjugation. The transconjugants were isolated by single colony picking and grown into a 96-deepwell plate. It was determined that in order to have similar growth profiles of clones, the size of the selected colonies had to be comparable. Growth in microscale culture was further optimized by using a lid able to prevent evaporation of the liquid.

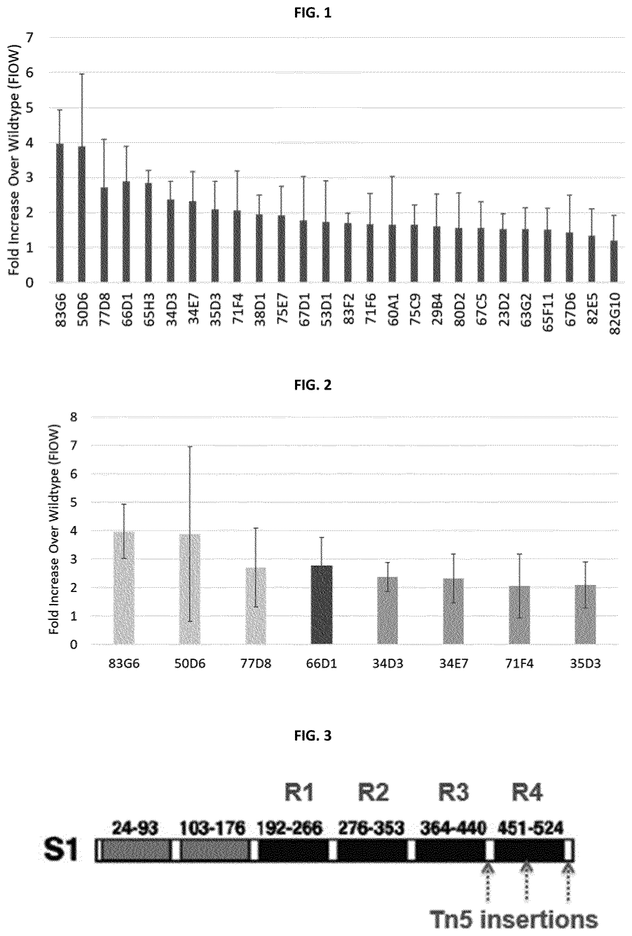

nOMV release in the culture supernatant was evaluated by FM4-64 fluorescence normalising each fluorescence value to the optical density of the corresponding clone and finally comparing it to that of a WT clone grown in the same plate (FIOW). In total 5,670 random clones were screened. Among these, 95 clones were selected with FIOW over 2-fold and re-evaluated by at least two further experiments in microscale cultures. As reported in FIG. 1, 26 clones out of the selected 95, confirmed a FIOW>1 during the subsequent microscale growths. Finally, eight clones among these resulted to have a FIOW>2 during multiple tests in micro-scale (FIG. 2).

Transposon insertions in Bp chromosome were mapped by RATE. Of interest, the insertion in the 3 top-scoring clones was associated to a single gene, rpsA (BP0950, 30S Ribosomal protein S1).

In order to precisely map the transposon insertion in these clones, the genomic DNA was purified and analysed by next generation sequencing (NGS), revealing that all transposon insertions into the rpsA gene (50D6, 83G6, 77D8 clones) occurred downstream of the R3 domain (FIG. 3).

Tn5 transposon insertion into the 50D6 mutant clone occurred before last 376 rpsA nucleotides and before last 308 and 77 rpsA nucleotides in 77D8 and 83G6 clones respectively. Duplicated sequences (GCTCGA for 50D6, CATCAATG for 77D8, TGTCCGAGG for 83G6 clone) were detected at the 5′ end of the transposon insertion.

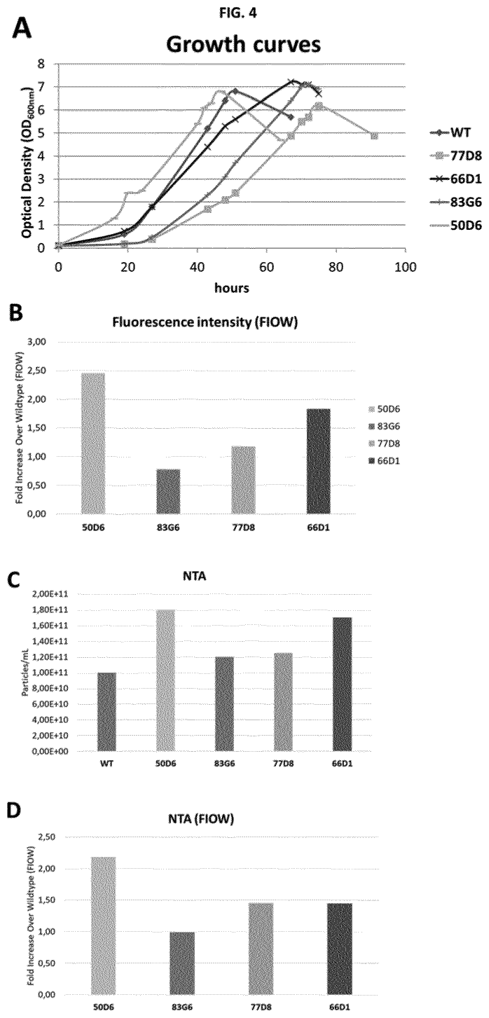

To further confirm the capability to overproduce GMMA with a sustainable growth profile, the the 3 rpsA mutant clones (50D6, 83G6, 77D8) and an additional clone identified by random mutagenesis but not related to rpsA (66D1) were grown in small scale culture. The bacterial growths of WT and mutant strains at 37° C. and 180 rpm were monitored until late stationary phase of growing. Released vesicles were purified from culture supernatants at the end of growth and quantified by fluorescence analysis. To evaluate the amount of purified released vesicles, NTA was performed. The results of two experiments are reported in FIG. 4 (A-B). The 50D6 and 66D1 clones exhibited a similar growth rate respective to their parent WT strain in small scale cultivation and released twice the amount of GMMA in the supernatant respective to their parent WT strain as confirmed by fluorescence results. Increased vesiculation was also confirmed by NTA of purified vesicles (FIG. 4 C-D).

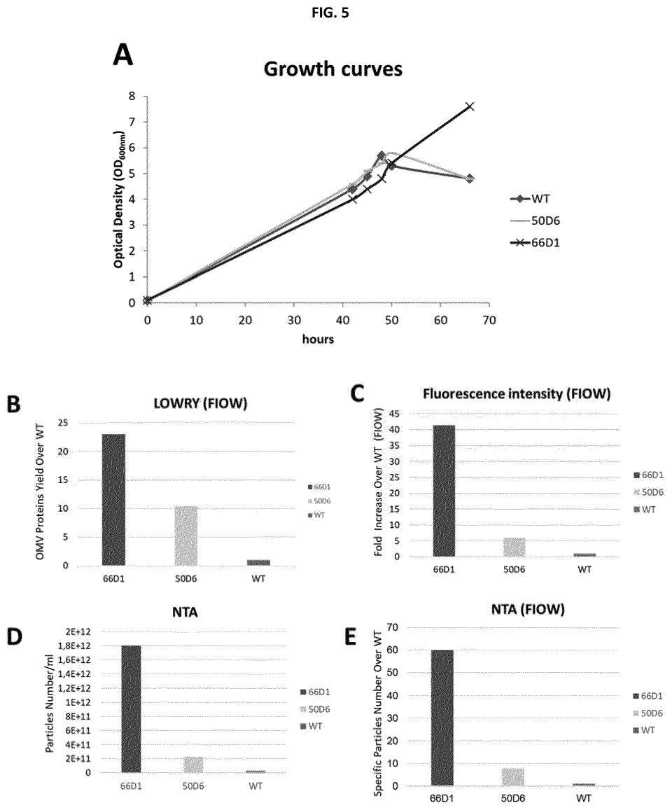

GMMA release was further investigated in large scale cultivation (300 ml). As schematically represented in FIG. 5, 50D6 and 66D1 clones exhibited similar growth rate of their parental WT strain. GMMA were isolated by TFF from cell culture supernatants, then quantified by Lowry, NTA and fluorescence analysis. All procedures confirmed the increased number of released particles in random mutant clones with respect to the WT strain.

### Example 3. Characterisation of Vesicles from rpsA Mutants

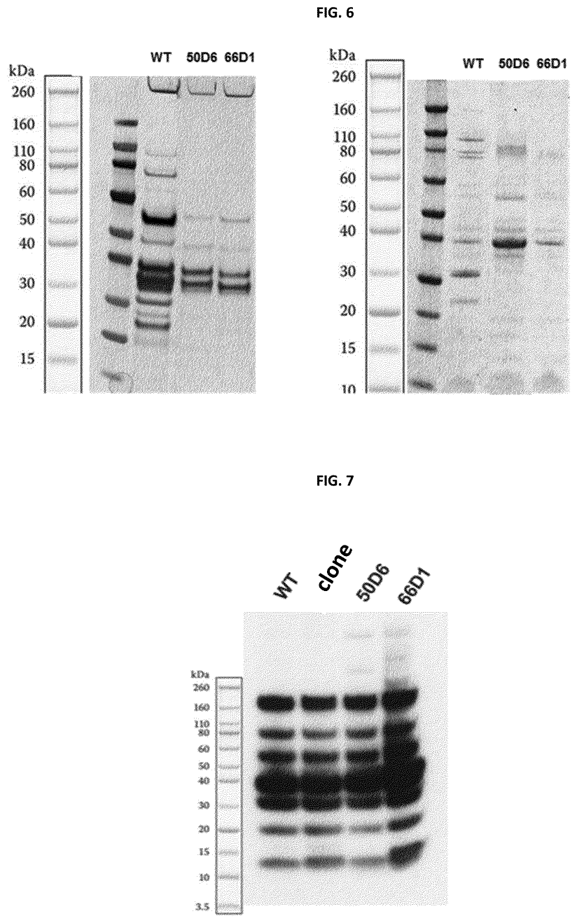

To evaluate the protein profile of both WT and random mutant clones, supernatants and purified vesicles were collected from late stationary phase of bacterial growth. The SDS-PAGE analysis of the supernatants revealed a similar pattern of bands, while differences could be observed in purified GMMA, potentially caused by a partial precipitation occurred during ultracentrifugation of samples (FIG. 6). The presence of membrane vesicles in culture supernatants was confirmed by analysing the protein profile by immunoblotting using an anti-OMV serum. The results reported in FIG. 7 show clear OMV protein ladders recognized by the serum.

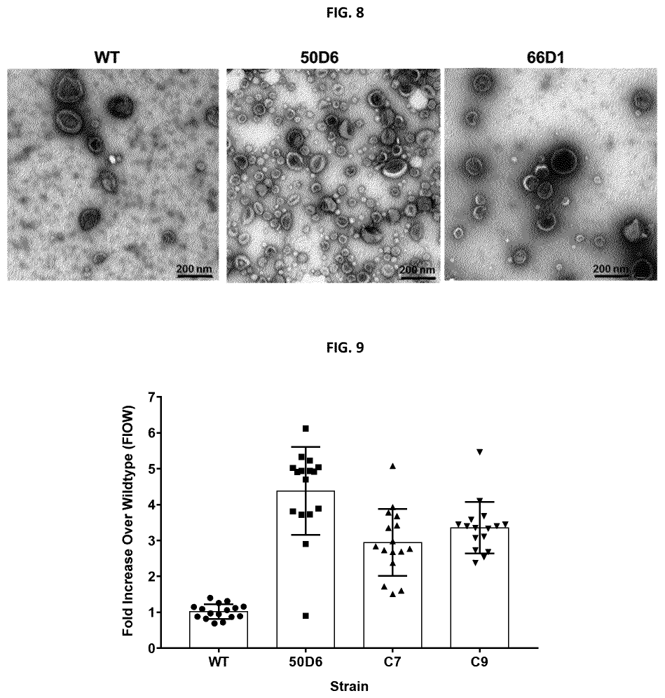

The morphology of vesicles from WT and random mutants was analysed by transmission electron microscopy (TEM). As shown in FIG. 8, circular vesicles could be visualized in all the samples. The diameters of vesicles were comparable in the range from 50 to 100 nm.

### Example 4. Validation of Tn5 mutants in B. pertussis

Clean mutants of the 50D6 clone were generated in Bp TohamaI Ptg strain. The C7 (rpsA 377-ins-kanR) mutant (SEQ ID NO: 51 and SEQ ID NO: 53) harboured a kanR insertion before the last 376 nucleotides of bp0950 gene (rpsA) i.e., at the same position of the Tn5 insertion in the 50D6 clone. The C8 mutant harboured the deletion of last 376 nucleotides of bp0950 gene (rpsA) i.e., the sequence downstream the position of the Tn5 insertion in the 50D6 clone were deleted. The deletion region was replaced by KanR and SpeI restriction site. The C9 (ihfB::kanR) mutant (SEQ ID NO: 52) harboured a deletion of the entire bp0951 gene (ihfB). The deletion region was replaced by kanR and SpeI restriction site.

The mutants were analysed as described in Example 2. Both C7 and C9 (ihfB::kanR) showed a significantly higher FIOW as compared to wild type (“WT”) as shown in FIG. 9. No significant difference was observed between C7 and C9 strains.

### Example 5: Genetic Engineering of E. coli: Materials and Methods

### Constructs Design

To confirm the increased nOMV release induced by rpsA mutation, another Gram-negative bacterium, non-pathogenic E. coli, was mutated by site-directed mutagenesis.

No intergenic region is present between the coding sequence of the rpsA and ihfB genes in the rpsA-ihfB locus from Bp Tohama I genome suggesting a possible polycistronic transcript. No canonical promoter sequence is predicted with BPROM, Softberry (Solovyev V, & A Salamov, Metagenomics and its Applications in Agriculture, Biomedicine and Environmental Studies, 2011, 61-78). The genomic sequence of the E. coli BL21(DE3) rpsA-ihfB locus displays a longer intergenic between the two genes compared to Bp. Additionally, in E. coli a functional promoter was predicted within the 159 base pair intergenic region as well as an ihf transcription factor (TF) bonding site (BFROM, Softberry).

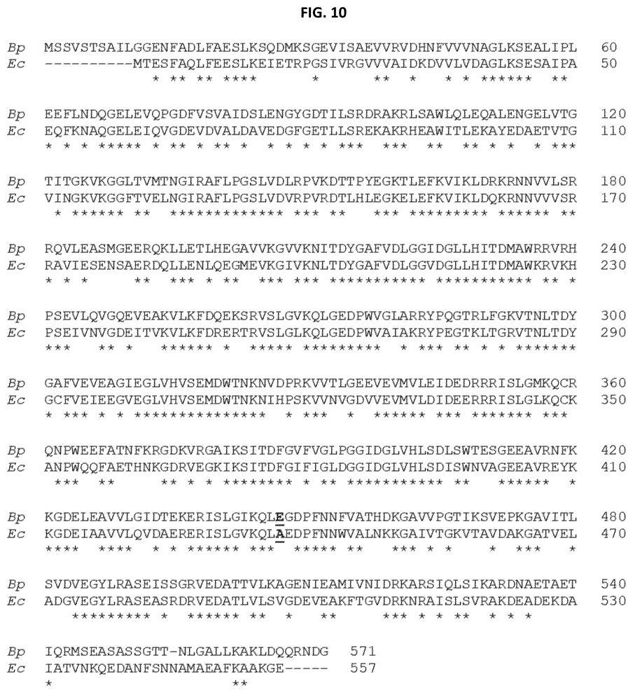

The amino acid sequence of Bp (SEQ ID NO: 13, not comprising its 5 N-terminal amino acids) and Ec S1 (SEQ ID NO: 14) were aligned (FIG. 10) using Clustal Omega online software (HyperTextTransferProtocolSecure://WorldWideWeb.ebi.ac.uk/Tools/msa/clustalo/) and shown to have 63% percent identity to each other. The Tn5 insertion point in the Bp 50D6 clone is indicated in bold in FIG. 10 and falls into the second base of the Glutamate encoding GAA codon. The first amino acid interrupted by a corresponding mutation in Ec is an alanine instead of a glutamate but the sequence immediately before (and that will be kept in the final strain) is well conserved (FIG. 10).

The ihfB gene is different between the two organisms. The Bp protein is longer (119aa vs 94aa) and the identity is 57% on the 94aa fragment.

Three different constructs were designed in Ec: (i) ins RpsA, reproducing the Tn5 insertion of the 50D6 Bp clone, with a KanR inserted before last 376 nucleotides of Bp0950 gene (rpsA); (ii) ΔrpsA 3′, having a deletion of the last R4 domain of rpsA (from the insertion point of the previous construct to the rpsA stop codon) with an inverted kanamycin cassette to reproduce the 80G6 and 77D8 Bp clones; (iii) ΔihfB, with a deletion of ihfB gene (downstream of rpsA).

Genomic mutations were also obtained in N. meningitidis and N. gonorrhoeae using the same approach. Genomic mutants were generated and tested for GMMA production at different temperatures (25° C., 30° C. and 37° C.) by direct detection of vesicles released in culture supernatants at least in two biological replicates. To confirm GMMA production and assess protein profiles in GMMA produced by mutant strains, GMMA were purified and analysed by Lowry and SDS-PAGE as described in the sections above.

### Mutants Generation

Genomic mutants of EcBL21(DE3) were generated by lambda Red recombination as previously described (Datsenko and Wanner Proc Natl Acad Sci USA, 2000 Jun. 6; 97(12):6640-5). An aliquot of Ec BL21(DE3) (chemically competent, NEB) was transformed with 50 ng of plasmid DNA pDK46, carrying the λ red recombinase gene and selected at 30° C. in the presence of ampicillin 100 mg/L. Recombinant pKD46-Ec BL21(DE3) were then made electrocompetent by cultivating at 30° C. in the presence of ampicillin 100 mg/L and 1 mM arabinose (to induce λ red recombinase expression) up to mid exponential growth phase (OD₆₀₀ₙₘ=0.4). Bacteria were then harvested and washed three times with sterile and ice-cold H₂O and finally resuspended in sterile ice cold glycerol 10% v/v. Bacteria were concentrated during washing steps to 50×.

50 μl of electrocompetent bacteria were then electroporated at 2.5 kV with 300-400 ng of purified PCR for each construct. Mutants selection was done in the presence of kanamycin 25 mg/L in LB agar at 37° C. for 24 h. Three clones for each transformation were screened with external primers by colony PCR.

### Small Scale Cultivation

Mutants were grown at different temperatures (25° C., 30° C. and 37° C.) and every condition was tested at least 2 times. Briefly, a pre-inoculum of 5 ml LB medium was incubated 16-18 h for the 25° C. and 30° C. conditions and 8 h for the 37° C. conditions at 37° C. and 180 rpm. Pre-cultures were diluted 1:100 in 50 mL HTMC medium in disposable baffled 250 mL flasks with vented cup (Corning) and incubated at the different temperatures and 180 rpm shaking until late stationary phase. OD₆₀₀ₙₘ was monitored during time spectrophotometrically. Cultures were harvested when late stationary growth phase was reached by centrifugation at 4000 rpm for 30′ at 4° C. The resulting supernatants were filtered through a 0.22 μm stericup (Millipore) and stored at 4° C. for further analysis.

### GMMA Purification

35 mL of supernatant was subjected to ultracentrifugation at 32000 rpm for 3 h at 4° C. (using 32Ti swinging rotor). One washing step was performed with 35 ml sterile PBS at 32000 rpm/4° C./2 h. Vesicle-containing pellets were resuspended in 0.15-0.6 mL of sterile PBS for 16 h at 4° C. while shaking.

### GMMA Quantification

Purified GMMA were quantified by Lowry assay following the protocol “DC Protein Assay” (CAT No 500-0114) from BIORAD. Serial step 2 dilutions of 1 mg/ml BSA were used for standard curve. GMMA was also quantified directly from cell-free supernatants by staining with the FM4-64 dye (Thermofisher Scientific, catalogue number: T13320) at a final dilution 1:100 Fluorescence intensities were also normalized on the OD₆₀₀ₙₘ value to calculate the specific GMMA yield for each mutant. Obtained values were then divided to wild type values and reported as Fold Increase Over Wildtype (FIOW).

### Example 6: Genetic Engineering of E. coli: Results and Discussion

GMMA Production from Mutant Strains at 37° C.

Mutant strains and the corresponding wild type strain were cultivated in shaking flask as described in materials and methods and growth profiles were recorded (Table 1). All the three mutants showed a slightly lower growth rate and final biomass yield as compared to wild type. Comparable growth profiles were observed between different mutants.

TABLE 1

OD₆₀₀

| Time (h) | 0 | 15 | 16 | 17 |
|---|---|---|---|---|
| WT | 0.15 | 21 | 21.5 | 21 |
| ins Rpsa #1 | 0.15 | 15 | 16 | 14.5 |
| ΔRpsA 3′ #2 | 0.15 | 15 | 18.5 | 15 |
| ΔihfB #3 | 0.15 | 13.5 | 14 | 13.5 |

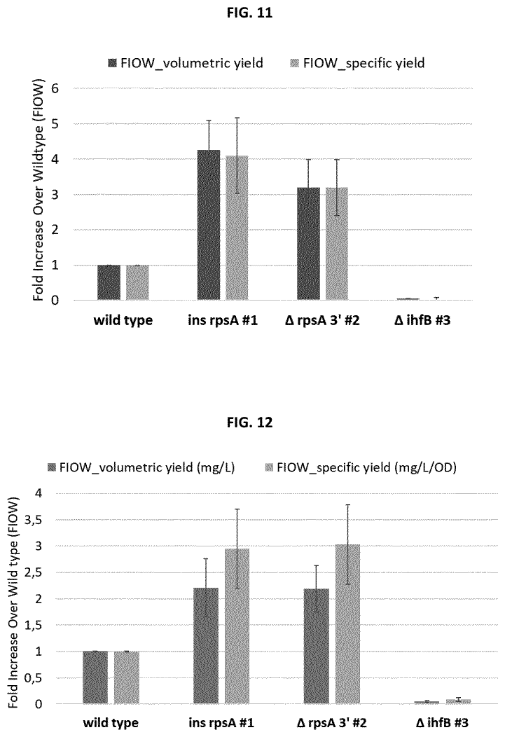

GMMA released in the supernatant were measured directly from cell-free culture supernatants revealing that both rpsA mutants (insertion or 3′ deletion mutant) produce 3- to 5-fold more GMMA than the wild type EcBL21(DE3) strain, whereas the IhfB knockout mutant produced an undetectable nOMV amount (FIG. 11).

GMMA were purified from culture supernatants by ultracentrifugation and quantified based on total protein content. The two rpsA mutants showed an increase in GMMA production compared to wild type strain between 3- and 4-fold in terms of volumetric yield (FIG. 12). This is slightly lower than what observed by direct measurement in the supernatants, probably due to different purification yields from different cultures and a partial lysis of the wild type strain that could have released intracellular proteins in the supernatant that could have affected the total protein quantification. The very low yield of the ΔihfB #3 mutant was confirmed.

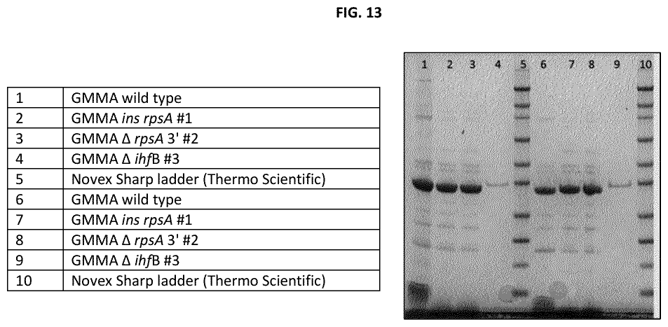

Purified GMMA were analysed in SDS-PAGE to compare protein pattern (FIG. 13). The protein profiles of the two rpsA mutants and WT are superimposable for and the prevalent bands of the ihfB mutant strain correspond to the protein profiles observed in the other strains. All GMMA preparation appear clean indicating that bacteria were still viable at the time of harvesting. A slight smear is detectable only for the wild type preparation in the first experiment suggesting that the culture was sampled when it started to lysate suggesting that the FIOW calculated on purified GMMA could be slightly underestimated.

GMMA Production from Mutant Strains at 25° C. and 30° C.

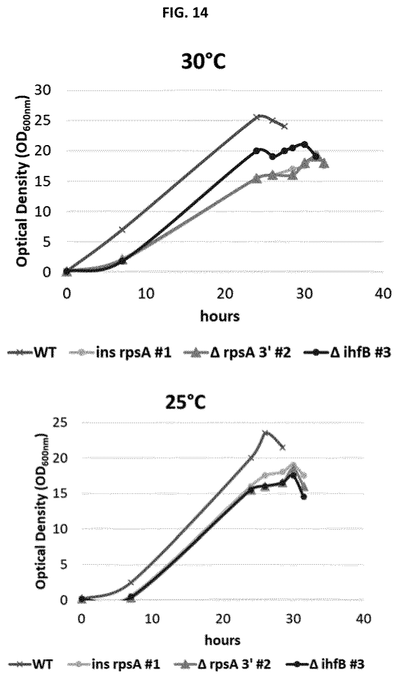

The capacity of E. coli mutants to release GMMA at temperatures that are frequently used for recombinant protein expression—such as for production of GMMA enriched of antigens of interest—was investigated. Mutant strains and the corresponding wild type strain were therefore cultivated in shaking flask as described in materials and methods at 25° C. and 30° C. The mutants' growth profiles were characterised by a lower growth rate and a slightly lower final biomass yield compared to wild type (FIG. 14). Different mutants showed comparable growth profiles.

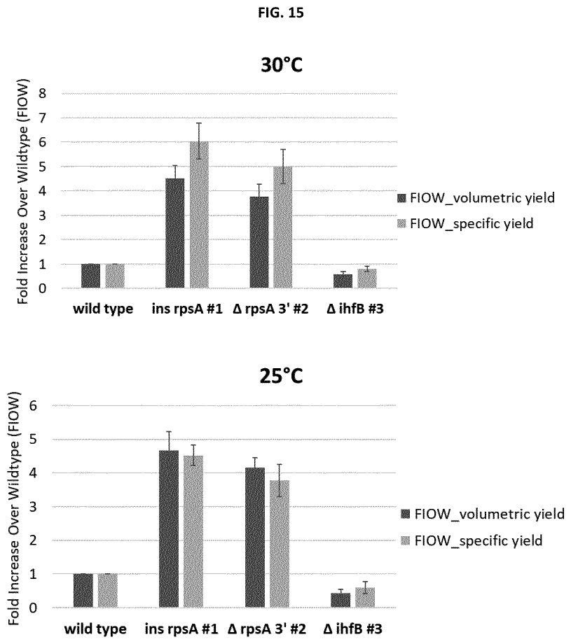

GMMA released in the supernatant were measured directly from cell-free culture supernatants revealing that both rpsA mutants (insertion or 3′ deletion mutant) produce 4- to 6-fold more GMMA than the wild type Ec BL21(DE3) strain at 30° C. and 4- to 5-fold more GMMA than the wild type Ec BL21(DE3) strain at 25° C. The highest specific yields were obtained at 30° C. Although the I/B knockout mutant produced a detectable amount of GMMA at lower temperatures, its GMMA yield were lower than one of the wild type (FIG. 15).

### Example 7: Genetic engineering of Moraxella catarrhalis

Further experiments were performed in another Gram-negative bacterium, M. catarrhalis, to confirm the increased nOMV release induced by rpsA and ihfB mutation. Specifically, a ΔrpsA-Ct mutation (deletion of the last 363 bp of rpsA) and ΔihfB mutation (deletion of ihfB completely) were evaluated.

M. catarrhalis Transformation

An M. catarrhalis BBH18 (GenBank assembly accession: GCA_000092265.1) strain, a seroresistant-lineage strain isolated from a sputum isolate from a COPD patient during an exacerbation, was used.

Directed gene deletion mutants of M. catarrhalis BBH18 were obtained by allelic exchange of the target gene with a kanamycin resistance cassette (shown by SEQ ID NOs: 56 and 58). A kanamycin resistance cassette substituting the coding sequence of the gene was fused to its 5′ and 3′ ˜600 bp flanking regions and transformed into naturally competent M. catarrhalis BBH18 bacteria. To this aim, ˜1 μg of the purified PCR product was mixed with 25 μl of bacteria resuspended in PBS, incubated for 5-6 h at 37° C. and transformants were selected twice on BHI+30 μg/ml kanamycin plates. Transformants were verified by PCR for proper recombination in the genome.

### Growth Conditions

Strains were streaked from glycerol stocks into BHI agar plates ON at 37° C.; single colonies were inoculated into 150 ml BHI at an OD600=0.1 and incubated ON at 37° C., 185 rpm. Cultures were pelleted at 4000 rpm for 20 min, and supernatants were filtered through a 0.22 μm filter.

### GMMA Purification

To purify vesicles, 140 ml of supernatant was subjected to ultracentrifugation at 32000 rpm for 3 h at 4° C. (using 32Ti swinging rotor). One washing step is performed with 140 ml sterile PBS at 32000 rpm for 2 h at 4° C. Finally vesicles containing pellets were resuspended in 1.5-2.2 ml of sterile PBS for 16 h at 4° C. while shaking.

### GMMA Quantification

Purified GMMA were quantified by Lowry assay following the protocol “DC Protein Assay” (CAT No 500-0114) from BIORAD. Serial step 2-dilutions of 2 mg/ml BSA were used for the standard curve.

### Negative Staining Electron Microscopy

5 μl of each sample (wt, ΔrpsA-Ct and ΔihfB GMMA) (2 OD/ml) was loaded for 30 seconds onto a glow discharged copper 300-square mesh grid. After blotting the excess, the grid was negatively stained using NanoW for 30 seconds. The samples were analyzed using a Tecnai G2 spirit and the images were acquired using a Tvips TemCam-F216 (EM-Menu software).

### Example 8: Genetic Engineering of Moraxella catarrhalis Results

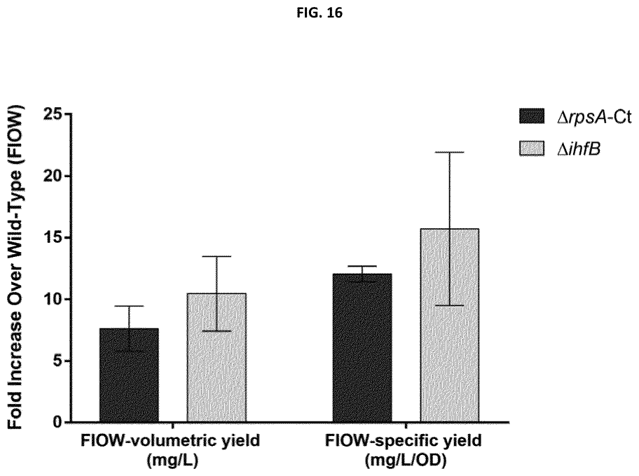

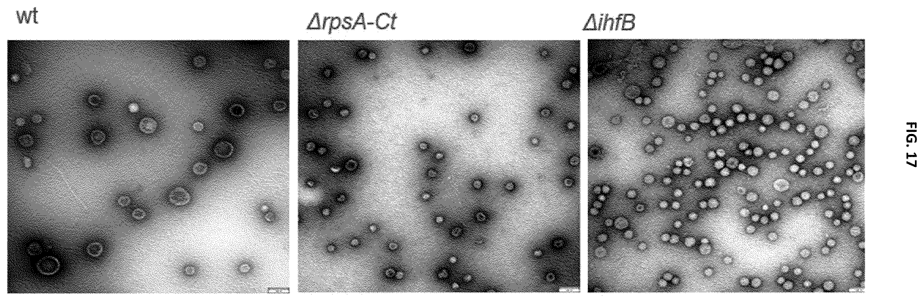

The rpsA and ihfB mutants showed an increase in GMMA production compared to wild type strain between 7.5 to 14-fold in terms of volumetric yield, and 12 to 22-fold in terms of specific yield (FIG. 16).

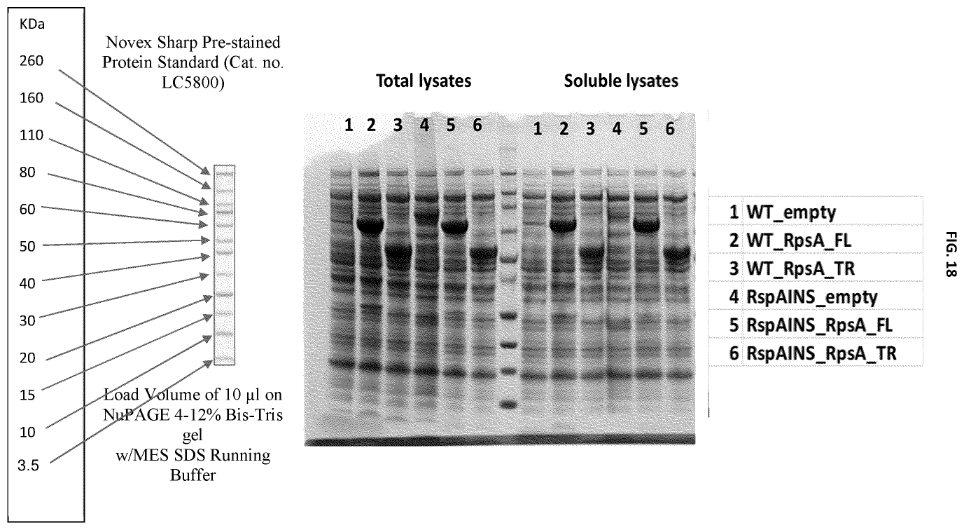

Negative staining electron microscopy (FIG. 17) also confirmed GMMA production. It also showed the sizes of the respective GMMA, with large GMMA for wt GMMA relative to ΔrpsA-Ct and ΔihfB GMMA, with ΔihfB GMMA being slightly larger than ΔrpsA-Ct GMMA. The ΔrpsA-Ct and ΔihfB GMMA also have a consistent size.

### Example 9: Determining Whether rpsA Truncation is Responsible for Hyperblebbing: Materials and Methods

To further investigate the mechanism behind the RpsA hyperblebbing mutation and particularly if it is due to the RpsA truncation or downregulation, E. coli strains overexpressing the full-length (“FL”) or the R4 truncated (“TR”) EcRpsA in wild type and RpsA mutant background were generated and tested for GMMA production yields.

### Primer Design and Constructs Generation

Expression vector pET15-TEV-ccdB plasmid (ampR) was used for RpsA overexpression, to allow PIPE cloning and use the strong inducible T7 promoter. No tag was added and therefore the His-tag and the TEV site were excluded from the final construct.

| Primer name | Sequence 5′-3′ | Use |
|---|---|---|
| V-PIPE Univ-F | TAACGCGACTTAATTGGCCAGTGTGCCGGTCTCC | pET15 vector amplification |
| | G | |
| pET15notagRv | CATGGTATATCTCCTTCTTAAAGTTAAAC | |
| rpsAfw | AAGGAGATATACCATGACTGAATCTTTTGCTCAA | Forward primer for rpsA FL and |
| | CTC | TR amplification |
| rpsAFLrv | AATTAAGTCGCGTTACTCGCCTTTAGCTGCTTTG | Reverse primer for rpsA FL |
| | | amplification |
| rpsATRrv | AATTAAGTCGCGTTATGCGAGCTGTTTAACGCCC | Reverse primer for rpsA TR and |
| | AG | TR amplification |
| T7prom | TAATACGACTCACTATAGGG | Colony screening |
| Seq Pet rv | GATATCCGGATATAGTTCCTC | |

For PCR amplification, purified pET15-TEV-ccdB plasmidic DNA and E. coli BL21(DE3) genomic DNA were used as template for vector and inserts amplification respectively. Kapa Hifi mastermix (Roche) was used for the reaction with the following conditions:

| | | |
|---|---|---|
| Template | 1 | μl |
| Primer BlaIgAUpfw 50 uM | 0.5 | μl |
| Primer BlaIgADorv 50 uM | 0.5 | μl |
| KapaHifi 2X | 25 | μl |
| H₂0 | 23 | μl |
| Tot | 50 | μL |

### Amplification Cycle

| | | |
|---|---|---|
| 95° C. | 5′ | |
| 98° C. | 30″ | 25x |
| 55° C. | 30″ | |
| 72° C. | 3′30″ (V-PCR) or | |
| | 45″ (I-PCR) | |

For cloning 1 μl of amplified vector and 1 μl of each amplified insert were mixed and transformed in 50 μl of Invitrogen™ One Shot™ Mach1™ T1 Phage-Resistant Chemically Competent E. coli cells (Thermo Scientific C862003)

Three clones for each transformation were screened with external primers by colony PCR using GoTaq2X Green Master Mix (Promega) according to manufacturer's instructions. Reaction was carried out in 20 μl.

Cycle: 95° C. for 5′-(95° C. for 30″; 55° C. for 30″; 72° C. 1′30″)×30-72° C. for 7′

Finally, selected clones were subjected to sequencing to confirm that no mutations were introduced into the coding sequence during cloning.

### Strain Generation

Home-made chemically competent E. coli BL21(DE3) INS rpsA (construct #7) mutant cells and wild type BL21(DE3) (One Shot™ BL21(DE3) Chemically Competent E. coli, thermo scientific cat #C600003) were transformed with the following plasmids:
a. pET21b+ empty (pET-21b(+) DNA—Novagen | 69741—Merck Millipore, lot #M00067689): empty vector as control
b. pET15-RpsAFL: overexpressing vector for RpsA full length
c. pET15-RpsATr: overexpressing vector for RpsA truncated

### Small Scale Cultivation

A pre-inoculum of 5 ml LB medium+ampicillin 100 mg/L was incubated 8 h at 37° C. and 180 rpm.

Pre-cultures were diluted 1:100 in 50 mL HTMC medium+ampicillin 100 mg/L in disposable baffled 250 mL flasks with vented cup (Corning) and incubated at 30° C. and 200 rpm for 15 h. OD600 nm was registered (1:40 dilution in HTMC medium). Cultures were then centrifuged at 2000 rpm for 10′ at 4° C. and supernatant was discarded. Pellets were resuspended in 50 mL of fresh HTMC medium supplemented with ampicillin 100 mg/L and IPTG 1 mM to induce recombinant RpsA expression. Since dead bacteria were discarded with the spent supernatant the new bacteria concentration in terms of OD600 nm was recorded after resuspension. Cultures were re-incubated at 30° C. and 200 rpm ad OD600 nm was monitored until stationary growth phase was reached (1:40 dilution in HTMC medium).

The cultures were finally harvested by centrifugation at 4000 rpm for 30′ at 4° C. The resulting supernatants were filtered through a 0.22 μm stericup (Millipore) and stored at 4° C. for further analysis. Pellets were stored at −20° C. until further characterization.

### Evaluation of RpsA Recombinant Expression and Solubility

To verify the expression of RpsA (FL and TR) a total lysate and soluble lysate was prepared from collected pellets to be compared in SDS-PAGE. Briefly, pellets from 50 mL cultures were resuspended in a volume corresponding to final OD600 nm value to obtain a 50 OD/ml suspension in filter sterilized PBS, 0.5 mL of each suspension were centrifuged for 5′ at 13000 rmp at 4° C. and supernatant discarded. Each pellet was then resuspended in 1 mL of CelLytic express (Sigma Aldrich) previously solubilized in PBS and incubated at room temperature for 30′ under mild agitation. 50 μl of total lysates were transferred in a new tube (total lysate) while remaining suspensions were centrifuged at 13000 rmp at 4° C. for 15′ to obtain soluble fraction. Total and soluble lysates were stored at −20° C. until further manipulation.

### SDS-PAGE Analysis

To check RpsA expression, 13 μl of each total and soluble lysates prepared as described above and previously diluted 1:5 in PBS (20 μl sample+80 μl PBS) were mixed with 5 μl of 4× loading sample buffer and 2 μl of 10× reducing agent in a total of 20 μl.

For GMMA protein pattern analysis, 5 μg of each GMMA preparation were mixed 5 μl of 4× loading sample buffer and 2 μl of 10× reducing agent in a total of 20 μl.

All samples were treated at 100° C. for 5′ in a thermoblock for denaturation. Samples were loaded in a 4-12% Bis-Tris polyacrylamide gel (NuPage, Thermo Scientific). As molecular weight marker, 10 μl of NovexSharp Pre-stained protein ladder (Thermo Scientific) were loaded. Gel was run at 200V for 40 min in MES buffer. Staining was performed with Coomassie (Giotto Biotech) 1 h at room temperature under agitation. De-staining was done overnight at room temperature under agitation. Gel image was acquired with GelDoc XR (BioRad) and ImageLab software.

### GMMA Purification

To purify vesicles, 35 mL of supernatant was subjected to ultracentrifugation at 32000 rpm for 3 h at 4° C. ultracentrifugated 35 ml supernatant at 32000 rpm/4° C./3 h (using 32Ti swinging rotor). One washing step is performed with 35 ml sterile PBS at 32000 rpm/4° C./2 h. Finally vesicles containing pellets were resuspended in 0.15-0.6 mL of sterile PBS for 16 h at 4° C. while shaking.

### GMMA Quantification

Purified GMMA were quantified by Lowry assay following the protocol “DC Protein Assay” (CAT No 500-0114) from BIORAD. Serial step 2 dilutions of 1 mg/ml BSA were used for standard curve.

GMMA are also quantified directly from the supernatants by a fluorescence assay that uses the FM4-64 dye (Thermofisher Scientific, catalog number: T13320). It stains the Outer Membrane Vesicles with red fluorescence (excitation/emission 515/640 nm). The dye is used with a final dilution 1:100.

### Example 10: Determining Whether rpsA Truncation is Responsible for Hyperblebbing: Results

### Strains Cultivation and RpsA Overexpression

Following strains were cultivated in small scale to induce RpsA overexpression and evaluate GMMA production:
1. BL21(DE3)-pET21b+empty (WT_empty)
2. BL21(DE3)-pET15-RpsAFL (WT_RpsA_FL)
3. BL21(DE3)-pET15-RpsATR (WT_RpsA_TR)
4. BL21(DE3) RpsAINS-pET21b+empty (RpsAINS_empty)
5. BL21(DE3) RpsAINS-pET15-RpsAFL (RpsAINS_RpsA_FL)
6. BL21(DE3) RpsAINS-pET15-RpsATR (RpsAINS_RpsA_TR)

The overexpression of both RpsA full length (FL) and truncated (TR) was induced in both BL21(DE3) wild type and BL21(DE3) RpsAINS hyperblebbing mutant. Strains transformed with empty pET vector were used as a negative control since the ampicillin in the medium could affect vesicle release during growth.

To evaluate RpsA expression and solubility in tested strains, an SDS-PAGE was run as shown in FIG. 18. Recombinant expression of both RpsA FL and TR was confirmed at high levels in both strains and fully soluble.

### Evaluation of GMMA Production Yield in Culture Supernatants

GMMA yield obtained for the different strains was evaluated directly from culture supernatant using the fluorescence assay with FM4-64 dye. This dye becomes fluorescent when it interacts with the double lipid layer and the fluorescence produced is proportional to the amount of vesicles in the tested sample.

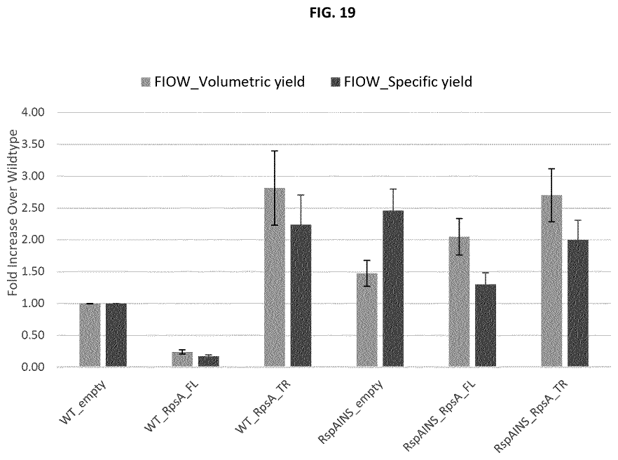

Fluorescence intensity recorded from culture supernatants was compared as absolute fluorescence as average of the three technical replicates (error bars were calculated as derived from the blank value subtraction) and reported as volumetric yield. Fluorescence intensities were also normalized on the OD₆₀₀ₙₘ value in order to calculate the specific GMMA yield for each mutant. The FIOW (Fold Increase Over Wildtype) was calculated using as baseline the average value of wild type strain transformed with empty plasmid as both volumetric and specific FIOW (FIG. 19).

### GMMA Purification and Characterization

To confirm what was observed in culture supernatants, GMMA were purified by ultracentrifugation and quantified based on total protein content by Lowry assay. Both volumetric and specific yields were calculated and reported as Fold Increase Over Wildtype using as baseline the average value of wild type strain transformed with empty plasmid. Values obtained are in the table below:

| | | Conc | Vol | mg tot | mg/L | OD | mg/L/OD | FIOW |
|---|---|---|---|---|---|---|---|---|
| 1 | WT_empty | 0.28 | 0.4 | 0.113 | 3.227126 | 22.2 | 0.145366 | 1 |
| 2 | WT_RpsA_FL | 0.28 | 0.25 | 0.070 | 2.003864 | 31.2 | 0.064226 | 0.441826 |
| 3 | WT_RpsA_TR | 0.83 | 0.33 | 0.275 | 7.865369 | 27.9 | 0.281913 | 1.939332 |
| 4 | RspAINS_empty | 2.44 | 0.27 | 0.659 | 18.82909 | 13.3 | 1.415721 | 9.739013 |
| 5 | RspAINS_RpsA_FL | 0.97 | 0.37 | 0.359 | 10.24764 | 35 | 0.29279 | 2.014156 |
| 6 | RspAINS_RpsA_TR | 0.84 | 0.31 | 0.260 | 7.422172 | 30 | 0.247406 | 1.70195 |

To confirm, the number in the box highlighted in the table above is 9.739013.

The obtained FIOW values resemble data obtained with direct GMMA quantification in culture supernatants by FM4-64 staining with the exception of RpsAINS_empty which shows a FIOW much higher than expected.

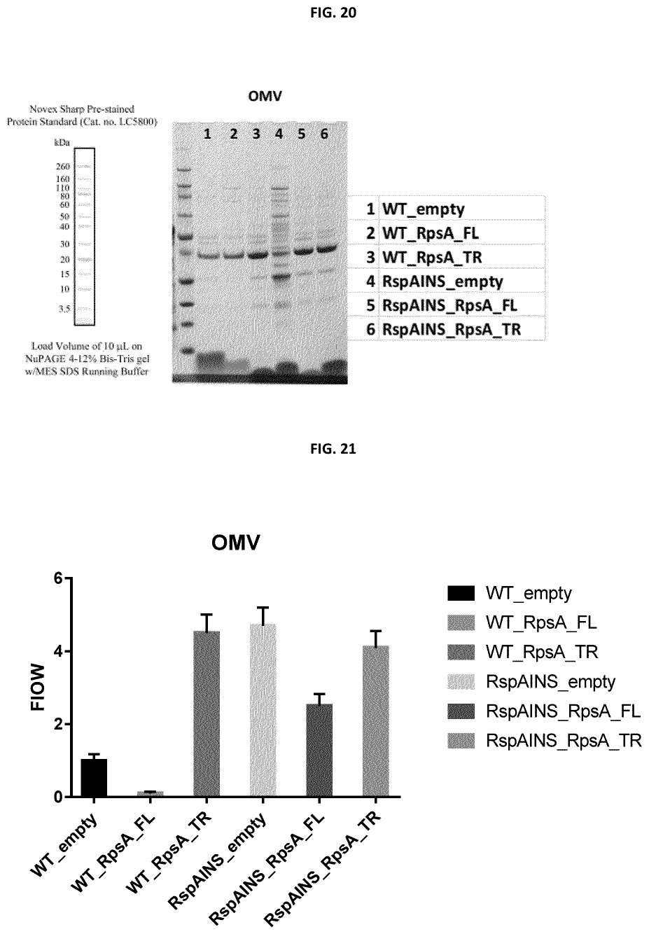

To verify the purity of the purified GMMA, an SDS-PAGE was run (FIG. 20).

GMMA preparations were all highly pure with the exception of RpsAINS_empty sample where a clear contamination of soluble protein is visible on the gel. This was expected because of the quantification results which was not in line with the fluorescence detected in culture supernatants. Since the RpsAINS_empty yield is hugely overestimated due to the low purity of obtained sample, the calculated GMMA yield calculated based on total protein content (by lowry) for this strain must be normalized.

To try to have a more accurate estimation of GMMA yield obtained through RpsA mutation/complementation, densitometry was used to evaluate the purity of the 30 kDa porin which amount is constant in different GMMA preparations using ImageLab software.

Then FIOW values were normalized on the purity ratio for different strains as reported in the table below:

| | | | | | | | | | | normalized | |
|---|---|---|---|---|---|---|---|---|---|---|---|
| | | Conc | Vol | mg tot | mg/L | OD | mg/L/OD | FIOW | Purity | mg/L/OD | FIOW |
| 1 | WT_empty | 0.28 | 0.4 | 0.113 | 3.227126 | 22.2 | 0.145366 | 1 | 1 | 0.145366 | 1.00 |
| 2 | WT_RpsA_FL | 0.28 | 0.25 | 0.070 | 2.003864 | 31.2 | 0.064226 | 0.441826 | 1.16246 | 0.074661 | 0.51 |
| 3 | WT_RpSA_TR | 0.83 | 0.33 | 0.275 | 7.865369 | 27.9 | 0.281913 | 1.939332 | 1.039205 | 0.292965 | 2.02 |
| 4 | RspAINS_empty | 2.44 | 0.27 | 0.659 | 18.82909 | 13.3 | 1.415721 | 9.739013 | 0.242513 | 0.343331 | 2.36 |
| 5 | RspAINS_RpsA_FL | 0.97 | 0.37 | 0.359 | 10.24764 | 35 | 0.29279 | 2.014156 | 1.051952 | 0.308001 | 2.12 |
| 6 | RspAINS_RpsA_TR | 0.84 | 0.31 | 0.260 | 7.422172 | 30 | 0.247406 | 1.70195 | 1.074819 | 0.265916 | 1.83 |
| | | | ref | | | | | | | | |
| | | % lane | 61.36302 | 71.332055 | 63.76877 | 14.88134 | 64.55094 | 65.95413 | | | |
| | | purity | 1 | 1.1624601 | 1.039205 | 0.242513 | 1.051952 | 1.074819 | | | |

To confirm, the number in the box highlighted in the table above is 9.739013.

With the normalization, the new data was more in line with what was observed with fluorescence intensity in culture supernatants.

To further confirm what was found by direct quantification of GMMA in culture supernatants and with purified GMMA quantified based on protein content and normalized on purity, the purified GMMA were also quantified using the fluorescent probe FM4-64. The rationale was that if low purity is a matter of a specific release of soluble intracellular proteins in culture supernatant which are co-purified with GMMA by ultracentrifugation, quantification based on lipid content will overcome this issue. There could anyway be an overestimation of the low purity sample due to the presence of cellular debris but it would not be different to what was observed in culture supernatants.

Specific yield (normalized on final OD values) was calculated based on recorded fluorescence intensities (FIG. 21). Using lipid based quantification, the FIOW profiles obtained are in line with what was found when analyzing the culture supernatants directly, confirming the effect of RpsA truncation on the hyperblebbing phenotype.

DESCRIPTION OF SEQUENCES

SEQ ID NO: 1
>Bordetella pertussis Tohama I rpsA gene [domains underlined]
ATGGATTCCAACCTAATGTCTTCCGTTTCCACCTCCGCCATCCTTGGCGGCGAAAACTTCGCCGACCTGTTCGC
AGAAAGCCTCAAGAGCCAGGACATGAAGTCCGGCGAGGTCATCAGCGCAGAAGTCGTGCGCGTCGACCACAACT
TCGTGGTCGTCAACGCCGGCCTGAAGTCCGAAGCGCTGATTCCCCTGGAAGAGTTCCTCAACGACCAGGGCGAA
CTCGAAGTTCAACCCGGCGACTTCGTCTCGGTGGCGATCGATTCGCTGGAGAACGGCTACGGCGACACCATCCT
GTCGCGCGACCGCGCCAAGCGTCTGTCGGCCTGGCTGCAACTGGAGCAGGCCCTCGAGAACGGCGAGCTGGTCA
CCGGCACGATCACCGGCAAGGTCAAGGGCGGCCTGACCGTCATGACCAACGGCATCCGCGCGTTCCTGCCCGGT
TCGCTGGTCGACCTGCGTCCGGTCAAGGACACCACGCCGTACGAAGGCAAGACCCTCGAATTCAAGGTCATCAA
GCTGGACCGCAAGCGCAACAACGTCGTGCTGTCGCGCCGCCAGGTGCTGGAAGCCAGCATGGGCGAAGAGCGCC
AGAAGCTGCTCGAGACGCTGCACGAAGGCGCGGTGGTCAAGGGCGTGGTCAAGAACATCACCGACTACGGCGCG
TTCGTCGACCTGGGCGGCATCGATGGCCTGCTGCACATCACCGACATGGCCTGGCGCCGTGTGCGTCACCCGTC
CGAAGTCCTGCAAGTGGGTCAGGAAGTCGAAGCCAAGGTGCTCAAGTTCGACCAGGAAAAGAGCCGCGTCTCCC
TGGGCGTCAAGCAGCTGGGCGAAGATCCGTGGGTGGGCCTGGCTCGCCGCTACCCGCAGGGCACCCGCCTGTTC
GGCAAGGTCACCAACCTGACCGACTACGGCGCGTTCGTCGAAGTCGAAGCCGGCATCGAAGGCCTGGTGCACGT
GTCCGAAATGGACTGGACCAACAAGAACGTCGATCCGCGCAAGGTCGTGACCCTGGGCGAAGAAGTCGAAGTCA
TGGTCCTGGAAATCGACGAAGACCGTCGCCGCATTTCGCTGGGCATGAAGCAGTGCCGCCAGAACCCGTGGGAA
GAGTTCGCCACCAACTTCAAGCGTGGTGACAAGGTCCGCGGCGCCATCAAGTCGATCACCGACTTCGGCGTGTT
CGTCGGCCTGCCCGGCGGCATCGACGGCCTGGTCCATCTGTCCGACCTGTCGTGGACGGAATCGGGCGAGGAAG
CCGTGCGCAACTTCAAGAAGGGCGACGAGCTGGAAGCCGTGGTGCTGGGCATCGATACCGAGAAAGAGCGCATC
TCGCTGGGTATCAAGCAGCTCGAAGGCGACCCGTTCAACAACTTCGTTGCCACGCACGACAAGGGCGCCGTTGT
TCCGGGCACCATCAAGTCGGTCGAGCCCAAGGGCGCCGTGATCACCCTGTCGGTGGATGTGGAAGGCTACCTGC
GCGCCTCCGAGATCTCCTCGGGCCGCGTCGAAGACGCTACCACCGTGCTGAAGGCTGGCGAGAACATCGAAGCC
ATGATCGTCAACATCGACCGCAAGGCGCGTTCGATCCAGCTGTCGATCAAGGCCCGCGATAACGCCGAGACGGC
CGAGACCATCCAGCGCATGTCCGAGGCGAGCGCTTCGTCGGGTACGACGAACTTGGGCGCGCTGCTCAAGGCCA
AGCTGGACCAACAGCGCAACGACGGTTGA
SEQ ID NO: 2
>Escherichia coli BL21 (DE3) rpsA gene
ATGACTGAATCTTTTGCTCAACTCTTTGAAGAGTCCTTAAAAGAAATCGAAACCCGCCCGGGTTCTATCGTTCG
TGGCGTTGTTGTTGCTATCGACAAAGACGTAGTACTGGTTGACGCTGGTCTGAAATCTGAGTCCGCCATCCCGG
CTGAGCAGTTCAAAAACGCCCAGGGCGAGCTGGAAATCCAGGTAGGTGACGAAGTTGACGTTGCTCTGGACGCA
GTAGAAGACGGCTTCGGTGAAACTCTGCTGTCCCGTGAGAAAGCTAAACGTCACGAAGCCTGGATCACGCTGGA
AAAAGCTTACGAAGATGCTGAAACTGTTACCGGTGTTATCAACGGCAAAGTTAAGGGCGGCTTCACTGTTGAGC
TGAACGGTATTCGTGCGTTCCTGCCAGGTTCTCTGGTAGACGTTCGTCCGGTGCGTGACACTCTGCACCTGGAA
GGCAAAGAGCTTGAATTTAAAGTAATCAAGCTGGATCAGAAGCGCAACAACGTTGTTGTTTCTCGTCGTGCCGT
TATCGAATCCGAAAACAGCGCAGAGCGCGATCAGCTGCTGGAAAACCTGCAGGAAGGCATGGAAGTTAAAGGTA
TCGTTAAGAACCTCACTGACTACGGTGCATTCGTTGATCTGGGCGGCGTTGACGGCCTGCTGCACATCACTGAC
ATGGCCTGGAAACGCGTTAAGCATCCGAGCGAAATCGTCAACGTGGGCGACGAAATCACTGTTAAAGTGCTGAA
GTTCGACCGCGAACGTACCCGTGTATCCCTGGGCCTGAAACAGCTGGGCGAAGATCCGTGGGTAGCTATCGCTA
AACGTTATCCGGAAGGTACCAAACTGACTGGTCGCGTGACCAACCTGACCGACTACGGCTGCTTCGTTGAAATC
GAAGAAGGCGTTGAAGGCCTGGTACACGTTTCCGAAATGGATTGGACCAACAAAAACATCCACCCGTCCAAAGT
TGTTAACGTTGGCGATGTAGTGGAAGTTATGGTTCTGGATATCGACGAAGAACGTCGTCGTATCTCCCTGGGTC
TGAAACAGTGCAAAGCTAACCCGTGGCAGCAGTTCGCGGAAACCCACAACAAGGGCGACCGTGTTGAAGGTAAA
ATCAAGTCTATCACTGACTTCGGTATCTTCATCGGCCTGGACGGCGGCATCGACGGCCTGGTTCACCTGTCTGA
CATCTCCTGGAACGTTGCAGGCGAAGAAGCAGTTCGTGAATACAAAAAAGGCGACGAAATCGCTGCAGTTGTTC
TGCAGGTTGACGCAGAACGTGAACGTATCTCCCTGGGCGTTAAACAGCTCGCAGAAGATCCGTTCAACAACTGG
GTTGCTCTGAACAAGAAAGGCGCTATCGTAACCGGTAAAGTAACTGCAGTTGACGCTAAAGGCGCAACCGTAGA
ACTGGCTGACGGCGTTGAAGGTTACCTGCGTGCTTCTGAAGCATCCCGTGACCGCGTTGAAGACGCTACCCTGG
TTCTGAGCGTTGGCGACGAAGTTGAAGCTAAATTCACCGGCGTTGATCGTAAAAACCGCGCAATCAGCCTGTCT
GTTCGTGCGAAAGACGAAGCTGACGAGAAAGATGCAATCGCAACTGTTAACAAACAGGAAGATGCAAACTTCTC
CAACAACGCAATGGCTGAAGCTTTCAAAGCAGCTAAAGGCGAGTAA
SEQ ID NO: 3
>Haemophilus influenzae Rd_KW20 HI1220 rpsA gene
ATGTCAGAATCTTTTGCTCAACTTTTTGAAGAATCATTAAAAGTCCTTGAAACTCGTCAAGGTTCAATCGTTAG
CGGTACTGTAGTAGCTATCCAAAAAGGCTTTGTGCTTGTTGATGCAGGTTTAAAATCTGAGTCTGCAATTCCAG
TTGCTGAATTCTTAAATGCACAAGGCGAACTTGAAATCCAAGTTGGCGATACTGTAAATGTTGCATTAGATGCA
GTTGAAGATGGTTTCGGTGAAACTAAACTTTCTCGTGAGAAAGCGGTTCGTCACGAATCTTGGATTGAATTAGA
AAAAGCTTACGAAGAAAAAGCGACCGTTATCGGTTTAATCANCGGCAAAGTGAAAGGTGGCTTCACAGTTGAGT
TAAACGGTGTTCGTGCATTCTTACCAGGTTCTTTAGTTGATACTCGTCCAGCGCGTGAAGCAGATCACTTACTT
GGTAAAGAATTAGAATTCAAAGTAATCAAATTAGATCAAAAACGTAACAACGTTGTTGTTTCTCGTCGTGCAGT
AATTGAATCTGAAAACAGCCAAGAACGTGAACAAGTATTAGAAAATCTTGTGGAAGGNTCAGAAGTTAAAGGTG
TCGTTAAAAACTTAACTGAGTACGGTGCATTCGTTGATCTTGGTGGTGTTGATGGTTTATTACACATCACAGAT
ATGGCTTGGAAACGTGTTAAACACCCAAGTGAAATCGTAAATGTTGGCGATGAAGTTACTGTTAAAGTATTAAA
ATTCGATAAAGATCGTACTCGTGTATCTTTAGGCTTAAAACAATTAGGTCAAGATCCATGGGCAGCAATTGCTG
AAAATCATCCAGTAAACAGCAAATTAACAGGTAAAGTAACTAACTTAACTGACTATGGCTGTTTCGTTGAAATC
TTAGATGGCGTTGAAGGTTTAGTTCACGTTTCTGAAATGGATTGGACTAATAAAAATATCCACCCATCTAAAGT
GGTTAGCTTAGGCGATACTGTAGAAGTAATGGTATTAGAAATTGATGAAGAACGTCGTCGTATTTCTTTAGGCT
TAAAACAATGTAAAGCTAACCCTTGGACTCAGTTTGCTGATACTCACAACAAAGGCGATAAAGTAACGGGTAAA
ATCAAGTCTATCACTGATTTCGGTATCTTCATCGGTCTTGAAGGTGGTATCGATGGTTTAGTTCACTTATCTGA
TATTTCTTGGAGTATTTCAGGCGAAGAAGCTGTTCGTCAATACAAAAAAGGTGACGAAGTTTCAGCTGTTGTAT
TAGCTGTTGATGCAGTAAAAGAACGTATCTCTTTAGGTATTAAACAACTTGAAGAAGATCCATTCAATAACTTT
GTCGCAATCAACAAAAAAGGTGCGGTAGTATCTGCAACTGTTGTTGAAGCAGATGCTAAAGGTGCTAAAGTTGA
ATTAGCAGGTGGTGTTGAAGGTTATATCCGTTCTGCTGATTTAACAAGTGAAGTAGCTGTTGGTGATGTCGTTG
AAGCGAAATACACTGGAGTAGATCGTAAATCTCGCATTGTTCACTTATCAGTGAAAGCAAAAGATCAAGCTGAA
GAAGCTGCGGCAGTTGCAAGTGTAAATAACAAACAAGAAGATATTGTTATTCCAAATGCAATGGCTGAAGCATT
TAAAGCAGCTAAAGGTGAATAA
SEQ ID NO: 4
>Neisseria gonorrhoeae FA1090 rpsA gene
ATGTCTATGGAAAATTTTGCTCAGCTGTTGGAAGAAAGCTTTACCCTGCAAGAAATGAACCCGGGTGAGGTGAT
TACCGCTGAAGTAGTGGCAATCGACCAAAACTTCGTTACCGTAAACGCAGGTCTGAAATCAGAATCCCTGATCG
ATGTAGCTGAATTCAAAAACGCTCAAGGCGAAATTGAAGTTAAAGTCGGCGACTTCGTTACCGTTACCATCGAA
TCCGTCGAAAACGGCTTCGGCGAAACCAAACTGTCCCGCGAAAAAGCCAAACGCGCAGCCGATTGGATCGCTTT
GGAAGAAGCCATGGAAAACGGCAACATCCTGTCCGGCATCATCAACGGTAAAGTCAAAGGCGGCCTGACCGTTA
TGATCAGCAGCATCCGCGCATTCCTGCCGGGTTCTTTGGTCGACGTACGTCCCGTTAAAGACACTTCCCATTTT
GAAGGCAAAGAGATCGAATTCAAAGTGATCAAACTGGACAAAAAACGCAACAACGTCGTTGTTTCCCGCCGCGC
CGTTTTGGAAGCCACTTTGGGTGAAGAACGCAAAGCCCTGCTGGAAAACCTGCAAGAAGGCTCCGTCATCAAAG
GCATCGTCAAAAATATCACCGACTACGGCGCATTCGTTGACCTGGGCGGCATCGACGGCCTGCTGCACATCACC
GATTTGGCATGGCGTCGCGTGAAACACCCGAGCGAAGTCTTGGAAGTCGGTCAGGAAGTTGAAGCCAAAGTATT
GAAATTCGACCAAGAAAAACAACGTGTTTCCTTGGGTATGAAACAACTGGGCGAAGATCCTTGGAGCGGTCTGA
CCCGCCGTTATCCGCAAGCCACCCGCCTGTTCGGCAAAGTATCCAACCTGACCGACTACGGCGCATTCGTCGAA
ATCGAACAAGGCATCGAAGGTTTGGTACACGTCTCCGAAATGGACTGGACCAACAAAAACGTACACCCGAGCAA
AGTCGTACAACTGGGTGACGAAGTCGAAGTCATGATTTTGGAAATCGACGAAGGCCGCCGCCGTATCTCTTTGG
GTATGAAACAATGCCAAGCCAATCCTTGGGAAGAATTTGCCGCCAACCACAACAAAGGAGACAAAATCTCCGGT
GCGGTTAAATCCATTACCGATTTCGGCGTATTCGTCGGCCTGCCCGGCGGCATCGACGGTCTGGTTCACCTGTC
CGACCTGTCTTGGACCGAATCCGGCGAAGAAGCCGTACGCAAATACAAAAAAGGAGAAGAAGTCGAAGCCGTCG
TATTGGCAATCGATGTGGAAAAAGAACGCATCTCCTTGGGTATCAAACAACTGGAAGGCGATCCTTTCGGCAAC
TTCATCAGCGTGAACGACAAAGGTTCTTTGGTTAAAGGTTCCGTGAAATCTGTTGACGCCAAAGGCGCTGTTAT
CGCCCTGTCTGACGAAGTAGAAGGCTACCTGCCTGCTTCCGAATTTGCAGCCGACCGCGTTGAAGACTTGACCA
CCAAACTGAAAGAAGGCGACGAAGTTGAAGCCGTCATCGTTACCGTTGACCGCAAAAACCGCAGCATCAAACTT
TCCGTTAAAGCCAAAGATGCCAAAGAAAGCCGCGAAGCACTGAACTCCGTCAATGCCGCCGCCAATGCGAATGC
CGGTACCACCAGCTTGGGCGACCTGCTGAAAGCCAAACTCTCCGGCGAACAAGAATAA
SEQ ID NO: 5
>Neisseria meningitidis MC58 rpsA gene
ATGTCTATGGAAAATTTTGCTCAGCTGTTGGAAGAAAGCTTTACCCTGCAAGAAATGAACCCGGGTGAGGTGAT
TACCGCTGAAGTAGTGGCAATCGACCAAAACTTCGTTACCGTAAACGCAGGTCTGAAATCAGAATCCCTGATTG
ATGTAGCTGAATTCAAAAACGCTCAAGGCGAAATTGAAGTTAAAGTCGGCGACTTCGTTACCGTTACCATCGAA
TCCGTCGAAAACGGCTTCGGCGAAACCAAACTGTCCCGCGAAAAAGCCAAACGTGCAGCCGATTGGATTGCCCT
GGAAGAAGCCATGGAAAACGGCGACATCCTGTCCGGCATCATCAACGGAAAAGTCAAAGGCGGCCTGACCGTTA
TGATTAGCAGCATCCGCGCATTCCTGCCGGGTTCTTTGGTCGACGTACGTCCTGTAAAAGACACTTCTCACTTC
GAAGGCAAAGAGATCGAATTCAAAGTGATCAAACTGGACAAAAAACGCAACAACGTCGTTGTTTCCCGCCGCGC
CGTTCTGGAAGCCACTTTGGGTGAAGAACGCAAAGCCCTGCTGGAAAACCTGCAAGAAGGCTCCGTCATCAAAG
GCATCGTTAAAAACATTACCGATTACGGTGCATTCGTTGACTTGGGCGGCATCGACGGTCTGTTGCACATCACC
GATTTGGCATGGCGGCGCGTGAAACACCCGAGTGAAGTCTTGGAAGTCGGTCAGGAAGTTGAAGCCAAAGTATT
GAAATTCGACCAAGAAAAACAACGCGTTTCCTTGGGTATGAAACAACTGGGCGAAGATCCTTGGAGCGGTCTGA
CCCGCCGTTATCCTCAAGGCACCCGCCTGTTCGGCAAAGTATCCAACCTGACCGACTACGGCGCATTCGTCGAA
ATCGAACAAGGCATCGAAGGTTTGGTACACGTCTCCGAAATGGACTGGACCAACAAAAACGTACACCCGAGCAA
AGTCGTACAACTGGGCGACGAAGTCGAAGTCATGATTTTGGAAATCGACGAAGGCCGCCGCCGTATCTCTTTGG
GTATGAAACAATGCCAAGCCAATCCTTGGGAAGAATTTGCCGCCAACCACAACAAAGGCGACAAAATCTCCGGC
GCGGTTAAATCCATTACCGATTTCGGCGTATTCGTCGGCCTGCCCGGCGGCATCGACGGTTTGGTTCACCTGTC
CGACCTGTCCTGGACCGAATCCGGCGAAGAAGCCGTACGCAAATACAAAAAAGGCGAAGAAGTCGAAGCCGTCG
TATTGGCAATCGACGTGGAAAAAGAACGCATCTCCTTGGGTATCAAACAACTGGAAGGCGATCCGTTCGGCAAC
TTCATCAGCGTGAACGACAAAGGTTCTTTGGTTAAAGGTTCCGTGAAATCTGTTGACGCCAAAGGTGCTGTTAT
CGCCCTGTCTGACGAAGTAGAAGGCTACCTGCCTGCTTCCGAATTTGCAGCCGACCGCGTTGAAGATTTGACCA
CCAAACTGAAAGAAGGCGACGAAGTTGAAGCCGTCATCGTTACCGTTGACCGCAAAAACCGCAGCATCAAACTT
TCCGTTAAAGCCAAAGATGCCAAAGAAAGCCGCGAAGCACTGAACTCCGTCAATGCCGCCGCCAATGCGAATGC
CGGCACCACCAGCTTGGGCGACCTGCTGAAAGCCAAACTCTCCGGCGAACAAGAATAA
SEQ ID NO: 6
>Pseudomonas aeruginosa PAO1 rpsA gene
ATGAGCGAAAGCTTCGCAGAACTCTTTGAAGAAAGTCTGAAATCCCTCGACATGCAGCCGGGTGCCATCATCAC
CGGCATCGTGGTCGACATCGATGGTGACTGGGTCACCGTCCATGCCGGTCTGAAATCCGAGGGCGTCATCCCGG
TCGAGCAGTTCTACAACGAACAGGGCGAGCTGACCATCAAGGTGGGTGACGAAGTCCACGTCGCACTGGACGCG
GTAGAAGACGGCTTTGGCGAGACCAAGCTGTCCCGCGAGAAAGCCAAGCGCGCCGAGAGCTGGATTGTTCTGGA
AGCGGCTTTCGCTGCCGACGAAGTGGTCAAGGGCGTCATCAACGGCAAGGTCAAGGGCGGTTTCACCGTCGACG
TCAACGGCATCCGCGCGTTCCTGCCGGGTTCTCTGGTCGACGTTCGTCCGGTTCGCGACACCACCCACCTGGAA
GGCAAAGAGCTCGAGTTCAAGGTCATCAAGCTCGACCAGAAGCGCAACAACGTTGTCGTTTCCCGCCGCAGCGT
CCTGGAAGCCGAGAACAGCGCCGAGCGTGAAGCTCTGCTGGAATCGCTGCAGGAAGGCCAGCAGGTCAAAGGTA
TCGTCAAGAACCTCACCGACTACGGCGCATTCGTGGACCTGGGCGGCGTAGACGGCCTGCTACACATCACCGAC
ATGGCCTGGAAGCGCATCAAGCATCCGTCCGAGATCGTCAACGTTGGCGACGAGATCGACGTCAAGGTCCTGAA
GTTCGACCGCGAGCGCAACCGTGTATCCCTGGGCCTGAAGCAACTGGGCGAAGACCCGTGGGTTGCCATCAAGG
CGCGTTACCCGGAAGGTACCCGCGTCATGGCCCGCGTCACCAACCTCACCGACTACGGCTGCTTCGCCGAGCTG
GAAGAGGGCGTGGAAGGCCTGGTACACGTCTCCGAAATGGACTGGACCAACAAGAACATCCATCCGTCGAAAGT
CGTCCAGGTTGGCGATGAAGTGGAAGTTCAGGTTCTGGACATCGACGAAGAGCGTCGTCGTATCTCCCTGGGTA
TCAAGCAGTGCAAATCCAACCCGTGGGAAGACTTCTCCAGCCAGTTCAACAAGGGTGACCGTATCTCCGGTACC
ATCAAGTCGATCACCGACTTCGGTATCTTCATCGGTCTGGACGGCGGCATCGACGGCCTGGTCCACCTGTCCGA
CATCTCCTGGAACGAAGTCGGCGAAGAAGCCGTACGTCGCTTCAAGAAGGGCGACGAGCTGGAAACCGTCATCC
TGTCGGTCGATCCGGAGCGCGAGCGCATCTCCCTGGGCATCAAGCAGCTGGAAGACGATCCGTTCTCCAACTAC
GCGTCCCTGCACGAGAAAGGCAGCATCGTCCGCGGTACCGTGAAGGAAGTCGACGCCAAGGGCGCTGTCATCAG
CCTGGGCGACGACATCGAAGGTATCCTGAAGGCTTCCGAAATCAGCCGTGACCGCGTCGAAGACGCGCGCAACG
TCCTGAAGGAAGGCGAGGAAGTCGAAGCCAAGATCATCAGCATCGACCGCAAGAGCCGCGTCATCAGCCTCTCC
GTCAAGTCCAAGGACGTCGACGACGAGAAGGACGCAATGAAAGAACTGCGTAAGCAGGAAGTAGAAAGCGCTGG
TCCGACCACCATCGGTGATCTGATCCGTGCTCAGATGGAGAATCAGGGCTAA
SEQ ID NO: 7
>Bordetella pertussis Tohama I rpsA operon (rpsA-ihfB locus) [50D6 clone
insertion site in bold, 77D8 insertion site underlined with single line,
83G6 underslined with double line]
ATGGATTCCAACCTAATGTCTTCCGTTTCCACCTCCGCCATCCTTGGCGGCGAAAACTTCGCCGACCTGTTCGC
AGAAAGCCTCAAGAGCCAGGACATGAAGTCCGGCGAGGTCATCAGCGCAGAAGTCGTGCGCGTCGACCACAACT
TCGTGGTCGTCAACGCCGGCCTGAAGTCCGAAGCGCTGATTCCCCTGGAAGAGTTCCTCAACGACCAGGGCGAA
CTCGAAGTTCAACCCGGCGACTTCGTCTCGGTGGCGATCGATTCGCTGGAGAACGGCTACGGCGACACCATCCT
GTCGCGCGACCGCGCCAAGCGTCTGTCGGCCTGGCTGCAACTGGAGCAGGCCCTCGAGAACGGCGAGCTGGTCA
CCGGCACGATCACCGGCAAGGTCAAGGGCGGCCTGACCGTCATGACCAACGGCATCCGCGCGTTCCTGCCCGGT
TCGCTGGTCGACCTGCGTCCGGTCAAGGACACCACGCCGTACGAAGGCAAGACCCTCGAATTCAAGGTCATCAA
GCTGGACCGCAAGCGCAACAACGTCGTGCTGTCGCGCCGCCAGGTGCTGGAAGCCAGCATGGGCGAAGAGCGCC
AGAAGCTGCTCGAGACGCTGCACGAAGGCGCGGTGGTCAAGGGCGTGGTCAAGAACATCACCGACTACGGCGCG
TTCGTCGACCTGGGCGGCATCGATGGCCTGCTGCACATCACCGACATGGCCTGGCGCCGTGTGCGTCACCCGTC
CGAAGTCCTGCAAGTGGGTCAGGAAGTCGAAGCCAAGGTGCTCAAGTTCGACCAGGAAAAGAGCCGCGTCTCCC
TGGGCGTCAAGCAGCTGGGCGAAGATCCGTGGGTGGGCCTGGCTCGCCGCTACCCGCAGGGCACCCGCCTGTTC
GGCAAGGTCACCAACCTGACCGACTACGGCGCGTTCGTCGAAGTCGAAGCCGGCATCGAAGGCCTGGTGCACGT
GTCCGAAATGGACTGGACCAACAAGAACGTCGATCCGCGCAAGGTCGTGACCCTGGGCGAAGAAGTCGAAGTCA
TGGTCCTGGAAATCGACGAAGACCGTCGCCGCATTTCGCTGGGCATGAAGCAGTGCCGCCAGAACCCGTGGGAA
GAGTTCGCCACCAACTTCAAGCGTGGTGACAAGGTCCGCGGCGCCATCAAGTCGATCACCGACTTCGGCGTGTT
CGTCGGCCTGCCCGGCGGCATCGACGGCCTGGTCCATCTGTCCGACCTGTCGTGGACGGAATCGGGCGAGGAAG
CCGTGCGCAACTTCAAGAAGGGCGACGAGCTGGAAGCCGTGGTGCTGGGCATCGATACCGAGAAAGAGCGCATC
TCGCTGGGTATCAAGCAGCTCGAAGGCGACCCGTTCAACAACTTCGTTGCCACGCACGACAAGGGCGCCGTTGT
TCCGGGCACCATCAAGTCGGTCGAGCCCAAGGGCGCCGTGATCACCCTGTCGGTGGATGTGGAAGGCTACCTGC
GCGCCTCCGAGATCTCCTCGGGCCGCGTCGAAGACGCTACCACCGTGCTGAAGGCTGGCGAGAACATCGAAGCC
ATGATCGTCAACATCGACCGCAAGGCGCGTTCGATCCAGCTGTCGATCAAGGCCCGCGATAACGCCGAGACGGC
CGAGACCATCCAGCGCATGTCCGAGGCGAGCGCTTCGTCGGGTACGACGAACTTGGGCGCGCTGCTCAAGGCCA
AGCTGGACCAACAGCGCAACGACGGTTGACGTGACCAAGTCGGAGCTGATCGCCGCGCTGGCGGCCCGCTATCC
TCAGCTGGCCGCTCGCGACACCGATTACGCTGTCAAGACCATGCTCGATGCAATGACCCAGGCCCTGGCCTCGG
GTCAGCGCATCGAAATCCGCGGGTTTGGCAGCTTTTCGCTGTCGCAGCGCTCTCCCCGTATCGGGCGCAATCCG
AAGTCGGGCGAACAAGTGCTGGTGCCTGGCAAGCAGGTGCCGCACTTCAAGGCCGGCAAGGAGCTGCGCGAGTG
GGTCGATCTGGTTGGCAACGATCAGGGCGACGACTCGTCCAACGGGTCGTCCGACCCGCTGCAATCGGTCATGG
ATATGCATGCCATGCACTGA
SEQ ID NO: 8
>Escherichia coli BL21 (DE3) rpsA operon (rpsA-ihfB locus) [intergenic region
underlined]
ATGACTGAATCTTTTGCTCAACTCTTTGAAGAGTCCTTAAAAGAAATCGAAACCCGCCCGGGTTCTATCG
TTCGTGGCGTTGTTGTTGCTATCGACAAAGACGTAGTACTGGTTGACGCTGGTCTGAAATCTGAGTCCGC
CATCCCGGCTGAGCAGTTCAAAAACGCCCAGGGCGAGCTGGAAATCCAGGTAGGTGACGAAGTTGACGTT
GCTCTGGACGCAGTAGAAGACGGCTTCGGTGAAACTCTGCTGTCCCGTGAGAAAGCTAAACGTCACGAAG
CCTGGATCACGCTGGAAAAAGCTTACGAAGATGCTGAAACTGTTACCGGTGTTATCAACGGCAAAGTTAA
GGGCGGCTTCACTGTTGAGCTGAACGGTATTCGTGCGTTCCTGCCAGGTTCTCTGGTAGACGTTCGTCCG
GTGCGTGACACTCTGCACCTGGAAGGCAAAGAGCTTGAATTTAAAGTAATCAAGCTGGATCAGAAGCGCA
ACAACGTTGTTGTTTCTCGTCGTGCCGTTATCGAATCCGAAAACAGCGCAGAGCGCGATCAGCTGCTGGA
AAACCTGCAGGAAGGCATGGAAGTTAAAGGTATCGTTAAGAACCTCACTGACTACGGTGCATTCGTTGAT
CTGGGCGGCGTTGACGGCCTGCTGCACATCACTGACATGGCCTGGAAACGCGTTAAGCATCCGAGCGAAA
TCGTCAACGTGGGCGACGAAATCACTGTTAAAGTGCTGAAGTTCGACCGCGAACGTACCCGTGTATCCCT
GGGCCTGAAACAGCTGGGCGAAGATCCGTGGGTAGCTATCGCTAAACGTTATCCGGAAGGTACCAAACTG
ACTGGTCGCGTGACCAACCTGACCGACTACGGCTGCTTCGTTGAAATCGAAGAAGGCGTTGAAGGCCTGG
TACACGTTTCCGAAATGGATTGGACCAACAAAAACATCCACCCGTCCAAAGTTGTTAACGTTGGCGATGT
AGTGGAAGTTATGGTTCTGGATATCGACGAAGAACGTCGTCGTATCTCCCTGGGTCTGAAACAGTGCAAA
GCTAACCCGTGGCAGCAGTTCGCGGAAACCCACAACAAGGGCGACCGTGTTGAAGGTAAAATCAAGTCTA
TCACTGACTTCGGTATCTTCATCGGCCTGGACGGCGGCATCGACGGCCTGGTTCACCTGTCTGACATCTC
CTGGAACGTTGCAGGCGAAGAAGCAGTTCGTGAATACAAAAAAGGCGACGAAATCGCTGCAGTTGTTCTG
CAGGTTGACGCAGAACGTGAACGTATCTCCCTGGGCGTTAAACAGCTCGCAGAAGATCCGTTCAACAACT
GGGTTGCTCTGAACAAGAAAGGCGCTATCGTAACCGGTAAAGTAACTGCAGTTGACGCTAAAGGCGCAAC
CGTAGAACTGGCTGACGGCGTTGAAGGTTACCTGCGTGCTTCTGAAGCATCCCGTGACCGCGTTGAAGAC
GCTACCCTGGTTCTGAGCGTTGGCGACGAAGTTGAAGCTAAATTCACCGGCGTTGATCGTAAAAACCGCG
CAATCAGCCTGTCTGTTCGTGCGAAAGACGAAGCTGACGAGAAAGATGCAATCGCAACTGTTAACAAACA
GGAAGATGCAAACTTCTCCAACAACGCAATGGCTGAAGCTTTCAAAGCAGCTAAAGGCGAGTAATTCTCT
GACTCTTCGGGATTTTTATTCCGAAGTTTGTTGAGTTTACTTGACAGATTGCAGGTTTCGTCCTGTAATC
AAGCACTAAGGGCGGCTACGGCCGCCCTTAATCAATGCAGCAACAGCAGCCGCTTAATTTGCCTTTAAGG
AACCGGAGGAATCATGACCAAGTCAGAATTGATAGAAAGACTTGCCACCCAGCAATCGCACATTCCCGCC
AAGACGGTTGAAGATGCAGTAAAAGAGATGCTGGAGCATATGGCCTCGACTCTTGCGCAGGGCGAGCGTA
TTGAAATCCGCGGTTTCGGCAGTTTCTCTTTGCACTACCGCGCACCACGTACCGGACGTAATCCGAAGAC
TGGCGATAAAGTAGAACTGGAAGGAAAATACGTTCCTCACTTTAAACCTGGTAAAGAACTGCGCGATCGC
GCCAATATTTACGGTTAA
SEQ ID NO: 9
>Neisseria meningitidis MC58 rpsA operon (rpsA-ihfB locus) [intergenic
region underlined; insertion site of cmR in bold]
ATGTCTATGGAAAATTTTGCTCAGCTGTTGGAAGAAAGCTTTACCCTGCAAGAAATGAACCCGGGTGAGGTGAT
TACCGCTGAAGTAGTGGCAATCGACCAAAACTTCGTTACCGTAAACGCAGGTCTGAAATCAGAATCCCTGATTG
ATGTAGCTGAATTCAAAAACGCTCAAGGCGAAATTGAAGTTAAAGTCGGCGACTTCGTTACCGTTACCATCGAA
TCCGTCGAAAACGGCTTCGGCGAAACCAAACTGTCCCGCGAAAAAGCCAAACGTGCAGCCGATTGGATTGCCCT
GGAAGAAGCCATGGAAAACGGCGACATCCTGTCCGGCATCATCAACGGAAAAGTCAAAGGCGGCCTGACCGTTA
TGATTAGCAGCATCCGCGCATTCCTGCCGGGTTCTTTGGTCGACGTACGTCCTGTAAAAGACACTTCTCACTTC
GAAGGCAAAGAGATCGAATTCAAAGTGATCAAACTGGACAAAAAACGCAACAACGTCGTTGTTTCCCGCCGCGC
CGTTCTGGAAGCCACTTTGGGTGAAGAACGCAAAGCCCTGCTGGAAAACCTGCAAGAAGGCTCCGTCATCAAAG
GCATCGTTAAAAACATTACCGATTACGGTGCATTCGTTGACTTGGGCGGCATCGACGGTCTGTTGCACATCACC
GATTTGGCATGGCGGCGCGTGAAACACCCGAGTGAAGTCTTGGAAGTCGGTCAGGAAGTTGAAGCCAAAGTATT
GAAATTCGACCAAGAAAAACAACGCGTTTCCTTGGGTATGAAACAACTGGGCGAAGATCCTTGGAGCGGTCTGA
CCCGCCGTTATCCTCAAGGCACCCGCCTGTTCGGCAAAGTATCCAACCTGACCGACTACGGCGCATTCGTCGAA
ATCGAACAAGGCATCGAAGGTTTGGTACACGTCTCCGAAATGGACTGGACCAACAAAAACGTACACCCGAGCAA
AGTCGTACAACTGGGCGACGAAGTCGAAGTCATGATTTTGGAAATCGACGAAGGCCGCCGCCGTATCTCTTTGG
GTATGAAACAATGCCAAGCCAATCCTTGGGAAGAATTTGCCGCCAACCACAACAAAGGCGACAAAATCTCCGGC
GCGGTTAAATCCATTACCGATTTCGGCGTATTCGTCGGCCTGCCCGGCGGCATCGACGGTTTGGTTCACCTGTC
CGACCTGTCCTGGACCGAATCCGGCGAAGAAGCCGTACGCAAATACAAAAAAGGCGAAGAAGTCGAAGCCGTCG
TATTGGCAATCGACGTGGAAAAAGAACGCATCTCCTTGGGTATCAAACAACTGGAAGGCGATCCGTTCGGCAAC
TTCATCAGCGTGAACGACAAAGGTTCTTTGGTTAAAGGTTCCGTGAAATCTGTTGACGCCAAAGGTGCTGTTAT
CGCCCTGTCTGACGAAGTAGAAGGCTACCTGCCTGCTTCCGAATTTGCAGCCGACCGCGTTGAAGATTTGACCA
CCAAACTGAAAGAAGGCGACGAAGTTGAAGCCGTCATCGTTACCGTTGACCGCAAAAACCGCAGCATCAAACTT
TCCGTTAAAGCCAAAGATGCCAAAGAAAGCCGCGAAGCACTGAACTCCGTCAATGCCGCCGCCAATGCGAATGC
CGGCACCACCAGCTTGGGCGACCTGCTGAAAGCCAAACTCTCCGGCGAACAAGAATAAGGTTGCAGACATGACA
AAGTCTGAGTTAATGGTTCGTTTGGCAGAAGTGTTTGCCGCCAAAAACGGCACGCATCTTCTGGCAAAAGACGT
AGAGTACAGCGTAAAAGTCTTGGTTGACACCATGACTAGATCGCTTGCCCGAGGTCAACGCATCGAAATCCGCG
GTTTCGGCAGCTTCGATTTGAACCATCGTCCTGCCCGCATCGGTCGCAATCCCAAAACCGGCGAGCGTGTGGAA
GTACCTGAAAAACATGTACCCCACTTCAAGCCCGGTAAAGAATTGCGCGAGCGGGTCGACTTGGCTTTAAAAGA
AAATGCCAATTAA
SEQ ID NO: 10
>Neisseria gonorrhoeae FA1090 rpsA operon (rpsA-ihfB locus) [intergenic
region underslined]
ATGTCTATGGAAAATTTTGCTCAGCTGTTGGAAGAAAGCTTTACCCTGCAAGAAATGAACCCGGGTGAGGTGAT
TACCGCTGAAGTAGTGGCAATCGACCAAAACTTCGTTACCGTAAACGCAGGTCTGAAATCAGAATCCCTGATCG
ATGTAGCTGAATTCAAAAACGCTCAAGGCGAAATTGAAGTTAAAGTCGGCGACTTCGTTACCGTTACCATCGAA
TCCGTCGAAAACGGCTTCGGCGAAACCAAACTGTCCCGCGAAAAAGCCAAACGCGCAGCCGATTGGATCGCTTT
GGAAGAAGCCATGGAAAACGGCAACATCCTGTCCGGCATCATCAACGGTAAAGTCAAAGGCGGCCTGACCGTTA
TGATCAGCAGCATCCGCGCATTCCTGCCGGGTTCTTTGGTCGACGTACGTCCCGTTAAAGACACTTCCCATTTT
GAAGGCAAAGAGATCGAATTCAAAGTGATCAAACTGGACAAAAAACGCAACAACGTCGTTGTTTCCCGCCGCGC
CGTTTTGGAAGCCACTTTGGGTGAAGAACGCAAAGCCCTGCTGGAAAACCTGCAAGAAGGCTCCGTCATCAAAG
GCATCGTCAAAAATATCACCGACTACGGCGCATTCGTTGACCTGGGCGGCATCGACGGCCTGCTGCACATCACC
GATTTGGCATGGCGTCGCGTGAAACACCCGAGCGAAGTCTTGGAAGTCGGTCAGGAAGTTGAAGCCAAAGTATT
GAAATTCGACCAAGAAAAACAACGTGTTTCCTTGGGTATGAAACAACTGGGCGAAGATCCTTGGAGCGGTCTGA
CCCGCCGTTATCCGCAAGCCACCCGCCTGTTCGGCAAAGTATCCAACCTGACCGACTACGGCGCATTCGTCGAA
ATCGAACAAGGCATCGAAGGTTTGGTACACGTCTCCGAAATGGACTGGACCAACAAAAACGTACACCCGAGCAA
AGTCGTACAACTGGGTGACGAAGTCGAAGTCATGATTTTGGAAATCGACGAAGGCCGCCGCCGTATCTCTTTGG
GTATGAAACAATGCCAAGCCAATCCTTGGGAAGAATTTGCCGCCAACCACAACAAAGGAGACAAAATCTCCGGT
GCGGTTAAATCCATTACCGATTTCGGCGTATTCGTCGGCCTGCCCGGCGGCATCGACGGTCTGGTTCACCTGTC
CGACCTGTCTTGGACCGAATCCGGCGAAGAAGCCGTACGCAAATACAAAAAAGGAGAAGAAGTCGAAGCCGTCG
TATTGGCAATCGATGTGGAAAAAGAACGCATCTCCTTGGGTATCAAACAACTGGAAGGCGATCCTTTCGGCAAC
TTCATCAGCGTGAACGACAAAGGTTCTTTGGTTAAAGGTTCCGTGAAATCTGTTGACGCCAAAGGCGCTGTTAT
CGCCCTGTCTGACGAAGTAGAAGGCTACCTGCCTGCTTCCGAATTTGCAGCCGACCGCGTTGAAGACTTGACCA
CCAAACTGAAAGAAGGCGACGAAGTTGAAGCCGTCATCGTTACCGTTGACCGCAAAAACCGCAGCATCAAACTT
TCCGTTAAAGCCAAAGATGCCAAAGAAAGCCGCGAAGCACTGAACTCCGTCAATGCCGCCGCCAATGCGAATGC
CGGTACCACCAGCTTGGGCGACCTGCTGAAAGCCAAACTCTCCGGCGAACAAGAATAAGGTTGCAGACATGACA
AAGTCTGAGTTAATGGTTCGCTTGGCAGAAGTATTTGCCGCCAAAAACGGCACGCATCTTCTGGCAAAAGACGT
AGAGTACAGCGTAAAAGTCTTGGTTGACACCATGACCCGATCGCTTGCCCGAGGTCAACGCATCGAAATTCGCG
GTTTCGGCAGCTTCGATTTGAACCATCGTCCTGCCCGCATCGGTCGCAATCCCAAAACCGGCGAGCGCGTGGAA
GTACCTGAAAAACATGTACCCCACTTCAAGCCTGGTAAAGAATTGCGCGAGCGGGTCGACTTGGCTTTAAAAGA
AAATGCCAATTAA
SEQ ID NO: 11 >Haemophilus influenzae Rd_KW20 HI1220 rpsA operon (rpsA-ihfB
locus) [intergenic region underlined]
ATGTCAGAATCTTTTGCTCAACTTTTTGAAGAATCATTAAAAGTCCTTGAAACTCGTCAAGGTTCAATCGTTAG
CGGTACTGTAGTAGCTATCCAAAAAGGCTTTGTGCTTGTTGATGCAGGTTTAAAATCTGAGTCTGCAATTCCAG
TTGCTGAATTCTTAAATGCACAAGGCGAACTTGAAATCCAAGTTGGCGATACTGTAAATGTTGCATTAGATGCA
GTTGAAGATGGTTTCGGTGAAACTAAACTTTCTCGTGAGAAAGCGGTTCGTCACGAATCTTGGATTGAATTAGA
AAAAGCTTACGAAGAAAAAGCGACCGTTATCGGTTTAATCANCGGCAAAGTGAAAGGTGGCTTCACAGTTGAGT
TAAACGGTGTTCGTGCATTCTTACCAGGTTCTTTAGTTGATACTCGTCCAGCGCGTGAAGCAGATCACTTACTT
GGTAAAGAATTAGAATTCAAAGTAATCAAATTAGATCAAAAACGTAACAACGTTGTTGTTTCTCGTCGTGCAGT
AATTGAATCTGAAAACAGCCAAGAACGTGAACAAGTATTAGAAAATCTTGTGGAAGGNTCAGAAGTTAAAGGTG
TCGTTAAAAACTTAACTGAGTACGGTGCATTCGTTGATCTTGGTGGTGTTGATGGTTTATTACACATCACAGAT
ATGGCTTGGAAACGTGTTAAACACCCAAGTGAAATCGTAAATGTTGGCGATGAAGTTACTGTTAAAGTATTAAA
ATTCGATAAAGATCGTACTCGTGTATCTTTAGGCTTAAAACAATTAGGTCAAGATCCATGGGCAGCAATTGCTG
AAAATCATCCAGTAAACAGCAAATTAACAGGTAAAGTAACTAACTTAACTGACTATGGCTGTTTCGTTGAAATC
TTAGATGGCGTTGAAGGTTTAGTTCACGTTTCTGAAATGGATTGGACTAATAAAAATATCCACCCATCTAAAGT
GGTTAGCTTAGGCGATACTGTAGAAGTAATGGTATTAGAAATTGATGAAGAACGTCGTCGTATTTCTTTAGGCT
TAAAACAATGTAAAGCTAACCCTTGGACTCAGTTTGCTGATACTCACAACAAAGGCGATAAAGTAACGGGTAAA
ATCAAGTCTATCACTGATTTCGGTATCTTCATCGGTCTTGAAGGTGGTATCGATGGTTTAGTTCACTTATCTGA
TATTTCTTGGAGTATTTCAGGCGAAGAAGCTGTTCGTCAATACAAAAAAGGTGACGAAGTTTCAGCTGTTGTAT
TAGCTGTTGATGCAGTAAAAGAACGTATCTCTTTAGGTATTAAACAACTTGAAGAAGATCCATTCAATAACTTT
GTCGCAATCAACAAAAAAGGTGCGGTAGTATCTGCAACTGTTGTTGAAGCAGATGCTAAAGGTGCTAAAGTTGA
ATTAGCAGGTGGTGTTGAAGGTTATATCCGTTCTGCTGATTTAACAAGTGAAGTAGCTGTTGGTGATGTCGTTG
AAGCGAAATACACTGGAGTAGATCGTAAATCTCGCATTGTTCACTTATCAGTGAAAGCAAAAGATCAAGCTGAA
GAAGCTGCGGCAGTTGCAAGTGTAAATAACAAACAAGAAGATATTGTTATTCCAAATGCAATGGCTGAAGCATT
TAAAGCAGCTAAAGGTGAATAATTAATTCACGTAATAAGGCTGGGCTGATGTCCAGCCTTATTTGTTAGGAGTT
ATTGCTAATATATTTGTGTAATTTATTATCAATAGATCTTAATTTAAGTAATATTAAGGAAGTATAGACGATGA
CTAAGTCAGAACTTATGGAAAAATTGTCAGCAAAACAGCCAACTTTACCTGCAAAAGAAATTGAAAATATGGTA
AAAGGTATTTTAGAGTTTATTTCTCAATCTCTTGAAAATGGTGATCGTGTTGAAGTTCGAGGTTTTGGTAGCTT
TTCTTTACATCATCGGCAACCACGTTTGGGAAGAAATCCGAAAACAGGCGATTCAGTAAACTTATCAGCTAAGT
CTGTTCCATATTTTAAAGCGGGTAAAGAATTAAAAGCTCGAGTGGATGTTCAGGCTTAA
SEQ ID NO: 12
>Pseudomonas aeruginosa PAO1 rpsA operon (rpsA-ihfB locus) [intergenic
region underlined]
ATGAGCGAAAGCTTCGCAGAACTCTTTGAAGAAAGTCTGAAATCCCTCGACATGCAGCCGGGTGCCATCA
TCACCGGCATCGTGGTCGACATCGATGGTGACTGGGTCACCGTCCATGCCGGTCTGAAATCCGAGGGCGT
CATCCCGGTCGAGCAGTTCTACAACGAACAGGGCGAGCTGACCATCAAGGTGGGTGACGAAGTCCACGTC
GCACTGGACGCGGTAGAAGACGGCTTTGGCGAGACCAAGCTGTCCCGCGAGAAAGCCAAGCGCGCCGAGA
GCTGGATTGTTCTGGAAGCGGCTTTCGCTGCCGACGAAGTGGTCAAGGGCGTCATCAACGGCAAGGTCAA
GGGCGGTTTCACCGTCGACGTCAACGGCATCCGCGCGTTCCTGCCGGGTTCTCTGGTCGACGTTCGTCCG
GTTCGCGACACCACCCACCTGGAAGGCAAAGAGCTCGAGTTCAAGGTCATCAAGCTCGACCAGAAGCGCA
ACAACGTTGTCGTTTCCCGCCGCAGCGTCCTGGAAGCCGAGAACAGCGCCGAGCGTGAAGCTCTGCTGGA
ATCGCTGCAGGAAGGCCAGCAGGTCAAAGGTATCGTCAAGAACCTCACCGACTACGGCGCATTCGTGGAC
CTGGGCGGCGTAGACGGCCTGCTACACATCACCGACATGGCCTGGAAGCGCATCAAGCATCCGTCCGAGA
TCGTCAACGTTGGCGACGAGATCGACGTCAAGGTCCTGAAGTTCGACCGCGAGCGCAACCGTGTATCCCT
GGGCCTGAAGCAACTGGGCGAAGACCCGTGGGTTGCCATCAAGGCGCGTTACCCGGAAGGTACCCGCGTC
ATGGCCCGCGTCACCAACCTCACCGACTACGGCTGCTTCGCCGAGCTGGAAGAGGGCGTGGAAGGCCTGG
TACACGTCTCCGAAATGGACTGGACCAACAAGAACATCCATCCGTCGAAAGTCGTCCAGGTTGGCGATGA
AGTGGAAGTTCAGGTTCTGGACATCGACGAAGAGCGTCGTCGTATCTCCCTGGGTATCAAGCAGTGCAAA
TCCAACCCGTGGGAAGACTTCTCCAGCCAGTTCAACAAGGGTGACCGTATCTCCGGTACCATCAAGTCGA
TCACCGACTTCGGTATCTTCATCGGTCTGGACGGCGGCATCGACGGCCTGGTCCACCTGTCCGACATCTC
CTGGAACGAAGTCGGCGAAGAAGCCGTACGTCGCTTCAAGAAGGGCGACGAGCTGGAAACCGTCATCCTG
TCGGTCGATCCGGAGCGCGAGCGCATCTCCCTGGGCATCAAGCAGCTGGAAGACGATCCGTTCTCCAACT
ACGCGTCCCTGCACGAGAAAGGCAGCATCGTCCGCGGTACCGTGAAGGAAGTCGACGCCAAGGGCGCTGT
CATCAGCCTGGGCGACGACATCGAAGGTATCCTGAAGGCTTCCGAAATCAGCCGTGACCGCGTCGAAGAC
GCGCGCAACGTCCTGAAGGAAGGCGAGGAAGTCGAAGCCAAGATCATCAGCATCGACCGCAAGAGCCGCG
TCATCAGCCTCTCCGTCAAGTCCAAGGACGTCGACGACGAGAAGGACGCAATGAAAGAACTGCGTAAGCA
GGAAGTAGAAAGCGCTGGTCCGACCACCATCGGTGATCTGATCCGTGCTCAGATGGAGAATCAGGGCTAA
GTCTCTGATCCATCATGAAAAAGGGCGGCCTAGGCCGCCCTTTTTCGTTTTCCCCTTCTTGGACCTGTTC
AAAGACTGATCAGCATGCTAAAAGAGACCTGAGCTGATCTAGCCGCTTGAAAAAGAAGGGAAAACCATGA
CCAAGTCGGAGTTGATCGAACGGATCGTTACCCATCAGGGGCAACTGTCCGCGAAGGATGTCGAGTTGGC
AATCAAGACCATGCTGGAGCAAATGTCCCAGGCCCTGGCGACCGGGGACCGGATCGAGATCCGTGGCTTC
GGCAGCTTTTCCTTGCACTACCGCGCCCCGCGCGTCGGTCGCAACCCCAAGACCGGGGAGTCGGTACGCC
TCGACGGCAAGTTCGTGCCGCACTTCAAGCCGGGCAAGGAGTTGCGGGATCGGGTCAACGAGCCGGAGTG
A
SEQ ID NO: 13
>Bordetella pertussis Tohama I S1 protein
MDSNLMSSVSTSAILGGENFADLFAESLKSQDMKSGEVISAEVVRVDHNFVVVNAGLKSEALIPLEEFLNDQGE
LEVQPGDFVSVAIDSLENGYGDTILSRDRAKRLSAWLQLEQALENGELVTGTITGKVKGGLTVMINGIRAFLPG
SLVDLRPVKDTTPYEGKTLEFKVIKLDRKRNNVVLSRRQVLEASMGEERQKLLETLHEGAVVKGVVKNITDYGA
FVDLGGIDGLLHITDMAWRRVRHPSEVLQVGQEVEAKVLKFDQEKSRVSLGVKQLGEDPWVGLARRYPQGTRLF
GKVTNLTDYGAFVEVEAGIEGLVHVSEMDWTNKNVDPRKVVTLGEEVEVMVLEIDEDRRRISLGMKQCRQNPWE
EFATNFKRGDKVRGAIKSITDFGVFVGLPGGIDGLVHLSDLSWTESGEEAVRNFKKGDELEAVVLGIDTEKERI
SLGIKQLEGDPFNNFVATHDKGAVVPGTIKSVEPKGAVITLSVDVEGYLRASEISSGRVEDATTVLKAGENIEA
MIVNIDRKARSIQLSIKARDNAETAETIQRMSEASASSGTTNLGALLKAKLDQQRNDG
SEQ ID NO: 14
>Escherichia coli BL21 (DE3) S1 protein
MTESFAQLFEESLKEIETRPGSIVRGVVVAIDKDVVLVDAGLKSESAIPAEQFKNAQGELEIQVGDEVDVALDA
VEDGFGETLLSREKAKRHEAWITLEKAYEDAETVTGVINGKVKGGFTVELNGIRAFLPGSLVDVRPVRDTLHLE
GKELEFKVIKLDQKRNNVVVSRRAVIESENSAERDQLLENLQEGMEVKGIVKNLTDYGAFVDLGGVDGLLHITD
MAWKRVKHPSEIVNVGDEITVKVLKFDRERTRVSLGLKQLGEDPWVAIAKRYPEGTKLTGRVTNLTDYGCFVEI
EEGVEGLVHVSEMDWTNKNIHPSKVVNVGDVVEVMVLDIDEERRRISLGLKQCKANPWQQFAETHNKGDRVEGK
IKSITDFGIFIGLDGGIDGLVHLSDISWNVAGEEAVREYKKGDEIAAVVLQVDAERERISLGVKQLAEDPENNW
VALNKKGAIVTGKVTAVDAKGATVELADGVEGYLRASEASRDRVEDATLVLSVGDEVEAKFTGVDRKNRAISLS
VRAKDEADEKDAIATVNKQEDANFSNNAMAEAFKAAKGE
SEQ ID NO: 15
>Haemophilus influenzae Rd_KW20 HI1220 S1 protein
MSESFAQLFEESLKVLETRQGSIVSGTVVAIQKGFVLVDAGLKSESAIPVAEFLNAQGELEIQVGDTVNVALDA
VEDGFGETKLSREKAVRHESWIELEKAYEEKATVIGLIXGKVKGGFTVELNGVRAFLPGSLVDTRPAREADHLL
GKELEFKVIKLDQKRNNVVVSRRAVIESENSQEREQVLENLVEGSEVKGVVKNLTEYGAFVDLGGVDGLLHITD
MAWKRVKHPSEIVNVGDEVTVKVLKFDKDRTRVSLGLKQLGQDPWAAIAENHPVNSKLTGKVTNLTDYGCFVEI
LDGVEGLVHVSEMDWTNKNIHPSKVVSLGDTVEVMVLEIDEERRRISLGLKQCKANPWTQFADTHNKGDKVTGK
IKSITDFGIFIGLEGGIDGLVHLSDISWSISGEEAVRQYKKGDEVSAVVLAVDAVKERISLGIKQLEEDPENNE
VAINKKGAVVSATVVEADAKGAKVELAGGVEGYIRSADLTSEVAVGDVVEAKYTGVDRKSRIVHLSVKAKDQAE
EAAAVASVNNKQEDIVIPNAMAEAFKAAKGE
SEQ ID NO: 16
>Neisseria gonorrhoeae FA1090 S1 protein
MSMENFAQLLEESFTLQEMNPGEVITAEVVAIDQNFVTVNAGLKSESLIDVAEFKNAQGEIEVKVGDFVTVTIE
SVENGFGETKLSREKAKRAADWIALEEAMENGNILSGIINGKVKGGLTVMISSIRAFLPGSLVDVRPVKDTSHF
EGKEIEFKVIKLDKKRNNVVVSRRAVLEATLGEERKALLENLQEGSVIKGIVKNITDYGAFVDLGGIDGLLHIT
DLAWRRVKHPSEVLEVGQEVEAKVLKFDQEKQRVSLGMKQLGEDPWSGLTRRYPQATRLFGKVSNLTDYGAFVE
IEQGIEGLVHVSEMDWTNKNVHPSKVVQLGDEVEVMILEIDEGRRRISLGMKQCQANPWEEFAANHNKGDKISG
AVKSITDFGVFVGLPGGIDGLVHLSDLSWTESGEEAVRKYKKGEEVEAVVLAIDVEKERISLGIKQLEGDPFGN
FISVNDKGSLVKGSVKSVDAKGAVIALSDEVEGYLPASEFAADRVEDLTTKLKEGDEVEAVIVTVDRKNRSIKL
SVKAKDAKESREALNSVNAAANANAGTTSLGDLLKAKLSGEQE
SEQ ID NO: 17
>Neisseria meningitidis MC58 S1 protein
MSMENFAQLLEESFTLQEMNPGEVITAEVVAIDQNFVTVNAGLKSESLIDVAEFKNAQGEIEVKVGDFVTVTIE
SVENGFGETKLSREKAKRAADWIALEEAMENGDILSGIINGKVKGGLTVMISSIRAFLPGSLVDVRPVKDTSHF
EGKEIEFKVIKLDKKRNNVVVSRRAVLEATLGEERKALLENLQEGSVIKGIVKNITDYGAFVDLGGIDGLLHIT
DLAWRRVKHPSEVLEVGQEVEAKVLKFDQEKQRVSLGMKQLGEDPWSGLTRRYPQGTRLFGKVSNLTDYGAFVE
IEQGIEGLVHVSEMDWTNKNVHPSKVVQLGDEVEVMILEIDEGRRRISLGMKQCQANPWEEFAANHNKGDKISG
AVKSITDFGVFVGLPGGIDGLVHLSDLSWTESGEEAVRKYKKGEEVEAVVLAIDVEKERISLGIKQLEGDPFGN
FISVNDKGSLVKGSVKSVDAKGAVIALSDEVEGYLPASEFAADRVEDLTTKLKEGDEVEAVIVTVDRKNRSIKL
SVKAKDAKESREALNSVNAAANANAGTTSLGDLLKAKLSGEQE
SEQ ID NO: 18
>Pseudomonas aeruginosa PAO1 S1 protein
MSESFAELFEESLKSLDMQPGAIITGIVVDIDGDWVTVHAGLKSEGVIPVEQFYNEQGELTIKVGDEVHVALDA
VEDGFGETKLSREKAKRAESWIVLEAAFAADEVVKGVINGKVKGGFTVDVNGIRAFLPGSLVDVRPVRDTTHLE
GKELEFKVIKLDQKRNNVVVSRRSVLEAENSAEREALLESLQEGQQVKGIVKNLTDYGAFVDLGGVDGLLHITD
MAWKRIKHPSEIVNVGDEIDVKVLKFDRERNRVSLGLKQLGEDPWVAIKARYPEGTRVMARVINLTDYGCFAEL
EEGVEGLVHVSEMDWTNKNIHPSKVVQVGDEVEVQVLDIDEERRRISLGIKQCKSNPWEDFSSQFNKGDRISGT
IKSITDFGIFIGLDGGIDGLVHLSDISWNEVGEEAVRRFKKGDELETVILSVDPERERISLGIKQLEDDPFSNY
ASLHEKGSIVRGTVKEVDAKGAVISLGDDIEGILKASEISRDRVEDARNVLKEGEEVEAKIISIDRKSRVISLS
VKSKDVDDEKDAMKELRKQEVESAGPTTIGDLIRAQMENQG
SEQ ID NO: 19
>Bordetella pertussis Tohama I rpsA gene, domain 1
GACATGAAGTCCGGCGAGGTCATCAGCGCAGAAGTCGTGCGCGTCGACCACAACTTCGTGGTCGTCAACGCCGG
CCTGAAGTCCGAAGCGCTGATTCCCCTGGAAGAGTTCCTCAACGACCAGGGCGAACTCGAAGTTCAACCCGGCG
ACTTCGTCTCGGTGGCGATCGATTCGCTGGAGAACGGCTACGGCGACACCATCCTGTCGCGCGAC
SEQ ID NO: 20
>Bordetella pertussis Tohama I rpsA gene, domain 2
GCCCTCGAGAACGGCGAGCTGGTCACCGGCACGATCACCGGCAAGGTCAAGGGCGGCCTGACCGTCATGACCAA
CGGCATCCGCGCGTTCCTGCCCGGTTCGCTGGTCGACCTGCGTCCGGTCAAGGACACCACGCCGTACGAAGGCA
AGACCCTCGAATTCAAGGTCATCAAGCTGGACCGCAAGCGCAACAACGTCGTGCTGTCGCGCCGC
SEQ ID NO: 21
>Bordetella pertussis Tohama I rpsA gene, R1
ACGCTGCACGAAGGCGCGGTGGTCAAGGGCGTGGTCAAGAACATCACCGACTACGGCGCGTTCGTCGACCTGGG
CGGCATCGATGGCCTGCTGCACATCACCGACATGGCCTGGCGCCGTGTGCGTCACCCGTCCGAAGTCCTGCAAG
TGGGTCAGGAAGTCGAAGCCAAGGTGCTCAAGTTCGACCAGGAAAAGAGCCGCGTCTCCCTGGGCGTCAAG
SEQ ID NO: 22
>Bordetella pertussis Tohama I rpsA gene, R2
CGCTACCCGCAGGGCACCCGCCTGTTCGGCAAGGTCACCAACCTGACCGACTACGGCGCGTTCGTCGAAGTCGA
AGCCGGCATCGAAGGCCTGGTGCACGTGTCCGAAATGGACTGGACCAACAAGAACGTCGATCCGCGCAAGGTCG
TGACCCTGGGCGAAGAAGTCGAAGTCATGGTCCTGGAAATCGACGAAGACCGTCGCCGCATTTCGCTGGGCATG
AAG
SEQ ID NO: 23
>Bordetella pertussis Tohama I rpsA gene, R3
AACTTCAAGCGTGGTGACAAGGTCCGCGGCGCCATCAAGTCGATCACCGACTTCGGCGTGTTCGTCGGCCTGCC
CGGCGGCATCGACGGCCTGGTCCATCTGTCCGACCTGTCGTGGACGGAATCGGGCGAGGAAGCCGTGCGCAACT
TCAAGAAGGGCGACGAGCTGGAAGCCGTGGTGCTGGGCATCGATACCGAGAAAGAGCGCATCTCGCTGGGTATC
AAG
SEQ ID NO: 24
>Bordetella pertussis Tohama I rpsA gene, R4
ACGCACGACAAGGGCGCCGTTGTTCCGGGCACCATCAAGTCGGTCGAGCCCAAGGGCGCCGTGATCACCCTGTC
GGTGGATGTGGAAGGCTACCTGCGCGCCTCCGAGATCTCCTCGGGCCGCGTCGAAGACGCTACCACCGTGCTGA
AGGCTGGCGAGAACATCGAAGCCATGATCGTCAACATCGACCGCAAGGCGCGTTCGATCCAGCTGTCGATCAAG
SEQ ID NO: 25
>Bordetella pertussis Tohama I ihfB gene
GTGACCAAGTCGGAGCTGATCGCCGCGCTGGCGGCCCGCTATCCTCAGCTGGCCGCTCGCGACACCGATTACGC
TGTCAAGACCATGCTCGATGCAATGACCCAGGCCCTGGCCTCGGGTCAGCGCATCGAAATCCGCGGGTTTGGCA
GCTTTTCGCTGTCGCAGCGCTCTCCCCGTATCGGGCGCAATCCGAAGTCGGGCGAACAAGTGCTGGTGCCTGGC
AAGCAGGTGCCGCACTTCAAGGCCGGCAAGGAGCTGCGCGAGTGGGTCGATCTGGTTGGCAACGATCAGGGCGA
CGACTCGTCCAACGGGTCGTCCGACCCGCTGCAATCGGTCATGGATATGCATGCCATGCACTGA
SEQ ID NO: 26
>Escherichia coli BL21 (DE3) ihfB gene
ATGACCAAGTCAGAATTGATAGAAAGACTTGCCACCCAGCAATCGCACATTCCCGCCAAGACGGTTGAAGATGC
AGTAAAAGAGATGCTGGAGCATATGGCCTCGACTCTTGCGCAGGGCGAGCGTATTGAAATCCGCGGTTTCGGCA
GTTTCTCTTTGCACTACCGCGCACCACGTACCGGACGTAATCCGAAGACTGGCGATAAAGTAGAACTGGAAGGA
AAATACGTTCCTCACTTTAAACCTGGTAAAGAACTGCGCGATCGCGCCAATATTTACGGTTAA
SEQ ID NO: 27
>Haemophilus influenzae Rd KW20 HI1220 ihfB gene
ATGACTAAGTCAGAACTTATGGAAAAATTGTCAGCAAAACAGCCAACTTTACCTGCAAAAGAAATTGAAAATAT
GGTAAAAGGTATTTTAGAGTTTATTTCTCAATCTCTTGAAAATGGTGATCGTGTTGAAGTTCGAGGTTTTGGTA
GCTTTTCTTTACATCATCGGCAACCACGTTTGGGAAGAAATCCGAAAACAGGCGATTCAGTAAACTTATCAGCT
AAGTCTGTTCCATATTTTAAAGCGGGTAAAGAATTAAAAGCTCGAGTGGATGTTCAGGCTTAA
SEQ ID NO: 28
>Neisseria gonorrhoeae FA1090 ihfB gene
ATGGTTCGCTTGGCAGAAGTATTTGCCGCCAAAAACGGCACGCATCTTCTGGCAAAAGACGTAGAGTACAGCGT
AAAAGTCTTGGTTGACACCATGACCCGATCGCTTGCCCGAGGTCAACGCATCGAAATTCGCGGTTTCGGCAGCT
TCGATTTGAACCATCGTCCTGCCCGCATCGGTCGCAATCCCAAAACCGGCGAGCGCGTGGAAGTACCTGAAAAA
CATGTACCCCACTTCAAGCCTGGTAAAGAATTGCGCGAGCGGGTCGACTTGGCTTTAAAAGAAAATGCCAATTA
A
SEQ ID NO: 29
>Neisseria meningitidis MC58 ihfB gene
ATGACAAAGTCTGAGTTAATGGTTCGTTTGGCAGAAGTGTTTGCCGCCAAAAACGGCACGCATCTTCTGGCAAA
AGACGTAGAGTACAGCGTAAAAGTCTTGGTTGACACCATGACTAGATCGCTTGCCCGAGGTCAACGCATCGAAA
TCCGCGGTTTCGGCAGCTTCGATTTGAACCATCGTCCTGCCCGCATCGGTCGCAATCCCAAAACCGGCGAGCGT
GTGGAAGTACCTGAAAAACATGTACCCCACTTCAAGCCCGGTAAAGAATTGCGCGAGCGGGTCGACTTGGCTTT
AAAAGAAAATGCCAATTAA
SEQ ID NO: 30
>Pseudomonas aeruginosa PAO1 ihfB gene
ATGACCAAGTCGGAGTTGATCGAACGGATCGTTACCCATCAGGGGCAACTGTCCGCGAAGGATGTCGAGTTGGC
AATCAAGACCATGCTGGAGCAAATGTCCCAGGCCCTGGCGACCGGGGACCGGATCGAGATCCGTGGCTTCGGCA
GCTTTTCCTTGCACTACCGCGCCCCGCGCGTCGGTCGCAACCCCAAGACCGGGGAGTCGGTACGCCTCGACGGC
AAGTTCGTGCCGCACTTCAAGCCGGGCAAGGAGTTGCGGGATCGGGTCAACGAGCCGGAGTGA
SEQ ID NO: 31
>Bordetella pertussis Tohama I IHF protein
MTKSELIAALAARYPQLAARDTDYAVKTMLDAMTQALASGQRIEIRGFGSFSLSQRSPRIGRNPKSGEQVLVPG
KQVPHFKAGKELREWVDLVGNDQGDDSSNGSSDPLQSVMDMHAMH
SEQ ID NO: 32
>Escherichia coli BL21 (DE3) IHF protein
MTKSELIERLATQQSHIPAKTVEDAVKEMLEHMASTLAQGERIEIRGFGSFSLHYRAPRTGRNPKTGDKVELEG
KYVPHFKPGKELRDRANIYG
SEQ ID NO: 33
>Haemophilus influenzae Rd_KW20 HI1220 IHF protein
MTKSELMEKLSAKQPTLPAKEIENMVKGILEFISQSLENGDRVEVRGFGSFSLHHRQPRLGRNPKTGDSVNLSA
KSVPYFKAGKELKARVDVQA
SEQ ID NO: 34
>Neisseria gonorrhoeae FA1090 IHF protein
MVRLAEVFAAKNGTHLLAKDVEYSVKVLVDTMTRSLARGQRIEIRGFGSFDLNHRPARIGRNPKTGERVEVPEK
HVPHFKPGKELRERVDLALKENAN
SEQ ID NO: 35
>Neisseria meningitidis MC58 IHF protein
MTKSELMVRLAEVFAAKNGTHLLAKDVEYSVKVLVDTMTRSLARGQRIEIRGFGSFDLNHRPARIGRNPKTGER
VEVPEKHVPHFKPGKELRERVDLALKENAN
SEQ ID NO: 36
>Pseudomonas aeruginosa PAO1 IHF protein
MTKSELIERIVTHQGQLSAKDVELAIKTMLEQMSQALATGDRIEIRGFGSFSLHYRAPRVGRNPKTGESVRLDG
KFVPHFKPGKELRDRVNEPE
SEQ ID NO: 37
>B. pertussis 50D6 clone nt sequence rpsA gene [Tn5 underlined]
ATGGATTCCAACCTAATGTCTTCCGTTTCCACCTCCGCCATCCTTGGCGGCGAAAACTTCGCCGACCTGTTCGC
AGAAAGCCTCAAGAGCCAGGACATGAAGTCCGGCGAGGTCATCAGCGCAGAAGTCGTGCGCGTCGACCACAACT
TCGTGGTCGTCAACGCCGGCCTGAAGTCCGAAGCGCTGATTCCCCTGGAAGAGTTCCTCAACGACCAGGGCGAA
CTCGAAGTTCAACCCGGCGACTTCGTCTCGGTGGCGATCGATTCGCTGGAGAACGGCTACGGCGACACCATCCT
GTCGCGCGACCGCGCCAAGCGTCTGTCGGCCTGGCTGCAACTGGAGCAGGCCCTCGAGAACGGCGAGCTGGTCA
CCGGCACGATCACCGGCAAGGTCAAGGGCGGCCTGACCGTCATGACCAACGGCATCCGCGCGTTCCTGCCCGGT
TCGCTGGTCGACCTGCGTCCGGTCAAGGACACCACGCCGTACGAAGGCAAGACCCTCGAATTCAAGGTCATCAA
GCTGGACCGCAAGCGCAACAACGTCGTGCTGTCGCGCCGCCAGGTGCTGGAAGCCAGCATGGGCGAAGAGCGCC
AGAAGCTGCTCGAGACGCTGCACGAAGGCGCGGTGGTCAAGGGCGTGGTCAAGAACATCACCGACTACGGCGCG
TTCGTCGACCTGGGCGGCATCGATGGCCTGCTGCACATCACCGACATGGCCTGGCGCCGTGTGCGTCACCCGTC
CGAAGTCCTGCAAGTGGGTCAGGAAGTCGAAGCCAAGGTGCTCAAGTTCGACCAGGAAAAGAGCCGCGTCTCCC
TGGGCGTCAAGCAGCTGGGCGAAGATCCGTGGGTGGGCCTGGCTCGCCGCTACCCGCAGGGCACCCGCCTGTTC
GGCAAGGTCACCAACCTGACCGACTACGGCGCGTTCGTCGAAGTCGAAGCCGGCATCGAAGGCCTGGTGCACGT
GTCCGAAATGGACTGGACCAACAAGAACGTCGATCCGCGCAAGGTCGTGACCCTGGGCGAAGAAGTCGAAGTCA
TGGTCCTGGAAATCGACGAAGACCGTCGCCGCATTTCGCTGGGCATGAAGCAGTGCCGCCAGAACCCGTGGGAA
GAGTTCGCCACCAACTTCAAGCGTGGTGACAAGGTCCGCGGCGCCATCAAGTCGATCACCGACTTCGGCGTGTT
CGTCGGCCTGCCCGGCGGCATCGACGGCCTGGTCCATCTGTCCGACCTGTCGTGGACGGAATCGGGCGAGGAAG
CCGTGCGCAACTTCAAGAAGGGCGACGAGCTGGAAGCCGTGGTGCTGGGCATCGATACCGAGAAAGAGCGCATC
TCGCTGGGTATCAAGCAGCTCGAAGGCTGTCTCTTATACACATCTGACGTCTTGTGTCTCAAAATCTCTGATGT
TACATTGCACAAGATAAAAATATATCATCATGAACAATAAAACTGTCTGCTTACATAAACAGTAATACAAGGGG
TGTTATGAGCCATATTCAGCGTGAAACGAGCTGTAGCCGTCCGCGTCTGAACAGCAACATGGATGCGGATCTGT
ATGGCTATAAATGGGCGCGTGATAACGTGGGTCAGAGCGGCGCGACCATTTATCGTCTGTATGGCAAACCGGAT
GCGCCGGAACTGTTTCTGAAACATGGCAAAGGCAGCGTGGCGAACGATGTGACCGATGAAATGGTGCGTCTGAA
CTGGCTGACCGAATTTATGCCGCTGCCGACCATTAAACATTTTATTCGCACCCCGGATGATGCGTGGCTGCTGA
CCACCGCGATTCCGGGCAAAACCGCGTTTCAGGTGCTGGAAGAATATCCGGATAGCGGCGAAAACATTGTGGAT
GCGCTGGCCGTGTTTCTGCGTCGTCTGCATAGCATTCCGGTGTGCAACTGCCCGTTTAACAGCGATCGTGTGTT
TCGTCTGGCCCAGGCGCAGAGCCGTATGAACAACGGCCTGGTGGATGCGAGCGATTTTGATGATGAACGTAACG
GCTGGCCGGTGGAACAGGTGTGGAAAGAAATGCATAAACTGCTGCCGTTTAGCCCGGATAGCGTGGTGACCCAC
GGCGATTTTAGCCTGGATAACCTGATTTTCGATGAAGGCAAACTGATTGGCTGCATTGATGTGGGCCGTGTGGG
CATTGCGGATCGTTATCAGGATCTGGCCATTCTGTGGAACTGCCTGGGCGAATTTAGCCCGAGCCTGCAAAAAC
GTCTGTTTCAGAAATATGGCATTGATAATCCGGATATGAACAAACTGCAATTTCATCTGATGCTGGATGAATTT
TTCTAATAATTAATTGGGGACCCTAGAGGTCCCCTTTTTTATTTTAAAAATTTTTTCACAAAACGGTTTACAAG
CATAACTAGTGCGGCCGCAAGCTTGCATGCCTGCAGGTCGACTCTAGAGGATCCCCGGGTACCGAGCTCGAATT
CGCGCGGCCGCGGCCTAGGCGGCCTTAATTAAAGATGTGTATAAGAGACAGGCTCGAAGGCGACCCGTTCAACA
ACTTCGTTGCCACGCACGACAAGGGCGCCGTTGTTCCGGGCACCATCAAGTCGGTCGAGCCCAAGGGCGCCGTG
ATCACCCTGTCGGTGGATGTGGAAGGCTACCTGCGCGCCTCCGAGATCTCCTCGGGCCGCGTCGAAGACGCTAC
CACCGTGCTGAAGGCTGGCGAGAACATCGAAGCCATGATCGTCAACATCGACCGCAAGGCGCGTTCGATCCAGC
TGTCGATCAAGGCCCGCGATAACGCCGAGACGGCCGAGACCATCCAGCGCATGTCCGAGGCGAGCGCTTCGTCG
GGTACGACGAACTTGGGCGCGCTGCTCAAGGCCAAGCTGGACCAACAGCGCAACGACGGTTGAC
SEQ ID NO: 38
>B. pertussis 50D6 clone nt sequence rpsA operon [Tn5 underlined]
ATGGATTCCAACCTAATGTCTTCCGTTTCCACCTCCGCCATCCTTGGCGGCGAAAACTTCGCCGACCTGTTCGC
AGAAAGCCTCAAGAGCCAGGACATGAAGTCCGGCGAGGTCATCAGCGCAGAAGTCGTGCGCGTCGACCACAACT
TCGTGGTCGTCAACGCCGGCCTGAAGTCCGAAGCGCTGATTCCCCTGGAAGAGTTCCTCAACGACCAGGGCGAA
CTCGAAGTTCAACCCGGCGACTTCGTCTCGGTGGCGATCGATTCGCTGGAGAACGGCTACGGCGACACCATCCT
GTCGCGCGACCGCGCCAAGCGTCTGTCGGCCTGGCTGCAACTGGAGCAGGCCCTCGAGAACGGCGAGCTGGTCA
CCGGCACGATCACCGGCAAGGTCAAGGGCGGCCTGACCGTCATGACCAACGGCATCCGCGCGTTCCTGCCCGGT
TCGCTGGTCGACCTGCGTCCGGTCAAGGACACCACGCCGTACGAAGGCAAGACCCTCGAATTCAAGGTCATCAA
GCTGGACCGCAAGCGCAACAACGTCGTGCTGTCGCGCCGCCAGGTGCTGGAAGCCAGCATGGGCGAAGAGCGCC
AGAAGCTGCTCGAGACGCTGCACGAAGGCGCGGTGGTCAAGGGCGTGGTCAAGAACATCACCGACTACGGCGCG
TTCGTCGACCTGGGCGGCATCGATGGCCTGCTGCACATCACCGACATGGCCTGGCGCCGTGTGCGTCACCCGTC
CGAAGTCCTGCAAGTGGGTCAGGAAGTCGAAGCCAAGGTGCTCAAGTTCGACCAGGAAAAGAGCCGCGTCTCCC
TGGGCGTCAAGCAGCTGGGCGAAGATCCGTGGGTGGGCCTGGCTCGCCGCTACCCGCAGGGCACCCGCCTGTTC
GGCAAGGTCACCAACCTGACCGACTACGGCGCGTTCGTCGAAGTCGAAGCCGGCATCGAAGGCCTGGTGCACGT
GTCCGAAATGGACTGGACCAACAAGAACGTCGATCCGCGCAAGGTCGTGACCCTGGGCGAAGAAGTCGAAGTCA
TGGTCCTGGAAATCGACGAAGACCGTCGCCGCATTTCGCTGGGCATGAAGCAGTGCCGCCAGAACCCGTGGGAA
GAGTTCGCCACCAACTTCAAGCGTGGTGACAAGGTCCGCGGCGCCATCAAGTCGATCACCGACTTCGGCGTGTT
CGTCGGCCTGCCCGGCGGCATCGACGGCCTGGTCCATCTGTCCGACCTGTCGTGGACGGAATCGGGCGAGGAAG
CCGTGCGCAACTTCAAGAAGGGCGACGAGCTGGAAGCCGTGGTGCTGGGCATCGATACCGAGAAAGAGCGCATC
TCGCTGGGTATCAAGCAGCTCGAAGGCTGTCTCTTATACACATCTGACGTCTTGTGTCTCAAAATCTCTGATGT
TACATTGCACAAGATAAAAATATATCATCATGAACAATAAAACTGTCTGCTTACATAAACAGTAATACAAGGGG
TGTTATGAGCCATATTCAGCGTGAAACGAGCTGTAGCCGTCCGCGTCTGAACAGCAACATGGATGCGGATCTGT
ATGGCTATAAATGGGCGCGTGATAACGTGGGTCAGAGCGGCGCGACCATTTATCGTCTGTATGGCAAACCGGAT
GCGCCGGAACTGTTTCTGAAACATGGCAAAGGCAGCGTGGCGAACGATGTGACCGATGAAATGGTGCGTCTGAA
CTGGCTGACCGAATTTATGCCGCTGCCGACCATTAAACATTTTATTCGCACCCCGGATGATGCGTGGCTGCTGA
CCACCGCGATTCCGGGCAAAACCGCGTTTCAGGTGCTGGAAGAATATCCGGATAGCGGCGAAAACATTGTGGAT
GCGCTGGCCGTGTTTCTGCGTCGTCTGCATAGCATTCCGGTGTGCAACTGCCCGTTTAACAGCGATCGTGTGTT
TCGTCTGGCCCAGGCGCAGAGCCGTATGAACAACGGCCTGGTGGATGCGAGCGATTTTGATGATGAACGTAACG
GCTGGCCGGTGGAACAGGTGTGGAAAGAAATGCATAAACTGCTGCCGTTTAGCCCGGATAGCGTGGTGACCCAC
GGCGATTTTAGCCTGGATAACCTGATTTTCGATGAAGGCAAACTGATTGGCTGCATTGATGTGGGCCGTGTGGG
CATTGCGGATCGTTATCAGGATCTGGCCATTCTGTGGAACTGCCTGGGCGAATTTAGCCCGAGCCTGCAAAAAC
GTCTGTTTCAGAAATATGGCATTGATAATCCGGATATGAACAAACTGCAATTTCATCTGATGCTGGATGAATTT
TTCTAATAATTAATTGGGGACCCTAGAGGTCCCCTTTTTTATTTTAAAAATTTTTTCACAAAACGGTTTACAAG
CATAACTAGTGCGGCCGCAAGCTTGCATGCCTGCAGGTCGACTCTAGAGGATCCCCGGGTACCGAGCTCGAATT
CGCGCGGCCGCGGCCTAGGCGGCCTTAATTAAAGATGTGTATAAGAGACAGGCTCGAAGGCGACCCGTTCAACA
ACTTCGTTGCCACGCACGACAAGGGCGCCGTTGTTCCGGGCACCATCAAGTCGGTCGAGCCCAAGGGCGCCGTG
ATCACCCTGTCGGTGGATGTGGAAGGCTACCTGCGCGCCTCCGAGATCTCCTCGGGCCGCGTCGAAGACGCTAC
CACCGTGCTGAAGGCTGGCGAGAACATCGAAGCCATGATCGTCAACATCGACCGCAAGGCGCGTTCGATCCAGC
TGTCGATCAAGGCCCGCGATAACGCCGAGACGGCCGAGACCATCCAGCGCATGTCCGAGGCGAGCGCTTCGTCG
GGTACGACGAACTTGGGCGCGCTGCTCAAGGCCAAGCTGGACCAACAGCGCAACGACGGTTGACGTGACCAAGT
CGGAGCTGATCGCCGCGCTGGCGGCCCGCTATCCTCAGCTGGCCGCTCGCGACACCGATTACGCTGTCAAGACC
ATGCTCGATGCAATGACCCAGGCCCTGGCCTCGGGTCAGCGCATCGAAATCCGCGGGTTTGGCAGCTTTTCGCT
GTCGCAGCGCTCTCCCCGTATCGGGCGCAATCCGAAGTCGGGCGAACAAGTGCTGGTGCCTGGCAAGCAGGTGC
CGCACTTCAAGGCCGGCAAGGAGCTGCGCGAGTGGGTCGATCTGGTTGGCAACGATCAGGGCGACGACTCGTCC
AACGGGTCGTCCGACCCGCTGCAATCGGTCATGGATATGCATGCCATGCACTGA
SEQ ID NO: 39
>B. pertussis 50D6 clone aa sequence S1 protein
MDSNLMSSVSTSAILGGENFADLFAESLKSQDMKSGEVISAEVVRVDHNFVVVNAGLKSEALIPLEEFLNDQGE
LEVQPGDFVSVAIDSLENGYGDTILSRDRAKRLSAWLQLEQALENGELVTGTITGKVKGGLTVMINGIRAFLPG
SLVDLRPVKDTTPYEGKTLEFKVIKLDRKRNNVVLSRRQVLEASMGEERQKLLETLHEGAVVKGVVKNITDYGA
FVDLGGIDGLLHITDMAWRRVRHPSEVLQVGQEVEAKVLKFDQEKSRVSLGVKQLGEDPWVGLARRYPQGTRLF
GKVTNLTDYGAFVEVEAGIEGLVHVSEMDWTNKNVDPRKVVTLGEEVEVMVLEIDEDRRRISLGMKQCRQNPWE
EFATNFKRGDKVRGAIKSITDFGVFVGLPGGIDGLVHLSDLSWTESGEEAVRNFKKGDELEAVVLGIDTEKERI
SLGIKQLEGCLLYTSDVLCLKISDVTLHKIKIYHHEQ*
SEQ ID NO: 40
>B. pertussis 77D8 clone aa sequence S1 protein
MDSNLMSSVSTSAILGGENFADLFAESLKSQDMKSGEVISAEVVRVDHNFVVVNAGLKSEALIPLEEFLNDQGE
LEVQPGDFVSVAIDSLENGYGDTILSRDRAKRLSAWLQLEQALENGELVTGTITGKVKGGLTVMINGIRAFLPG
SLVDLRPVKDTTPYEGKTLEFKVIKLDRKRNNVVLSRRQVLEASMGEERQKLLETLHEGAVVKGVVKNITDYGA
FVDLGGIDGLLHITDMAWRRVRHPSEVLQVGQEVEAKVLKFDQEKSRVSLGVKQLGEDPWVGLARRYPQGTRLF
GKVTNLTDYGAFVEVEAGIEGLVHVSEMDWTNKNVDPRKVVTLGEEVEVMVLEIDEDRRRISLGMKQCRQNPWE
EFATNFKRGDKVRGAIKSITDFGVFVGLPGGIDGLVHLSDLSWTESGEEAVRNFKKGDELEAVVLGIDTEKERI
SLGIKQLEGDPFNNFVATHDKGAVVPGTIKSVSYTHL*
SEQ ID NO: 41
>B. pertussis 83G6 clone aa sequence S1 protein
MDSNLMSSVSTSAILGGENFADLFAESLKSQDMKSGEVISAEVVRVDHNFVVVNAGLKSEALIPLEEFLNDQGE
LEVQPGDFVSVAIDSLENGYGDTILSRDRAKRLSAWLQLEQALENGELVTGTITGKVKGGLTVMINGIRAFLPG
SLVDLRPVKDTTPYEGKTLEFKVIKLDRKRNNVVLSRRQVLEASMGEERQKLLETLHEGAVVKGVVKNITDYGA
FVDLGGIDGLLHITDMAWRRVRHPSEVLQVGQEVEAKVLKFDQEKSRVSLGVKQLGEDPWVGLARRYPQGTRLF
GKVTNLTDYGAFVEVEAGIEGLVHVSEMDWTNKNVDPRKVVTLGEEVEVMVLEIDEDRRRISLGMKQCRQNPWE
EFATNFKRGDKVRGAIKSITDFGVFVGLPGGIDGLVHLSDLSWTESGEEAVRNFKKGDELEAVVLGIDTEKERI
SLGIKQLEGDPFNNFVATHDKGAVVPGTIKSVEPKGAVITLSVDVEGYLRASEISSGRVEDATTVLKAGENIEA
MIVNIDRKARSIQLSIKARDNAETAETIQRMSEAVSYTHL*
SEQ ID NO: 42
>Escherichia coli mutant Ins RpsA #1 [KanR insertion mapping the 50D6 clone
underlined]
ATGACTGAATCTTTTGCTCAACTCTTTGAAGAGTCCTTAAAAGAAATCGAAACCCGCCCGGGTTCTATCGTTCG
TGGCGTTGTTGTTGCTATCGACAAAGACGTAGTACTGGTTGACGCTGGTCTGAAATCTGAGTCCGCCATCCCGG
CTGAGCAGTTCAAAAACGCCCAGGGCGAGCTGGAAATCCAGGTAGGTGACGAAGTTGACGTTGCTCTGGACGCA
GTAGAAGACGGCTTCGGTGAAACTCTGCTGTCCCGTGAGAAAGCTAAACGTCACGAAGCCTGGATCACGCTGGA
AAAAGCTTACGAAGATGCTGAAACTGTTACCGGTGTTATCAACGGCAAAGTTAAGGGCGGCTTCACTGTTGAGC
TGAACGGTATTCGTGCGTTCCTGCCAGGTTCTCTGGTAGACGTTCGTCCGGTGCGTGACACTCTGCACCTGGAA
GGCAAAGAGCTTGAATTTAAAGTAATCAAGCTGGATCAGAAGCGCAACAACGTTGTTGTTTCTCGTCGTGCCGT
TATCGAATCCGAAAACAGCGCAGAGCGCGATCAGCTGCTGGAAAACCTGCAGGAAGGCATGGAAGTTAAAGGTA
TCGTTAAGAACCTCACTGACTACGGTGCATTCGTTGATCTGGGCGGCGTTGACGGCCTGCTGCACATCACTGAC
ATGGCCTGGAAACGCGTTAAGCATCCGAGCGAAATCGTCAACGTGGGCGACGAAATCACTGTTAAAGTGCTGAA
GTTCGACCGCGAACGTACCCGTGTATCCCTGGGCCTGAAACAGCTGGGCGAAGATCCGTGGGTAGCTATCGCTA
AACGTTATCCGGAAGGTACCAAACTGACTGGTCGCGTGACCAACCTGACCGACTACGGCTGCTTCGTTGAAATC
GAAGAAGGCGTTGAAGGCCTGGTACACGTTTCCGAAATGGATTGGACCAACAAAAACATCCACCCGTCCAAAGT
TGTTAACGTTGGCGATGTAGTGGAAGTTATGGTTCTGGATATCGACGAAGAACGTCGTCGTATCTCCCTGGGTC
TGAAACAGTGCAAAGCTAACCCGTGGCAGCAGTTCGCGGAAACCCACAACAAGGGCGACCGTGTTGAAGGTAAA
ATCAAGTCTATCACTGACTTCGGTATCTTCATCGGCCTGGACGGCGGCATCGACGGCCTGGTTCACCTGTCTGA
CATCTCCTGGAACGTTGCAGGCGAAGAAGCAGTTCGTGAATACAAAAAAGGCGACGAAATCGCTGCAGTTGTTC
TGCAGGTTGACGCAGAACGTGAACGTATCTCCCTGGGCGTTAAACAGCTCGCGTGTAGGCTGGAGCTGCTTCGA
AGTTCCTATACTTTCTAGAGAATAGGAACTTCGGAATAGGAACTTCAAGATCCCCTCACGCTGCCGCAAGCACT
CAGGGCGCAAGGGCTGCTAAAGGAAGCGGAACACGTAGAAAGCCAGTCCGCAGAAACGGTGCTGACCCCGGATG
AATGTCAGCTACTGGGCTATCTGGACAAGGGAAAACGCAAGCGCAAAGAGAAAGCAGGTAGCTTGCAGTGGGCT
TACATGGCGATAGCTAGACTGGGCGGTTTTATGGACAGCAAGCGAACCGGAATTGCCAGCTGGGGCGCCCTCTG
GTAAGGTTGGGAAGCCCTGCAAAGTAAACTGGATGGCTTTCTTGCCGCCAAGGATCTGATGGCGCAGGGGATCA
AGATCTGATCAAGAGACAGGATGAGGATCGTTTCGCATGATTGAACAAGATGGATTGCACGCAGGTTCTCCGGC
CGCTTGGGTGGAGAGGCTATTCGGCTATGACTGGGCACAACAGACAATCGGCTGCTCTGATGCCGCCGTGTTCC
GGCTGTCAGCGCAGGGGCGCCCGGTTCTTTTTGTCAAGACCGACCTGTCCGGTGCCCTGAATGAACTGCAGGAC
GAGGCAGCGCGGCTATCGTGGCTGGCCACGACGGGCGTTCCTTGCGCAGCTGTGCTCGACGTTGTCACTGAAGC
GGGAAGGGACTGGCTGCTATTGGGCGAAGTGCCGGGGCAGGATCTCCTGTCATCTCACCTTGCTCCTGCCGAGA
AAGTATCCATCATGGCTGATGCAATGCGGCGGCTGCATACGCTTGATCCGGCTACCTGCCCATTCGACCACCAA
GCGAAACATCGCATCGAGCGAGCACGTACTCGGATGGAAGCCGGTCTTGTCGATCAGGATGATCTGGACGAAGA
GCATCAGGGGCTCGCGCCAGCCGAACTGTTCGCCAGGCTCAAGGCGCGCATGCCCGACGGCGAGGATCTCGTCG
TGACCCATGGCGATGCCTGCTTGCCGAATATCATGGTGGAAAATGGCCGCTTTTCTGGATTCATCGACTGTGGC
CGGCTGGGTGTGGCGGACCGCTATCAGGACATAGCGTTGGCTACCCGTGATATTGCTGAAGAGCTTGGCGGCGA
ATGGGCTGACCGCTTCCTCGTGCTTTACGGTATCGCCGCTCCCGATTCGCAGCGCATCGCCTTCTATCGCCTTC
TTGACGAGTTCTTCTGAGCGGGACTCTGGGGTTCGAAATGACCGACCAAGCGACGCCCAACCTGCCATCACGAG
ATTTCGATTCCACCGCCGCCTTCTATGAAAGGTTGGGCTTCGGAATCGTTTTCCGGGACGCCGGCTGGATGATC
CTCCAGCGCGGGGATCTCATGCTGGAGTTCTTCGCCCACCCCAGCTTCAAAAGCGCTCTGAAGTTCCTATACTT
TCTAGAGAATAGGAACTTCGGAATAGGAACTAAGGAGGATATTCATATGAGAAGATCCGTTCAACAACTGGGTT
GCTCTGAACAAGAAAGGCGCTATCGTAACCGGTAAAGTAACTGCAGTTGACGCTAAAGGCGCAACCGTAGAACT
GGCTGACGGCGTTGAAGGTTACCTGCGTGCTTCTGAAGCATCCCGTGACCGCGTTGAAGACGCTACCCTGGTTC
TGAGCGTTGGCGACGAAGTTGAAGCTAAATTCACCGGCGTTGATCGTAAAAACCGCGCAATCAGCCTGTCTGTT
CGTGCGAAAGACGAAGCTGACGAGAAAGATGCAATCGCAACTGTTAACAAACAGGAAGATGCAAACTTCTCCAA
CAACGCAATGGCTGAAGCTTTCAAAGCAGCTAAAGGCGAGTAATTCTCTGACTCTTCGGGATTTTTATTCCGAA
GTTTGTTGAGTTTACTTGACAGATTGCAGGTTTCGTCCTGTAATCAAGCACTAAGGGCGGCTACGGCCGCCCTT
AATCAATGCAGCAACAGCAGCCGCTTAATTTGCCTTTAAGGAACCGGAGGAATCATGACCAAGTCAGAATTGAT
AGAAAGACTTGCCACCCAGCAATCGCACATTCCCGCCAAGACGGTTGAAGATGCAGTAAAAGAGATGCTGGAGC
ATATGGCCTCGACTCTTGCGCAGGGCGAGCGTATTGAAATCCGCGGTTTCGGCAGTTTCTCTTTGCACTACCGC
GCACCACGTACCGGACGTAATCCGAAGACTGGCGATAAAGTAGAACTGGAAGGAAAATACGTTCCTCACTTTAA
ACCTGGTAAAGAACTGCGCGATCGCGCCAATATTTACGGTTAAGTTTTTTACTCAAACTTGAACGAGAGAAAAG
CACCTGTCGGGTGCTTTTTTCATTTCTCTAATCTGGAACTGGAAGCTGCCTCGCAGAGTTTTGAACAGTTTTCA
CCCTTTCGTTAAATTCTTCTGAATATGCCTCGGGGAACGCAAAATTCCCAC
SEQ ID NO: 43
>Escherichia coli mutant RpsA 3′ #2 [KanR insertion underlined]
ATGACTGAATCTTTTGCTCAACTCTTTGAAGAGTCCTTAAAAGAAATCGAAACCCGCCCGGGTTCTATCGTTCG
TGGCGTTGTTGTTGCTATCGACAAAGACGTAGTACTGGTTGACGCTGGTCTGAAATCTGAGTCCGCCATCCCGG
CTGAGCAGTTCAAAAACGCCCAGGGCGAGCTGGAAATCCAGGTAGGTGACGAAGTTGACGTTGCTCTGGACGCA
GTAGAAGACGGCTTCGGTGAAACTCTGCTGTCCCGTGAGAAAGCTAAACGTCACGAAGCCTGGATCACGCTGGA
AAAAGCTTACGAAGATGCTGAAACTGTTACCGGTGTTATCAACGGCAAAGTTAAGGGCGGCTTCACTGTTGAGC
TGAACGGTATTCGTGCGTTCCTGCCAGGTTCTCTGGTAGACGTTCGTCCGGTGCGTGACACTCTGCACCTGGAA
GGCAAAGAGCTTGAATTTAAAGTAATCAAGCTGGATCAGAAGCGCAACAACGTTGTTGTTTCTCGTCGTGCCGT
TATCGAATCCGAAAACAGCGCAGAGCGCGATCAGCTGCTGGAAAACCTGCAGGAAGGCATGGAAGTTAAAGGTA
TCGTTAAGAACCTCACTGACTACGGTGCATTCGTTGATCTGGGCGGCGTTGACGGCCTGCTGCACATCACTGAC
ATGGCCTGGAAACGCGTTAAGCATCCGAGCGAAATCGTCAACGTGGGCGACGAAATCACTGTTAAAGTGCTGAA
GTTCGACCGCGAACGTACCCGTGTATCCCTGGGCCTGAAACAGCTGGGCGAAGATCCGTGGGTAGCTATCGCTA
AACGTTATCCGGAAGGTACCAAACTGACTGGTCGCGTGACCAACCTGACCGACTACGGCTGCTTCGTTGAAATC
GAAGAAGGCGTTGAAGGCCTGGTACACGTTTCCGAAATGGATTGGACCAACAAAAACATCCACCCGTCCAAAGT
TGTTAACGTTGGCGATGTAGTGGAAGTTATGGTTCTGGATATCGACGAAGAACGTCGTCGTATCTCCCTGGGTC
TGAAACAGTGCAAAGCTAACCCGTGGCAGCAGTTCGCGGAAACCCACAACAAGGGCGACCGTGTTGAAGGTAAA
ATCAAGTCTATCACTGACTTCGGTATCTTCATCGGCCTGGACGGCGGCATCGACGGCCTGGTTCACCTGTCTGA
CATCTCCTGGAACGTTGCAGGCGAAGAAGCAGTTCGTGAATACAAAAAAGGCGACGAAATCGCTGCAGTTGTTC
TGCAGGTTGACGCAGAACGTGAACGTATCTCCCTGGGCGTTAAACAGCTCGCCATATGAATATCCTCCTTAGTT
CCTATTCCGAAGTTCCTATTCTCTAGAAAGTATAGGAACTTCAGAGCGCTTTTGAAGCTGGGGTGGGCGAAGAA
CTCCAGCATGAGATCCCCGCGCTGGAGGATCATCCAGCCGGCGTCCCGGAAAACGATTCCGAAGCCCAACCTTT
CATAGAAGGCGGCGGTGGAATCGAAATCTCGTGATGGCAGGTTGGGCGTCGCTTGGTCGGTCATTTCGAACCCC
AGAGTCCCGCTCAGAAGAACTCGTCAAGAAGGCGATAGAAGGCGATGCGCTGCGAATCGGGAGCGGCGATACCG
TAAAGCACGAGGAAGCGGTCAGCCCATTCGCCGCCAAGCTCTTCAGCAATATCACGGGTAGCCAACGCTATGTC
CTGATAGCGGTCCGCCACACCCAGCCGGCCACAGTCGATGAATCCAGAAAAGCGGCCATTTTCCACCATGATAT
TCGGCAAGCAGGCATCGCCATGGGTCACGACGAGATCCTCGCCGTCGGGCATGCGCGCCTTGAGCCTGGCGAAC
AGTTCGGCTGGCGCGAGCCCCTGATGCTCTTCGTCCAGATCATCCTGATCGACAAGACCGGCTTCCATCCGAGT
ACGTGCTCGCTCGATGCGATGTTTCGCTTGGTGGTCGAATGGGCAGGTAGCCGGATCAAGCGTATGCAGCCGCC
GCATTGCATCAGCCATGATGGATACTTTCTCGGCAGGAGCAAGGTGAGATGACAGGAGATCCTGCCCCGGCACT
TCGCCCAATAGCAGCCAGTCCCTTCCCGCTTCAGTGACAACGTCGAGCACAGCTGCGCAAGGAACGCCCGTCGT
GGCCAGCCACGATAGCCGCGCTGCCTCGTCCTGCAGTTCATTCAGGGCACCGGACAGGTCGGTCTTGACAAAAA
GAACCGGGCGCCCCTGCGCTGACAGCCGGAACACGGCGGCATCAGAGCAGCCGATTGTCTGTTGTGCCCAGTCA
TAGCCGAATAGCCTCTCCACCCAAGCGGCCGGAGAACCTGCGTGCAATCCATCTTGTTCAATCATGCGAAACGA
TCCTCATCCTGTCTCTTGATCAGATCTTGATCCCCTGCGCCATCAGATCCTTGGCGGCAAGAAAGCCATCCAGT
TTACTTTGCAGGGCTTCCCAACCTTACCAGAGGGCGCCCCAGCTGGCAATTCCGGTTCGCTTGCTGTCCATAAA
ACCGCCCAGTCTAGCTATCGCCATGTAAGCCCACTGCAAGCTACCTGCTTTCTCTTTGCGCTTGCGTTTTCCCT
TGTCCAGATAGCCCAGTAGCTGACATTCATCCGGGGTCAGCACCGTTTCTGCGGACTGGCTTTCTACGTGTTCC
GCTTCCTTTAGCAGCCCTTGCGCCCTGAGTGCTTGCGGCAGCGTGAGGGGATCTTGAAGTTCCTATTCCGAAGT
TCCTATTCTCTAGAAAGTATAGGAACTTCGAAGCAGCTCCAGCCTACACGACTCTTCGGGATTTTTATTCCGAA
GTTTGTTGAGTTTACTTGACAGATTGCAGGTTTCGTCCTGTAATCAAGCACTAAGGGCGGCTACGGCCGCCCTT
AATCAATGCAGCAACAGCAGCCGCTTAATTTGCCTTTAAGGAACCGGAGGAATCATGACCAAGTCAGAATTGAT
AGAAAGACTTGCCACCCAGCAATCGCACATTCCCGCCAAGACGGTTGAAGATGCAGTAAAAGAGATGCTGGAGC
ATATGGCCTCGACTCTTGCGCAGGGCGAGCGTATTGAAATCCGCGGTTTCGGCAGTTTCTCTTTGCACTACCGC
GCACCACGTACCGGACGTAATCCGAAGACTGGCGATAAAGTAGAACTGGAAGGAAAATACGTTCCTCACTTTAA
ACCTGGTAAAGAACTGCGCGATCGCGCCAATATTTACGGTTAAGTTTTTTACTCAAACTTGAACGAGAGAAAAG
CACCTGTCGGGTGCTTTTTTCATTTCTCTAATCTGGAACTGGAAGCTGCCTCGCAGAGTTTTGAACAGTTTTCA
CCCTTTCGTTAAATTCTTCTGAATATGCCTCGGGGAACGCAAAATTCCCAC
SEQ ID NO: 44
>Escherichia coli mutant Ins RpsA #1 S1 protein
MTESFAQLFEESLKEIETRPGSIVRGVVVAIDKDVVLVDAGLKSESAIPAEQFKNAQGELEIQVGDEVDVALDA
VEDGFGETLLSREKAKRHEAWITLEKAYEDAETVTGVINGKVKGGFTVELNGIRAFLPGSLVDVRPVRDTLHLE
GKELEFKVIKLDQKRNNVVVSRRAVIESENSAERDQLLENLQEGMEVKGIVKNLTDYGAFVDLGGVDGLLHITD
MAWKRVKHPSEIVNVGDEITVKVLKFDRERTRVSLGLKQLGEDPWVAIAKRYPEGTKLTGRVTNLTDYGCFVEI
EEGVEGLVHVSEMDWTNKNIHPSKVVNVGDVVEVMVLDIDEERRRISLGLKQCKANPWQQFAETHNKGDRVEGK
IKSITDFGIFIGLDGGIDGLVHLSDISWNVAGEEAVREYKKGDEIAAVVLQVDAERERISLGVKQLACRLELLR
SSYTF
SEQ ID NO: 45
>Escherichia coli mutant ARpsA 3′ #2 S1 protein
MTESFAQLFEESLKEIETRPGSIVRGVVVAIDKDVVLVDAGLKSESAIPAEQFKNAQGELEIQVGDEVDVALDA
VEDGFGETLLSREKAKRHEAWITLEKAYEDAETVTGVINGKVKGGFTVELNGIRAFLPGSLVDVRPVRDTLHLE
GKELEFKVIKLDQKRNNVVVSRRAVIESENSAERDQLLENLQEGMEVKGIVKNLTDYGAFVDLGGVDGLLHITD
MAWKRVKHPSEIVNVGDEITVKVLKFDRERTRVSLGLKQLGEDPWVAIAKRYPEGTKLTGRVTNLTDYGCFVEI
EEGVEGLVHVSEMDWTNKNIHPSKVVNVGDVVEVMVLDIDEERRRISLGLKQCKANPWQQFAETHNKGDRVEGK
IKSITDFGIFIGLDGGIDGLVHLSDISWNVAGEEAVREYKKGDEIAAVVLQVDAERERISLGVKQLAI
SEQ ID NO: 46
>Neisseria meningitidis NG7 construct [Insertion of chloramphenicol
resistance cassette cmR underlined]
ATGTCTATGGAAAATTTTGCTCAGCTGTTGGAAGAAAGCTTTACCCTGCAAGAAATGAACCCGGGTGAGGTGAT
TACCGCTGAAGTAGTGGCAATCGACCAAAACTTCGTTACCGTAAACGCAGGTCTGAAATCAGAATCCCTGATTG
ATGTAGCTGAATTCAAAAACGCTCAAGGCGAAATTGAAGTTAAAGTCGGCGACTTCGTTACCGTTACCATCGAA
TCCGTCGAAAACGGCTTCGGCGAAACCAAACTGTCCCGCGAAAAAGCCAAACGTGCAGCCGATTGGATTGCCCT
GGAAGAAGCCATGGAAAACGGCGACATCCTGTCCGGCATCATCAACGGAAAAGTCAAAGGCGGCCTGACCGTTA
TGATTAGCAGCATCCGCGCATTCCTGCCGGGTTCTTTGGTCGACGTACGTCCTGTAAAAGACACTTCTCACTTC
GAAGGCAAAGAGATCGAATTCAAAGTGATCAAACTGGACAAAAAACGCAACAACGTCGTTGTTTCCCGCCGCGC
CGTTCTGGAAGCCACTTTGGGTGAAGAACGCAAAGCCCTGCTGGAAAACCTGCAAGAAGGCTCCGTCATCAAAG
GCATCGTTAAAAACATTACCGATTACGGTGCATTCGTTGACTTGGGCGGCATCGACGGTCTGTTGCACATCACC
GATTTGGCATGGCGGCGCGTGAAACACCCGAGTGAAGTCTTGGAAGTCGGTCAGGAAGTTGAAGCCAAAGTATT
GAAATTCGACCAAGAAAAACAACGCGTTTCCTTGGGTATGAAACAACTGGGCGAAGATCCTTGGAGCGGTCTGA
CCCGCCGTTATCCTCAAGGCACCCGCCTGTTCGGCAAAGTATCCAACCTGACCGACTACGGCGCATTCGTCGAA
ATCGAACAAGGCATCGAAGGTTTGGTACACGTCTCCGAAATGGACTGGACCAACAAAAACGTACACCCGAGCAA
AGTCGTACAACTGGGCGACGAAGTCGAAGTCATGATTTTGGAAATCGACGAAGGCCGCCGCCGTATCTCTTTGG
GTATGAAACAATGCCAAGCCAATCCTTGGGAAGAATTTGCCGCCAACCACAACAAAGGCGACAAAATCTCCGGC
GCGGTTAAATCCATTACCGATTTCGGCGTATTCGTCGGCCTGCCCGGCGGCATCGACGGTTTGGTTCACCTGTC
CGACCTGTCCTGGACCGAATCCGGCGAAGAAGCCGTACGCAAATACAAAAAAGGCGAAGAAGTCGAAGCCGTCG
TATTGGCAATCGACGTGGAAAAAGAACGCATCTCCTTGGGTATCAAACAACTGGAAGTCAACCGTGATATAGAT
TGAAAAGTGGATAGATTTATGATATAGTGGATAGATTTATGATATAATGAGTTATCAACAAATCGGAATTTACG
GAGGATAAATGATGCAATTCACAAAGATTGATATAAATAATTGGACACGAAAAGAGTATTTCGACCACTATTTT
GGCAATACGCCCTGCACATATAGTATGACGGTAAAACTCGATATTTCTAAGTTGAAAAAGGATGGAAAAAAGTT
ATACCCAACTCTTTTATATGGAGTTACAACGATCATCAATCGACATGAAGAGTTCAGGACCGCATTAGATGAAA
ACGGACAGGTAGGCGTTTTTTCAGAAATGCTGCCTTGCTACACAGTTTTTCATAAGGAAACTGAAACCTTTTCG
AGTATTTGGACTGAGTTTACAGCAGACTATACTGAGTTTCTTCAGAACTATCAAAAGGATATAGACGCTTTTGG
TGAACGAATGGGAATGTCCGCAAAGCCTAATCCTCCGGAAAACACTTTCCCTGTTTCTATGATACCGTGGACAA
GCTTTGAAGGCTTTAACTTAAATCTAAAAAAAGGATATGACTATCTACTGCCGATATTTACGTTTGGGAAGTAT
TATGAGGAGGGCGGAAAATACTATATTCCCTTATCGATTCAAGTGCATCATGCCGTTTGTGACGGCTTTCATGT
TTGCCGTTTTTTGGATGAATTACAAGACTTGCTGAATAAATAAAATCCCAGTTTGTCGCACTGATAAAAACCCT
TTAGGAACTAAAGGGCGCACTTCTATACTCTCTGTCGAGAGTAGTGCGTCCTGCAGATCTGTTGACGGGCCCGG
TACCGCGGCCGCCTTAAGACGCGTGGATCATGCCGTCTGAACATATTTCAGACGGCTAACTGGAAGGCGATCCG
TTCGGCAACTTCATCAGCGTGAACGACAAAGGTTCTTTGGTTAAAGGTTCCGTGAAATCTGTTGACGCCAAAGG
TGCTGTTATCGCCCTGTCTGACGAAGTAGAAGGCTACCTGCCTGCTTCCGAATTTGCAGCCGACCGCGTTGAAG
ATTTGACCACCAAACTGAAAGAAGGCGACGAAGTTGAAGCCGTCATCGTTACCGTTGACCGCAAAAACCGCAGC
ATCAAACTTTCCGTTAAAGCCAAAGATGCCAAAGAAAGCCGCGAAGCACTGAACTCCGTCAATGCCGCCGCCAA
TGCGAATGCCGGCACCACCAGCTTGGGCGACCTGCTGAAAGCCAAACTCTCCGGCGAACAAGAATAAGGTTGCA
GACATGACAAAGTCTGAGTTAATGGTTCGTTTGGCAGAAGTGTTTGCCGCCAAAAACGGCACGCATCTTCTGGC
AAAAGACGTAGAGTACAGCGTAAAAGTCTTGGTTGACACCATGACTAGATCGCTTGCCCGAGGTCAACGCATCG
AAATCCGCGGTTTCGGCAGCTTCGATTTGAACCATCGTCCTGCCCGCATCGGTCGCAATCCCAAAACCGGCGAG
CGTGTGGAAGTACCTGAAAAACATGTACCCCACTTCAAGCCCGGTAAAGAATTGCGCGAGCGGGTCGACTTGGC
TTTAAAAGAAAATGCCAATTAA
SEQ ID NO: 47
>Neisseria meningitidis NG8 construct [Insertion of chloramphenicol
resistance cassette cmR underlined; deletion of S1 C-terminus]
ATGTCTATGGAAAATTTTGCTCAGCTGTTGGAAGAAAGCTTTACCCTGCAAGAAATGAACCCGGGTGAGGTGAT
TACCGCTGAAGTAGTGGCAATCGACCAAAACTTCGTTACCGTAAACGCAGGTCTGAAATCAGAATCCCTGATTG
ATGTAGCTGAATTCAAAAACGCTCAAGGCGAAATTGAAGTTAAAGTCGGCGACTTCGTTACCGTTACCATCGAA
TCCGTCGAAAACGGCTTCGGCGAAACCAAACTGTCCCGCGAAAAAGCCAAACGTGCAGCCGATTGGATTGCCCT
GGAAGAAGCCATGGAAAACGGCGACATCCTGTCCGGCATCATCAACGGAAAAGTCAAAGGCGGCCTGACCGTTA
TGATTAGCAGCATCCGCGCATTCCTGCCGGGTTCTTTGGTCGACGTACGTCCTGTAAAAGACACTTCTCACTTC
GAAGGCAAAGAGATCGAATTCAAAGTGATCAAACTGGACAAAAAACGCAACAACGTCGTTGTTTCCCGCCGCGC
CGTTCTGGAAGCCACTTTGGGTGAAGAACGCAAAGCCCTGCTGGAAAACCTGCAAGAAGGCTCCGTCATCAAAG
GCATCGTTAAAAACATTACCGATTACGGTGCATTCGTTGACTTGGGCGGCATCGACGGTCTGTTGCACATCACC
GATTTGGCATGGCGGCGCGTGAAACACCCGAGTGAAGTCTTGGAAGTCGGTCAGGAAGTTGAAGCCAAAGTATT
GAAATTCGACCAAGAAAAACAACGCGTTTCCTTGGGTATGAAACAACTGGGCGAAGATCCTTGGAGCGGTCTGA
CCCGCCGTTATCCTCAAGGCACCCGCCTGTTCGGCAAAGTATCCAACCTGACCGACTACGGCGCATTCGTCGAA
ATCGAACAAGGCATCGAAGGTTTGGTACACGTCTCCGAAATGGACTGGACCAACAAAAACGTACACCCGAGCAA
AGTCGTACAACTGGGCGACGAAGTCGAAGTCATGATTTTGGAAATCGACGAAGGCCGCCGCCGTATCTCTTTGG
GTATGAAACAATGCCAAGCCAATCCTTGGGAAGAATTTGCCGCCAACCACAACAAAGGCGACAAAATCTCCGGC
GCGGTTAAATCCATTACCGATTTCGGCGTATTCGTCGGCCTGCCCGGCGGCATCGACGGTTTGGTTCACCTGTC
CGACCTGTCCTGGACCGAATCCGGCGAAGAAGCCGTACGCAAATACAAAAAAGGCGAAGAAGTCGAAGCCGTCG
TATTGGCAATCGACGTGGAAAAAGAACGCATCTCCTTGGGTATCAAACAACTGGAAGTCAACCGTGATATAGAT
TGAAAAGTGGATAGATTTATGATATAGTGGATAGATTTATGATATAATGAGTTATCAACAAATCGGAATTTACG
GAGGATAAATGATGCAATTCACAAAGATTGATATAAATAATTGGACACGAAAAGAGTATTTCGACCACTATTTT
GGCAATACGCCCTGCACATATAGTATGACGGTAAAACTCGATATTTCTAAGTTGAAAAAGGATGGAAAAAAGTT
ATACCCAACTCTTTTATATGGAGTTACAACGATCATCAATCGACATGAAGAGTTCAGGACCGCATTAGATGAAA
ACGGACAGGTAGGCGTTTTTTCAGAAATGCTGCCTTGCTACACAGTTTTTCATAAGGAAACTGAAACCTTTTCG
AGTATTTGGACTGAGTTTACAGCAGACTATACTGAGTTTCTTCAGAACTATCAAAAGGATATAGACGCTTTTGG
TGAACGAATGGGAATGTCCGCAAAGCCTAATCCTCCGGAAAACACTTTCCCTGTTTCTATGATACCGTGGACAA
GCTTTGAAGGCTTTAACTTAAATCTAAAAAAAGGATATGACTATCTACTGCCGATATTTACGTTTGGGAAGTAT
TATGAGGAGGGCGGAAAATACTATATTCCCTTATCGATTCAAGTGCATCATGCCGTTTGTGACGGCTTTCATGT
TTGCCGTTTTTTGGATGAATTACAAGACTTGCTGAATAAATAAAATCCCAGTTTGTCGCACTGATAAAAACCCT
TTAGGAACTAAAGGGCGCACTTCTATACTCTCTGTCGAGAGTAGTGCGTCCTGCAGATCTGTTGACGGGCCCGG
TACCGCGGCCGCCTTAAGACGCGTGGATCATGCCGTCTGAACATATTTCAGACGGCTAGGTTGCAGACATGACA
AAGTCTGAGTTAATGGTTCGTTTGGCAGAAGTGTTTGCCGCCAAAAACGGCACGCATCTTCTGGCAAAAGACGT
AGAGTACAGCGTAAAAGTCTTGGTTGACACCATGACTAGATCGCTTGCCCGAGGTCAACGCATCGAAATCCGCG
GTTTCGGCAGCTTCGATTTGAACCATCGTCCTGCCCGCATCGGTCGCAATCCCAAAACCGGCGAGCGTGTGGAA
GTACCTGAAAAACATGTACCCCACTTCAAGCCCGGTAAAGAATTGCGCGAGCGGGTCGACTTGGCTTTAAAAGA
AAATGCCAATTAA
SEQ ID NO: 48
>Neisseria meningitidis NG9 construct [Insertion of chloramphenicol
resistance cassette cmR underlined; deletion of downstream gene]
ATGTCTATGGAAAATTTTGCTCAGCTGTTGGAAGAAAGCTTTACCCTGCAAGAAATGAACCCGGGTGAGGTGAT
TACCGCTGAAGTAGTGGCAATCGACCAAAACTTCGTTACCGTAAACGCAGGTCTGAAATCAGAATCCCTGATTG
ATGTAGCTGAATTCAAAAACGCTCAAGGCGAAATTGAAGTTAAAGTCGGCGACTTCGTTACCGTTACCATCGAA
TCCGTCGAAAACGGCTTCGGCGAAACCAAACTGTCCCGCGAAAAAGCCAAACGTGCAGCCGATTGGATTGCCCT
GGAAGAAGCCATGGAAAACGGCGACATCCTGTCCGGCATCATCAACGGAAAAGTCAAAGGCGGCCTGACCGTTA
TGATTAGCAGCATCCGCGCATTCCTGCCGGGTTCTTTGGTCGACGTACGTCCTGTAAAAGACACTTCTCACTTC
GAAGGCAAAGAGATCGAATTCAAAGTGATCAAACTGGACAAAAAACGCAACAACGTCGTTGTTTCCCGCCGCGC
CGTTCTGGAAGCCACTTTGGGTGAAGAACGCAAAGCCCTGCTGGAAAACCTGCAAGAAGGCTCCGTCATCAAAG
GCATCGTTAAAAACATTACCGATTACGGTGCATTCGTTGACTTGGGCGGCATCGACGGTCTGTTGCACATCACC
GATTTGGCATGGCGGCGCGTGAAACACCCGAGTGAAGTCTTGGAAGTCGGTCAGGAAGTTGAAGCCAAAGTATT
GAAATTCGACCAAGAAAAACAACGCGTTTCCTTGGGTATGAAACAACTGGGCGAAGATCCTTGGAGCGGTCTGA
CCCGCCGTTATCCTCAAGGCACCCGCCTGTTCGGCAAAGTATCCAACCTGACCGACTACGGCGCATTCGTCGAA
ATCGAACAAGGCATCGAAGGTTTGGTACACGTCTCCGAAATGGACTGGACCAACAAAAACGTACACCCGAGCAA
AGTCGTACAACTGGGCGACGAAGTCGAAGTCATGATTTTGGAAATCGACGAAGGCCGCCGCCGTATCTCTTTGG
GTATGAAACAATGCCAAGCCAATCCTTGGGAAGAATTTGCCGCCAACCACAACAAAGGCGACAAAATCTCCGGC
GCGGTTAAATCCATTACCGATTTCGGCGTATTCGTCGGCCTGCCCGGCGGCATCGACGGTTTGGTTCACCTGTC
CGACCTGTCCTGGACCGAATCCGGCGAAGAAGCCGTACGCAAATACAAAAAAGGCGAAGAAGTCGAAGCCGTCG
TATTGGCAATCGACGTGGAAAAAGAACGCATCTCCTTGGGTATCAAACAACTGGAAGGCGATCCGTTCGGCAAC
TTCATCAGCGTGAACGACAAAGGTTCTTTGGTTAAAGGTTCCGTGAAATCTGTTGACGCCAAAGGTGCTGTTAT
CGCCCTGTCTGACGAAGTAGAAGGCTACCTGCCTGCTTCCGAATTTGCAGCCGACCGCGTTGAAGATTTGACCA
CCAAACTGAAAGAAGGCGACGAAGTTGAAGCCGTCATCGTTACCGTTGACCGCAAAAACCGCAGCATCAAACTT
TCCGTTAAAGCCAAAGATGCCAAAGAAAGCCGCGAAGCACTGAACTCCGTCAATGCCGCCGCCAATGCGAATGC
CGGCACCACCAGCTTGGGCGACCTGCTGAAAGCCAAACTCTCCGGCGAACAAGAATAAGGTTGCAGACAGTCAA
CCGTGATATAGATTGAAAAGTGGATAGATTTATGATATAGTGGATAGATTTATGATATAATGAGTTATCAACAA
ATCGGAATTTACGGAGGATAAATGATGCAATTCACAAAGATTGATATAAATAATTGGACACGAAAAGAGTATTT
CGACCACTATTTTGGCAATACGCCCTGCACATATAGTATGACGGTAAAACTCGATATTTCTAAGTTGAAAAAGG
ATGGAAAAAAGTTATACCCAACTCTTTTATATGGAGTTACAACGATCATCAATCGACATGAAGAGTTCAGGACC
GCATTAGATGAAAACGGACAGGTAGGCGTTTTTTCAGAAATGCTGCCTTGCTACACAGTTTTTCATAAGGAAAC
TGAAACCTTTTCGAGTATTTGGACTGAGTTTACAGCAGACTATACTGAGTTTCTTCAGAACTATCAAAAGGATA
TAGACGCTTTTGGTGAACGAATGGGAATGTCCGCAAAGCCTAATCCTCCGGAAAACACTTTCCCTGTTTCTATG
ATACCGTGGACAAGCTTTGAAGGCTTTAACTTAAATCTAAAAAAAGGATATGACTATCTACTGCCGATATTTAC
GTTTGGGAAGTATTATGAGGAGGGCGGAAAATACTATATTCCCTTATCGATTCAAGTGCATCATGCCGTTTGTG
ACGGCTTTCATGTTTGCCGTTTTTTGGATGAATTACAAGACTTGCTGAATAAATAAAATCCCAGTTTGTCGCAC
TGATAAAAACCCTTTAGGAACTAAAGGGCGCACTTCTATACTCTCTGTCGAGAGTAGTGCGTCCTGCAGATCTG
TTGACGGGCCCGGTACCGCGGCCGCCTTAAGACGCGTGGATCATGCCGTCTGAACATATTTCAGACGGCTAAAC
CATCGTCCTGCCCGCATCGGTCGCAATCCCAAAACCGGCGAGCGTGTGGAAGTACCTGAAAAACATGTACCCCA
CTTCAAGCCCGGTAAAGAATTGCGCGAGCGGGTCGACTTGGCTTTAAAAGAAAATGCCAATTAA
SEQ ID NO: 49
>S1 protein sequence in Neisseria meningitidis NG7 construct
MSMENFAQLLEESFTLQEMNPGEVITAEVVAIDQNFVTVNAGLKSESLIDVAEFKNAQGEIEVKVGDFVTVTIE
SVENGFGETKLSREKAKRAADWIALEEAMENGDILSGIINGKVKGGLTVMISSIRAFLPGSLVDVRPVKDTSHF
EGKEIEFKVIKLDKKRNNVVVSRRAVLEATLGEERKALLENLQEGSVIKGIVKNITDYGAFVDLGGIDGLLHIT
DLAWRRVKHPSEVLEVGQEVEAKVLKFDQEKQRVSLGMKQLGEDPWSGLTRRYPQGTRLFGKVSNLTDYGAFVE
IEQGIEGLVHVSEMDWTNKNVHPSKVVQLGDEVEVMILEIDEGRRRISLGMKQCQANPWEEFAANHNKGDKISG
AVKSITDFGVFVGLPGGIDGLVHLSDLSWTESGEEAVRKYKKGEEVEAVVLAIDVEKERISLGIKQLEVNRDID
*
SEQ ID NO: 50
>S1 protein sequence in Neisseria meningitidis NG8 construct
MSMENFAQLLEESFTLQEMNPGEVITAEVVAIDQNFVTVNAGLKSESLIDVAEFKNAQGEIEVKVGDFVTVTIE
SVENGFGETKLSREKAKRAADWIALEEAMENGDILSGIINGKVKGGLTVMISSIRAFLPGSLVDVRPVKDTSHF
EGKEIEFKVIKLDKKRNNVVVSRRAVLEATLGEERKALLENLQEGSVIKGIVKNITDYGAFVDLGGIDGLLHIT
DLAWRRVKHPSEVLEVGQEVEAKVLKFDQEKQRVSLGMKQLGEDPWSGLTRRYPQGTRLFGKVSNLTDYGAFVE
IEQGIEGLVHVSEMDWTNKNVHPSKVVQLGDEVEVMILEIDEGRRRISLGMKQCQANPWEEFAANHNKGDKISG
AVKSITDFGVFVGLPGGIDGLVHLSDLSWTESGEEAVRKYKKGEEVEAVVLAIDVEKERISLGIKQLEVNRDID
*
SEQ ID NO: 51
>Bordetella pertussis mutant C7 [KanR insertion mapping the 50D6 clone
underlined]
ATGGATTCCAACCTAATGTCTTCCGTTTCCACCTCCGCCATCCTTGGCGGCGAAAACTTCGCCGACCTGTTCGC
AGAAAGCCTCAAGAGCCAGGACATGAAGTCCGGCGAGGTCATCAGCGCAGAAGTCGTGCGCGTCGACCACAACT
TCGTGGTCGTCAACGCCGGCCTGAAGTCCGAAGCGCTGATTCCCCTGGAAGAGTTCCTCAACGACCAGGGCGAA
CTCGAAGTTCAACCCGGCGACTTCGTCTCGGTGGCGATCGATTCGCTGGAGAACGGCTACGGCGACACCATCCT
GTCGCGCGACCGCGCCAAGCGTCTGTCGGCCTGGCTGCAACTGGAGCAGGCCCTCGAGAACGGCGAGCTGGTCA
CCGGCACGATCACCGGCAAGGTCAAGGGCGGCCTGACCGTCATGACCAACGGCATCCGCGCGTTCCTGCCCGGT
TCGCTGGTCGACCTGCGTCCGGTCAAGGACACCACGCCGTACGAAGGCAAGACCCTCGAATTCAAGGTCATCAA
GCTGGACCGCAAGCGCAACAACGTCGTGCTGTCGCGCCGCCAGGTGCTGGAAGCCAGCATGGGCGAAGAGCGCC
AGAAGCTGCTCGAGACGCTGCACGAAGGCGCGGTGGTCAAGGGCGTGGTCAAGAACATCACCGACTACGGCGCG
TTCGTCGACCTGGGCGGCATCGATGGCCTGCTGCACATCACCGACATGGCCTGGCGCCGTGTGCGTCACCCGTC
CGAAGTCCTGCAAGTGGGTCAGGAAGTCGAAGCCAAGGTGCTCAAGTTCGACCAGGAAAAGAGCCGCGTCTCCC
TGGGCGTCAAGCAGCTGGGCGAAGATCCGTGGGTGGGCCTGGCTCGCCGCTACCCGCAGGGCACCCGCCTGTTC
GGCAAGGTCACCAACCTGACCGACTACGGCGCGTTCGTCGAAGTCGAAGCCGGCATCGAAGGCCTGGTGCACGT
GTCCGAAATGGACTGGACCAACAAGAACGTCGATCCGCGCAAGGTCGTGACCCTGGGCGAAGAAGTCGAAGTCA
TGGTCCTGGAAATCGACGAAGACCGTCGCCGCATTTCGCTGGGCATGAAGCAGTGCCGCCAGAACCCGTGGGAA
GAGTTCGCCACCAACTTCAAGCGTGGTGACAAGGTCCGCGGCGCCATCAAGTCGATCACCGACTTCGGCGTGTT
CGTCGGCCTGCCCGGCGGCATCGACGGCCTGGTCCATCTGTCCGACCTGTCGTGGACGGAATCGGGCGAGGAAG
CCGTGCGCAACTTCAAGAAGGGCGACGAGCTGGAAGCCGTGGTGCTGGGCATCGATACCGAGAAAGAGCGCATC
TCGCTGGGTATCAAGCAGCTCGAACTAGTGTCGACCTGCAGGGGGGGGGGGGAAAGCCACGTTGTGTCTCAAAA
TCTCTGATGTTACATTGCACAAGATAAAAATATATCATCATGAACAATAAAACTGTCTGCTTACATAAACAGTA
ATACAAGGGGTGTTATGAGCCATATTCAACGGGAAACGTCTTGCTCGAGGCCGCGATTAAATTCCAACATGGAT
GCTGATTTATATGGGTATAAATGGGCTCGCGATAATGTCGGGCAATCAGGTGCGACAATCTATCGATTGTATGG
GAAGCCCGATGCGCCAGAGTTGTTTCTGAAACATGGCAAAGGTAGCGTTGCCAATGATGTTACAGATGAGATGG
TCAGACTAAACTGGCTGACGGAATTTATGCCTCTTCCGACCATCAAGCATTTTATCCGTACTCCTGATGATGCA
TGGTTACTCACCACTGCGATCCCCGGGAAAACAGCATTCCAGGTATTAGAAGAATATCCTGATTCAGGTGAAAA
TATTGTTGATGCGCTGGCAGTGTTCCTGCGCCGGTTGCATTCGATTCCTGTTTGTAATTGTCCTTTTAACAGCG
ATCGCGTATTTCGTCTCGCTCAGGCGCAATCACGAATGAATAACGGTTTGGTTGATGCGAGTGATTTTGATGAC
GAGCGTAATGGCTGGCCTGTTGAACAAGTCTGGAAAGAAATGCATAAGCTGTTGCCATTCTCACCGGATTCAGT
CGTCACTCATGGTGATTTCTCACTTGATAACCTTATTTTTGACGAGGGGAAATTAATAGGTTGTATTGATGTTG
GACGAGTCGGAATCGCAGACCGATACCAGGATCTTGCCATCCTATGGAACTGCCTCGGTGAGTTTTCTCCTTCA
TTACAGAAACGGCTTTTTCAAAAATATGGTATTGATAATCCTGATATGAATAAATTGCAGTTTCATTTGATGCT
CGATGAGTTTTTCTAATCAGAATTGGTTAATTGGTTGTAACACTGGCAGAGCATTACGCTGACTTGACGGGACG
GCGGCTTTGTTGAATAAATCGAACTTTTGCTGAGTTGAAGGATCAGATCACGCATCTTCCCGACAACGCAGACC
GTTCCGTGGCAAAGCAAAAGTTCAAAATCACCAACTGGTCCACCTACAACAAAGCTCTCATCAACCGTGGCTCC
CTCACTTTCTGGCTGGATGATGGGGCGATTCAGGCCTGGTATGAGTCAGCAACACCTTCTTCACGAGGCAGACC
TCAGCGCCCCCCCCCCCCTGCAGGTCGACACTAGTAGGCGACCCGTTCAACAACTTCGTTGCCACGCACGACAA
GGGCGCCGTTGTTCCGGGCACCATCAAGTCGGTCGAGCCCAAGGGCGCCGTGATCACCCTGTCGGTGGATGTGG
AAGGCTACCTGCGCGCCTCCGAGATCTCCTCGGGCCGCGTCGAAGACGCTACCACCGTGCTGAAGGCTGGCGAG
AACATCGAAGCCATGATCGTCAACATCGACCGCAAGGCGCGTTCGATCCAGCTGTCGATCAAGGCCCGCGATAA
CGCCGAGACGGCCGAGACCATCCAGCGCATGTCCGAGGCGAGCGCTTCGTCGGGTACGACGAACTTGGGCGCGC
TGCTCAAGGCCAAGCTGGACCAACAGCGCAACGACGGTTGACGTGACCAAGTCGGAGCTGATCGCCGCGCTGGC
GGCCCGCTATCCTCAGCTGGCCGCTCGCGACACCGATTACGCTGTCAAGACCATGCTCGATGCAATGACCCAGG
CCCTGGCCTCGGGTCAGCGCATCGAAATCCGCGGGTTTGGCAGCTTTTCGCTGTCGCAGCGCTCTCCCCGTATC
GGGCGCAATCCGAAGTCGGGCGAACAAGTGCTGGTGCCTGGCAAGCAGGTGCCGCACTTCAAGGCCGGCAAGGA
GCTGCGCGAGTGGGTCGATCTGGTTGGCAACGATCAGGGCGACGACTCGTCCAACGGGTCGTCCGACCCGCTGC
AATCGGTCATGGATATGCATGCCATGCACTGA
SEQ ID NO: 52
>Bordetella pertussis mutant C9 [deletion of ihfb by substitution with KanR
underlined]
ATGGATTCCAACCTAATGTCTTCCGTTTCCACCTCCGCCATCCTTGGCGGCGAAAACTTCGCCGACCTGTTCGC
AGAAAGCCTCAAGAGCCAGGACATGAAGTCCGGCGAGGTCATCAGCGCAGAAGTCGTGCGCGTCGACCACAACT
TCGTGGTCGTCAACGCCGGCCTGAAGTCCGAAGCGCTGATTCCCCTGGAAGAGTTCCTCAACGACCAGGGCGAA
CTCGAAGTTCAACCCGGCGACTTCGTCTCGGTGGCGATCGATTCGCTGGAGAACGGCTACGGCGACACCATCCT
GTCGCGCGACCGCGCCAAGCGTCTGTCGGCCTGGCTGCAACTGGAGCAGGCCCTCGAGAACGGCGAGCTGGTCA
CCGGCACGATCACCGGCAAGGTCAAGGGCGGCCTGACCGTCATGACCAACGGCATCCGCGCGTTCCTGCCCGGT
TCGCTGGTCGACCTGCGTCCGGTCAAGGACACCACGCCGTACGAAGGCAAGACCCTCGAATTCAAGGTCATCAA
GCTGGACCGCAAGCGCAACAACGTCGTGCTGTCGCGCCGCCAGGTGCTGGAAGCCAGCATGGGCGAAGAGCGCC
AGAAGCTGCTCGAGACGCTGCACGAAGGCGCGGTGGTCAAGGGCGTGGTCAAGAACATCACCGACTACGGCGCG
TTCGTCGACCTGGGCGGCATCGATGGCCTGCTGCACATCACCGACATGGCCTGGCGCCGTGTGCGTCACCCGTC
CGAAGTCCTGCAAGTGGGTCAGGAAGTCGAAGCCAAGGTGCTCAAGTTCGACCAGGAAAAGAGCCGCGTCTCCC
TGGGCGTCAAGCAGCTGGGCGAAGATCCGTGGGTGGGCCTGGCTCGCCGCTACCCGCAGGGCACCCGCCTGTTC
GGCAAGGTCACCAACCTGACCGACTACGGCGCGTTCGTCGAAGTCGAAGCCGGCATCGAAGGCCTGGTGCACGT
GTCCGAAATGGACTGGACCAACAAGAACGTCGATCCGCGCAAGGTCGTGACCCTGGGCGAAGAAGTCGAAGTCA
TGGTCCTGGAAATCGACGAAGACCGTCGCCGCATTTCGCTGGGCATGAAGCAGTGCCGCCAGAACCCGTGGGAA
GAGTTCGCCACCAACTTCAAGCGTGGTGACAAGGTCCGCGGCGCCATCAAGTCGATCACCGACTTCGGCGTGTT
CGTCGGCCTGCCCGGCGGCATCGACGGCCTGGTCCATCTGTCCGACCTGTCGTGGACGGAATCGGGCGAGGAAG
CCGTGCGCAACTTCAAGAAGGGCGACGAGCTGGAAGCCGTGGTGCTGGGCATCGATACCGAGAAAGAGCGCATC
TCGCTGGGTATCAAGCAGCTCGAAGGCGACCCGTTCAACAACTTCGTTGCCACGCACGACAAGGGCGCCGTTGT
TCCGGGCACCATCAAGTCGGTCGAGCCCAAGGGCGCCGTGATCACCCTGTCGGTGGATGTGGAAGGCTACCTGC
GCGCCTCCGAGATCTCCTCGGGCCGCGTCGAAGACGCTACCACCGTGCTGAAGGCTGGCGAGAACATCGAAGCC
ATGATCGTCAACATCGACCGCAAGGCGCGTTCGATCCAGCTGTCGATCAAGGCCCGCGATAACGCCGAGACGGC
CGAGACCATCCAGCGCATGTCCGAGGCGAGCGCTTCGTCGGGTACGACGAACTTGGGCGCGCTGCTCAAGGCCA
AGCTGGACCAACAGCGCAACGACGGTTGACACTAGTGTCGACCTGCAGGGGGGGGGGGGAAAGCCACGTTGTGT
CTCAAAATCTCTGATGTTACATTGCACAAGATAAAAATATATCATCATGAACAATAAAACTGTCTGCTTACATA
AACAGTAATACAAGGGGTGTTATGAGCCATATTCAACGGGAAACGTCTTGCTCGAGGCCGCGATTAAATTCCAA
CATGGATGCTGATTTATATGGGTATAAATGGGCTCGCGATAATGTCGGGCAATCAGGTGCGACAATCTATCGAT
TGTATGGGAAGCCCGATGCGCCAGAGTTGTTTCTGAAACATGGCAAAGGTAGCGTTGCCAATGATGTTACAGAT
GAGATGGTCAGACTAAACTGGCTGACGGAATTTATGCCTCTTCCGACCATCAAGCATTTTATCCGTACTCCTGA
TGATGCATGGTTACTCACCACTGCGATCCCCGGGAAAACAGCATTCCAGGTATTAGAAGAATATCCTGATTCAG
GTGAAAATATTGTTGATGCGCTGGCAGTGTTCCTGCGCCGGTTGCATTCGATTCCTGTTTGTAATTGTCCTTTT
AACAGCGATCGCGTATTTCGTCTCGCTCAGGCGCAATCACGAATGAATAACGGTTTGGTTGATGCGAGTGATTT
TGATGACGAGCGTAATGGCTGGCCTGTTGAACAAGTCTGGAAAGAAATGCATAAGCTGTTGCCATTCTCACCGG
ATTCAGTCGTCACTCATGGTGATTTCTCACTTGATAACCTTATTTTTGACGAGGGGAAATTAATAGGTTGTATT
GATGTTGGACGAGTCGGAATCGCAGACCGATACCAGGATCTTGCCATCCTATGGAACTGCCTCGGTGAGTTTTC
TCCTTCATTACAGAAACGGCTTTTTCAAAAATATGGTATTGATAATCCTGATATGAATAAATTGCAGTTTCATT
TGATGCTCGATGAGTTTTTCTAATCAGAATTGGTTAATTGGTTGTAACACTGGCAGAGCATTACGCTGACTTGA
CGGGACGGCGGCTTTGTTGAATAAATCGAACTTTTGCTGAGTTGAAGGATCAGATCACGCATCTTCCCGACAAC
GCAGACCGTTCCGTGGCAAAGCAAAAGTTCAAAATCACCAACTGGTCCACCTACAACAAAGCTCTCATCAACCG
TGGCTCCCTCACTTTCTGGCTGGATGATGGGGCGATTCAGGCCTGGTATGAGTCAGCAACACCTTCTTCACGAG
GCAGACCTCAGCGCCCCCCCCCTGCAGGTCGACACTAGT
SEQ ID NO: 53
>S1 protein sequence in Bordetella pertussis C7 construct
MDSNLMSSVSTSAILGGENFADLFAESLKSQDMKSGEVISAEVVRVDHNFVVVNAGLKSEALIPLEEFLN
DQGELEVQPGDFVSVAIDSLENGYGDTILSRDRAKRLSAWLQLEQALENGELVTGTITGKVKGGLTVMTN
GIRAFLPGSLVDLRPVKDTTPYEGKTLEFKVIKLDRKRNNVVLSRRQVLEASMGEERQKLLETLHEGAVV
KGVVKNITDYGAFVDLGGIDGLLHITDMAWRRVRHPSEVLQVGQEVEAKVLKFDQEKSRVSLGVKQLGED
PWVGLARRYPQGTRLFGKVTNLTDYGAFVEVEAGIEGLVHVSEMDWTNKNVDPRKVVTLGEEVEVMVLEI
DEDRRRISLGMKQCRQNPWEEFATNFKRGDKVRGAIKSITDFGVFVGLPGGIDGLVHLSDLSWTESGEEA
VRNFKKGDELEAVVLGIDTEKERISLGIKQLELVSTCRGGGESHVVSQNL*
SEQ ID NO: 54
>Bordetella pertussis
MSSVSTSAILGGENFADLFAESLKSQDMKSGEVISAEVVRVDHNFVVVNAGLKSEALIPEEFLNDQGEL
EVQPGDFVSVAIDSLENGYGDTILSRDRAKRLSAWLQLEQALENGELVTGTITGKVKGGLTVMINGIRAF
LPGSLVDLRPVKDTTPYEGKTLEFKVIKLDRKRNNVVLSRRQVLEASMGEERQKLLETLHEGAVVKGVVK
NITDYGAFVDLGGIDGLLHITDMAWRRVRHPSEVLQVGQEVEAKVLKFDQEKSRVSLGVKQLGEDPWVGL
ARRYPQGTRLFGKVTNLTDYGAFVEVEAGIEGLVHVSEMDWTNKNVDPRKVVTLGEEVEVMVLEIDEDRR
RISLGMKQCRQNPWEEFATNFKRGDKVRGAIKSITDFGVFVGLPGGIDGLVHLSDLSWTESGEEAVRNFK
KGDELEAVVLGIDTEKERISLGIKQLEGDPFNNFVATHDKGAVVPGTIKSVEPKGAVITLSVDVEGYLRA
SEISSGRVEDATTVLKAGENIEAMIVNIDRKARSIQLSIKARDNAETAETIQRMSEASASSGTTNLGALL
KAKLDQQRNDG
SEQ ID NO: 55
>Moraxella catarrhalis BBH18 rpsA gene
ATGGTATCATTTGCTGAACTGTTTGAAGCAAGCCAATCAGAACAAGGTCTTAACATTGAGCGTGGCTCAGTGAT
TTCAGGAACAGTCGTTGCTATTGACAGCGATTGGATTACAGTTGATACAGGTCTTAAATCTGAAGGTATCGTTG
CTCGTGAAGAGTTCTTAAACGAAGCTGGCGAGCTTGAAGTGGCAGTAGGCGACAGCATTGATGTTGTAGTTGAA
GCTGTAGATAACGGCATGGGTCAAACTGTGCTTTCACGCGAAAAAGCGAAGCGTGAAGAAAGCTGGCGTGCGCT
TGAACAACTACACGAAAATGATGAAATCGTTAAAGGCGTTATTTCATCTAAAGTTAAAGGCGGTTTCACTGTTG
AGATCGGTTCAGTGCGTGCATTCCTTCCAGGGTCATTGGTAGATGTTCGCCCAGTTCGTGAAACGACACACCTA
GAAGGTAAAGAGCTTGAATTTAAAGTCATTAAAATTGATAAACAACGCAACAATATCGTTGTATCACGCCGTGC
TGTTATGGAAGCTGAAAATTCAGCAGAGCGTGATGAGCTACTAGCCAAGCTAGAAGAGGGCATGGAAGTAGAAG
GTATCGTTAAGAATCTAACTGAATATGGTGCATTTGTTGATTTAGGCGGTATTGATGGTCTATTGCACATCACC
GATATGGCGTGGAAGCGTATCAAGCACCCATCAGAAGCGGTAGAAGTTGGTCAAGACCTTAAAGTAAAAGTTCT
TAAATTTGACCGTGAGCGTAACCGTGTTAGCTTGGGTCTAAAACAATTGGGTGCTGACCCATGGACTGAAGTTG
AGCAAACTTATCCAGTGGGTACCATCACCAAAGCACGTGTAACCAACCTAACTGATTATGGCTGCTTTGCTGAA
ATCGCTGAAGGTATTGAAGGTTTGGTGCATGTCTCTGAAATGGATCATACGAACAAAAATATTCACCCATCAAA
AGTTGTTCAAGTGGGCGATGAGGTGAATGTGATGGTTCTTGAGATTGATGGTGAGCGTCGCCGTATTAGCCTTG
GTATCAAACAAACCATGCCAAATCCATGGGCTGAGTTTGATAAAAACCATGAAAAAGGTCAAAAAATCAGCGGT
ACCATCAAATCAATCACTGACTTTGGTCTGTTTATTGGCCTAGAAGGTGGTATTGATGGTTTGGTACACCTGTC
TGATATTTCATGGACTGAGTCTGGTGAAGAAGCCATCCGCAATTACTCAAAAGGTGATACTGTTGAGGCGGTCG
TATTGTCTGTTGATGCAGAAGCAAACCGTATCAGCCTAGGCATTAAGCAGCTAAACTCTGATCCATTTAATGAA
TACTTGCTATCAAATGATCGTGGTGCTATCGTTAAAGGTACAGTAAAAGAAGTGGATGCTAAAGGTGCGGTGAT
TACTTTGGCCGATGAAGTTGAAGGTTATTTGCGTGTTGCAGATATTGCTCGTGAGCGTACCGAAGATGCCTCTA
AAGTCTTGAGCGTTGGTGATGAAGTAGAAGCTAAAATTGTCGGCGTTGACCGCAAATCTCGCAATATCAGCCTA
TCAATCAAGGCAAAAGATGAAGCTGATGAGCGTCAAGCCATTAAAGAGCTTTCTAATACAGCAGCTGAAACACA
GCCAAAAACACTTGGCGATATTTTCGGTGAGCAGCTTAAAGGTTGA
SEQ ID NO: 56
>Moraxella catarrhalis BBH18 rpsA gene mutated (ArpsA-Ct)
ATGGTATCATTTGCTGAACTGTTTGAAGCAAGCCAATCAGAACAAGGTCTTAACATTGAGCGTGGCTCAGTGAT
TTCAGGAACAGTCGTTGCTATTGACAGCGATTGGATTACAGTTGATACAGGTCTTAAATCTGAAGGTATCGTTG
CTCGTGAAGAGTTCTTAAACGAAGCTGGCGAGCTTGAAGTGGCAGTAGGCGACAGCATTGATGTTGTAGTTGAA
GCTGTAGATAACGGCATGGGTCAAACTGTGCTTTCACGCGAAAAAGCGAAGCGTGAAGAAAGCTGGCGTGCGCT
TGAACAACTACACGAAAATGATGAAATCGTTAAAGGCGTTATTTCATCTAAAGTTAAAGGCGGTTTCACTGTTG
AGATCGGTTCAGTGCGTGCATTCCTTCCAGGGTCATTGGTAGATGTTCGCCCAGTTCGTGAAACGACACACCTA
GAAGGTAAAGAGCTTGAATTTAAAGTCATTAAAATTGATAAACAACGCAACAATATCGTTGTATCACGCCGTGC
TGTTATGGAAGCTGAAAATTCAGCAGAGCGTGATGAGCTACTAGCCAAGCTAGAAGAGGGCATGGAAGTAGAAG
GTATCGTTAAGAATCTAACTGAATATGGTGCATTTGTTGATTTAGGCGGTATTGATGGTCTATTGCACATCACC
GATATGGCGTGGAAGCGTATCAAGCACCCATCAGAAGCGGTAGAAGTTGGTCAAGACCTTAAAGTAAAAGTTCT
TAAATTTGACCGTGAGCGTAACCGTGTTAGCTTGGGTCTAAAACAATTGGGTGCTGACCCATGGACTGAAGTTG
AGCAAACTTATCCAGTGGGTACCATCACCAAAGCACGTGTAACCAACCTAACTGATTATGGCTGCTTTGCTGAA
ATCGCTGAAGGTATTGAAGGTTTGGTGCATGTCTCTGAAATGGATCATACGAACAAAAATATTCACCCATCAAA
AGTTGTTCAAGTGGGCGATGAGGTGAATGTGATGGTTCTTGAGATTGATGGTGAGCGTCGCCGTATTAGCCTTG
GTATCAAACAAACCATGCCAAATCCATGGGCTGAGTTTGATAAAAACCATGAAAAAGGTCAAAAAATCAGCGGT
ACCATCAAATCAATCACTGACTTTGGTCTGTTTATTGGCCTAGAAGGTGGTATTGATGGTTTGGTACACCTGTC
TGATATTTCATGGACTGAGTCTGGTGAAGAAGCCATCCGCAATTACTCAAAAGGTGATACTGTTGAGGCGGTCG
TATTGTCTGTTGATGCAGAAGCAAACCGTATCAGCCTAGGCATTAAGCAGCTA
SEQ ID NO: 57
>Moraxella catarrhalis BBH18 S1 protein
MVSFAELFEASQSEQGLNIERGSVISGTVVAIDSDWITVDTGLKSEGIVAREEFLNEAGELEVAVGDSIDVVVE
AVDNGMGQTVLSREKAKREESWRALEQLHENDEIVKGVISSKVKGGFTVEIGSVRAFLPGSLVDVRPVRETTHL
EGKELEFKVIKIDKQRNNIVVSRRAVMEAENSAERDELLAKLEEGMEVEGIVKNLTEYGAFVDLGGIDGLLHIT
DMAWKRIKHPSEAVEVGQDLKVKVLKFDRERNRVSLGLKQLGADPWTEVEQTYPVGTITKARVINLTDYGCFAE
IAEGIEGLVHVSEMDHTNKNIHPSKVVQVGDEVNVMVLEIDGERRRISLGIKQTMPNPWAEFDKNHEKGQKISG
TIKSITDFGLFIGLEGGIDGLVHLSDISWTESGEEAIRNYSKGDTVEAVVLSVDAEANRISLGIKQLNSDPENE
YLLSNDRGAIVKGTVKEVDAKGAVITLADEVEGYLRVADIARERTEDASKVLSVGDEVEAKIVGVDRKSRNISL
SIKAKDEADERQAIKELSNTAAETQPKTLGDIFGEQLKG
SEQ ID NO: 58
>Moraxella catarrhalis BBH18 S1 protein mutated (ΔrpsA-Ct)
MVSFAELFEASQSEQGLNIERGSVISGTVVAIDSDWITVDTGLKSEGIVAREEFLNEAGELEVAVGDSIDVVVE
AVDNGMGQTVLSREKAKREESWRALEQLHENDEIVKGVISSKVKGGFTVEIGSVRAFLPGSLVDVRPVRETTHL
EGKELEFKVIKIDKQRNNIVVSRRAVMEAENSAERDELLAKLEEGMEVEGIVKNLTEYGAFVDLGGIDGLLHIT
DMAWKRIKHPSEAVEVGQDLKVKVLKFDRERNRVSLGLKQLGADPWTEVEQTYPVGTITKARVINLTDYGCFAE
IAEGIEGLVHVSEMDHTNKNIHPSKVVQVGDEVNVMVLEIDGERRRISLGIKQTMPNPWAEFDKNHEKGQKISG
TIKSITDFGLFIGLEGGIDGLVHLSDISWTESGEEAIRNYSKGDTVEAVVLSVDAEANRISLGIKQL_Kanamycin
resistance cassette
SEQ ID NO: 59
>Moraxella catarrhalis BBH18 rpsA operon (rpsA-ihfB locus) [intergenic region
underlined]
ATGGTATCATTTGCTGAACTGTTTGAAGCAAGCCAATCAGAACAAGGTCTTAACATTGAGCGTGGCTCAGTGAT
TTCAGGAACAGTCGTTGCTATTGACAGCGATTGGATTACAGTTGATACAGGTCTTAAATCTGAAGGTATCGTTG
CTCGTGAAGAGTTCTTAAACGAAGCTGGCGAGCTTGAAGTGGCAGTAGGCGACAGCATTGATGTTGTAGTTGAA
GCTGTAGATAACGGCATGGGTCAAACTGTGCTTTCACGCGAAAAAGCGAAGCGTGAAGAAAGCTGGCGTGCGCT
TGAACAACTACACGAAAATGATGAAATCGTTAAAGGCGTTATTTCATCTAAAGTTAAAGGCGGTTTCACTGTTG
AGATCGGTTCAGTGCGTGCATTCCTTCCAGGGTCATTGGTAGATGTTCGCCCAGTTCGTGAAACGACACACCTA
GAAGGTAAAGAGCTTGAATTTAAAGTCATTAAAATTGATAAACAACGCAACAATATCGTTGTATCACGCCGTGC
TGTTATGGAAGCTGAAAATTCAGCAGAGCGTGATGAGCTACTAGCCAAGCTAGAAGAGGGCATGGAAGTAGAAG
GTATCGTTAAGAATCTAACTGAATATGGTGCATTTGTTGATTTAGGCGGTATTGATGGTCTATTGCACATCACC
GATATGGCGTGGAAGCGTATCAAGCACCCATCAGAAGCGGTAGAAGTTGGTCAAGACCTTAAAGTAAAAGTTCT
TAAATTTGACCGTGAGCGTAACCGTGTTAGCTTGGGTCTAAAACAATTGGGTGCTGACCCATGGACTGAAGTTG
AGCAAACTTATCCAGTGGGTACCATCACCAAAGCACGTGTAACCAACCTAACTGATTATGGCTGCTTTGCTGAA
ATCGCTGAAGGTATTGAAGGTTTGGTGCATGTCTCTGAAATGGATCATACGAACAAAAATATTCACCCATCAAA
AGTTGTTCAAGTGGGCGATGAGGTGAATGTGATGGTTCTTGAGATTGATGGTGAGCGTCGCCGTATTAGCCTTG
GTATCAAACAAACCATGCCAAATCCATGGGCTGAGTTTGATAAAAACCATGAAAAAGGTCAAAAAATCAGCGGT
ACCATCAAATCAATCACTGACTTTGGTCTGTTTATTGGCCTAGAAGGTGGTATTGATGGTTTGGTACACCTGTC
TGATATTTCATGGACTGAGTCTGGTGAAGAAGCCATCCGCAATTACTCAAAAGGTGATACTGTTGAGGCGGTCG
TATTGTCTGTTGATGCAGAAGCAAACCGTATCAGCCTAGGCATTAAGCAGCTAAACTCTGATCCATTTAATGAA
TACTTGCTATCAAATGATCGTGGTGCTATCGTTAAAGGTACAGTAAAAGAAGTGGATGCTAAAGGTGCGGTGAT
TACTTTGGCCGATGAAGTTGAAGGTTATTTGCGTGTTGCAGATATTGCTCGTGAGCGTACCGAAGATGCCTCTA
AAGTCTTGAGCGTTGGTGATGAAGTAGAAGCTAAAATTGTCGGCGTTGACCGCAAATCTCGCAATATCAGCCTA
TCAATCAAGGCAAAAGATGAAGCTGATGAGCGTCAAGCCATTAAAGAGCTTTCTAATACAGCAGCTGAAACACA
GCCAAAAACACTTGGCGATATTTTCGGTGAGCAGCTTAAAGGTTGATTGTCCTGAATATGATTAAGCCTATTAA
AAAGAGCGGTGTTGGTCATCGCTCTTTTTATTGCAGTAAATTGATTCAATATCATTTTTAACGGTTTGAATGTA
TTTTGAATGTGTGACATTTTAACAAAAAAATTATTTTCTTTTTTTGTGTTTTAAGTTATTATTTCCAGCTTTTT
TGATAAAAAGCTTGATTTGTGATTGAGGTAGGTCGTATGCAAGCGGTAATAAATAAATCTAATTTGATTGCAAA
TTTGGCATCAGTCTGTGAAGAGTTAGAAGAAGATGTTGTTGATGAAGCAGTACGCCTAATGATTGCGATGATGG
TCAATGAGTTGGTGTATGATGGGCGTATTGAAGTTAGAGGTTTTGGTAGCTTTTGTTTACATCACCGTTCTGCA
CGCATTGCTCGTAACCCACGCACAGGCGAGAGTGTCTCTGTTAAGGCCAAAGCCACTCCTTACTTTAAGCCTGG
TAAGGCACTGCGTGAATCGGTGAATTTGGTTAATGACTGA
SEQ ID NO: 60
>Moraxella catarrhalis BBH18 rpsA gene
ATGGTATCATTTGCTGAACTGTTTGAAGCAAGCCAATCAGAACAAGGTCTTAACATTGAGCGTGGCTCAGTGAT
TTCAGGAACAGTCGTTGCTATTGACAGCGATTGGATTACAGTTGATACAGGTCTTAAATCTGAAGGTATCGTTG
CTCGTGAAGAGTTCTTAAACGAAGCTGGCGAGCTTGAAGTGGCAGTAGGCGACAGCATTGATGTTGTAGTTGAA
GCTGTAGATAACGGCATGGGTCAAACTGTGCTTTCACGCGAAAAAGCGAAGCGTGAAGAAAGCTGGCGTGCGCT
TGAACAACTACACGAAAATGATGAAATCGTTAAAGGCGTTATTTCATCTAAAGTTAAAGGCGGTTTCACTGTTG
AGATCGGTTCAGTGCGTGCATTCCTTCCAGGGTCATTGGTAGATGTTCGCCCAGTTCGTGAAACGACACACCTA
GAAGGTAAAGAGCTTGAATTTAAAGTCATTAAAATTGATAAACAACGCAACAATATCGTTGTATCACGCCGTGC
TGTTATGGAAGCTGAAAATTCAGCAGAGCGTGATGAGCTACTAGCCAAGCTAGAAGAGGGCATGGAAGTAGAAG
GTATCGTTAAGAATCTAACTGAATATGGTGCATTTGTTGATTTAGGCGGTATTGATGGTCTATTGCACATCACC
GATATGGCGTGGAAGCGTATCAAGCACCCATCAGAAGCGGTAGAAGTTGGTCAAGACCTTAAAGTAAAAGTTCT
TAAATTTGACCGTGAGCGTAACCGTGTTAGCTTGGGTCTAAAACAATTGGGTGCTGACCCATGGACTGAAGTTG
AGCAAACTTATCCAGTGGGTACCATCACCAAAGCACGTGTAACCAACCTAACTGATTATGGCTGCTTTGCTGAA
ATCGCTGAAGGTATTGAAGGTTTGGTGCATGTCTCTGAAATGGATCATACGAACAAAAATATTCACCCATCAAA
AGTTGTTCAAGTGGGCGATGAGGTGAATGTGATGGTTCTTGAGATTGATGGTGAGCGTCGCCGTATTAGCCTTG
GTATCAAACAAACCATGCCAAATCCATGGGCTGAGTTTGATAAAAACCATGAAAAAGGTCAAAAAATCAGCGGT
ACCATCAAATCAATCACTGACTTTGGTCTGTTTATTGGCCTAGAAGGTGGTATTGATGGTTTGGTACACCTGTC
TGATATTTCATGGACTGAGTCTGGTGAAGAAGCCATCCGCAATTACTCAAAAGGTGATACTGTTGAGGCGGTCG
TATTGTCTGTTGATGCAGAAGCAAACCGTATCAGCCTAGGCATTAAGCAGCTAAACTCTGATCCATTTAATGAA
TACTTGCTATCAAATGATCGTGGTGCTATCGTTAAAGGTACAGTAAAAGAAGTGGATGCTAAAGGTGCGGTGAT
TACTTTGGCCGATGAAGTTGAAGGTTATTTGCGTGTTGCAGATATTGCTCGTGAGCGTACCGAAGATGCCTCTA
AAGTCTTGAGCGTTGGTGATGAAGTAGAAGCTAAAATTGTCGGCGTTGACCGCAAATCTCGCAATATCAGCCTA
TCAATCAAGGCAAAAGATGAAGCTGATGAGCGTCAAGCCATTAAAGAGCTTTCTAATACAGCAGCTGAAACACA
GCCAAAAACACTTGGCGATATTTTCGGTGAGCAGCTTAAAGGTTGA
SEQ ID NO: 61
>Moraxella catarrhalis BBH18 ihfB gene
ATGCAAGCGGTAATAAATAAATCTAATTTGATTGCAAATTTGGCATCAGTCTGTGAAGAGTTAGAAGAAGATGT
TGTTGATGAAGCAGTACGCCTAATGATTGCGATGATGGTCAATGAGTTGGTGTATGATGGGCGTATTGAAGTTA
GAGGTTTTGGTAGCTTTTGTTTACATCACCGTTCTGCACGCATTGCTCGTAACCCACGCACAGGCGAGAGTGTC
TCTGTTAAGGCCAAAGCCACTCCTTACTTTAAGCCTGGTAAGGCACTGCGTGAATCGGTGAATTTGGTTAATGA
CTGA
SEQ ID NO: 62
>Moraxella catarrhalis BBH18 IHF protein
MQAVINKSNLIANLASVCEELEEDVVDEAVRLMIAMMVNELVYDGRIEVRGFGSFCLHHRSARIARNPRTGESV
SVKAKATPYFKPGKALRESVNLVND
SEQ ID NO: 63
>RpsAFL (full-length RpsA) (gene)
atgactgaatcttttgctcaactctttgaagagtccttaaaagaaatcgaaacccgcccgggttctatcgttcg
tggcgttgttgttgctatcgacaaagacgtagtactggttgacgctggtctgaaatctgagtccgccatcccgg
ctgagcagttcaaaaacgcccagggcgagctggaaatccaggtaggtgacgaagttgacgttgctctggacgca
gtagaagacggcttcggtgaaactctgctgtcccgtgagaaagctaaacgtcacgaagcctggatcacgctgga
aaaagcttacgaagatgctgaaactgttaccggtgttatcaacggcaaagttaagggcggcttcactgttgagc
tgaacggtattcgtgcgttcctgccaggttctctggtagacgttcgtccggtgcgtgacactctgcacctggaa
ggcaaagagcttgaatttaaagtaatcaagctggatcagaagcgcaacaacgttgttgtttctcgtcgtgccgt
tatcgaatccgaaaacagcgcagagcgcgatcagctgctggaaaacctgcaggaaggcatggaagttaaaggta
tcgttaagaacctcactgactacggtgcattcgttgatctgggcggcgttgacggcctgctgcacatcactgac
atggcctggaaacgcgttaagcatccgagcgaaatcgtcaacgtgggcgacgaaatcactgttaaagtgctgaa
gttcgaccgcgaacgtacccgtgtatccctgggcctgaaacagctgggcgaagatccgtgggtagctatcgcta
aacgttatccggaaggtaccaaactgactggtcgcgtgaccaacctgaccgactacggctgcttcgttgaaatc
gaagaaggcgttgaaggcctggtacacgtttccgaaatggattggaccaacaaaaacatccacccgtccaaa
gttgttaacgttggcgatgtagtggaagttatggttctggatatcgacgaagaacgtcgtcgtatctccctggg
tctgaaacagtgcaaagctaacccgtggcagcagttcgcggaaacccacaacaagggcgaccgtgttgaaggta
aaatcaagtctatcactgacttcggtatcttcatcggcctggacggcggcatcgacggcctggttcacctgtct
gacatctcctggaacgttgcaggcgaagaagcagttcgtgaatacaaaaaaggcgacgaaatcgctgcagttgt
tctgcaggttgacgcagaacgtgaacgtatctccctgggcgttaaacagctcgcagaagatccg
ttcaacaactgggttgctctgaacaagaaaggcgctatcgtaaccggtaaagtaactgcagttgacgctaaagg
cgcaaccgtagaactggctgacggcgttgaaggttacctgcgtgcttctgaagcatcccgtgaccgcgttgaag
acgctaccctggttctgagcgttggcgacgaagttgaagctaaattcaccggcgttgatcgtaaaaaccgcgca
atcagcctgtctgttcgtgcgaaagacgaagctgacgagaaagatgcaatcgcaactgttaacaaacaggaaga
tgcaaacttctccaacaacgcaatggctgaagctttcaaagcagctaaaggcgagtaa
SEQ ID NO: 64
>RpsAFL (full-length RpsA) (protein)
MTESFAQLFEESLKEIETRPGSIVRGVVVAIDKDVVLVDAGLKSESAIPAEQFKNAQGELEIQVGDEVDVALDA
VEDGFGETLLSREKAKRHEAWITLEKAYEDAETVTGVINGKVKGGFTVELNGIRAFLPGSLVDVRPVRDTLHLE
GKELEFKVIKLDQKRNNVVVSRRAVIESENSAERDQLLENLQEGMEVKGIVKNLTDYGAFVDLGGVDGLLHITD
MAWKRVKHPSEIVNVGDEITVKVLKFDRERTRVSLGLKQLGEDPWVAIAKRYPEGTKLTGRVTNLTDYGCFVEI
EEGVEGLVHVSEMDWTNKNIHPSKVVNVGDVVEVMVLDIDEERRRISLGLKQCKANPWQQFAETHNKGDRVEGK
IKSITDFGIFIGLDGGIDGLVHLSDISWNVAGEEAVREYKKGDEIAAVVLQVDAERERISLGVKQLAEDPENNW
VALNKKGAIVTGKVTAVDAKGATVELADGVEGYLRASEASRDRVEDATLVLSVGDEVEAKFTGVDRKNRAISLS
VRAKDEADEKDAIATVNKQEDANFSNNAMAEAFKAAKGE-
SEQ ID NO: 65
>RpsATR (truncated RpsA) (gene)
atgactgaatcttttgctcaactctttgaagagtccttaaaagaaatcgaaaccccccgggttctatcgttcg
tggcgttgttgttgctatcgacaaagacgtagtactggttgacgctggtctgaaatctgagtccgccatcccgg
ctgagcagttcaaaaacgcccagggcgagctggaaatccaggtaggtgacgaagttgacgttgctctggacgca
gtagaagacggcttcggtgaaactctgctgtcccgtgagaaagctaaacgtcacgaagcctggatcacgctgga
aaaagcttacgaagatgctgaaactgttaccggtgttatcaacggcaaagttaagggcggcttcactgttgagc
tgaacggtattcgtgcgttcctgccaggttctctggtagacgttcgtccggtgcgtgacactctgcacctggaa
ggcaaagagcttgaatttaaagtaatcaagctggatcagaagcgcaacaacgttgttgtttctcgtcgtgccgt
tatcgaatccgaaaacagcgcagagcgcgatcagctgctggaaaacctgcaggaaggcatggaagttaaaggta
tcgttaagaacctcactgactacggtgcattcgttgatctgggcggcgttgacggcctgctgcacatcactgac
atggcctggaaacgcgttaagcatccgagcgaaatcgtcaacgtgggcgacgaaatcactgttaaagtgctgaa
gttcgaccgcgaacgtacccgtgtatccctgggcctgaaacagctgggcgaagatccgtgggtagctatcgcta
aacgttatccggaaggtaccaaactgactggtcgcgtgaccaacctgaccgactacggctgcttcgttgaaatc
gaagaaggcgttgaaggcctggtacacgtttccgaaatggattggaccaacaaaaacatccacccgtccaaa
gttgttaacgttggcgatgtagtggaagttatggttctggatatcgacgaagaacgtcgtcgtatctccctggg
tctgaaacagtgcaaagctaacccgtggcagcagttcgcggaaacccacaacaagggcgaccgtgttgaaggta
aaatcaagtctatcactgacttcggtatcttcatcggcctggacggcggcatcgacggcctggttcacctgtct
gacatctcctggaacgttgcaggcgaagaagcagttcgtgaatacaaaaaaggcgacgaaatcgctgcagttgt
tctgcaggttgacgcagaacgtgaacgtatctccctgggcgttaaacagctcgcataa
SEQ ID NO: 66
>RpsATR (truncated RpsA) (protein)
MTESFAQLFEESLKEIETRPGSIVRGVVVAIDKDVVLVDAGLKSESAIPAEQFKNAQGELEIQVGDEVDVALDA
VEDGFGETLLSREKAKRHEAWITLEKAYEDAETVTGVINGKVKGGFTVELNGIRAFLPGSLVDVRPVRDTLHLE
GKELEFKVIKLDQKRNNVVVSRRAVIESENSAERDQLLENLQEGMEVKGIVKNLTDYGAFVDLGGVDGLLHITD
MAWKRVKHPSEIVNVGDEITVKVLKFDRERTRVSLGLKQLGEDPWVAIAKRYPEGTKLTGRVTNLTDYGCFVEI
EEGVEGLVHVSEMDWTNKNIHPSKVVNVGDVVEVMVLDIDEERRRISLGLKQCKANPWQQFAETHNKGDRVEGK
IKSITDFGIFIGLDGGIDGLVHLSDISWNVAGEEAVREYKKGDEIAAVVLQVDAERERISLGVKQLA-
SEQ ID NO: 67
>V-PIPE Univ-F primer (pET15 vector amplification)
TAACGCGACTTAATTGGCCAGTGTGCCGGTCTCCG
SEQ ID NO: 68
>pET15notagRv (pET15 vector amplification)
CATGGTATATCTCCTTCTTAAAGTTAAAC
SEQ ID NO: 69
>rpsAfw (Forward primer for rpsA FL and TR amplification)
AAGGAGATATACCATGACTGAATCTTTTGCTCAACTC
SEQ ID NO: 70
>rpsAFLrv (Reverse primer for rpsA FL amplification)
AATTAAGTCGCGTTACTCGCCTTTAGCTGCTTTG
SEQ ID NO: 71
>rpsATRrv (Reverse primer for rpsA TR and TR amplification)
AATTAAGTCGCGTTATGCGAGCTGTTTAACGCCCAG
SEQ ID NO: 72
>T7prom (Colony screening)
TAATACGACTCACTATAGGG
SEQ ID NO: 73
>Seq Pet rv (Colony screening)
GATATCCGGATATAGTTCCTC

### Embodiments

1. A genetically modified Gram-negative bacterial cell comprising a modified rpsA gene, a modified rpsA operon and/or a modified 30S ribosomal protein S1 protein.
2. The genetically modified Gram-negative bacterial cell of embodiment 1, wherein the genetically modified bacterial cell is capable of secreting native outer membrane vesicles (nOMV).
3. The genetically modified Gram-negative bacterial cell of embodiment 2, wherein the genetically modified Gram-negative bacterial cell is capable of secreting greater quantities of nOMV compared to an unmodified bacterial cell.
4. The genetically modified Gram-negative bacterial cell of embodiment 3, wherein the genetically modified Gram-negative bacterial cell is capable of secreting greater quantities of nOMV compared to a wild-type bacterial cell.
5. The genetically modified Gram-negative bacterial cell of any one of the preceding embodiments, wherein the genetically modified Gram-negative bacterial cell comprises a modified rpsA gene, wherein the modified rpsA gene(s) comprises one or more mutation(s) relative to a wild-type rpsA gene, wherein the one or more mutation(s) are located within the coding region of the rpsA gene and/or within the non-coding region of the rpsA gene.
6. The genetically modified Gram-negative bacterial cell of embodiment 5, where in the one or more mutation(s) comprises or consists of the insertion of one or more nucleotide(s), wherein the one or more nucleotide(s) inserted is selected from the group consisting of:
   a. a transposon mobile element;
   b. a selection marker, for example, an antibiotic resistance cassette; a gene encoding a fluorescent protein; or a gene encoding an antitoxin; and/or
   c. a fragment of the rpsA gene.
7. The genetically modified Gram-negative bacterial cell of any one of embodiments 5 and 6, wherein the one or more mutation(s) was made using, or is obtained or obtainable by a method selected from the group consisting of site directed mutagenesis, a recombinase-mediated method, a λ-red recombinase-mediated method, a prophage-based approach, a mobile group II introns knock out, transposon-mediated genome editing and CRISPR-Cas genome editing.
8. The genetically modified Gram-negative bacterial cell of any one of embodiments 6(a) and 7, wherein the insertion of a transposon mobile element is made using, or is obtained or obtainable by transposon-mediated genome editing.
9. The genetically modified Gram-negative bacterial cell of any one of embodiment 6(a) to 8, wherein the transposon mobile element is selected from the group consisting of a Tn5 transposon, a Tn10 transposon, a Tn3 transposon, a Tn7 transposon, an IS5376 transposon, a bacteriophage MuA transposon, an IS200/IS605 transposon, and an IS91 transposon.
10. The genetically modified Gram-negative bacterial cell of embodiment 9, wherein the transposon mobile element is a Tn5 transposon.
11. The genetically modified Gram-negative bacterial cell of any one of the preceding embodiments, wherein the genetically modified bacterial cell comprises a modified 30S ribosomal protein S1, wherein the modified 30S ribosomal protein(s) S1:
   a. comprises one or more mutation(s) relative to a wild-type 30S ribosomal protein S1; and/or
   b. comprises one or more post-translational modification(s) relative to a wild-type 30S ribosomal protein S1.
12. The genetically modified Gram-negative bacterial cell of any one of the preceding embodiments, wherein the genetically modified bacterial cell comprises a modified 30S ribosomal protein S1, wherein the modified 30S ribosomal protein(s) S1 is downregulated relative to the 30S ribosomal protein S1 of an unmodified bacterial cell.
13. The genetically modified Gram-negative bacterial cell of any one of the preceding embodiments, wherein the genetically modified Gram-negative bacterial cell comprises a modified 30S ribosomal protein S1, wherein the modified 30S ribosomal protein(s) S1 is truncated relative to a wild-type 30S ribosomal protein S1.
14. The genetically modified Gram-negative bacterial cell of claim anyone of embodiments 11 to 13, wherein the modified 30S ribosomal protein(s) S1 is encoded by a modified rpsA gene(s).
15. The genetically modified Gram-negative bacterial cell of any one of embodiments 5 to 10, wherein the modified rpsA gene(s) is i) chromosomic, or ii) extra-chromosomic, for example, the modified rpsA gene(s) is encoded by a plasmid or by a cosmid.
16. The genetically modified Gram-negative bacterial cell of any one of embodiments 1 to 4 and 11 to 14, wherein the genetically modified Gram-negative bacterial cell comprises a modified 30S ribosomal protein S1, wherein the modified 30S ribosomal protein(s) S1 comprises or consists of an amino acid sequence having at least 60% sequence identity to a wild-type 30S ribosomal protein S1, for example, at least 70%, 80%, 85%, 90%, 92%, 95%, 96%, 97%, 98%, 99%, or 100% sequence identity to a wild-type 30S ribosomal protein S1.
17. The genetically modified Gram-negative bacterial cell of any one of embodiments 1 to 4, 11 to 14, and 16 wherein the genetically modified Gram-negative bacterial cell comprises a modified 30S ribosomal protein S1, wherein the modified 30S ribosomal protein(s) S1 comprises or consists of “R” domains selected from the group consisting of:
   a. an R1 domain or a fragment thereof, an R2 domain or a fragment thereof, an R3 domain or a fragment thereof and an R4 domain or a fragment thereof;
   b. an R1 domain or a fragment thereof, an R2 domain or a fragment thereof, and an R3 domain or a fragment thereof;
   c. an R2 domain or a fragment thereof, an R3 domain or a fragment thereof, and an R4 domain or a fragment thereof;
   d. an R2 domain or a fragment thereof, and an R3 domain or a fragment thereof;
   e. an R3 domain or a fragment thereof, and an R4 domain or a fragment thereof; and
   f. an R3 domain or a fragment thereof.
18. The genetically modified Gram-negative bacterial cell of any one of embodiments 1 to 4, 11 to 14, and 16 to 17, wherein the genetically Gram-negative modified bacterial cell comprises a modified 30S ribosomal protein S1, wherein the modified 30S ribosomal protein(s) S1 comprises or consists of an amino acid sequence having:
   a. an R1 domain, when present, with least 60% sequence identity to a wild-type S1, for example, at least 70%, 80%, 85%, 90%, 92%, 95%, 96%, 97%, 98%, 99%, or 100% sequence identity to a wild-type R1 domain;
   b. an R2 domain, when present, with least 60% sequence identity to a wild-type S1, for example, at least 70%, 80%, 85%, 90%, 92%, 95%, 96%, 97%, 98%, 99%, or 100% sequence identity to a wild-type R2 domain;
   c. an R3 domain with least 60% sequence identity to a wild-type S1, for example, at least 70%, 80%, 85%, 90%, 92%, 95%, 96%, 97%, 98%, 99%, or 100% sequence identity to a wild-type R3 domain; and/or
   d. an R4 domain, when present, with least 40% sequence identity to a wild-type S1, for example, at least 50%, 60%, 70%, 80%, 85%, 90%, 92%, 95%, 96%, 97%, 98%, 99%, or 100% sequence identity to a wild-type R4 domain.
19. The genetically modified Gram-negative bacterial cell of any one of embodiments 1 to 4, 11 to 14, and 16 to 18, wherein the genetically Gram-negative modified bacterial cell comprises a modified 30S ribosomal protein S1, wherein the modified 30S ribosomal protein(s) S1 comprises an amino acid sequence upstream of the first and/or the last amino acid of the R1, R2, R3 and/or R4 domain, when present, having at least 60%, 70%, 80%, 85%, 90%, 92%, 95%, 96%, 97%, 98%, 99%, or 100% sequence identity to the amino acid sequence upstream of the first and/or the last residue of the R1, R2, R3 and/or R4 domain of a wild-type 30S ribosomal protein S1.
20. The genetically modified Gram-negative bacterial cell of any one of embodiments 1 to 4, 11 to 14, and 16 to 19, wherein the genetically modified Gram-negative bacterial cell comprises a modified 30S ribosomal protein S1, wherein the modified 30S ribosomal protein(s) S1 was modified using, or is obtained or obtainable by genome editing, gene silencing, fragmenting the RNA post-transcriptionally and/or post-translational modification.
21. The genetically modified Gram-negative bacterial cell of any one of the preceding embodiments, wherein the bacterial cell comprises a wild-type rpsA gene.
22. The genetically modified Gram-negative bacterial cell of any one of the preceding embodiments, wherein the Gram-negative bacterial cell comprises a wild-type 30S ribosomal protein S1 and a modified 30S ribosomal protein S1 protein.
23. The genetically modified Gram-negative bacterial cell of any one of embodiments 1 to 20 and 22, wherein the genetically modified Gram-negative bacterial cell does not comprise a wild-type rpsA gene.
24. The genetically modified Gram-negative bacterial cell of any one of embodiments 1 to 21 and 23, wherein the genetically modified Gram-negative bacterial cell does not comprise a wild-type 30S ribosomal protein S1.
25. The genetically modified Gram-negative bacterial cell of any one of embodiments 3 to 24, wherein the genetically modified Gram-negative bacterial cell, compared to an unmodified bacterial cell, is capable of releasing at least 2.0-fold more nOMVs when grown in liquid culture, for example, 2.5-fold more, 3.0-fold more, 3.5-fold more, 4.0-fold more, 4.5-fold more, 5.0-fold more, 5.5-fold more, 6.0-fold more, 10-fold more, 20-fold more, 30-fold more, 40-fold more, 50-fold more, 60-fold more, 70-fold more, 80-fold more, 90-fold more, or 100-fold more nOMVs when growing in liquid culture.
26. The genetically modified Gram-negative bacterial cell of any one of the preceding embodiments, characterised in that it is capable of proliferation.
27. The genetically modified Gram-negative bacterial cell of embodiment 26, wherein a culture of the genetically modified Gram-negative bacterial cell is capable of generating a biomass which is at least 10% of the biomass of a culture of the unmodified bacterial cell grown in the same culture conditions, for example, 20%, 30%, 40%, 50%, 60%, 70%, 80%, 85%, 90%, 92%, 95%, 96%, 97%, 98%, 99% or 100% of the biomass of a culture of the unmodified bacterial cell grown in the same culture conditions.
28. The genetically modified Gram-negative bacterial cell of any one of embodiments 26 and 27, wherein a culture of the genetically modified Gram-negative bacterial cell is capable of achieving a turbidity which is at least 10% of the turbidity of a culture of the unmodified bacterial cell grown in the same culture conditions, for example, 20%, 30%, 40%, 50%, 60%, 70%, 80%, 85%, 90%, 92%, 95%, 96%, 97%, 98%, 99% or 100% of the turbidity of a culture of the unmodified bacterial cell grown in the same culture conditions.
29. The genetically modified Gram-negative bacterial cell of embodiment 28, wherein the turbidity is measured as OD at 600 nm.
30. The genetically modified Gram-negative bacterial cell of embodiment 26, wherein a culture of the genetically modified Gram-negative bacterial cell is capable of achieving a nutrient uptake which is at least 10% of the nutrient uptake of a culture of the unmodified bacterial cell grown in the same culture conditions, for example, 20%, 30%, 40%, 50%, 60%, 70%, 80%, 85%, 90%, 92%, 95%, 96%, 97%, 98%, 99% or 100% of the nutrient uptake of a culture of the unmodified bacterial cell grown in the same culture conditions.
31. The genetically modified Gram-negative bacterial cell of any one of embodiments 26 to 30, wherein the genetically modified Gram-negative bacterial cell is capable of reaching stationary phase no later than 120 hours after the unmodified bacterial cell grown in the same culture conditions, for example, no later than 1, 2, 3, 4, 5, 6, 7, 8, 9, 10, 12, 15, 20, 25, 30, 40, 50, 60, 70, 80, 90, 100, or 110 hours after the unmodified bacterial cell grown in the same culture conditions.
32. The genetically modified Gram-negative bacterial cell of embodiment 26, wherein growth of the genetically modified Gram-negative bacterial cell is determined according to biomass, turbidity and/or nutrient uptake.
33. The genetically modified Gram-negative bacterial cell of any one of the preceding embodiments, wherein the one or more mutation(s) relative to a wild-type rpsA gene occur in a designated nucleotide sequence comprising or consisting of:
   a. from 1 to 10 nucleotides upstream of the 3′-end of the R3 domain relative to the wild-type rpsA gene, for example, from 11 to 20, from 21 to 50, or from 51 and 100 nucleotides upstream of the 3′-end of the R3 domain relative to the wild-type rpsA gene;
   b. from 1 to 100 nucleotides upstream of the 3′-end of the R3 domain relative to the wild-type rpsA gene, for example, from 10 to 50, or from 20 to 30 nucleotides upstream of the 3′-end of the R3 domain relative to the wild-type rpsA gene;
   c. from 1% to 10% of the nucleotides relative to the wild-type rpsA gene, for example, from 11% to 20%, from 21% to 30% relative the nucleotides of the wild-type rpsA gene, wherein the designated region is located between the 5′-end relative to the wild-type rpsA gene and the 3′-end of the R3 domain relative to the wild-type RpsA gene;
   d. from 1% to 30% of the nucleotides relative to the wild-type rpsA gene, for example, from 5% to 25%, from 10% to 20% of the nucleotides relative to the wild-type rpsA gene, wherein the designated region is located between the 5′-end relative the wild-type RpsA gene and the 3′-end of the R3 domain relative to the wild-type rpsA gene;
   e. from 1 and 10 nucleotides downstream of the 3′-end of the R3 domain relative to the wild-type rpsA gene, for example, from 11 and 20, from 21 to 50, or from 51 to 100 nucleotides downstream of the 3′-end of the R3 domain relative to wild-type rpsA gene;
   f. from 1 to 100 nucleotides downstream of the 3′-end of the R3 domain relative to the wild-type rpsA gene, for example, from 10 to 50, or from 20 to 30, nucleotides downstream of the 3′-end of the R3 domain relative to the wild-type rpsA gene;
   g. from 1 and 10 nucleotides downstream of the 5′-end of the R4 domain relative to the wild-type rpsA gene, for example, from 11 and 20, from 21 to 50, or from 51 to 100 nucleotides downstream of the 5′-end of the R4 domain relative to wild-type rpsA gene;
   h. from 1 to 100 nucleotides downstream of the 5′-end of the R4 domain relative to the wild-type rpsA gene, for example, from 10 to 50, or from 20 to 30, nucleotides downstream of the 5′-end of the R4 domain relative to the wild-type rpsA gene;
   i. from 1 and 10 nucleotides downstream of the 3′-end of the R4 domain relative to the wild-type rpsA gene, for example, from 11 and 20, from 21 to 50, or from 51 to 100 nucleotides downstream of the 3′-end of the R4 domain relative to wild-type rpsA gene;
   j. from 1 to 100 nucleotides downstream of the 3′-end of the R4 domain relative to the wild-type rpsA gene, for example, from 10 to 50, or from 20 to 30, nucleotides downstream of the 3′-end of the R4 domain relative to the wild-type rpsA gene;
   k. from 1% to 10% of the nucleotides relative to the wild-type rpsA gene, for example, from 11% to 20%, from 21% to 30% of the nucleotides relative to the wild-type rpsA gene, wherein the designated region is located between the 3′-end relative to the R3 domain of the wild-type rpsA gene and the 3′-end relative to the wild-type rpsA gene;
   l. from 1% to 30%, of the nucleotides relative to the wild-type rpsA gene, for example, from 5% to 25%, from 10% to 20% of the nucleotides relative to the wild-type rpsA gene, wherein the designated region is located between the 3′-end relative to the R3 domain of the wild-type rpsA gene and the 3′-end relative to the wild-type rpsA gene;
   m. from 1 to 5 nucleotides upstream of the 3′-end relative to the wild-type rpsA gene, for example, from 6 to 10, from 11 to 20, from 21 to 30, from 31 to 40, from 41 to 50, from 51 to 100, from 101 to 150, from 151 to 200, from 201 to 250, from 251 to 300, from 301 to 350, from 351 to 400, from 401 to 450, from 451 to 500 nucleotides upstream of the 3′-end relative to the wild-type rpsA gene; and/or
   n. from 1 to 500 nucleotides upstream the 3′-end relative to the wild-type rpsA gene, for example, from 50 to 450, from 100 to 400, from 150 to 380, from 200 to 378, or from 250 to 376 nucleotides upstream the 3′-end relative to the wild-type rpsA gene.
34. The genetically modified Gram-negative bacterial cell of any one of the preceding embodiments, wherein the genetically modified Gram-negative bacterial cell comprises a modified ihfB gene and/or a modified IHF protein.
35. The genetically modified Gram-negative bacterial cell of embodiment 34, wherein:
   a. the modified ihfB gene(s) comprises one or more mutation(s) relative to a wild-type ihfB gene, wherein the one or more mutation(s) are located within the coding region of the ihfB gene and/or within the non-coding region of the ihfB gene;
   b. the modified ihfB gene(s) is knocked-out relative to a wild-type ihfB gene;
   c. the modified IHF protein(s) comprises one or more mutation(s) relative to a wild-type IHF protein;
   d. The modified IHF protein(s) comprises one or more post-translational modification(s) relative to a wild-type IHF protein;
   e. the modified IHF protein(s) is downregulated relative to the IHF protein of an unmodified bacterial cell; and/or
   f. the modified IHF protein(s) is encoded by the modified ihfB gene(s).
36. The genetically modified Gram-negative bacterial cell of any one of embodiments 34 and 35, wherein the mutant IHF protein(s) comprises or consists of an amino acid sequence having at least 60% sequence identity to a wild-type IHF protein, for example, at least 70%, 80%, 85%, 90%, 92%, 95%, 96%, 97%, 98%, 99%, or 100% sequence identity to a wild-type IHF protein.
37. The genetically modified Gram-negative bacterial cell of any one of the preceding embodiments, wherein the wild-type rpsA gene is selected from the group consisting of: SEQ ID NO: 1, SEQ ID NO: 2, SEQ ID NO: 3, SEQ ID NO: 4, SEQ ID NO: 5, and SEQ ID NO: 6.
   38. The genetically modified Gram-negative bacterial cell of any one of the preceding embodiments, wherein the wild-type S1 is selected from the group consisting of: SEQ ID NO: 13, SEQ ID NO: 14, SEQ ID NO: 15, SEQ ID NO: 16, SEQ ID NO: 17, and SEQ ID NO: 18.
   39. The genetically modified Gram-negative bacterial cell of any one of the preceding embodiments, wherein the wild-type IHF protein is selected from the group consisting of: SEQ ID NO: 31, SEQ ID NO: 32, SEQ ID NO: 33, SEQ ID NO: 34, SEQ ID NO: 35, and SEQ ID NO: 36.
   40. The genetically modified Gram-negative bacterial cell of any one of the preceding embodiments which is selected from the group consisting of Enterobacteriaceae, Neisseriaceae, Helicobacteraceae, Campylobacteraceae, Yersiniaceae, Vibrionaceae, Pasteurellaceae, Alcaligenaceae, Pseudomonadaceae and Moraxellaceae.
41. The genetically modified Gram-negative bacterial cell of any one of the preceding embodiments which is selected from the group consisting of Escherichia coli, Neisseria meningitidis, Neisseria lactamica, Neisseria gonorrhoeae, Helicobacter pylori, Salmonella typhi, Salmonella typhimurium, Vibrio cholerae, Shigella spp., Haemophilus influenzae, Bordetella pertussis, Pseudomonas aeruginosa and Moraxella catarrhalis.
42. The genetically modified Gram-negative bacterial cell of embodiment 41, wherein the Gram-negative bacterial cell is Bordetella pertussis.
43. The genetically modified Gram-negative bacterial cell of any one of the preceding embodiments, wherein the genetically modified Gram-negative bacterial cell comprises a modified rpsA gene, wherein the modified rpsA gene comprises or consists of a nucleotide sequences having at least 60% sequence identity to mutants SEQ ID NO: 37, for example, at least 70%, 80%, 85%, 90%, 92%, 95%, 96%, 97%, 98%, 99%, or 100% sequence identity to SEQ ID NO: 37.
44. The genetically modified Gram-negative bacterial cell of any one of the preceding embodiments, wherein the genetically modified Gram-negative bacterial cell comprises a modified rpsA operon, wherein the modified rpsA operon comprises or consists of a nucleotide sequences having at least 60% sequence identity to SEQ ID NO: 38 or SEQ ID NO: 51, for example, at least 70%, 80%, 85%, 90%, 92%, 95%, 96%, 97%, 98%, 99%, or 100% sequence identity to SEQ ID NO: 38 or SEQ ID NO: 51.
45. The genetically modified Gram-negative bacterial cell of any one of the preceding embodiments, wherein the genetically modified Gram-negative bacterial cell comprises a modified 30S ribosomal protein S1, wherein the modified 30S ribosomal protein S1 comprises or consists of an amino acid sequence having at least 60% sequence identity to a polypeptide selected from the group consisting of SEQ ID NO: 39, SEQ ID NO: 40, SEQ ID NO: 41, and SEQ ID NO: 53, for example, at least 70%, 80%, 85%, 90%, 92%, 95%, 96%, 97%, 98%, 99%, or 100% sequence identity to a polypeptide selected from the group consisting of SEQ ID NO: 39, SEQ ID NO: 40, SEQ ID NO: 41, and SEQ ID NO: 53.
46. The genetically modified Gram-negative bacterial cell of embodiment 41, wherein the bacterial cell is E coli.
47. The genetically modified Gram-negative bacterial cell of any one of the preceding embodiments, wherein the genetically modified Gram-negative bacterial cell comprises a modified rpsA operon, wherein the modified rpsA operon comprises or consists of a nucleotide sequences having at least 60% sequence identity to SEQ ID NO: 42 or SEQ ID NO: 43, for example, at least 70%, 80%, 85%, 90%, 92%, 95%, 96%, 97%, 98%, 99%, or 100% sequence identity to SEQ ID NO: 42 or SEQ ID NO: 43.
48. The genetically modified Gram-negative bacterial cell of any one of the preceding embodiments, wherein the genetically modified Gram-negative bacterial cell comprises a modified 30S ribosomal protein S1, wherein the modified 30S ribosomal protein S1 comprises or consists of an amino acid sequence having at least 60% sequence identity to SEQ ID NO: 44 or SEQ ID NO: 45, for example, at least 70%, 80%, 85%, 90%, 92%, 95%, 96%, 97%, 98%, 99%, or 100% sequence identity to SEQ ID NO: 44 or SEQ ID NO: 45.
49. The genetically modified Gram-negative bacterial cell of any one of the preceding embodiments, wherein the genetically modified Gram-negative bacterial cell expresses one or more exogenous antigens.
50. The genetically modified Gram-negative bacterial cell of any preceding embodiments which has been further genetically engineered by one or more processes selected from the following group: (a) a process of down-regulating expression of immunodominant variable or non-protective antigens, (b) a process of up-regulating expression of protective OMP antigens, (c) a process of down-regulating a gene involved in rendering the lipid A portion of LPS toxic, (d) a process of up-regulating a gene involved in rendering the lipid A portion of LPS less toxic, and (e) a process of genetically modifying the bacterial cell to express a heterologous antigen.
51. A method of generating a genetically modified Gram-negative bacterial cell, comprising a step of modifying a wild-type rpsA gene, operon, RNA and/or 30S ribosomal protein S1 protein, such that the modification causes the genetically modified Gram-negative bacterial cell, when grown in culture medium, to release greater quantities of nOMVs into the medium than the unmodified bacterial cell.
52. A method of generating a genetically modified Gram-negative bacterial cell, comprising a step of providing a modified 30S ribosomal protein S1, such that the modification causes the genetically modified Gram-negative bacterial cell, when grown in culture medium, to release greater quantities of nOMVs into the medium than the unmodified bacterial cell.
53. A process for preparing nOMVs, comprising the steps of:
   a. inoculating a culture vessel containing a nutrient medium suitable for growth of the genetically modified Gram-negative bacterial cell of any one of embodiments 1 to 50;
   b. culturing the genetically modified Gram-negative bacterial cell under conditions which permit the release of nOMVs into the medium by the bacteria; and
   c. recovering nOMVs from the medium; and
   d. mixing the nOMVs with a pharmaceutically acceptable diluent or carrier.
54. The process of embodiment 53 which further comprises a step after step (c) comprising sterile-filtering the preparation of nOMVs.
55. An nOMV obtained or obtainable from the genetically modified Gram-negative bacterial cell of any one of embodiments 1 to 50, or from a genetically modified Gram-negative bacterial cell obtained or obtainable by the method of embodiments 51 and 52, or by the process of embodiments 53 and 54.
56. An immunogenic composition comprising the nOMV of embodiment 55.
57. The immunogenic composition of embodiment 55, further comprising one or more additional antigens from the same or different pathogen.
58. A vaccine comprising the nOMV of embodiment 55.
59. The nOMV of embodiment 55, the immunogenic composition of any one of embodiments 56 and 57 or the vaccine of embodiment 58 for use in medicine.
60. The nOMV of embodiment 55, the immunogenic composition of any one of embodiments 56 and 57 or the vaccine of embodiment 58 for use in inducing an immune response in a vertebrate, preferably a mammal.
61. The nOMV of embodiment 55, the immunogenic composition of any one of embodiments 56 and 57 or the vaccine of embodiment 58 for use in the treatment of prevention of a disease which is caused by a bacterial cell of the same genus or species of the genetically modified Gram-negative bacterial cell from which the nOMV was obtained or obtainable.
62. The nOMV of embodiment 55, the immunogenic composition of any one of embodiments 56 and 57 or the vaccine of embodiment 58 for use in the treatment of prevention of a disease which is caused by Bordetella, preferably by B. pertussis.
63. The nOMV of embodiment 55, the immunogenic composition of any one of embodiments 56 and 57 or the vaccine of embodiment 58 for use in the treatment of prevention of a disease which is caused by Escherichia coli.
64. A method of immunising a subject in need thereof against a bacterial cell by administering the nOMV of embodiment 55, the immunogenic composition of any one of embodiments 56 and 57 or the vaccine of embodiment 58.
65. The use of the nOMV of embodiment 55, the immunogenic composition of any one of embodiments 56 and 57 or the vaccine of embodiment 58 in the manufacture of a medicament for immunising a subject in need thereof against a bacterial cell.

### Further Embodiments

1. A genetically modified Gram-negative bacterial cell comprising a modified rpsA gene, a modified rpsA operon and/or a modified 30S ribosomal protein S1 protein.
2. The genetically modified Gram-negative bacterial cell of embodiment 1, wherein the genetically modified bacterial cell is capable of secreting native outer membrane vesicles (nOMV).
3. The genetically modified Gram-negative bacterial cell of embodiment 2, wherein the genetically modified Gram-negative bacterial cell is capable of secreting greater quantities of nOMV compared to an unmodified bacterial cell.
4. The genetically modified Gram-negative bacterial cell of embodiment 3, wherein the genetically modified Gram-negative bacterial cell is capable of secreting greater quantities of nOMV compared to a wild-type bacterial cell.
5. The genetically modified Gram-negative bacterial cell of any one of the preceding embodiments, wherein the genetically modified Gram-negative bacterial cell comprises a modified rpsA gene, wherein the modified rpsA gene(s) comprises one or more mutation(s) relative to a wild-type rpsA gene, wherein the one or more mutation(s) are located within the coding region of the rpsA gene and/or within the non-coding region of the rpsA gene.
6. The genetically modified Gram-negative bacterial cell of embodiment 5, wherein the one or more mutation(s) comprises or consists of the insertion of one or more nucleotide(s), wherein the one or more nucleotide(s) inserted is selected from the group consisting of:
   a. a transposon mobile element;
   b. a selection marker, for example, an antibiotic resistance cassette; a gene encoding a fluorescent protein; or a gene encoding an antitoxin; and/or
   c. a fragment of the rpsA gene.
7. The genetically modified Gram-negative bacterial cell of embodiment 5 or 6, wherein the one or more mutation(s) was made using, or is obtained or obtainable by a method selected from the group consisting of site directed mutagenesis, a recombinase-mediated method, a λ-red recombinase-mediated method, a prophage-based approach, a mobile group II introns knock out, transposon-mediated genome editing and CRISPR-Cas genome editing.
8. The genetically modified Gram-negative bacterial cell of embodiment 6(a) or 7, wherein the insertion of a transposon mobile element is made using, or is obtained or obtainable by transposon-mediated genome editing.
9. The genetically modified Gram-negative bacterial cell of any one of embodiments 6(a) to 8, wherein the transposon mobile element is selected from the group consisting of a Tn5 transposon, a Tn10 transposon, a Tn3 transposon, a Tn7 transposon, an IS5376 transposon, a bacteriophage MuA transposon, an IS200/IS605 transposon, and an IS91 transposon.
10. The genetically modified Gram-negative bacterial cell of embodiment 9, wherein the transposon mobile element is a Tn5 transposon.
11. The genetically modified Gram-negative bacterial cell of any one of the preceding embodiments, wherein the genetically modified Gram-negative bacterial cell comprises a modified 30S ribosomal protein S1, wherein the modified 30S ribosomal protein(s) S1:
   a. comprises one or more mutation(s) relative to a wild-type 30S ribosomal protein S1; and/or
   b. comprises one or more post-translational modification(s) relative to a wild-type 30S ribosomal protein S1.
12. The genetically modified Gram-negative bacterial cell of any one of the preceding embodiments, wherein the genetically modified Gram-negative bacterial cell comprises a modified 30S ribosomal protein S1, wherein the modified 30S ribosomal protein(s) S1 is downregulated relative to the 30S ribosomal protein S1 of an unmodified bacterial cell.
13. The genetically modified Gram-negative bacterial cell of embodiment 11 or 12, wherein the one more mutation(s) relative to a wild-type 30S ribosomal protein S1 comprises one or more mutation(s) in a region corresponding to the R4 domain of B. pertussis 30S ribosomal protein S1 and/or a region corresponding to the portion of the B. pertussis 30S ribosomal protein S1 between the R3 and R4 domains.
14. The genetically modified Gram-negative bacterial cell of any one of embodiments 11 to 13, wherein the one or more mutation(s) relative to a wild-type 30S ribosomal protein S1 comprises mutation or deletion of at least 5, at least 10, at least 25, at least 50, at least 75, at least 100, at least 110, at least 125, between 5 and 150, between 25 and 130, between 100 and 130, or between 125 and 130 amino acids.
15. The genetically modified Gram-negative bacterial cell of any one of embodiments 11 to 14, wherein the one or more mutation(s) relative to a wild-type 30S ribosomal protein S1 comprises mutation or deletion of at least 5, at least 10, at least 15, at least 20, or at least 25, amino acids of the region corresponding to amino acids 550 to 576 of the B. pertussis 30S ribosomal protein S1.
16. The genetically modified Gram-negative bacterial cell of any one of embodiments 11 to 15, wherein the one or more mutation(s) relative to a wild-type 30S ribosomal protein S1 comprises mutation or deletion of at least 20, at least 30, at least 50, at least 75, or at least 100 amino acids of the region corresponding to amino acids 473 to 576 of the B. pertussis 30S ribosomal protein S1.
17. The genetically modified Gram-negative bacterial cell of any one of embodiments 11 to 16, wherein the one or more mutation(s) relative to a wild-type 30S ribosomal protein S1 comprises mutation or deletion of at least 20, at least 30, at least 50, at least 100, or at least 120 amino acids of the region corresponding to amino acids 450 to 576 of the B. pertussis 30S ribosomal protein S1.
18. The genetically modified Gram-negative bacterial cell of any one of embodiments 11 to 17, wherein the one or more mutation(s) relative to a wild-type 30S ribosomal protein S1 comprises truncation of at least 20, at least 30, at least 50, at least 100, or at least 120 consecutive amino acids from the C-terminal end of the 30S ribosomal protein S1.
19. The genetically modified Gram-negative bacterial cell of any one of embodiments 11 to 18, wherein the one or more mutation(s) relative to a wild-type 30S ribosomal protein S1 comprises mutation or deletion of amino acids corresponding to amino acids 560 to 576, 552 to 576, 500 to 576 485 to 576, 460 to 576, or 453 to 576 of the B. pertussis 30S ribosomal protein S1.
20. The genetically modified Gram-negative bacterial cell of any one of the preceding embodiments, wherein the genetically modified Gram-negative bacterial cell comprises a modified 30S ribosomal protein S1, wherein the modified 30S ribosomal protein(s) S1 is truncated relative to a wild-type 30S ribosomal protein S1.
21. The genetically modified Gram-negative bacterial cell of any one of embodiments 11 to 20, wherein the modified 30S ribosomal protein(s) S1 is encoded by a modified rpsA gene(s).
22. The genetically modified Gram-negative bacterial cell of any one of embodiments 1 to 21, wherein the modified rpsA gene(s) is i) chromosomic, or ii) extra-chromosomic, for example, the modified rpsA gene(s) is encoded by a plasmid or by a cosmid.
23. The genetically modified Gram-negative bacterial cell of any one of embodiments 1 to 22, wherein the genetically modified Gram-negative bacterial cell comprises a modified 30S ribosomal protein S1, wherein the modified 30S ribosomal protein(s) S1 comprises or consists of an amino acid sequence having at least 60% sequence identity to a wild-type 30S ribosomal protein S1, for example, at least 70%, 80%, 85%, 90%, 92%, 95%, 96%, 97%, 98%, 99%, or 100% sequence identity to a wild-type 30S ribosomal protein S1.
24. The genetically modified Gram-negative bacterial cell of any one of embodiments 1 to 23, wherein the genetically modified Gram-negative bacterial cell comprises a modified 30S ribosomal protein S1, wherein the modified 30S ribosomal protein(s) S1 comprises or consists of an amino acid sequence having at least 90%, at least 95%, at least 98%, at least 99%, or 100% identity to a fragment of a wild-type 30S ribosomal protein S1 corresponding to amino acids 1 to 452 of the B. pertussis 30S ribosomal protein S1.
25. The genetically modified Gram-negative bacterial cell of any one of embodiments 1 to 24, wherein the genetically modified Gram-negative bacterial cell comprises a modified 30S ribosomal protein S1, wherein the modified 30S ribosomal protein(s) S1 comprises or consists of an amino acid sequence having at least at least 98%, at least 99%, or 100% identity to a fragment of a wild-type 30S ribosomal protein S1 corresponding to amino acids 1 to 453 of the B. pertussis 30S ribosomal protein S1.
26. The genetically modified Gram-negative bacterial cell of any one of embodiments 1 to 25 wherein the genetically modified Gram-negative bacterial cell comprises a modified 30S ribosomal protein S1, wherein the modified 30S ribosomal protein(s) S1 comprises or consists of “R” domains selected from the group consisting of:
   a. an R1 domain or a fragment thereof, an R2 domain or a fragment thereof, an R3 domain or a fragment thereof and an R4 domain or a fragment thereof;
   b. an R1 domain or a fragment thereof, an R2 domain or a fragment thereof, and an R3 domain or a fragment thereof;
   c. an R2 domain or a fragment thereof, an R3 domain or a fragment thereof, and an R4 domain or a fragment thereof;
   d. an R2 domain or a fragment thereof, and an R3 domain or a fragment thereof;
   e. an R3 domain or a fragment thereof, and an R4 domain or a fragment thereof; and
   f. an R3 domain or a fragment thereof.
27. The genetically modified Gram-negative bacterial cell of any one of embodiments 1 to 26, wherein the genetically Gram-negative modified bacterial cell comprises a modified 30S ribosomal protein S1, wherein the modified 30S ribosomal protein(s) S1 comprises or consists of an amino acid sequence having:
   a. an R1 domain, when present, with least 60% sequence identity to a wild-type S1, for example, at least 70%, 80%, 85%, 90%, 92%, 95%, 96%, 97%, 98%, 99%, or 100% sequence identity to a wild-type R1 domain;
   b. an R2 domain, when present, with least 60% sequence identity to a wild-type S1, for example, at least 70%, 80%, 85%, 90%, 92%, 95%, 96%, 97%, 98%, 99%, or 100% sequence identity to a wild-type R2 domain;
   c. an R3 domain with least 60% sequence identity to a wild-type S1, for example, at least 70%, 80%, 85%, 90%, 92%, 95%, 96%, 97%, 98%, 99%, or 100% sequence identity to a wild-type R3 domain; and/or
   d. an R4 domain, when present, with least 40% sequence identity to a wild-type S1, for example, at least 50%, 60%, 70%, 80%, 85%, 90%, 92%, 95%, 96%, 97%, 98%, 99%, or 100% sequence identity to a wild-type R4 domain.
28. The genetically modified Gram-negative bacterial cell of any one of embodiments 1 to 27, wherein the genetically Gram-negative modified bacterial cell comprises a modified 30S ribosomal protein S1, wherein the modified 30S ribosomal protein(s) S1 comprises or consists of an amino acid sequence having:
   a. an R1 domain, when present, with least 60% sequence identity to a wild-type S1, for example, at least 98%, 99%, or 100% sequence identity to a wild-type R1 domain;
   b. an R2 domain, when present, with least 60% sequence identity to a wild-type S1, for example, at least 98%, 99%, or 100% sequence identity to a wild-type R2 domain;
   c. an R3 domain with least 60% sequence identity to a wild-type S1, for example, at least 98%, 99%, or 100% sequence identity to a wild-type R3 domain; and/or
   d. an R4 domain, when present, with least 40% sequence identity to a wild-type S1, for example, at least 98%, 99%, or 100% sequence identity to a wild-type R4 domain.
29. The genetically modified Gram-negative bacterial cell of any one of embodiments 1 to 28, wherein the genetically Gram-negative modified bacterial cell comprises a modified 30S ribosomal protein S1, wherein the modified 30S ribosomal protein(s) S1 comprises an amino acid sequence upstream of the first and/or the last amino acid of the R1, R2, R3 and/or R4 domain, when present, having at least 60%, 70%, 80%, 85%, 90%, 92%, 95%, 96%, 97%, 98%, 99%, or 100% sequence identity to the amino acid sequence upstream of the first and/or the last residue of the R1, R2, R3 and/or R4 domain of a wild-type 30S ribosomal protein S1.
30. The genetically modified Gram-negative bacterial cell of any one of embodiments 1 to 29, wherein the genetically Gram-negative modified bacterial cell comprises a modified 30S ribosomal protein S1, wherein the modified 30S ribosomal protein(s) S1 comprises an amino acid sequence upstream of the first and/or the last amino acid of the R1, R2, R3 and/or R4 domain, when present, having at least 95%, 98%, 99%, or 100% sequence identity to the amino acid sequence upstream of the first and/or the last residue of the R1, R2, R3 and/or R4 domain of a wild-type 30S ribosomal protein S1.
31. The genetically modified Gram-negative bacterial cell of any one of embodiments 1 to 30, wherein the genetically modified Gram-negative bacterial cell comprises a modified 30S ribosomal protein S1, wherein the modified 30S ribosomal protein(s) S1 was modified using, or is obtained or obtainable by genome editing, gene silencing, fragmenting the RNA post-transcriptionally and/or post-translational modification.
32. The genetically modified Gram-negative bacterial cell of any one of the preceding embodiments, wherein the Gram-negative bacterial cell comprises a wild-type rpsA gene.
33. The genetically modified Gram-negative bacterial cell of any one of the preceding embodiments, wherein the Gram-negative bacterial cell comprises a wild-type 30S ribosomal protein S1 and a modified 30S ribosomal protein S1 protein.
34. The genetically modified Gram-negative bacterial cell of any one of embodiments 1 to 31, wherein the genetically modified Gram-negative bacterial cell does not comprise a wild-type rpsA gene.
35. The genetically modified Gram-negative bacterial cell of any one of embodiments 1 to 31 and 34, wherein the genetically modified Gram-negative bacterial cell does not comprise a wild-type 30S ribosomal protein S1.
36. The genetically modified Gram-negative bacterial cell of any one of embodiments 1 to 35, wherein the genetically modified Gram-negative bacterial cell, compared to an unmodified bacterial cell, is capable of releasing at least 2.0-fold more nOMVs when grown in liquid culture, for example, at least 2.5-fold more, 3.0-fold more, 3.5-fold more, 4.0-fold more, 4.5-fold more, 5.0-fold more, 5.5-fold more, 6.0-fold more, 10-fold more, 20-fold more, 30-fold more, 40-fold more, 50-fold more, 60-fold more, 70-fold more, 80-fold more, 90-fold more, or 100-fold more nOMVs when growing in liquid culture.
37. The genetically modified Gram-negative bacterial cell of any one of embodiments 1 to 36, wherein the genetically modified Gram-negative bacterial cell, compared to an unmodified bacterial cell, is capable of releasing at least 2.0-fold more nOMVs when grown in liquid culture, for example, at least 2.5-fold more, 3.0-fold more, 3.5-fold more, 4.0-fold more, 4.5-fold more, 5.0-fold more, 5.5-fold more, 6.0-fold more, 10-fold more, 20-fold more, 30-fold more, 40-fold more, 50-fold more, 60-fold more, 70-fold more, 80-fold more, 90-fold more, or 100-fold more nOMVs when growing in liquid culture.
38. The genetically modified Gram-negative bacterial cell of any one of embodiments 1 to 37, wherein the genetically modified Gram-negative bacterial cell, compared to an unmodified bacterial cell, is capable of releasing at least 1.2-fold, at least 1.4-fold, at least 1.6-fold, at least 1.8-fold or at least 2.0-fold more nOMVs when growing in liquid culture.
39. The genetically modified Gram-negative bacterial cell of any one of embodiments 1 to 38, wherein the genetically modified Gram-negative bacterial cell, compared to an unmodified bacterial cell, is capable of releasing at least 1.2-fold, at least 1.4-fold, at least 1.6-fold, at least 1.8-fold or at least 2.0-fold more nOMVs when growing in liquid culture.
40. The genetically modified Gram-negative bacterial cell of any one of the preceding embodiments, characterised in that it is capable of proliferation.
41. The genetically modified Gram-negative bacterial cell of embodiment 40, wherein a culture of the genetically modified Gram-negative bacterial cell is capable of generating a biomass which is at least 10% of the biomass of a culture of the unmodified bacterial cell grown in the same culture conditions, for example, at least 20%, 30%, 40%, 50%, 60%, 70%, 80%, 85%, 90%, 92%, 95%, 96%, 97%, 98%, 99% or 100% of the biomass of a culture of the unmodified bacterial cell grown in the same culture conditions.
42. The genetically modified Gram-negative bacterial cell of any one of embodiments 40 to 41, wherein a culture of the genetically modified Gram-negative bacterial cell is capable of achieving a turbidity which is at least 10% of the turbidity of a culture of the unmodified bacterial cell grown in the same culture conditions, for example, 20%, 30%, 40%, 50%, 60%, 70%, 80%, 85%, 90%, 92%, 95%, 96%, 97%, 98%, 99% or 100% of the turbidity of a culture of the unmodified bacterial cell grown in the same culture conditions.
43. The genetically modified Gram-negative bacterial cell of embodiment 42, wherein the turbidity is measured as OD at 600 nm.
44. The genetically modified Gram-negative bacterial cell of embodiment 40, wherein a culture of the genetically modified Gram-negative bacterial cell is capable of achieving a nutrient uptake which is at least 10% of the nutrient uptake of a culture of the unmodified bacterial cell grown in the same culture conditions, for example, 20%, 30%, 40%, 50%, 60%, 70%, 80%, 85%, 90%, 92%, 95%, 96%, 97%, 98%, 99% or 100% of the nutrient uptake of a culture of the unmodified bacterial cell grown in the same culture conditions.
45. The genetically modified Gram-negative bacterial cell of any one of embodiments 40 to 44, wherein the genetically modified Gram-negative bacterial cell is capable of reaching stationary phase no later than 120 hours after the unmodified bacterial cell grown in the same culture conditions, for example, no later than 1, 2, 3, 4, 5, 6, 7, 8, 9, 10, 12, 15, 20, 25, 30, 40, 50, 60, 70, 80, 90, 100, or 110 hours after the unmodified bacterial cell grown in the same culture conditions.
46. The genetically modified Gram-negative bacterial cell of any one of embodiments 40 to 45, wherein growth of the genetically modified Gram-negative bacterial cell is determined according to biomass, turbidity and/or nutrient uptake.
47. The genetically modified Gram-negative bacterial cell of any one of embodiments 5 to 46, wherein the one or more mutation(s) relative to a wild-type rpsA gene occur in a designated nucleotide sequence comprising or consisting of:
a. from 1 to 10 nucleotides upstream of the 3′-end of the R3 domain relative to the wild-type rpsA gene, for example, from 11 to 20, from 21 to 50, or from 51 and 100 nucleotides upstream of the 3′-end of the R3 domain relative to the wild-type rpsA gene;
b. from 1 to 100 nucleotides upstream of the 3′-end of the R3 domain relative to the wild-type rpsA gene, for example, from 10 to 50, or from 20 to 30 nucleotides upstream of the 3′-end of the R3 domain relative to the wild-type rpsA gene;
c. from 1% to 10% of the nucleotides relative to the wild-type rpsA gene, for example, from 11% to 20%, from 21% to 30% relative the nucleotides of the wild-type rpsA gene, wherein the designated region is located between the 5′-end relative to the wild-type rpsA gene and the 3′-end of the R3 domain relative to the wild-type RpsA gene;
d. from 1% to 30% of the nucleotides relative to the wild-type rpsA gene, for example, from 5% to 25%, from 10% to 20% of the nucleotides relative to the wild-type rpsA gene, wherein the designated region is located between the 5′-end relative the wild-type RpsA gene and the 3′-end of the R3 domain relative to the wild-type rpsA gene;
e. from 1 and 10 nucleotides downstream of the 3′-end of the R3 domain relative to the wild-type rpsA gene, for example, from 11 and 20, from 21 to 50, or from 51 to 100 nucleotides downstream of the 3′-end of the R3 domain relative to wild-type rpsA gene;
f. from 1 to 100 nucleotides downstream of the 3′-end of the R3 domain relative to the wild-type rpsA gene, for example, from 10 to 50, or from 20 to 30, nucleotides downstream of the 3′-end of the R3 domain relative to the wild-type rpsA gene;
g. from 1 and 10 nucleotides downstream of the 5′-end of the R4 domain relative to the wild-type rpsA gene, for example, from 11 and 20, from 21 to 50, or from 51 to 100 nucleotides downstream of the 5′-end of the R4 domain relative to wild-type rpsA gene;
h. from 1 to 100 nucleotides downstream of the 5′-end of the R4 domain relative to the wild-type rpsA gene, for example, from 10 to 50, or from 20 to 30, nucleotides downstream of the 5′-end of the R4 domain relative to the wild-type rpsA gene;
i. from 1 and 10 nucleotides downstream of the 3′-end of the R4 domain relative to the wild-type rpsA gene, for example, from 11 and 20, from 21 to 50, or from 51 to 100 nucleotides downstream of the 3′-end of the R4 domain relative to wild-type rpsA gene;
j. from 1 to 100 nucleotides downstream of the 3′-end of the R4 domain relative to the wild-type rpsA gene, for example, from 10 to 50, or from 20 to 30, nucleotides downstream of the 3′-end of the R4 domain relative to the wild-type rpsA gene;
k. from 1% to 10% of the nucleotides relative to the wild-type rpsA gene, for example, from 11% to 20%, from 21% to 30% of the nucleotides relative to the wild-type rpsA gene, wherein the designated region is located between the 3′-end relative to the R3 domain of the wild-type rpsA gene and the 3′-end relative to the wild-type rpsA gene;
l. from 1% to 30%, of the nucleotides relative to the wild-type rpsA gene, for example, from 5% to 25%, from 10% to 20% of the nucleotides relative to the wild-type rpsA gene, wherein the designated region is located between the 3′-end relative to the R3 domain of the wild-type rpsA gene and the 3′-end relative to the wild-type rpsA gene;
m. from 1 to 5 nucleotides upstream of the 3′-end relative to the wild-type rpsA gene, for example, from 6 to 10, from 11 to 20, from 21 to 30, from 31 to 40, from 41 to 50, from 51 to 100, from 101 to 150, from 151 to 200, from 201 to 250, from 251 to 300, from 301 to 350, from 351 to 400, from 401 to 450, from 451 to 500 nucleotides upstream of the 3′-end relative to the wild-type rpsA gene; and/or
n. from 1 to 500 nucleotides upstream the 3′-end relative to the wild-type rpsA gene, for example, from 50 to 450, from 100 to 400, from 150 to 380, from 200 to 378, or from 250 to 376 nucleotides upstream the 3′-end relative to the wild-type rpsA gene.
48. The genetically modified Gram-negative bacterial cell of any one of embodiments 5 to 47, wherein the one or more mutation(s) relative to a wild-type rpsA gene comprises one or more mutation(s) in a region corresponding to a region encoding the R4 domain of the B. pertussis 30S ribosomal protein S1 protein and/or a region encoding the portion of the B. pertussis 30S ribosomal S1 protein between the R3 and R4 domains.
49. The genetically modified Gram-negative bacterial cell of any one of embodiments 5 to 48, wherein the one or more mutation(s) relative to a wild-type rpsA gene comprises one or more mutation(s) that causes an increased release of nOMVs compared to an unmodified Gram-negative bacterial cell.
50. The genetically modified Gram-negative bacterial cell of embodiment 49, wherein the one or more mutation(s) causes an at least 1.2-fold, 1.4-fold, 1.6-fold, 1.8-fold, 2.0-fold or 2.5-fold increased release of nOMVs compared to an unmodified Gram-negative bacterial cell.
51. The genetically modified Gram-negative bacterial cell of embodiment 50, wherein the one or more mutation(s) causes an at least 2.0-fold increase in release of nOMVs compared to an unmodified Gram-negative bacterial cell.
52. The genetically modified Gram-negative bacterial cell of any one of embodiments 5 to 51, wherein the one or more mutation(s) comprises mutation(s) that change the amino acid sequence of the encoded 30S ribosomal protein S1 protein.
53. The genetically modified Gram-negative bacterial cell of embodiment 52, wherein the one or more mutation(s) comprises mutations that change the encoded amino acid sequence of a region of the 30S ribosomal protein S1 protein that corresponds to the R4 domain of B. pertussis.
54. The genetically modified Gram-negative bacterial cell of any one of embodiments 47 to 53, wherein the one or more mutation(s) comprises mutations that change the encoded 30S ribosomal protein S1 protein to modify and/or delete at least 5, at least 10, at least 25, at least 50, at least 75, at least 100, at least 110, at least 125, between 5 and 150, between 25 and 130, between 100 and 130, or between 125 and 130 amino acids.
55. The genetically modified Gram-negative bacterial cell of any one of embodiments 47 to 54, wherein the one or more mutation(s) comprises mutations that change the encoded 30S ribosomal protein S1 protein to delete at least 5, at least 10, at least 25, at least 50, at least 75, at least 100, at least 110, at least 125, between 5 and 150, between 25 and 130, between 100 and 130, or between 125 and 130 amino acids.
56. The genetically modified Gram-negative bacterial cell of any one of embodiments 47 to 55, wherein the modified rpsA gene encodes a modified 30S ribosomal protein S1 protein according to any one of embodiments 13 to 36.
57. The genetically modified Gram-negative bacterial cell of any one of embodiments 47 to 56, wherein the one or more mutation(s) comprises mutation or deletion of at least 15, at least 30, at least 45, at least 60, or at least 75 nucleotides of the region corresponding to nucleotides 1655 to 1731 of the B. pertussis rpsA gene.
58. The genetically modified Gram-negative bacterial cell of any one of embodiments 47 to 57, wherein the one or more mutation(s) comprises mutation or deletion of at least 60, at least 90, at least 150, at least 225, or at least 300 nucleotides of the region corresponding to nucleotides 1424 to 1731 of the B. pertussis rpsA gene.
59. The genetically modified Gram-negative bacterial cell of any one of embodiments 47 to 58, wherein the one or more mutation(s) comprises mutation or deletion of at least 60, at least 90, at least 150, at least 225, at least 300, or at least 360 nucleotides of the region corresponding to nucleotides 1355 to 1731 of the B. pertussis rpsA gene.
60. The genetically modified Gram-negative bacterial cell of any one of the preceding embodiments, wherein the genetically modified Gram-negative bacterial cell comprises a modified ihfB gene and/or a modified IHF protein.
61. A genetically modified Gram-negative bacterial cell comprising a modified ihfB gene and/or a modified IHF protein.
62. The genetically modified Gram-negative bacterial cell of embodiment 60 or 61, wherein:
a. the modified ihfB gene(s) comprises one or more mutation(s) relative to a wild-type ihfB gene, wherein the one or more mutation(s) are located within the coding region of the ihfB gene and/or within the non-coding region of the ihfB gene;
b. the modified ihfB gene(s) is knocked-out relative to a wild-type ihfB gene;
c. the modified IHF protein(s) comprises one or more mutation(s) relative to a wild-type IHF protein;
d. The modified IHF protein(s) comprises one or more post-translational modification(s) relative to a wild-type IHF protein;
e. the modified IHF protein(s) is downregulated relative to the IHF protein of an unmodified bacterial cell; and/or
f. the modified IHF protein(s) is encoded by the modified ihfB gene(s).
63. The genetically modified Gram-negative bacterial cell of embodiment 62, wherein the one or more mutation(s) relative to a wild-type IHF protein comprises a deletion of at least 20, at least 30, at least 40, at least 50, at least 60, at least 70, at least 80, at least 90, at least 100, at least 110, between 50 and 119, or between 80 and 119 amino acids.
64. The genetically modified Gram-negative bacterial cell of embodiment 63, wherein the one or more mutation(s) relative to a wild-type IHF protein comprises a deletion of at least 20, at least 30, at least 40, at least 50, at least 60, at least 70, at least 80, at least 100, at least 110, between 50 and 119, or between 80 and 119 contiguous amino acids.
65. The genetically modified Gram-negative bacterial cell of any one of embodiments 62 to 64, wherein the one or more mutation(s) relative to a wild-type IHF protein causes an increased release of nOMVs compared to an unmodified Gram-negative bacterial cell.
66. The genetically modified Gram-negative bacterial cell of any one of embodiments 62 to 65, wherein the one or more mutation(s) relative to a wild-type IHF protein causes an at least 1.2-fold, at least 1.4-fold, at least 1.6-fold, at least 1.8-fold, at least 2.0-fold, or at least 2.5-fold increased release of nOMVs compared to an unmodified Gram-negative bacterial cell.
67. The genetically modified Gram-negative bacterial cell of any one of embodiments 62 to 66, wherein the modified ihfB gene encodes the modified IHF protein according to any one of embodiments 62 to 66.
68. The genetically modified Gram-negative bacterial cell of any one of the preceding embodiments, wherein the Gram-negative bacterial cell is a species of bacteria which naturally co-transcribes an ihfB gene and an rpsA gene.
69. The genetically modified Gram-negative bacterial cell of any one of embodiments 62 to 68, wherein the modified IHF protein(s) comprises or consists of an amino acid sequence having at least 60% sequence identity to a wild-type IHF protein, for example, at least 70%, 80%, 85%, 90%, 92%, 95%, 96%, 97%, 98%, 99%, or 100% sequence identity to a wild-type IHF protein.
70. The genetically modified Gram-negative bacterial cell of any one of the preceding embodiments, wherein the wild-type rpsA gene is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 55, and SEQ ID NO: 60.
71. The genetically modified Gram-negative bacterial cell of any one of the preceding embodiments, wherein the wild-type S1 is selected from the group consisting of: SEQ ID NO: 13, SEQ ID NO: 14, SEQ ID NO: 15, SEQ ID NO: 16, SEQ ID NO: 17, SEQ ID NO: 18, and SEQ ID NO: 57.
72. The genetically modified Gram-negative bacterial cell of any one of the preceding embodiments, wherein the wild-type IHF protein is selected from the group consisting of: SEQ ID NO: 31, SEQ ID NO: 32, SEQ ID NO: 33, SEQ ID NO: 34, SEQ ID NO: 35, SEQ ID NO: 36, and SEQ ID NO: 62.
73. The genetically modified Gram-negative bacterial cell of any one of the preceding embodiments which is selected from the group consisting of Enterobacteriaceae, Neisseriaceae, Helicobacteraceae, Campylobacteraceae, Yersiniaceae, Vibrionaceae, Pasteurellaceae, Alcaligenaceae, Pseudomonadaceae and Moraxellaceae.
74. The genetically modified Gram-negative bacterial cell of any one of the preceding embodiments which is selected from the group consisting of Escherichia coli, Neisseria meningitidis, Neisseria lactamica, Neisseria gonorrhoeae, Helicobacter pylori, Salmonella typhi, Salmonella typhimurium, Vibrio cholerae, Shigella spp., Haemophilus influenzae, Bordetella pertussis, Pseudomonas aeruginosa and Moraxella catarrhalis.
75. The genetically modified Gram-negative bacterial cell of any one of the preceding embodiments which is selected from the group consisting of Escherichia coli, Bordetella pertussis, and Moraxella catarrhalis.
76. The genetically modified Gram-negative bacterial cell of embodiment 74 or 75, wherein the Gram-negative bacterial cell is Bordetella pertussis.
77. The genetically modified Gram-negative bacterial cell of any one of the preceding embodiments, wherein the genetically modified Gram-negative bacterial cell comprises a modified rpsA gene, wherein the modified rpsA gene comprises or consists of a nucleotide sequences having at least 60% sequence identity to mutants SEQ ID NO: 37 or SEQ ID NO: 56, for example, at least 70%, 80%, 85%, 90%, 92%, 95%, 96%, 97%, 98%, 99%, or 100% sequence identity to SEQ ID NO: 37 or SEQ ID NO: 56.
78. The genetically modified Gram-negative bacterial cell of any one of the preceding embodiments, wherein the genetically modified Gram-negative bacterial cell comprises a modified rpsA gene, wherein the modified rpsA gene comprises or consists of a nucleotide sequences having at least 95% sequence identity to mutants SEQ ID NO: 37 or SEQ ID NO: 56.
79. The genetically modified Gram-negative bacterial cell of any one of the preceding embodiments, wherein the genetically modified Gram-negative bacterial cell comprises a modified rpsA operon, wherein the modified rpsA operon comprises or consists of a nucleotide sequences having at least 60% sequence identity to SEQ ID NO: 38, SEQ ID NO: 51 or SEQ ID NO: 59, for example, at least 70%, 80%, 85%, 90%, 92%, 95%, 96%, 97%, 98%, 99%, or 100% sequence identity to SEQ ID NO: 38, SEQ ID NO: 51 or SEQ ID NO: 59.
80. The genetically modified Gram-negative bacterial cell of any one of the preceding embodiments, wherein the genetically modified Gram-negative bacterial cell comprises a modified rpsA operon, wherein the modified rpsA operon comprises or consists of a nucleotide sequences having at least 95% sequence identity to SEQ ID NO: 38, SEQ ID NO: 51 or SEQ ID NO: 59.
81. The genetically modified Gram-negative bacterial cell of any one of the preceding embodiments, wherein the genetically modified Gram-negative bacterial cell comprises a modified 30S ribosomal protein S1, wherein the modified 30S ribosomal protein S1 comprises or consists of an amino acid sequence having at least 60% sequence identity to a polypeptide selected from the group consisting of SEQ ID NO: 39, SEQ ID NO: 40, SEQ ID NO: 41, and SEQ ID NO: 53, for example, at least 70%, 80%, 85%, 90%, 92%, 95%, 96%, 97%, 98%, 99%, or 100% sequence identity to a polypeptide selected from the group consisting of SEQ ID NO: 39, SEQ ID NO: 40, SEQ ID NO: 41, and SEQ ID NO: 53.
82. The genetically modified Gram-negative bacterial cell of any one of the preceding embodiments, wherein the genetically modified Gram-negative bacterial cell comprises a modified 30S ribosomal protein S1, wherein the modified 30S ribosomal protein S1 comprises or consists of an amino acid sequence having at least 98% sequence identity to a polypeptide selected from the group consisting of SEQ ID NO: 39, SEQ ID NO: 40, SEQ ID NO: 41, and SEQ ID NO: 53.
83. The genetically modified Gram-negative bacterial cell of embodiment 81 or 82, wherein the bacterial cell is E. coli or B. pertussis.
84. The genetically modified Gram-negative bacterial cell of any one of the preceding embodiments, wherein the genetically modified Gram-negative bacterial cell comprises a modified rpsA operon, wherein the modified rpsA operon comprises or consists of a nucleotide sequences having at least 60% sequence identity to SEQ ID NO: 42 or SEQ ID NO: 43, for example, at least 70%, 80%, 85%, 90%, 92%, 95%, 96%, 97%, 98%, 99%, or 100% sequence identity to SEQ ID NO: 42 or SEQ ID NO: 43.
85. The genetically modified Gram-negative bacterial cell of any one of the preceding embodiments, wherein the genetically modified Gram-negative bacterial cell comprises a modified rpsA operon, wherein the modified rpsA operon comprises or consists of a nucleotide sequences having at least 98% sequence identity to SEQ ID NO: 42 or SEQ ID NO: 43.
86. The genetically modified Gram-negative bacterial cell of any one of the preceding embodiments, wherein the genetically modified Gram-negative bacterial cell comprises a modified 30S ribosomal protein S1, wherein the modified 30S ribosomal protein S1 comprises or consists of an amino acid sequence having at least 60% sequence identity to SEQ ID NO: 44 or SEQ ID NO: 45, for example, at least 70%, 80%, 85%, 90%, 92%, 95%, 96%, 97%, 98%, 99%, or 100% sequence identity to SEQ ID NO: 44 or SEQ ID NO: 45.
87. The genetically modified Gram-negative bacterial cell of any one of the preceding embodiments, wherein the genetically modified Gram-negative bacterial cell comprises a modified 30S ribosomal protein S1, wherein the modified 30S ribosomal protein S1 comprises or consists of an amino acid sequence having at least 98% sequence identity to SEQ ID NO: 44 or SEQ ID NO: 45.
88. The genetically modified Gram-negative bacterial cell of any one of the preceding embodiments, wherein the genetically modified Gram-negative bacterial cell expresses one or more exogenous antigens.
89. The genetically modified Gram-negative bacterial cell of any one of the preceding embodiments which has been further genetically engineered by one or more processes selected from the following group: (a) a process of down-regulating expression of immunodominant variable or non-protective antigens, (b) a process of up-regulating expression of protective OMP antigens, (c) a process of down-regulating a gene involved in rendering the lipid A portion of LPS toxic, (d) a process of up-regulating a gene involved in rendering the lipid A portion of LPS less toxic, and (e) a process of genetically modifying the bacterial cell to express a heterologous antigen.
90. A method of generating a genetically modified Gram-negative bacterial cell, comprising a step of modifying a wild-type rpsA gene, operon, RNA and/or 30S ribosomal protein S1 protein, such that the modification causes the genetically modified Gram-negative bacterial cell, when grown in culture medium, to release greater quantities of nOMVs into the medium than the unmodified bacterial cell.
91. A method of generating a genetically modified Gram-negative bacterial cell, comprising a step of providing a modified 30S ribosomal protein S1, such that the modification causes the genetically modified Gram-negative bacterial cell, when grown in culture medium, to release greater quantities of nOMVs into the medium than the unmodified bacterial cell.
92. The method of embodiment 90 or 91, wherein the genetically modified Gram-negative cell that is generated is a Gram-negative bacterial cell according to any one of embodiments 1 to 98.
93. A process for preparing nOMVs, comprising the steps of:
a. inoculating a culture vessel containing a nutrient medium suitable for growth of the genetically modified Gram-negative bacterial cell of any one of embodiments 1 to 89;
b. culturing the genetically modified Gram-negative bacterial cell under conditions which permit the release of nOMVs into the medium by the bacteria; and
c. recovering nOMVs from the medium; and
d. mixing the nOMVs with a pharmaceutically acceptable diluent or carrier.
94. A process for preparing nOMVs, comprising the steps of:
a. inoculating a culture vessel containing a nutrient medium suitable for growth of a genetically modified Gram-negative bacterial cell;
b. culturing the genetically modified Gram-negative bacterial cell under conditions which permit the release of nOMVs into the medium by the bacteria, wherein the conditions comprise addition of the modified 30S ribosomal protein S1 according to any one of embodiments 11 to 31;
c. recovering nOMVs from the medium; and
d. mixing the nOMVs with a pharmaceutically acceptable diluent or carrier.
95. The process of embodiment 94, wherein the Gram-negative bacterial cell is a Gram-negative bacterial cell of any one of embodiments 1 to 89.
96. The process of any one of embodiments 93 to 95 which further comprises a step after step (c) comprising sterile-filtering the preparation of nOMVs.
97. An nOMV obtained or obtainable from the genetically modified Gram-negative bacterial cell of any one of embodiments 1 to 89, or from a genetically modified Gram-negative bacterial cell obtained or obtainable by the method of any one of embodiments 90 to 92, or by the process of any one of embodiments 93 to 96.
98. An immunogenic composition comprising the nOMV of embodiment 97.
99. The immunogenic composition of embodiment 98, further comprising one or more additional antigens from the same or different pathogen.
100. A vaccine comprising the nOMV of embodiment 97.
101. The nOMV of embodiment 97, the immunogenic composition of any one of embodiments 98 and 99 or the vaccine of embodiment 100 for use in medicine.
102. The nOMV of embodiment 97, the immunogenic composition of any one of embodiments 98 and 99 or the vaccine of embodiment 100 for use in inducing an immune response in a vertebrate, preferably a mammal.
103. The nOMV of embodiment 97, the immunogenic composition of any one of embodiments 98 and 99 or the vaccine of embodiment 100 for use in the treatment or prevention of a disease which is caused by a bacterial cell of the same genus or species of the genetically modified Gram-negative bacterial cell from which the nOMV was obtained or obtainable.
104. The nOMV of embodiment 97, the immunogenic composition of any one of embodiments 98 and 99 or the vaccine of embodiment 100 for use in the treatment or prevention of a disease which is caused by Bordetella, preferably by B. pertussis.
105. The nOMV of embodiment 97, the immunogenic composition of any one of embodiments 98 and 99 or the vaccine of embodiment 100 for use in the treatment or prevention of a disease which is caused by Escherichia coli.
106. The nOMV of embodiment 97, the immunogenic composition of any one of embodiments 98 and 99 or the vaccine of embodiment 100 for use in the treatment or prevention of a disease which is caused by Moraxella catarrhalis.
107. A method of immunising a subject in need thereof against a bacterial cell by administering the nOMV of embodiment 97, the immunogenic composition of any one of embodiments 98 and 99 or the vaccine of embodiment 100.
108. The use of the nOMV of embodiment 97, the immunogenic composition of any one of embodiments 98 and 99 or the vaccine of embodiment 100 in the manufacture of a medicament for immunising a subject in need thereof against a bacterial cell.

1. A genetically modified Gram-negative bacterial cell comprising a modified rpsA gene, a modified rpsA operon and/or a modified 30S ribosomal protein S1 protein.

2. The genetically modified Gram-negative bacterial cell of claim 1, wherein the genetically modified Gram-negative bacterial cell is capable of secreting greater quantities of nOMV compared to an unmodified bacterial cell.

3. The genetically modified Gram-negative bacterial cell of claim 1 or 2, wherein the genetically modified Gram-negative bacterial cell comprises a modified 30S ribosomal protein S1 comprising one or more mutation(s) relative to a wild-type 30S ribosomal protein S1, wherein:
(i) the one or more mutation(s) relative to a wild-type 30S ribosomal protein S1 causes an increased release of nOMVs compared to an unmodified Gram-negative bacterial cell;
(ii) the one or more mutation(s) relative to a wild-type 30S ribosomal protein S1 causes an at least 1.2-fold, at least 1.4-fold, at least 1.6-fold, at least 1.8-fold, at least 2.0-fold, or at least 2.5-fold increased release of nOMVs compared to an unmodified Gram-negative bacterial cell;
(iii) the one or more mutation(s) relative to a wild-type 30S ribosomal protein S1 causes an at least 2.0-fold increased release of nOMVs compared to an unmodified Gram-negative bacterial cell;
(iv) the one or more mutation(s) relative to a wild-type 30S ribosomal protein S1 comprises mutation or deletion of at least 5, at least 10, at least 25, at least 50, at least 75, at least 100, at least 110, at least 125, between 5 and 150, between 25 and 130, between 100 and 130, or between 125 and 130 amino acids;
(v) the one or more mutation(s) relative to a wild-type 30S ribosomal protein S1 comprises mutation or deletion of at least 5, at least 10, at least 15, at least 20, or at least 25, amino acids of the region corresponding to amino acids 550 to 576 of the B. pertussis 30S ribosomal protein S1;
(vi) the one or more mutation(s) relative to a wild-type 30S ribosomal protein S1 comprises mutation or deletion of at least 20, at least 30, at least 50, at least 75, or at least 100 amino acids of the region corresponding to amino acids 473 to 576 of the B. pertussis 30S ribosomal protein S1;
(vii) the one or more mutation(s) relative to a wild-type 30S ribosomal protein S1 comprises mutation or deletion of at least 20, at least 30, at least 50, at least 100, or at least 120 amino acids of the region corresponding to amino acids 450 to 576 of the B. pertussis 30S ribosomal protein S1;
(viii) the one or more mutation(s) relative to a wild-type 30S ribosomal protein S1 comprises truncation of at least 20, at least 30, at least 50, at least 100, or at least 120 consecutive amino acids from the C-terminal end of the 30S ribosomal protein S1;
(ix) the one or more mutation(s) relative to a wild-type 30S ribosomal protein S1 comprises mutation or deletion of amino acids corresponding to amino acids 560 to 576, 552 to 576, 500 to 576 485 to 576, 460 to 576, or 453 to 576 of the B. pertussis 30S ribosomal protein S1; and/or
(x) the genetically modified Gram-negative bacterial cell comprises a modified 30S ribosomal protein S1, wherein the modified 30S ribosomal protein(s) S1 is truncated relative to a wild-type 30S ribosomal protein S1.

4. The genetically modified Gram-negative bacterial cell of any one of claims 1 to 3, wherein:
(i) the genetically modified Gram-negative bacterial cell, compared to an unmodified bacterial cell, is capable of releasing at least 2.0-fold more nOMVs when grown in liquid culture, for example, at least 2.5-fold more, 3.0-fold more, 3.5-fold more, 4.0-fold more, 4.5-fold more, 5.0-fold more, 5.5-fold more, 6.0-fold more, 10-fold more, 20-fold more, 30-fold more, 40-fold more, 50-fold more, 60-fold more, 70-fold more, 80-fold more, 90-fold more, or 100-fold more nOMVs when growing in liquid culture; and/or
(ii) the genetically modified Gram-negative bacterial cell, compared to an unmodified bacterial cell, is capable of releasing at least 1.2-fold, at least 1.4-fold, at least 1.6-fold, at least 1.8-fold or at least 2.0-fold more nOMVs when growing in liquid culture.

5. The genetically modified Gram-negative bacterial cell of any one of the preceding claims, wherein:
(i) a culture of the genetically modified Gram-negative bacterial cell is capable of generating a biomass which is at least 10% of the biomass of a culture of the unmodified bacterial cell grown in the same culture conditions, for example, at least 20%, 30%, 40%, 50%, 60%, 70%, 80%, 85%, 90%, 92%, 95%, 96%, 97%, 98%, 99% or 100% of the biomass of a culture of the unmodified bacterial cell grown in the same culture conditions;
(ii) a culture of the genetically modified Gram-negative bacterial cell is capable of achieving a turbidity which is at least 10% of the turbidity of a culture of the unmodified bacterial cell grown in the same culture conditions, for example, 20%, 30%, 40%, 50%, 60%, 70%, 80%, 85%, 90%, 92%, 95%, 96%, 97%, 98%, 99% or 100% of the turbidity of a culture of the unmodified bacterial cell grown in the same culture conditions;
(iii) a culture of the genetically modified Gram-negative bacterial cell is capable of achieving a nutrient uptake which is at least 10% of the nutrient uptake of a culture of the unmodified bacterial cell grown in the same culture conditions, for example, 20%, 30%, 40%, 50%, 60%, 70%, 80%, 85%, 90%, 92%, 95%, 96%, 97%, 98%, 99% or 100% of the nutrient uptake of a culture of the unmodified bacterial cell grown in the same culture conditions; and/or
(iv) the genetically modified Gram-negative bacterial cell is capable of reaching stationary phase no later than 120 hours after the unmodified bacterial cell grown in the same culture conditions, for example, no later than 1, 2, 3, 4, 5, 6, 7, 8, 9, 10, 12, 15, 20, 25, 30, 40, 50, 60, 70, 80, 90, 100, or 110 hours after the unmodified bacterial cell grown in the same culture conditions.

6. The genetically modified Gram-negative bacterial cell of any one of claims 1 to 5, wherein the genetically modified Gram-negative bacterial cell comprises a modified rpsA gene, wherein the modified rpsA gene(s) comprises one or more mutation(s) relative to a wild-type rpsA gene and the one or more mutation(s) relative to a wild-type rpsA gene occur in a designated nucleotide sequence comprising or consisting of:
a. from 1 to 10 nucleotides upstream of the 3′-end of the R3 domain relative to the wild-type rpsA gene, for example, from 11 to 20, from 21 to 50, or from 51 and 100 nucleotides upstream of the 3′-end of the R3 domain relative to the wild-type rpsA gene;
b. from 1 to 100 nucleotides upstream of the 3′-end of the R3 domain relative to the wild-type rpsA gene, for example, from 10 to 50, or from 20 to 30 nucleotides upstream of the 3′-end of the R3 domain relative to the wild-type rpsA gene;
c. from 1% to 10% of the nucleotides relative to the wild-type rpsA gene, for example, from 11% to 20%, from 21% to 30% relative the nucleotides of the wild-type rpsA gene, wherein the designated region is located between the 5′-end relative to the wild-type rpsA gene and the 3′-end of the R3 domain relative to the wild-type RpsA gene;
d. from 1% to 30% of the nucleotides relative to the wild-type rpsA gene, for example, from 5% to 25%, from 10% to 20% of the nucleotides relative to the wild-type rpsA gene, wherein the designated region is located between the 5′-end relative the wild-type RpsA gene and the 3′-end of the R3 domain relative to the wild-type rpsA gene;
e. from 1 and 10 nucleotides downstream of the 3′-end of the R3 domain relative to the wild-type rpsA gene, for example, from 11 and 20, from 21 to 50, or from 51 to 100 nucleotides downstream of the 3′-end of the R3 domain relative to wild-type rpsA gene;
f. from 1 to 100 nucleotides downstream of the 3′-end of the R3 domain relative to the wild-type rpsA gene, for example, from 10 to 50, or from 20 to 30, nucleotides downstream of the 3′-end of the R3 domain relative to the wild-type rpsA gene;
g. from 1 and 10 nucleotides downstream of the 5′-end of the R4 domain relative to the wild-type rpsA gene, for example, from 11 and 20, from 21 to 50, or from 51 to 100 nucleotides downstream of the 5′-end of the R4 domain relative to wild-type rpsA gene;
h. from 1 to 100 nucleotides downstream of the 5′-end of the R4 domain relative to the wild-type rpsA gene, for example, from 10 to 50, or from 20 to 30, nucleotides downstream of the 5′-end of the R4 domain relative to the wild-type rpsA gene;
i. from 1 and 10 nucleotides downstream of the 3′-end of the R4 domain relative to the wild-type rpsA gene, for example, from 11 and 20, from 21 to 50, or from 51 to 100 nucleotides downstream of the 3′-end of the R4 domain relative to wild-type rpsA gene;
j. from 1 to 100 nucleotides downstream of the 3′-end of the R4 domain relative to the wild-type rpsA gene, for example, from 10 to 50, or from 20 to 30, nucleotides downstream of the 3′-end of the R4 domain relative to the wild-type rpsA gene;
k. from 1% to 10% of the nucleotides relative to the wild-type rpsA gene, for example, from 11% to 20%, from 21% to 30% of the nucleotides relative to the wild-type rpsA gene, wherein the designated region is located between the 3′-end relative to the R3 domain of the wild-type rpsA gene and the 3′-end relative to the wild-type rpsA gene;
l. from 1% to 30%, of the nucleotides relative to the wild-type rpsA gene, for example, from 5% to 25%, from 10% to 20% of the nucleotides relative to the wild-type rpsA gene, wherein the designated region is located between the 3′-end relative to the R3 domain of the wild-type rpsA gene and the 3′-end relative to the wild-type rpsA gene;
m. from 1 to 5 nucleotides upstream of the 3′-end relative to the wild-type rpsA gene, for example, from 6 to 10, from 11 to 20, from 21 to 30, from 31 to 40, from 41 to 50, from 51 to 100, from 101 to 150, from 151 to 200, from 201 to 250, from 251 to 300, from 301 to 350, from 351 to 400, from 401 to 450, from 451 to 500 nucleotides upstream of the 3′-end relative to the wild-type rpsA gene; and/or
n. from 1 to 500 nucleotides upstream the 3′-end relative to the wild-type rpsA gene, for example, from 50 to 450, from 100 to 400, from 150 to 380, from 200 to 378, or from 250 to 376 nucleotides upstream the 3′-end relative to the wild-type rpsA gene.

7. The genetically modified Gram-negative bacterial cell of any one of claims 1 to 6, wherein the genetically modified Gram-negative bacterial cell comprises a modified rpsA gene, wherein the modified rpsA gene(s) comprises one or more mutation(s) relative to a wild-type rpsA gene and wherein the one or more mutation(s) relative to a wild-type rpsA gene:
(i) comprises one or more mutation(s) in a region corresponding to a region encoding the R4 domain of the B. pertussis 30S ribosomal protein S1 protein and/or a region encoding the portion of the B. pertussis 30S ribosomal S1 protein between the R3 and R4 domains;
(ii) comprises one or more mutation(s) that causes an increased release of nOMVs compared to an unmodified Gram-negative bacterial cell;
(iii) causes an at least 1.2-fold, 1.4-fold, 1.6-fold, 1.8-fold, 2.0-fold or 2.5-fold increased release of nOMVs compared to an unmodified Gram-negative bacterial cell;
(iv) causes an at least 2.0-fold increase in release of nOMVs compared to an unmodified Gram-negative bacterial cell;
(v) comprises mutation(s) that change the amino acid sequence of the encoded 30S ribosomal protein S1 protein;
(vi) comprises mutations that change the encoded amino acid sequence of a region of the 30S ribosomal protein S1 protein that corresponds to the R4 domain of B. pertussis;
(vii) comprises mutations that change the encoded 30S ribosomal protein S1 protein to modify and/or delete at least 5, at least 10, at least 25, at least 50, at least 75, at least 100, at least 110, at least 125, between 5 and 150, between 25 and 130, between 100 and 130, or between 125 and 130 amino acids;
(viii) comprises mutations that change the encoded 30S ribosomal protein S1 protein to delete at least 5, at least 10, at least 25, at least 50, at least 75, at least 100, at least 110, at least 125, between 5 and 150, between 25 and 130, between 100 and 130, or between 125 and 130 amino acids;
(ix) comprises mutation or deletion of at least 15, at least 30, at least 45, at least 60, or at least 75 nucleotides of the region corresponding to nucleotides 1655 to 1731 of the B. pertussis rpsA gene;
(x) comprises mutation or deletion of at least 60, at least 90, at least 150, at least 225, or at least 300 nucleotides of the region corresponding to nucleotides 1424 to 1731 of the B. pertussis rpsA gene; and/or
(xi) comprises mutation or deletion of at least 60, at least 90, at least 150, at least 225, at least 300, or at least 360 nucleotides of the region corresponding to nucleotides 1355 to 1731 of the B. pertussis rpsA gene.

8. The genetically modified Gram-negative bacterial cell of any one of the preceding claims, wherein the genetically modified Gram-negative bacterial cell comprises a modified ihfB gene and/or a modified IHF protein.

9. A genetically modified Gram-negative bacterial cell comprising a modified ihfB gene and/or a modified IHF protein.

10. The genetically modified Gram-negative bacterial cell of claim 8 or 9, wherein:
a. the modified ihfB gene(s) comprises one or more mutation(s) relative to a wild-type IhfB gene, wherein the one or more mutation(s) are located within the coding region of the ihfB gene and/or within the non-coding region of the ihfB gene;
b. the modified ihfB gene(s) is knocked-out relative to a wild-type ihfB gene;
c. the modified IHF protein(s) comprises one or more mutation(s) relative to a wild-type IHF protein;
d. the modified IHF protein(s) comprises one or more post-translational modification(s) relative to a wild-type IHF protein;
e. the modified IHF protein(s) is downregulated relative to the IHF protein of an unmodified bacterial cell; and/or
f. the modified IHF protein(s) is encoded by the modified ihfB gene(s).

11. The genetically modified Gram-negative bacterial cell of claim 10, wherein:
(i) the one or more mutation(s) relative to a wild-type IHF protein comprises a deletion of at least 20, at least 30, at least 40, at least 50, at least 60, at least 70, at least 80, at least 90, at least 100, at least 110, between 50 and 119, or between 80 and 119 amino acids;
(ii) the one or more mutation(s) relative to a wild-type IHF protein comprises a deletion of at least 20, at least 30, at least 40, at least 50, at least 60, at least 70, at least 80, at least 100, at least 110, between 50 and 119, or between 80 and 119 contiguous amino acids;
(iii) the one or more mutation(s) relative to a wild-type IHF protein causes an increased release of nOMVs compared to an unmodified Gram-negative bacterial cell;
(iv) the one or more mutation(s) relative to a wild-type IHF protein causes an at least 1.2-fold, at least 1.4-fold, at least 1.6-fold, at least 1.8-fold, at least 2.0-fold, or at least 2.5-fold increased release of nOMVs compared to an unmodified Gram-negative bacterial cell; and/or
(v) the modified ihfB gene encodes the modified IHF protein according to (i) to (iv).

12. The genetically modified Gram-negative bacterial cell of any one of the preceding claims which is selected from the group consisting of Escherichia col, Neisseria meningitidis, Neisseria lactamica, Neisseria gonorrhoeae, Helicobacter pylori, Salmonella typhi, Salmonella typhimurium, Vibrio cholerae, Shigella spp., Haemophilus influenzae, Bordetella pertussis, Pseudomonas aeruginosa and Moraxella catarrhalis.

13. A method of generating a genetically modified Gram-negative bacterial cell, comprising:
(i) a step of modifying a wild-type rpsA gene, operon, RNA and/or 30S ribosomal protein S1 protein, such that the modification causes the genetically modified Gram-negative bacterial cell, when grown in culture medium, to release greater quantities of nOMVs into the medium than the unmodified bacterial cell; and/or
(ii) a step of providing a modified 30S ribosomal protein S1, such that the modification causes the genetically modified Gram-negative bacterial cell, when grown in culture medium, to release greater quantities of nOMVs into the medium than the unmodified bacterial cell.

14. A process for preparing nOMVs, comprising the steps of:
a. inoculating a culture vessel containing a nutrient medium suitable for growth of the genetically modified Gram-negative bacterial cell of any one of claims 1 to 13;
b culturing the genetically modified Gram-negative bacterial cell under conditions which permit the release of nOMVs into the medium by the bacteria; and
c. recovering nOMVs from the medium; and
d. mixing the nOMVs with a pharmaceutically acceptable diluent or carrier.

15. A process for preparing nOMVs, comprising the steps of:
a. inoculating a culture vessel containing a nutrient medium suitable for growth of a genetically modified Gram-negative bacterial cell;
b. culturing the genetically modified Gram-negative bacterial cell under conditions which permit the release of nOMVs into the medium by the bacteria, wherein the conditions comprise addition of the modified 30S ribosomal protein S1 according to claim 3; and
c. recovering nOMVs from the medium; and
d. mixing the nOMVs with a pharmaceutically acceptable diluent or carrier.

16. The process of claim 15, wherein the Gram-negative bacterial cell is a Gram-negative bacterial cell of any one of claims 1 to 12.

17. An nOMV obtained or obtainable from the genetically modified Gram-negative bacterial cell of any one of claims 1 to 12, or from a genetically modified Gram-negative bacterial cell obtained or obtainable by the method of claim 13, or by the process of claim 14 or 15.

18. A vaccine comprising the nOMV of claim 17.

19. The nOMV of claim 17, or the vaccine of claim 18 for use in medicine.
